SEQUENCE LISTING (374 sequences)

SEQ ID NO: 1 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 495 aa, synthetic construct
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 3 - protein, 495 aa, synthetic construct
KFTIVFPHNQKGNWKNVPSNYHYCPSSSDLNWHNDLIGTAIQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 5 - protein, 495 aa, synthetic construct
KFTIVFPHNQKGNWKNVPSNYHYCPSSSDLNWHNDLIGTALQVKMPKSHKAIQADGWMCHASKWITTCDFRWYGPKYITHSIQSFTPSVEQCKESIEQTKQGTWLNPGFPPQSCGYATVTDAEAVIVQVTPHHVLVDEYTGEWVDSQFINGKCSNDICLTVHNSTTWHSDYKVKGLCDSNLISMDITFFSEDGELSSLGKAGTGFRSNYFAYETGDKACKMQYCKHWGVRLPSGVWFEMADKDLFAAAKFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTNTERELWEDWAPYEDVEIGPNGVLRTSSGYKFPLYMIGHGMLDSDLHLSSKVQVFEHPHIQDAASQLPDDETLFFGDTGLSKNPIELVEGWFSGWKSSIASFFFIIGLIIGLFLVLRVGIYLCIKLKHTRKRKIYADIEMNRLGK

SEQ ID NO: 6 - protein, 495 aa, synthetic construct
KFTTVFPHNKKGDWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSDDICPTVHNSTTWHSDYKVKGLCDSNLISMDITFFSEDGELSSLGKEGTGFRSNYFAYETGDKACKMQYCKHWGVRLPSGVWFEMADKNLFAAAKFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWEDWAPYEDVEIGPNGVLRTSSGYKFPLYMIGHGMLDSDLHLSSKAQVFEHPHIPDATSQLPDDETLFFGDTGLSKDPIELVEGWFSGWKSSIASFFFIIGLIIGLFFVLRIGVYLCIKLKHTNKRQIYTDIEMNRLGK

SEQ ID NO: 7 - protein, 495 aa, synthetic construct
KFTIVFPHNQKGTWKNVPSNYHYCPSSSDLNWHNDLIGTALQVKMPKSHKAIQADGWMCHASKWVTTCDFRWYGPKYITHSIRSFTPSVEQCRESIEQTKQGTWLNPGFPPQSCGYATVTDAEAVIVQVTPHHVLVDEYTGEWVDSQFINGKCSNDICPTVHNSTTWHSDYKVKGLCDSNLISMDITFFSEDGELSSLGKEGTGFRSNHFAYETGDKACKMQYCKHWGVRLPSGVWFEMADQDLFAAARFPECPEGSSISAPSQTSVDVSLIQDVERILDYSLCQETWSKIGAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDETLFFGDTGLSKNPIELVEGWFSGWKSSIASFFFIIGLIIGLFLVLRVGIYLCIKLKHTKKRQIYTDIEMNRLGK

SEQ ID NO: 8 - protein, 495 aa, synthetic construct
KFTIVFPHNQKGNWKNVPSNYHYCPSSSDLNWHNDLIGTGLQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDRELSSLGKEGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSGLHLSSKAQVFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 9 - protein, 495 aa, synthetic construct
KFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 10 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 11 - protein, 118 aa, synthetic construct
QITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCARDEYGGFDIWGQGTMVTVSS

SEQ ID NO: 12 - protein, 7 aa, synthetic construct
TSGVGVG

SEQ ID NO: 13 - protein, 16 aa, synthetic construct
LIYWNDEKRYSPSLKS

SEQ ID NO: 14 - protein, 8 aa, synthetic construct
DEYGGFDI

SEQ ID NO: 15 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRVVYPITFGGGTKVEIK

SEQ ID NO: 16 - protein, 11 aa, synthetic construct
RASQSVSSYLA

SEQ ID NO: 17 - protein, 7 aa, synthetic construct
DASNRAT

SEQ ID NO: 18 - protein, 9 aa, synthetic construct
QQRVVYPIT

SEQ ID NO: 19 - protein, 245 aa, synthetic construct
QITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCARDEYGGFDIWGQGTMVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRVVYPITFGGGTKVEIK

SEQ ID NO: 20 - protein, 245 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRVVYPITFGGGTKVEIKGGGGSGGGGSGGGGSGGGGSQITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCARDEYGGFDIWGQGTMVTVSS

SEQ ID NO: 21 - protein, 118 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTAVYYCARDEYGGFDIWGQGTMVTVSS

SEQ ID NO: 22 - protein, 7 aa, synthetic construct
TSGVGVG

SEQ ID NO: 23 - protein, 16 aa, synthetic construct
LIYWNDDKRYSPSLKS

SEQ ID NO: 24 - protein, 8 aa, synthetic construct
DEYGGFDI

SEQ ID NO: 25 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRFDYPITFGGGTKVEIK

SEQ ID NO: 26 - protein, 11 aa, synthetic construct
RASQSVSSYLA

SEQ ID NO: 27 - protein, 7 aa, synthetic construct
DASNRAT

SEQ ID NO: 28 - protein, 9 aa, synthetic construct
QQRFDYPIT

SEQ ID NO: 29 - protein, 245 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTAVYYCARDEYGGFDIWGQGTMVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRFDYPITFGGGTKVEIK

SEQ ID NO: 30 - protein, 245 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRFDYPITFGGGTKVEIKGGGGSGGGGSGGGGSGGGGSQITLKESGPTLVKPTQTLTLTCTFSGFSLSTSGVGVGWIRQPPGKALEWLALIYWNDDKRYSPSLKSRLTITKDTSKNQVVLTMTNMDPVDTAVYYCARDEYGGFDIWGQGTMVTVSS

SEQ ID NO: 31 - protein, 117 aa, synthetic construct
QVQLVESGGGVVQPGRSLRLSCAASGFTFSSYGMHWVRQAPGKGLEWVAVISYEGSNKYYADSVKGRFTISRDNSKNTLYLQMNSLRAEDTAVYYCARELGDGMDVWGQGTTVTVSS

SEQ ID NO: 32 - protein, 5 aa, synthetic construct
SYGMH

SEQ ID NO: 33 - protein, 17 aa, synthetic construct
VISYEGSNKYYADSVKG

SEQ ID NO: 34 - protein, 8 aa, synthetic construct
ELGDGMDV

SEQ ID NO: 35 - protein, 106 aa, synthetic construct
EIVLTQSPATLSLSPGERATLSCRASQSVSSYLAWFQQKPGQAPRLLIYDASNRATGIPARFSGSGSGTDFTLTISSLEPEDFAVYYCQQRVDLWTFGGGTKVEIK

SEQ ID NO: 36 - protein, 11 aa, synthetic construct
RASQSVSSYLA

SEQ ID NO: 37 - protein, 7 aa, synthetic construct
DASNRAT

SEQ ID NO: 38 - protein, 8 aa, synthetic construct
QQRVDLWT

SEQ ID NO: 39 - protein, 243 aa, synthetic construct
QVQLVESGGGVVQPGRSLRLSCAASGFTFSSYGMHWVRQAPGKGLEWVAVISYEGSNKYYADSVKGRFTISRDNSKNTLYLQMNSLRAEDTAVYYCARELGDGMDVWGQGTTVTVSSGGGGSGGGGSGGGGSGGGGSEIVLTQSPATLSLSPGERATLSCRASQSVSSYLAWFQQKPGQAPRLLIYDASNRATGIPARFSGSGSGTDFTLTISSLEPEDFAVYYCQQRVDLWTFGGGTKVEIK

SEQ ID NO: 40 - protein, 243 aa, synthetic construct
EIVLTQSPATLSLSPGERATLSCRASQSVSSYLAWFQQKPGQAPRLLIYDASNRATGIPARFSGSGSGTDFTLTISSLEPEDFAVYYCQQRVDLWTFGGGTKVEIKGGGGSGGGGSGGGGSGGGGSQVQLVESGGGVVQPGRSLRLSCAASGFTFSSYGMHWVRQAPGKGLEWVAVISYEGSNKYYADSVKGRFTISRDNSKNTLYLQMNSLRAEDTAVYYCARELGDGMDVWGQGTTVTVSS

SEQ ID NO: 41 - protein, 118 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSS

SEQ ID NO: 42 - protein, 5 aa, synthetic construct
DYYMS

SEQ ID NO: 43 - protein, 17 aa, synthetic construct
YISSSGSTIYYADSVKG

SEQ ID NO: 44 - protein, 9 aa, synthetic construct
DQGNYGVDV

SEQ ID NO: 45 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQVSSLPPTFGGGTKVEIK

SEQ ID NO: 46 - protein, 11 aa, synthetic construct
RASQSISSWLA

SEQ ID NO: 47 - protein, 7 aa, synthetic construct
DASSLES

SEQ ID NO: 48 - protein, 9 aa, synthetic construct
QQVSSLPPT

SEQ ID NO: 49 - protein, 245 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSGGGGSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQVSSLPPTFGGGTKVEIK

SEQ ID NO: 50 - protein, 245 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQVSSLPPTFGGGTKVEIKGGGGSGGGGSGGGGSGGGGSEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSS

SEQ ID NO: 51 - protein, 118 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSS

SEQ ID NO: 52 - protein, 5 aa, synthetic construct
DYYMS

SEQ ID NO: 53 - protein, 17 aa, synthetic construct
YISSSGSTIYYADSVKG

SEQ ID NO: 54 - protein, 9 aa, synthetic construct
DQGNYGVDV

SEQ ID NO: 55 - protein, 107 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQSDSHPITFGGGTKVEIK

SEQ ID NO: 56 - protein, 11 aa, synthetic construct
RASQSISSWLA

SEQ ID NO: 57 - protein, 7 aa, synthetic construct
EASSLES

SEQ ID NO: 58 - protein, 9 aa, synthetic construct
QQSDSHPIT

SEQ ID NO: 59 - protein, 245 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQSDSHPITFGGGTKVEIK

SEQ ID NO: 60 - protein, 245 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQSDSHPITFGGGTKVEIKGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSS

SEQ ID NO: 61 - protein, 118 aa, synthetic construct; identical to SEQ ID NO: 41

SEQ ID NO: 62 - protein, 5 aa, synthetic construct
DYYMS

SEQ ID NO: 63 - protein, 17 aa, synthetic construct
YISSSGSTIYYADSVKG

SEQ ID NO: 64 - protein, 9 aa, synthetic construct
DQGNYGVDV

SEQ ID NO: 65 - protein, 107 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQANSHPITFGGGTKVEIK

SEQ ID NO: 66 - protein, 11 aa, synthetic construct
RASQSISSWLA

SEQ ID NO: 67 - protein, 7 aa, synthetic construct
EASSLES

SEQ ID NO: 68 - protein, 9 aa, synthetic construct
QQANSHPIT

SEQ ID NO: 69 - protein, 245 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQANSHPITFGGGTKVEIK

SEQ ID NO: 70 - protein, 245 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQANSHPITFGGGTKVEIKGGGGSGGGGSGGGGSGGGGSEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSS

SEQ ID NO: 71 - protein, 122 aa, synthetic construct
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCARPGDGYYEGVYFDYWGQGTLVTVSS

SEQ ID NO: 72 - protein, 5 aa, synthetic construct
NYAMS

SEQ ID NO: 73 - protein, 17 aa, synthetic construct
AISGSGGSTYYADSVKG

SEQ ID NO: 74 - protein, 13 aa, synthetic construct
PGDGYYEGVYFDY

SEQ ID NO: 75 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQAHSSPITFGGGTKVEIK

SEQ ID NO: 76 - protein, 11 aa, synthetic construct
RASQSISSYLN

SEQ ID NO: 77 - protein, 7 aa, synthetic construct
AASSLQS

SEQ ID NO: 78 - protein, 9 aa, synthetic construct
QQAHSSPIT

SEQ ID NO: 79 - protein, 249 aa, synthetic construct
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCARPGDGYYEGVYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQAHSSPITFGGGTKVEIK

SEQ ID NO: 80 - protein, 249 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQAHSSPITFGGGTKVEIKGGGGSGGGGSGGGGSGGGGSEVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCARPGDGYYEGVYFDYWGQGTLVTVSS

SEQ ID NO: 81 - protein, 124 aa, synthetic construct
QITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCAREGSHDYKSSNWFDPWGQGTLVTVSS

SEQ ID NO: 82 - protein, 7 aa, synthetic construct
TSGVGVG

SEQ ID NO: 83 - protein, 16 aa, synthetic construct
LIYWNDEKRYSPSLKS

SEQ ID NO: 84 - protein, 14 aa, synthetic construct
EGSHDYKSSNWFDP

SEQ ID NO: 85 - protein, 107 aa, synthetic construct
DIQMTQSPSSLSASVGDRVTITCQASQDIANYLNWYQQKPGKAPKLLIYDASNLETGVPSRFSGSGSGTDFTFTISSLQPEDIATYYCQQHFNLPLTFGGGTKVEIK

SEQ ID NO: 86 - protein, 11 aa, synthetic construct
QASQDIANYLN

SEQ ID NO: 87 - protein, 7 aa, synthetic construct
DASNLET

SEQ ID NO: 88 - protein, 10 aa, synthetic construct
QQHFNLPLTF

SEQ ID NO: 89 - protein, 251 aa, synthetic construct
QITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCAREGSHDYKSSNWFDPWGQGTLVTVSSGGGGSGGGGSGGGGSGGGGSDIQMTQSPSSLSASVGDRVTITCQASQDIANYLNWYQQKPGKAPKLLIYDASNLETGVPSRFSGSGSGTDFTFTISSLQPEDIATYYCQQHFNLPLTFGGGTKVEIK

SEQ ID NO: 90 - protein, 251 aa, synthetic construct
DIQMTQSPSSLSASVGDRVTITCQASQDIANYLNWYQQKPGKAPKLLIYDASNLETGVPSRFSGSGSGTDFTFTISSLQPEDIATYYCQQHFNLPLTFGGGTKVEIKGGGGSGGGGSGGGGSGGGGSQITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCAREGSHDYKSSNWFDPWGQGTLVTVSS

SEQ ID NO: 91 - protein, 124 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARAGDTYSAADYYYMDVWGKGTTVTVSS

SEQ ID NO: 92 - protein, 5 aa, synthetic construct
SYSMN

SEQ ID NO: 93 - protein, 17 aa, synthetic construct
SISSSSSYIYYADSVKG

SEQ ID NO: 94 - protein, 15 aa, synthetic construct
AGDTYSAADYYYMDV

SEQ ID NO: 95 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGLITFGGGTKVEIK

SEQ ID NO: 96 - protein, 16 aa, synthetic construct
RSSQSLLHSNGYNYLD

SEQ ID NO: 97 - protein, 7 aa, synthetic construct
LGSNRAS

SEQ ID NO: 98 - protein, 8 aa, synthetic construct
MQALGLIT

SEQ ID NO: 99 - protein, 255 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARAGDTYSAADYYYMDVWGKGTTVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGLITFGGGTKVEIK

SEQ ID NO: 100 - protein, 255 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGLITFGGGTKVEIKGGGGSGGGGSGGGGSGGGGSEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARAGDTYSAADYYYMDVWGKGTTVTVSS

SEQ ID NO: 101 - protein, 116 aa, synthetic construct
EVQLLESGGGLVQPGGSLRLSCAASGFTFGSEAMSWVRQAPGKERELVSAISGSGEVTYYADSVKGRFTISRDNSKNTLYLQMNSLRAEDTAVYYCQRLVEAKRHWGQGTQVTVSS

SEQ ID NO: 102 - protein, 5 aa, synthetic construct
SEAMS

SEQ ID NO: 103 - protein, 17 aa, synthetic construct
AISGSGEVTYYADSVKG

SEQ ID NO: 104 - protein, 7 aa, synthetic construct
LVEAKRH

SEQ ID NO: 105 - protein, 116 aa, synthetic construct
EVQLLESGGGLVQPGGSLRLSCAASGFTFESEAMSWYRQAPGKERELVSVITSEGSTYYADSVKGRFTISRDNSKNTLYLQMNSLRAEDTAVYYCAHIEWETRLNWGQGTQVTVSS

SEQ ID NO: 106 - protein, 5 aa, synthetic construct
SEAMS

SEQ ID NO: 107 - protein, 16 aa, synthetic construct
VITSEGSTYYADSVKG

SEQ ID NO: 108 - protein, 8 aa, synthetic construct
IEWETRLN

SEQ ID NO: 109 - protein, 118 aa, synthetic construct
EVQLLESGGGLVQPGGSLRLSCAASGFTFDEYTMHWFRQAPGKEREFVSAISGGGSETYYADSVKGRFTISRDNSKNTLYLQMNSLRAEDTAVYYCAAGGEEAGVGYWGQGTQVTVSS

SEQ ID NO: 110 - protein, 5 aa, synthetic construct
EYTMH

SEQ ID NO: 111 - protein, 17 aa, synthetic construct
AISGGGSETYYADSVKG

SEQ ID NO: 112 - protein, 9 aa, synthetic construct
GGEEAGVGY

SEQ ID NO: 113 - protein, 120 aa, synthetic construct
EVQLLESGGGLVQPGGSLRLSCAASGFTFEDYAMSWFRQAPGKEREGVSAISGKGGSTYYADSVKGRFTISRDNSKNTLYLQMNSLRAEDTAVYYCAVLDEEAGAEGGYWGQGTQVTVSS

SEQ ID NO: 114 - protein, 5 aa, synthetic construct
DYAMS

SEQ ID NO: 115 - protein, 17 aa, synthetic construct
AISGKGGSTYYADSVKG

SEQ ID NO: 116 - protein, 11 aa, synthetic construct
LDEEAGAEGGY

SEQ ID NO: 117 - protein, 120 aa, synthetic construct
EVQLLESGGGLVQPGGSLRLSCAASGFTFDRYAMSWFRQAPGKEREGVSAISTSGDSTYYADSVKGRFTISRDNSKNTLYLQMNSLRAEDTAVYYCAVLDEEAGAEGGYWGQGTQVTVSS

SEQ ID NO: 118 - protein, 5 aa, synthetic construct
RYAMS

SEQ ID NO: 119 - protein, 17 aa, synthetic construct
AISTSGDSTYYADSVKG

SEQ ID NO: 120 - protein, 11 aa, synthetic construct
LDEEAGAEGGY

SEQ ID NO: 121 - protein, 121 aa, synthetic construct
EVQLLESGGGLVQPGGSLRLSCAASGFTFASDAMSWYRQAPGKERELVSAISGSGGSTYYADSVKGRFTISRDNSKNTLYLQMNSLRAEDTAVYYCAAHDSGEAYLAFDYWGQGTQVTVSS

SEQ ID NO: 122 - protein, 5 aa, synthetic construct
SDAMS

SEQ ID NO: 123 - protein, 17 aa, synthetic construct
AISGSGGSTYYADSVKG

SEQ ID NO: 124 - protein, 12 aa, synthetic construct
HDSGEAYLAFDY

SEQ ID NO: 125 - protein, 121 aa, synthetic construct
EVQLLESGGGLVQPGGSLRLSCAASGFTFDSYTMSWYRQAPGKERELVSAISGHGDSTYYADSVKGRFTISRDNSKNTLYLQMNSLRAEDTAVYYCTRISITTEWLAGDYWGQGTQVTVSS

SEQ ID NO: 126 - protein, 5 aa, synthetic construct
SYTMS

SEQ ID NO: 127 - protein, 17 aa, synthetic construct
AISGHGDSTYYADSVKG

SEQ ID NO: 128 - protein, 12 aa, synthetic construct
ISITTEWLAGDY

SEQ ID NO: 129 - protein, 123 aa, synthetic construct
EVQLLESGGGLVQPGGSLRLSCAASGFTFSSYAMSWFRQAPGKEREFVSFISGSGDSTYYADSVKGRFTISRDNSKNTLYLQMNSLRAEDTAVYYCTRWPYDFEEPSEPGVYWGQGTQVTVSS

SEQ ID NO: 130 - protein, 5 aa, synthetic construct
SYAMS

SEQ ID NO: 131 - protein, 17 aa, synthetic construct
FISGSGDSTYYADSVKG

SEQ ID NO: 132 - protein, 14 aa, synthetic construct
WPYDFEEPSEPGVY

SEQ ID NO: 133 - protein, 123 aa, synthetic construct
EVQLLESGGGLVQPGGSLRLSCAASGFTFTDYDMSWYRQAPGKERELVSVIHSGGSTYYADSVKGRFTISRDNSKNTLYLQMNSLRAEDTAVYYCAPGYYSDLSFDYYNFDYWGQGTQVTVSS

SEQ ID NO: 134 - protein, 5 aa, synthetic construct
DYDMS

SEQ ID NO: 135 - protein, 16 aa, synthetic construct
VIHSGGSTYYADSVKG

SEQ ID NO: 136 - protein, 15 aa, synthetic construct
GYYSDLSFDYYNFDY

SEQ ID NO: 137 - protein, 123 aa, synthetic construct
EVQLLESGGGLVQPGGSLRLSCAASGFTFSDYAMHWFRQAPGKERVLVSSIDSGGSTYYADSVKGRFTISRDNSKNTLYLQMNSLRAEDTAVYYCNAGFKGDHPHPKDAFDIWGQGTQVTVSS

SEQ ID NO: 138 - protein, 5 aa, synthetic construct
DYAMH

SEQ ID NO: 139 - protein, 16 aa, synthetic construct
SIDSGGSTYYADSVKG

SEQ ID NO: 140 - protein, 15 aa, synthetic construct
GFKGDHPHPKDAFDI

SEQ ID NO: 141 - protein, 125 aa, synthetic construct
EVQLLESGGGLVQPGGSLRLSCAASGFTFSSEGMSWVRQAPGKERELVSAISGSGDHTYYADSVRGRFTISRDNSKNTLYLQMNSLRAEDTAVYYCNALEGGPTTAIQPGGPDYWGQGTQVTVSS

SEQ ID NO: 142 - protein, 5 aa, synthetic construct
SEGMS

SEQ ID NO: 143 - protein, 17 aa, synthetic construct
AISGSGDHTYYADSVRG

SEQ ID NO: 144 - protein, 16 aa, synthetic construct
LEGGPTTAIQPGGPDY

SEQ ID NO: 145 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 146 - protein, 5 aa, synthetic construct
RYTMH

SEQ ID NO: 147 - protein, 17 aa, synthetic construct
YINPSRGYTNYADSVKG

SEQ ID NO: 148 - protein, 10 aa, synthetic construct
YYDDHYCLDY

SEQ ID NO: 149 - protein, 106 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 150 - protein, 10 aa, synthetic construct
RASQSVSYMN

SEQ ID NO: 151 - protein, 7 aa, synthetic construct
DTSKVAS

SEQ ID NO: 152 - protein, 9 aa, synthetic construct
QQWSSNPLT

SEQ ID NO: 153 - protein, 241 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 154 - protein, 241 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 155 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDPYCLDYWGQGTTVTVSS

SEQ ID NO: 156 - protein, 5 aa, synthetic construct
RYTMH

SEQ ID NO: 157 - protein, 17 aa, synthetic construct
YINPSRGYTNYADSVKG

SEQ ID NO: 158 - protein, 10 aa, synthetic construct
YYDDPYCLDY

SEQ ID NO: 159 - protein, 106 aa, synthetic construct; identical to SEQ ID NO: 149

SEQ ID NO: 160 - protein, 10 aa, synthetic construct
RASQSVSYMN

SEQ ID NO: 161 - protein, 7 aa, synthetic construct
DTSKVAS

SEQ ID NO: 162 - protein, 9 aa, synthetic construct
QQWSSNPLT

SEQ ID NO: 163 - protein, 241 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDPYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 164 - protein, 241 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDPYCLDYWGQGTTVTVSS

SEQ ID NO: 165 - protein, 120 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYNDQYCLDYWGQGTTVTVSSS

SEQ ID NO: 166 - protein, 5 aa, synthetic construct
RYTMH

SEQ ID NO: 167 - protein, 17 aa, synthetic construct
YINPSRGYTNYADSVKG

SEQ ID NO: 168 - protein, 10 aa, synthetic construct
YYNDQYCLDY

SEQ ID NO: 169 - protein, 106 aa, synthetic construct; identical to SEQ ID NO: 149

SEQ ID NO: 170 - protein, 10 aa, synthetic construct
RASQSVSYMN

SEQ ID NO: 171 - protein, 7 aa, synthetic construct
DTSKVAS

SEQ ID NO: 172 - protein, 9 aa, synthetic construct
QQWSSNPLT

SEQ ID NO: 173 - protein, 242 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYNDQYCLDYWGQGTTVTVSSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 174 - protein, 242 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYNDQYCLDYWGQGTTVTVSSS

SEQ ID NO: 175 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDAHNCLDYWGQGTTVTVSS

SEQ ID NO: 176 - protein, 5 aa, synthetic construct
RYTMH

SEQ ID NO: 177 - protein, 17 aa, synthetic construct
YINPSRGYTNYADSVKG

SEQ ID NO: 178 - protein, 10 aa, synthetic construct
YYDAHNCLDY

SEQ ID NO: 179 - protein, 106 aa, synthetic construct; identical to SEQ ID NO: 149

SEQ ID NO: 180 - protein, 10 aa, synthetic construct
RASQSVSYMN

SEQ ID NO: 181 - protein, 7 aa, synthetic construct
DTSKVAS

SEQ ID NO: 182 - protein, 9 aa, synthetic construct
QQWSSNPLT

SEQ ID NO: 183 - protein, 241 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDAHNCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 184 - protein, 241 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDAHNCLDYWGQGTTVTVSS

SEQ ID NO: 185 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDHYCLDYWGQGTTVTVSS

SEQ ID NO: 186 - protein, 5 aa, synthetic construct
RYTMH

SEQ ID NO: 187 - protein, 17 aa, synthetic construct
YINPSRGYTNYADSVKG

SEQ ID NO: 188 - protein, 10 aa, synthetic construct
YSDDHYCLDY

SEQ ID NO: 189 - protein, 106 aa, synthetic construct; identical to SEQ ID NO: 149

SEQ ID NO: 190 - protein, 10 aa, synthetic construct
RASQSVSYMN

SEQ ID NO: 191 - protein, 7 aa, synthetic construct
DTSKVAS

SEQ ID NO: 192 - protein, 9 aa, synthetic construct
QQWSSNPLT

SEQ ID NO: 193 - protein, 241 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDHYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 194 - protein, 241 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDHYCLDYWGQGTTVTVSS

SEQ ID NO: 195 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDRYCLDYWGQGTTVTVSS

SEQ ID NO: 196 - protein, 5 aa, synthetic construct
RYTMH

SEQ ID NO: 197 - protein, 17 aa, synthetic construct
YINPSRGYTNYADSVKG

SEQ ID NO: 198 - protein, 10 aa, synthetic construct
YSDDRYCLDY

SEQ ID NO: 199 - protein, 106 aa, synthetic construct; identical to SEQ ID NO: 149

SEQ ID NO: 200 - protein, 10 aa, synthetic construct
RASQSVSYMN

SEQ ID NO: 201 - protein, 7 aa, synthetic construct
DTSKVAS

SEQ ID NO: 202 - protein, 9 aa, synthetic construct
QQWSSNPLT

SEQ ID NO: 203 - protein, 241 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDRYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 204 - protein, 241 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDRYCLDYWGQGTTVTVSS

SEQ ID NO: 205 - protein, 120 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDNYCLDYWGQGTTVTVSSS

SEQ ID NO: 206 - protein, 5 aa, synthetic construct
RYTMH

SEQ ID NO: 207 - protein, 17 aa, synthetic construct
YINPSRGYTNYADSVKG

SEQ ID NO: 208 - protein, 10 aa, synthetic construct
YYDDNYCLDY

SEQ ID NO: 209 - protein, 106 aa, synthetic construct; identical to SEQ ID NO: 149

SEQ ID NO: 210 - protein, 10 aa, synthetic construct
RASQSVSYMN

SEQ ID NO: 211 - protein, 7 aa, synthetic construct
DTSKVAS

SEQ ID NO: 212 - protein, 9 aa, synthetic construct
QQWSSNPLT

SEQ ID NO: 213 - protein, 242 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDNYCLDYWGQGTTVTVSSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 214 - protein, 242 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDNYCLDYWGQGTTVTVSSS

SEQ ID NO: 215 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDQYCLDYWGQGTTVTVSS

SEQ ID NO: 216 - protein, 5 aa, synthetic construct
RYTMH

SEQ ID NO: 217 - protein, 17 aa, synthetic construct
YINPSRGYTNYADSVKG

SEQ ID NO: 218 - protein, 10 aa, synthetic construct
YYDDQYCLDY

SEQ ID NO: 219 - protein, 106 aa, synthetic construct; identical to SEQ ID NO: 149

SEQ ID NO: 220 - protein, 10 aa, synthetic construct
RASQSVSYMN

SEQ ID NO: 221 - protein, 7 aa, synthetic construct
DTSKVAS

SEQ ID NO: 222 - protein, 9 aa, synthetic construct
QQWSSNPLT

SEQ ID NO: 223 - protein, 241 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDQYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 224 - protein, 241 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDQYCLDYWGQGTTVTVSS

SEQ ID NO: 225 - protein, 45 aa, synthetic construct
TTTPAPRPPTPAPTIASQPLSLRPEACRPAAGGAVHTRGLDFACD

SEQ ID NO: 226 - protein, 39 aa, synthetic construct
IEVMYPPPYLDNEKSNGTIIHVKGKHLCPSPLFPGPSKP

SEQ ID NO: 227 - protein, 22 aa, synthetic construct
SGQVLLESNIKVLPTWSTPVQP

SEQ ID NO: 228 - protein, 12 aa, synthetic construct
ESKYGPPCPPCP

SEQ ID NO: 229 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 230 - protein, 16 aa, synthetic construct
LEPKSCDKTHTCPPCP

SEQ ID NO: 231 - protein, 24 aa, synthetic construct
IYIWAPLAGTCGVLLLSLVITLYC

SEQ ID NO: 232 - protein, 27 aa, synthetic construct
IISFFLALTSTALLFLLFFLTLRFSVV

SEQ ID NO: 233 - protein, 27 aa, synthetic construct
FWVLVVVGGVLACYSLLVTVAFIIFWV

SEQ ID NO: 234 - protein, 21 aa, synthetic construct
VAAILGLGLVLGLLGPLAILL

SEQ ID NO: 235 - protein, 23 aa, synthetic construct
WLPIGCAAFVVVCILGCILICWL

SEQ ID NO: 236 - protein, 26 aa, synthetic construct
VMSVATIVIVDICITGGLLLLVYYWS

SEQ ID NO: 237 - protein, 22 aa, synthetic construct
MALIVLGGVAGLLLFIGLGIFF

SEQ ID NO: 238 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 239 - protein, 42 aa, synthetic construct
KRGRKKLLYIFKQPFMRPVQTTQEEDGCSCRFPEEEEGGCEL

SEQ ID NO: 240 - protein, 41 aa, synthetic construct
RSKRSRLLHSDYMNMTPRRPGPTRKHYQPYAPPRDFAAYRS

SEQ ID NO: 241 - protein, 42 aa, synthetic construct
ALYLLRRDQRLPPDAHKPPGGGSFRTPIQEEQADAHSTLAKI

SEQ ID NO: 242 - protein, 35 aa, synthetic construct
TKKKYSSSVHDPNGEYMFMRAVNTAKKSRLTDVTL

SEQ ID NO: 243 - protein, 48 aa, synthetic construct
QRRKYRSNKGESPVEPAEPCHYSCPREEEGSTIPIQEDYRKPEPACSP

SEQ ID NO: 244 - protein, 174 aa, synthetic construct
KKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 245 - protein, 21 aa, synthetic construct
MALPVTALLLPLALLLHAARP

SEQ ID NO: 246 - protein, 20 aa, synthetic construct
METDTLLLWVLLLWVPGSTG

SEQ ID NO: 247 - protein, 22 aa, synthetic construct
MDMRVPAQLLGLLLLWLRGARC

SEQ ID NO: 248 - protein, 16 aa, synthetic construct
MPLLLLLPLLWAGALA

SEQ ID NO: 249 - protein, 23 aa, synthetic construct
MDAMKRGLCCVLLLCGAVFVSPS

SEQ ID NO: 250 - protein, 17 aa, synthetic construct
MLLLLLLLGLRLQLSLG

SEQ ID NO: 251 - protein, 17 aa, synthetic construct
MWLQSLLLLGTVACSIS

SEQ ID NO: 252 - protein, 22 aa, synthetic construct
MLLLVTSLLLCELPHPAFLLIP

SEQ ID NO: 253 - protein, 20 aa, synthetic construct
MSRSVALAVLALLSLSGLEA

SEQ ID NO: 254 - protein, 16 aa, synthetic construct
MLLLLLLLLLLALALA

SEQ ID NO: 255 - protein, 468 aa, synthetic construct
QITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCARDEYGGFDIWGQGTMVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRVVYPITFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 256 - protein, 468 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRVVYPITFGGGTKVEIKGGGGSGGGGSGGGGSGGGGSQITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCARDEYGGFDIWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 257 - protein, 468 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTAVYYCARDEYGGFDIWGQGTMVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRFDYPITFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 258 - protein, 468 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRFDYPITFGGGTKVEIKGGGGSGGGGSGGGGSGGGGSQITLKESGPTLVKPTQTLTLTCTFSGFSLSTSGVGVGWIRQPPGKALEWLALIYWNDDKRYSPSLKSRLTITKDTSKNQVVLTMTNMDPVDTAVYYCARDEYGGFDIWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 259 - protein, 466 aa, synthetic construct
QVQLVESGGGVVQPGRSLRLSCAASGFTFSSYGMHWVRQAPGKGLEWVAVISYEGSNKYYADSVKGRFTISRDNSKNTLYLQMNSLRAEDTAVYYCARELGDGMDVWGQGTTVTVSSGGGGSGGGGSGGGGSGGGGSEIVLTQSPATLSLSPGERATLSCRASQSVSSYLAWFQQKPGQAPRLLIYDASNRATGIPARFSGSGSGTDFTLTISSLEPEDFAVYYCQQRVDLWTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 260 - protein, 466 aa, synthetic construct
EIVLTQSPATLSLSPGERATLSCRASQSVSSYLAWFQQKPGQAPRLLIYDASNRATGIPARFSGSGSGTDFTLTISSLEPEDFAVYYCQQRVDLWTFGGGTKVEIKGGGGSGGGGSGGGGSGGGGSQVQLVESGGGVVQPGRSLRLSCAASGFTFSSYGMHWVRQAPGKGLEWVAVISYEGSNKYYADSVKGRFTISRDNSKNTLYLQMNSLRAEDTAVYYCARELGDGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 261 - protein, 468 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSGGGGSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQVSSLPPTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 262 - protein, 468 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQVSSLPPTFGGGTKVEIKGGGGSGGGGSGGGGSGGGGSEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 263 - protein, 468 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQSDSHPITFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 264 - protein, 468 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQSDSHPITFGGGTKVEIKGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 265 - protein, 468 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQANSHPITFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 266 - protein, 468 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQANSHPITFGGGTKVEIKGGGGSGGGGSGGGGSGGGGSEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDQGNYGVDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 267 - protein, 472 aa, synthetic construct
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCARPGDGYYEGVYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQAHSSPITFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 268 - protein, 472 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQAHSSPITFGGGTKVEIKGGGGSGGGGSGGGGSGGGGSEVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCARPGDGYYEGVYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 269 - protein, 474 aa, synthetic construct
QITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCAREGSHDYKSSNWFDPWGQGTLVTVSSGGGGSGGGGSGGGGSGGGGSDIQMTQSPSSLSASVGDRVTITCQASQDIANYLNWYQQKPGKAPKLLIYDASNLETGVPSRFSGSGSGTDFTFTISSLQPEDIATYYCQQHFNLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 270 - protein, 474 aa, synthetic construct
DIQMTQSPSSLSASVGDRVTITCQASQDIANYLNWYQQKPGKAPKLLIYDASNLETGVPSRFSGSGSGTDFTFTISSLQPEDIATYYCQQHFNLPLTFGGGTKVEIKGGGGSGGGGSGGGGSGGGGSQITLKESGPTLVKPTQTLTLTCTFSGFSLSTSGVGVGWIRQPPGKALEWLALIYWNDEKRYSPSLKSRLTITKDTSKNQVVLTMTNMDPVDTAVYYCAREGSHDYKSSNWFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 271 - protein, 478 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARAGDTYSAADYYYMDVWGKGTTVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGLITFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 272 - protein, 478 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGLITFGGGTKVEIKGGGGSGGGGSGGGGSGGGGSEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARAGDTYSAADYYYMDVWGKGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 273 - protein, 339 aa, synthetic construct
EVQLLESGGGLVQPGGSLRLSCAASGFTFGSEAMSWVRQAPGKERELVSAISGSGEVTYYADSVKGRFTISRDNSKNTLYLQMNSLRAEDTAVYYCQRLVEAKRHWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 274 - protein, 339 aa, synthetic construct
EVQLLESGGGLVQPGGSLRLSCAASGFTFESEAMSWYRQAPGKERELVSVITSEGSTYYADSVKGRFTISRDNSKNTLYLQMNSLRAEDTAVYYCAHIEWETRLNWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 275 - protein, 341 aa, synthetic construct
EVQLLESGGGLVQPGGSLRLSCAASGFTFDEYTMHWFRQAPGKEREFVSAISGGGSETYYADSVKGRFTISRDNSKNTLYLQMNSLRAEDTAVYYCAAGGEEAGVGYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 276 - protein, 343 aa, synthetic construct
EVQLLESGGGLVQPGGSLRLSCAASGFTFEDYAMSWFRQAPGKEREGVSAISGKGGSTYYADSVKGRFTISRDNSKNTLYLQMNSLRAEDTAVYYCAVLDEEAGAEGGYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 277 - protein, 343 aa, synthetic construct
EVQLLESGGGLVQPGGSLRLSCAASGFTFDRYAMSWFRQAPGKEREGVSAISTSGDSTYYADSVKGRFTISRDNSKNTLYLQMNSLRAEDTAVYYCAVLDEEAGAEGGYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 278 - protein, 344 aa, synthetic construct
EVQLLESGGGLVQPGGSLRLSCAASGFTFASDAMSWYRQAPGKERELVSAISGSGGSTYYADSVKGRFTISRDNSKNTLYLQMNSLRAEDTAVYYCAAHDSGEAYLAFDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 279 - protein, 344 aa, synthetic construct
EVQLLESGGGLVQPGGSLRLSCAASGFTFDSYTMSWYRQAPGKERELVSAISGHGDSTYYADSVKGRFTISRDNSKNTLYLQMNSLRAEDTAVYYCTRISITTEWLAGDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 280 - protein, 346 aa, synthetic construct
EVQLLESGGGLVQPGGSLRLSCAASGFTFSSYAMSWFRQAPGKEREFVSFISGSGDSTYYADSVKGRFTISRDNSKNTLYLQMNSLRAEDTAVYYCTRWPYDFEEPSEPGVYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 281 - protein, 346 aa, synthetic construct
EVQLLESGGGLVQPGGSLRLSCAASGFTFTDYDMSWYRQAPGKERELVSVIHSGGSTYYADSVKGRFTISRDNSKNTLYLQMNSLRAEDTAVYYCAPGYYSDLSFDYYNFDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 282 - protein, 346 aa, synthetic construct
EVQLLESGGGLVQPGGSLRLSCAASGFTFSDYAMHWFRQAPGKERVLVSSIDSGGSTYYADSVKGRFTISRDNSKNTLYLQMNSLRAEDTAVYYCNAGFKGDHPHPKDAFDIWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 283 - protein, 348 aa, synthetic construct
EVQLLESGGGLVQPGGSLRLSCAASGFTFSSEGMSWVRQAPGKERELVSAISGSGDHTYYADSVRGRFTISRDNSKNTLYLQMNSLRAEDTAVYYCNALEGGPTTAIQPGGPDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 284 - DNA, 135 bp, synthetic construct
  source 1..135: mol_type = other DNA
accacaacacctgctccaaggccccccacacccgctccaactatagccagccaaccattgagcctcagacctgaagcttgcaggcccgcagcaggaggcgccgtccatacgcgaggcctggacttcgcgtgtgat

SEQ ID NO: 285 - DNA, 117 bp, synthetic construct
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 286 - DNA, 36 bp, synthetic construct
  source 1..36: mol_type = other DNA
gagtccaaatatggtcccccgtgcccaccatgccca

SEQ ID NO: 287 - DNA, 48 bp, synthetic construct
  source 1..48: mol_type = other DNA
cttgagcccaaatcttgtgacaaaactcacacatgcccaccgtgccca

SEQ ID NO: 288 - DNA, 72 bp, synthetic construct
  source 1..72: mol_type = other DNA
atttatatttgggcacctttggccggaacatgtggggtgttgcttctctcccttgtgatcactctgtattgt

SEQ ID NO: 289 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 290 - DNA, 126 bp, synthetic construct
  source 1..126: mol_type = other DNA
aagcgcgggagaaagaagctcctgtacatcttcaagcagccttttatgcgacctgtgcaaaccactcaggaagaagatgggtgttcatgccgcttccccgaggaggaagaaggagggtgtgaactg

SEQ ID NO: 291 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 292 - DNA, 105 bp, synthetic construct
  source 1..105: mol_type = other DNA
acaaaaaagaagtattcatccagtgtgcacgaccctaacggtgaatacatgttcatgagagcagtgaacacagccaaaaaatctagactcacagatgtgacccta

SEQ ID NO: 293 - DNA, 336 bp, synthetic construct
  source 1..336: mol_type = other DNA
agggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 294 - DNA, 63 bp, synthetic construct
  source 1..63: mol_type = other DNA
atggctcttcccgtaacagcccttttgttgccccttgcactccttctgcatgcagcacgaccg

SEQ ID NO: 295 - DNA, 1404 bp, synthetic construct
  source 1..1404: mol_type = other DNA
gagatcgtgctgacacagtctcccgccacactgtcactgtctccaggcgaaagagccacactgagctgtagagccagccagagcgtgtcctcttacctggcctggtatcagcagaagcctggacaggctccccggctgctgatctacgatgccagcaatagagccacaggcatccccgccagattttctggcagcggctctggcaccgatttcaccctgaccataagcagcctggaacctgaggacttcgccgtgtactactgccagcagagagtggtgtaccccatcacctttggcggaggcaccaaggtggaaatcaaaggcggcggaggaagcggaggcggaggatctggtggtggtggatctggcggaggtggcagccagatcacactgaaagagtctggccccacactggtcaagcccacacagaccctgacactgacctgcaccttcagcggctttagcctgagcacatctggcgtcggcgttggctggattagacagcctcctggaaaggccctggaatggctggccctgatctactggaacgacgagaagagatacagccccagcctgaagtcccggctgaccatcaccaaggacaccagcaagaaccaggtggtgctgaccatgacaaacatggaccccgtggacaccgccgtgtattattgcgccagagatgagtacggcggcttcgacatttggggccagggcacaatggtcaccgtgtctagtaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 296 - DNA, 1404 bp, synthetic construct
  source 1..1404: mol_type = other DNA
gagatcgtgctgacccagtcccctgctaccctgagcctgtctccaggcgagcgggccacactgagctgtagagcttctcagagcgtgtccagctacctggcctggtatcagcagaaacctggccaggcccctagactgctgatctacgacgccagcaaccgggccaccggcatccccgccagattcagcggatctggcagcggcacagattttaccctcaccatcagcagcctggaacctgaggacttcgccgtctactactgccagcaaagattcgactaccccatcaccttcggcggcggaacaaaggtggaaattaagggtggtgggggcagcggtggaggtgggagcggaggcgggggtagcggaggcgggggtagccaaatcacactgaaagagagcggccctacactcgtgaaacctacccagaccctgacactgacatgtaccttcagcggcttctccctgagcacctctggcgtcggcgttggatggatcagacagcctccaggcaaggccctggaatggctggctctgatctattggaacgacgacaagcggtacagccccagcctgaagtctagactgaccatcacaaaggacaccagcaagaaccaggtggtgctgaccatgacaaatatggaccccgtggacaccgccgtgtactactgcgccagagatgagtacggcggatttgatatctggggccagggcaccatggtgaccgtgtccagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 297 - DNA, 1398 bp, synthetic construct
  source 1..1398: mol_type = other DNA
caagtgcagctcgtggaaagcggcggcggagtggtgcagcccggccggagcctgagactgtcctgcgccgcttctggatttaccttcagcagctacggcatgcactgggtcagacaggcccctggcaaaggcctggagtgggtggccgttatcagctacgagggcagcaacaagtattacgccgacagcgtgaagggccgcttcacaatctctagagataatagcaagaacaccctgtacctgcagatgaacagcctgcgggccgaagataccgccgtgtactactgtgctagagagctgggcgacggcatggacgtgtggggacagggcacaaccgtgaccgtgtcctctggtggtgggggcagcggtggaggtgggagcggaggcgggggtagcggaggcgggggtagcgagatcgtgctgacccagtcccctgctacactgagcctgtctccaggcgagcgggccacactgagctgtagagcttctcagagcgtgtccagctatctggcctggttccagcagaaacctggccaggcccctagactgctgatctacgacgccagcaaccgggccaccggcatccccgccagattcagcggctctggcagcggcaccgacttcaccctcaccatcagcagcctggaacccgaggattttgccgtctactactgccagcaaagagtggacctgtggaccttcggcggaggaacaaaggtggaaatcaagaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 298 - DNA, 1404 bp, synthetic construct
  source 1..1404: mol_type = other DNA
gacatccagatgacccagagcccttcgaccctatccgcttccgtgggtgaccgtgtgaccatcacctgtcgcgcgtcgcagagcatctcctcctggctcgcgtggtaccaacagaagcctggcaaggcccccaagctgctgatttacgacgccagttccctggagtctggcgtgccatcccgcttctccggcagcggcagcggtaccgagttcaccctgacgatcagctccctgcagccggatgactttgctacctactactgtcagcaggtctcctccctcccccccaccttcggtggcggtaccaaggtggagatcaagggcggcggcggctctggtggcggaggttctggcgggggaggttcgggggggggaggctccgaggtgcaactggtagagagcggcgggggactggtaaaacccggcggctccctgcggctgtcatgcgctgctagcggcttcacgttcagcgattactacatgagttggatccgccaggcccccgggaagggtttggagtgggtctcgtatatctcttccagcggatctaccatttactatgcggacagcgtgaaggggcgcttcaccatatctcgggacaacgccaagaactccctgtacctgcagatgaattccctgcgtgccgaggacacggccgtgtattactgtgcccgcgaccagggcaactacggcgtcgacgtgtggggccagggtacaaccgtcaccgtgtccagtaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 299 - DNA, 1404 bp, synthetic construct
  source 1..1404: mol_type = other DNA
caagtgcagctggtcgagagcggaggaggcctggttaagcccggcggatctctcagactgagctgcgccgctagcggctttacattcagcgactactacatgagctggatccggcaggcccctggcaagggcctggaatgggtgtcctacatcagctcctccggcagcaccatctactacgccgacagcgtgaaaggcagattcacaatctctagagataatgccaagaacagcctgtacctgcagatgaacagcctgcgggccgaggacaccgccgtgtactattgtgctagagatcagggcaactacggcgtggacgtgtggggccagggcaccaccgtgaccgtgtctagcggtggtgggggcagcggtggaggtgggagcggaggcgggggtagcggaggcgggggtagcgatatccagatgacccagtccccatctacactgagcgcctctgtgggcgaccgggtgaccattacatgtagagccagccagagcatcagcagctggctggcttggtatcagcagaaacctggcaaggcccctaagctgctgatctacgaggccagcagcctggaaagcggcgtccccagcagattcagcggcagcggctctggaacagagttcaccctgaccatctcctccctgcagcctgacgacttcgccacctactactgccagcaatctgatagccaccccatcacctttggcggaggcaccaaggtggaaatcaagaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 300 - DNA, 1404 bp, synthetic construct
  source 1..1404: mol_type = other DNA
gatatccagatgacccagtccccatctacactgagcgcctctgtgggcgaccgggtgacaattacctgtagagctagccagagcatctcctcctggctggcttggtaccagcaaaaacctggcaaggcccctaagctgctgatctacgaggccagcagcctggaaagcggcgtcccctctagattcagcggcagcggctctggaaccgagttcaccctgacaatcagcagcctgcagcctgacgacttcgccacctattactgccagcaggccaacagccaccccatcacctttggcggaggcaccaaggtggaaatcaagggtggtgggggcagcggtggaggtgggagcggaggcgggggtagcggaggcgggggtagcgaggtgcagctggtggaaagcggcggaggactcgttaagcccggcggcagcctgagactgagctgcgccgctagcggatttaccttcagcgactactacatgagctggatccggcaggcccctggcaagggcctggaatgggtcagctacatcagctcctctggctctacaatctactacgccgacagcgtgaaaggcagattcaccatctctagagataatgccaagaacagcctgtacctgcaaatgaacagcctgcgggccgaggacaccgccgtgtactattgtgctagagatcagggcaactacggcgtggacgtgtggggccagggcaccaccgtgacagtgtcctccaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 301 - DNA, 1416 bp, synthetic construct
  source 1..1416: mol_type = other DNA
gacatccagatgacccagagccctagctccctgagcgccagcgtgggcgatagagtgaccattacctgtagagcctctcagagcatctcctcctacctgaactggtatcagcagaaacccggcaaggcccctaagctgctgatctacgccgctagcagcctgcagtctggcgtccccagccggttcagcggcagcggatctggcaccgacttcaccctgacaatcagcagcctgcaacctgaggactttgctacatactactgccagcaggcccacagctctccaatcaccttcggcggcggaacaaaggtggaaatcaagggtggtgggggcagcggtggaggtgggagcggaggcgggggtagcggaggcgggggtagcgaggtgcagctgctggaaagcggaggcggactcgttcaacctggcggcagcctgagactgagctgcgccgcttctggatttaccttcagcaactacgccatgagctgggtgcggcaggcccctggcaaaggcctggaatgggtctccgccatcagcggctctggcggctccacctactacgccgacagcgtgaagggcagattcaccatctctagagataatagcaagaacaccctgtacctgcagatgaacagcctgcgggccgaggacaccgccgtgtactattgtgctagacccggagatggctactacgagggcgtgtacttcgactactggggccagggcacactggtgacagtgtccagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 302 - DNA, 1422 bp, synthetic construct
  source 1..1422: mol_type = other DNA
gatattcagatgacccagagcccatctagcctgagcgccagcgtgggcgatagagtgaccatcacctgtcaggcctctcaggacatcgctaattacctgaactggtatcagcagaaacccggcaaggcccctaagctgctgatctacgacgcctccaacctggaaaccggcgtgcccagccggttcagcggcagcggatctggcacagacttcacctttaccatcagctccctccagcctgaggacatcgccacatactactgccagcaacacttcaacctgcctctgaccttcggcggcggaacaaaggtcgagatcaagggtggtgggggcagcggtggaggtgggagcggaggcgggggtagcggaggcgggggtagccaaatcaccctgaaagagagcggacctacactggtcaagcctacccagacactgaccctcacatgtacattcagcggctttagcctgagcacctccggcgtgggagtgggctggatcagacagccccccggcaaggccctggaatggctggctctgatctattggaatgacgagaagcggtacagccctagcctgaaatctagactgacaatcaccaaggacaccagcaagaaccaggtggtgctgaccatgaccaacatggatcctgtggataccgccgtgtactactgcgccagagaaggctctcacgactacaagagctccaactggttcgacccatggggccagggcaccctggttacagtgtctagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 303 - DNA, 1434 bp, synthetic construct
  source 1..1434: mol_type = other DNA
gatatcgtgatgacccaatctccactgagcctgcctgtgacacctggcgagcctgcttctatcagctgtagaagcagccagtccctgctgcacagcaacggctacaactacctggactggtatctgcagaaacccggccagagcccccagctgctgatctacctcggctctaatcgggccagcggagtgcctgatagattcagcggaagcggctccggcaccgacttcaccctgaagatcagcagagtggaagccgaggacgtgggcgtctactactgcatgcaggccctgggcctgattacatttggcggcggaaccaaggtggaaatcaagggtggtgggggcagcggtggaggtgggagcggaggcgggggtagcggaggcgggggtagcgaagtgcagctggttgagagcggcggcggactggtgaagcccggaggcagcctcagactgagctgtgctgcttctggctttaccttcagctcttatagcatgaactgggtgcggcaggcccctggcaagggcctggaatgggtcagctccatcagctcttctagcagctacatctactacgccgacagcgtgaagggcagattcaccatcagcagagataacgccaagaacagcctgtacctgcagatgaatagcctgcgggccgaggacaccgccgtgtactactgcgccagagccggcgacacctacagcgccgccgattactactacatggacgtgtggggcaaaggaacaaccgtgacagtgtcctccaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 304 - DNA, 1017 bp, synthetic construct
  source 1..1017: mol_type = other DNA
gaagtgcaactgctggaaagcggcggaggcctggtccagcccggcggctctctgcggctcagctgcgccgcttctggatttaccttcggcagcgaggctatgagctgggtgcggcaggcccctggaaaagagagagagctggtgtccgccatcagcggcagcggcgaggtgacctactacgccgacagcgtgaagggcagattcaccatctctagagataatagcaagaacaccctgtacctgcagatgaacagcctgagagccgaggacaccgccgtgtactattgtcagagactggtggaagccaagcggcactggggccagggcacacaggttacagtgtccagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 305 - DNA, 1017 bp, synthetic construct
  source 1..1017: mol_type = other DNA
gaagtgcaactgctggaatctggcggaggactggtgcagcccggcggcagcctgcggctgagctgtgctgcttctggctttaccttcgagtctgaggccatgagctggtatagacaggcccctggcaaggaaagagagctggtcagcgtgatcaccagcgagggctccacctactacgccgacagcgtgaaaggcagattcacaatcagccgggacaatagcaagaacaccctgtacctgcagatgaacagcctgcgcgccgaagatacagccgtgtactactgcgcccacatcgagtgggagacaagactcaactggggccagggcacccaggtgaccgtgtccagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 306 - DNA, 1023 bp, synthetic construct
  source 1..1023: mol_type = other DNA
gaggtgcagctgctggaaagcggagggggcctggtccaacccggcgggtctcttcgcctaagctgtgccgcttctggcttcaccttcgacgagtacaccatgcactggttcagacaggcccccggcaaggagcgcgagttcgtcagtgcaatcagcggaggcggtagcgagacttattacgcggactccgtgaagggccgcttcaccattagccgcgacaactccaagaacacgctgtacctgcagatgaattcgctgcgcgccgaagatacggccgtgtactactgtgccgctggtggggaggaggctggcgtgggctattggggccagggcacccaggtcaccgtgtcgtccaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 307 - DNA, 1029 bp, synthetic construct
  source 1..1029: mol_type = other DNA
gaggtgcagctgctggagagcggaggcggcctcgtgcagccaggaggttccctacgactctcctgtgccgccagcggcttcaccttcgaggactacgccatgagttggttccgccaggccccggggaaggagcgcgagggcgtgagcgcgatttctggaaagggcggctccacctattacgcggactccgtgaagggtcgctttaccatctctcgcgacaactccaagaacacgctgtacctgcagatgaatagcctgcgcgctgaggacactgccgtgtactactgtgctgtcttggacgaggaggccggcgcagagggcggctattggggccagggtacccaggtcaccgtgtcgtccaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 308 - DNA, 1029 bp, synthetic construct
  source 1..1029: mol_type = other DNA
gaggtgcaactgctggaaagcggcggtggactggtgcagcccggcggcagcctgagactgtcttgtgctgcttctggatttacattcgacagatacgccatgagctggttccgccaggcccctggcaaagagcgggaaggcgtgtccgccatctccacaagcggagatagcacatactatgccgacagcgtgaagggcagattcaccatcagcagagataatagcaagaacaccctgtacctgcagatgaacagcctccgggccgaggacaccgccgtctactactgcgccgtgctggacgaggaagccggcgccgagggcggctactggggccagggcacccaggtgaccgtgtctagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 309 - DNA, 1032 bp, synthetic construct
  source 1..1032: mol_type = other DNA
gaggtgcaactgctggaaagcggcggaggactcgtccagcccggcggcagcctgcggctgagctgtgctgcttctggatttaccttcgccagcgacgccatgagctggtatagacaggcccctggcaaagagcgggaactggtgtccgccatcagcggctctggcggctccacctactacgccgatagcgtgaagggcagattcacaatctctagagataatagcaagaacaccctgtacctgcagatgaacagcctgagagccgaggacaccgccgtgtactactgcgccgctcacgacagcggcgaggcctacctggccttcgactactggggccagggcacacaggtgaccgtgtctagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 310 - DNA, 1032 bp, synthetic construct
  source 1..1032: mol_type = other DNA
gaggtgcaactgctggaaagcggcggaggactcgtccagcccggcggcagcctgaggctgagctgtgctgcttctggctttaccttcgactcctacacaatgagctggtatagacaggcccctggcaaggagcgggaactggtgtccgccatcagcggccacggcgactctacatactacgccgacagcgtgaaaggcagattcacaatctctagagataatagcaagaacaccctgtacctgcagatgaacagcctgcgggccgaggacaccgccgtgtactactgcaccagaatcagcatcaccaccgagtggctggccggagattactggggccagggcacccaggtgacagtgtccagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 311 - DNA, 1038 bp, synthetic construct
  source 1..1038: mol_type = other DNA
gaggtgcagctgctggaaagcggaggaggcctggtccaacctggcggcagcctgcggctgagctgcgccgcttctggcttcaccttcagcagctacgccatgagctggttccggcaggcccctggcaaggaaagagagttcgtgtcttttatcagcggatctggcgactccacctactacgctgatagcgtgaaaggcagatttaccatctctagagataatagcaagaacaccctgtacctccagatgaacagcctgcgcgccgaggacacagccgtgtactattgtaccagatggccttacgacttcgaggaaccaagcgagcccggcgtgtactggggccagggcacacaggtgacagtgtcctccaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 312 - DNA, 1038 bp, synthetic construct
  source 1..1038: mol_type = other DNA
gaggtgcagctgctggaaagcggcggaggcctggtgcaacctggcggatctctcagactgagctgtgctgcttctggcttcacattcaccgactacgacatgagctggtatagacaggcccctggaaaagagcgggaactggtctccgtgatccacagcggcggctccacctactacgccgatagcgtgaagggcagattcaccatcagcagagataatagcaagaacaccctgtacctgcagatgaacagcctgcgggccgaggacaccgccgtgtactactgcgcccccggctactacagcgacctgtcttttgattattacaacttcgactactggggccagggcacacaggtgacagtgtccagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 313 - DNA, 1038 bp, synthetic construct
  source 1..1038: mol_type = other DNA
gaggtgcagctgctggagagcggtggagggttggtgcagcccgggggtagcctgcgtctgtcgtgcgccgcttccggcttcacgttctctgattacgccatgcactggttccggcaggcccccggtaaggagcgcgtgctggtgtcgtctattgactccggcggctccacttactacgcagacagtgtcaagggccgtttcaccatcagccgcgacaacagcaagaacacgctgtacctgcagatgaactcccttcgagcagaggacaccgcggtgtactactgtaatgcgggcttcaagggcgatcacccccaccccaaggatgccttcgacatttggggccagggcacccaggtcaccgtgtcgtccaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 314 - DNA, 1044 bp, synthetic construct
  source 1..1044: mol_type = other DNA
gaggtgcaactgctggaatccggcggaggcctggtgcagcccggcggcagcctcagactgagctgtgccgcttctggctttaccttcagcagcgagggcatgagctgggtgcggcaggcccctggcaaggaaagagagctggtctccgccatcagcggatctggcgaccacacctactatgccgatagcgtgcgcggaagattcacaatctctagagataatagcaagaacaccctgtacctgcagatgaacagcctgcgggccgaggacaccgccgtgtactactgcaacgccctggaaggcggccctacaacagctatccagccaggaggccctgactactggggccagggcacccaggtgaccgtgtccagcaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcacctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaaaagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 315 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 316 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaggtctgctgagactcggggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 317 - DNA, 389 bp, synthetic construct
  source 1..389: mol_type = other DNA
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 318 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 319 - DNA, 1184 bp, synthetic construct
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 320 - DNA, 388 bp, synthetic construct
  source 1..388: mol_type = other DNA
aatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggc

SEQ ID NO: 321 - DNA, 500 bp, synthetic construct
  source 1..500: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 322 - DNA, 443 bp, synthetic construct
  source 1..443: mol_type = other DNA
tgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccc

SEQ ID NO: 323 - DNA, 1212 bp, synthetic construct
  source 1..1212: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 324 - protein, 310 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 325 - protein, 310 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDPYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 326 - protein, 311 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYNDQYCLDYWGQGTTVTVSSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 327 - protein, 310 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDAHNCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 328 - protein, 310 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDHYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 329 - protein, 310 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYSDDRYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 330 - protein, 311 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDNYCLDYWGQGTTVTVSSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 331 - protein, 310 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDQYCLDYWGQGTTVTVSSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKTTTPAPRPPTPAPTIASQPLSLRPEACRPAAGGAVHTRGLDFACDIYIWAPLAGTCGVLLLSLVITLYC

SEQ ID NO: 332 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 333 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 334 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 335 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 336 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 337 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 338 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 339 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 340 - protein, 472 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 341 - protein, 467 aa, synthetic construct
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 342 - protein, 5 aa, synthetic construct
TGEKP

SEQ ID NO: 343 - protein, 5 aa, synthetic construct
GGGGS

SEQ ID NO: 344 - protein, 10 aa, synthetic construct
GGGGSGGGGS

SEQ ID NO: 345 - protein, 15 aa, synthetic construct
GGGGSGGGGSGGGGS

SEQ ID NO: 346 - protein, 20 aa, synthetic construct
GGGGSGGGGSGGGGSGGGGS

SEQ ID NO: 347 - protein, 25 aa, synthetic construct
GGGGSGGGGSGGGGSGGGGSGGGGS

SEQ ID NO: 348 - protein, 14 aa, synthetic construct
EGKSSGSGSESKVD

SEQ ID NO: 349 - protein, 18 aa, synthetic construct
KESGSVSSEQLAQFRSLD

SEQ ID NO: 350 - protein, 9 aa, synthetic construct
LRQRDGERP

SEQ ID NO: 351 - protein, 12 aa, synthetic construct
LRQKDGGGSERP

SEQ ID NO: 352 - protein, 16 aa, synthetic construct
LRQKDGGGSGGGSERP

SEQ ID NO: 353 - protein, 18 aa, synthetic construct
GEGTSTGSGGSGGSGGAD

SEQ ID NO: 354 - protein, 18 aa, synthetic construct
GSTSGSGKPGSGEGSTKG

SEQ ID NO: 355 - protein, 22 aa, synthetic construct
GSGATNFSLLKQAGDVEENPGP

SEQ ID NO: 356 - protein, 19 aa, synthetic construct
ATNFSLLKQAGDVEENPGP

SEQ ID NO: 357 - protein, 14 aa, synthetic construct
LLKQAGDVEENPGP

SEQ ID NO: 358 - protein, 21 aa, synthetic construct
GSGEGRGSLLTCGDVEENPGP

SEQ ID NO: 359 - protein, 18 aa, synthetic construct
EGRGSLLTCGDVEENPGP

SEQ ID NO: 360 - protein, 13 aa, synthetic construct
LLTCGDVEENPGP

SEQ ID NO: 361 - protein, 23 aa, synthetic construct
GSGQCTNYALLKLAGDVESNPGP

SEQ ID NO: 362 - protein, 20 aa, synthetic construct
QCTNYALLKLAGDVESNPGP

SEQ ID NO: 363 - protein, 14 aa, synthetic construct
LLKLAGDVESNPGP

SEQ ID NO: 364 - protein, 25 aa, synthetic construct
GSGVKQTLNFDLLKLAGDVESNPGP

SEQ ID NO: 365 - protein, 22 aa, synthetic construct
VKQTLNFDLLKLAGDVESNPGP

SEQ ID NO: 366 - protein, 19 aa, synthetic construct
LLNFDLLKLAGDVESNPGP

SEQ ID NO: 367 - protein, 19 aa, synthetic construct
TLNFDLLKLAGDVESNPGP

SEQ ID NO: 368 - protein, 17 aa, synthetic construct
NFDLLKLAGDVESNPGP

SEQ ID NO: 369 - protein, 20 aa, synthetic construct
QLLNFDLLKLAGDVESNPGP

SEQ ID NO: 370 - protein, 24 aa, synthetic construct
APVKQTLNFDLLKLAGDVESNPGP

SEQ ID NO: 371 - protein, 40 aa, synthetic construct
VTELLYRMKRAETYCPRPLLAIHPTEARHKQKIVAPVKQT

SEQ ID NO: 372 - protein, 18 aa, synthetic construct
LNFDLLKLAGDVESNPGP

SEQ ID NO: 373 - protein, 40 aa, synthetic construct
LLAIHPTEARHKQKIVAPVKQTLNFDLLKLAGDVESNPGP

SEQ ID NO: 374 - protein, 33 aa, synthetic construct
EARHKQKIVAPVKQTLNFDLLKLAGDVESNPGP

(19) United States
(12) Patent Application Publication
(10) Pub. No.: US 2025/0099615 A1
(43) Pub. Date: Mar. 27, 2025

(54) COMPOSITIONS FOR TREATING CANCER

(71) Applicant: Kelonia Therapeutics, Inc., Boston, MA (US)

(72) Inventors: Kevin M. Friedman, Boston, MA (US); Molly R. Perkins, Milton, MA (US); Connor S. Dobson, Washington, DC (US); Stephen L. Sazinsky, Winchester, MA (US); Shannon G. Contrastano, Auburndale, MA (US); Emily Thompson Beura, Mansfield, MA (US); Cory Ahonen, Lebanon, NH (US); Andrew Avery, Lebanon, NH (US)

(21) Appl. No.: 18/919,103

(22) Filed: Oct. 17, 2024

Related U.S. Application Data
(63) Continuation of application No. PCT/US2024/048301, filed on Sep. 25, 2024.
(60) Provisional application No. 63/618,878, filed on Jan. 8, 2024.
(60) Provisional application No. 63/540,336, filed on Sep. 25, 2023.

(51) Int. Cl. A61K 48/00; A61K 39/00; C07K 16/28; C12N 15/86

(52) U.S. Cl. CPC A61K 48/005; A61K 40/11; A61K 40/15; A61K 40/31; A61K 40/4215; C07K 16/2809; C07K 16/2878; C12N 15/86; A61K 2239/13; C07K 2317/622; C12N 2740/15022; C12N 2740/15023; C12N 2740/15043; C12N 2830/48; C12N 2830/50

(57) ABSTRACT

The present disclosure provides compositions and methods comprising recombinant particles suitable for specifically delivering one or more chimeric antigen receptors to immune effector cells in vivo.

### CROSS REFERENCE TO RELATED APPLICATIONS

This application is a continuation application of International Application No. PCT/US2024/048301, filed Sep. 25, 2024, which claims the benefit of and priority to U.S. Provisional Application No. 63/540,336, filed Sep. 25, 2023, and U.S. Provisional Application No. 63/618,878, filed Jan. 8, 2024. The entire teachings of the applications are incorporated herein by reference.

### STATEMENT REGARDING SEQUENCE LISTING

The Sequence Listing associated with this application is provided in XML format in lieu of a paper copy and is hereby incorporated by reference into the specification. The name of the XML file containing the Sequence Listing is KELO-012-102X_ST26.xml. The XML file is 385 KB, was created on Sep. 20, 2024, and is being submitted electronically via Patent Center, concurrent with the filing of the specification.

### TECHNICAL FIELD

The present disclosure relates to recombinant particles engineered to deliver a chimeric antigen receptor to a cell. More particularly, the disclosure relates to recombinant particles engineered to deliver a chimeric antigen receptor to cells in vivo.

### DESCRIPTION OF THE RELATED ART

B cell maturation antigen (BCMA) is a member of the tumor necrosis factor receptor superfamily and is also known as tumor necrosis factor receptor superfamily member 17 (TNFRSF17). BCMA is normally expressed in mature B lymphocytes and plasma cells. BCMA expression is also detected in various lymphomas and multiple myelomas. Multiple myeloma is an incurable plasma cell malignancy that originates in the bone marrow.

Multiple myeloma is the second most prevalent hematological malignancy after non-lymphoma. In 2020, an estimated 176,404 people world-wide were diagnosed with multiple myeloma and about 117,077 patients succumbed to the disease. In 2023, an estimated 35,730 people in the United States alone will be diagnosed with multiple myeloma and an estimated 12,590 multiple myeloma patients will pass from the disease or associated complications. The 5-year relative survival rate for multiple myeloma in the United States is only about 58%

Multiple myeloma may initially be treated with an autologous stem cell transplantation (ASCT) and/or various drug combinations (e.g., proteasome inhibitors including bortezomib, carfilzomib, ixazomib; immunomodulatory drugs (IMiDs) including pomalidomide, lenalidomide, thalidomide; and corticosteroids like dexamethasone) but patients eventually relapse with the disease becoming refractory to treatment. Subsequent lines of treatment include monoclonal antibodies, bispecific antibodies, e.g., BiTEs, antibody-drug conjugates, and finally chimeric antigen receptor T cell therapy.

Ex vivo gene therapies are potentially one-time therapeutic modalities that generally involve harvesting cells from a subject, modifying the cells by culturing them with a gene therapy vector, and delivering the modified cells back to the subject. Because ex vivo gene therapies are manufactured in a controlled environment, they do not generally require specialized targeting moieties and instead, targeting moieties with a broad tropism and that are highly efficient in delivering a gene therapy to most cell types are used.

In contrast, in vivo gene therapies are manufactured in the patient, in an uncontrolled environment. Accordingly, in vivo delivery of gene therapy vectors to specific cell types is orders of magnitude more complex than ex vivo delivery. In vivo gene therapy vectors encounter many non-target or off-target cells and may require a narrower or more specific tropism to deliver therapeutic payloads to a particular cell type. The potential of in vivo gene therapies has yet to be realized primarily due to inefficient delivery to desired cell types in combination with substantial off-target delivery. Use of specialized targeting moieties to deliver gene therapies in vivo has proven difficult in abrogating the off-target delivery to undesired cell types. In addition, on-target delivery of in vivo gene therapies using such specialized targeting moieties is often inefficient.

### BRIEF SUMMARY

The present disclosure generally relates, in part, to a recombinant particle comprising a mutated vesiculovirus envelope glycoprotein, a tropism polypeptide that binds to an immune effector cell, and a lentiviral vector encoding or comprising a promoter operably linked to a polynucleotide encoding a chimeric antigen receptor that binds B cell maturation antigen (BCMA).

In various embodiments, the disclosure contemplates, in part, a recombinant lentiviral particle comprising: (a) a viral envelope comprising (i) a mutated cocal virus envelope glycoprotein (COCV-G) or a mutated vesicular stomatitis Indiana virus envelope glycoprotein (VSIV-G), wherein the mutated COCV-G or VSIV-G comprises amino acid substitutions at positions 47 and 354; and (ii) a non-viral membrane-bound tropism polypeptide comprising an anti-CD3ε scFv and a human CD8α hinge and transmembrane domain; and (b) a recombinant lentiviral vector comprising a polynucleotide encoding a myeloproliferative sarcoma virus enhancer, negative control region deleted, dl587rev primer-binding site substituted (MND) U3 promoter or an EF1α promoter operably linked to a polynucleotide encoding an anti-BCMA chimeric antigen receptor comprising an anti-BCMA scFv or anti-BCMA VHH, a CD8α hinge and transmembrane domain, a CD137 costimulatory domain, a CD3ζ primary signaling domain.

In particular embodiments, the mutated COCV-G or the mutated VSIV-G comprises amino acid substitutions selected from the group consisting of: K47A and R354A; K47A and R354Q; K47Q and R354A; and K47Q and R354Q.

In some embodiments, the mutated COCV-G or the mutated VSIV-G comprises the amino acid substitutions K47A and R354A.

In certain embodiments, the mutated COCV-G or the mutated VSIV-G comprises the amino acid substitutions K47A and R354Q.

In particular embodiments, the mutated COCV-G or the mutated VSIV-G comprises the amino acid substitutions K47Q and R354A.

In some embodiments, the mutated COCV-G or the mutated VSIV-G comprises the amino acid substitutions K47Q and R354Q.

In additional embodiments, the mutated COCV-G or the mutated VSIV-G comprises the amino acid sequence set forth in any one of SEQ ID NOs: 332, 333, 334, 335, 336, 337, 338, and 339.

In particular embodiments, the mutated VSIV-G comprises the amino acid sequence set forth in any one of SEQ ID NOs: 332, 333, 334, and 335.

In further embodiments, the mutated COCV-G comprises the amino acid sequence set forth in any one of SEQ ID NOs: 336, 337, 338, and 339.

In certain embodiments, the anti-CD3ε scFv is isolated from an antibody selected from the group consisting of: OKT3, UCHT1, YTH12.5, TR66, and variants thereof.

In particular embodiments, the anti-CD3ε scFv is isolated from OKT3.

In additional embodiments, the anti-CD3p scFv is isolated from UCHT1.

In some embodiments, the anti-CD3p scFv is isolated from YTH12.5.

In further embodiments, the anti-CD3ε scFv is isolated from TR66.

In certain embodiments, the anti-CD3ε scFv comprises an amino acid sequence set forth in any one of SEQ ID NOs: 153, 154, 163, 164, 173, 174, 183, 184, 193, 194, 203, 204, 213, 214, 223, and 224.

In particular embodiments, the non-viral membrane-bound tropism polypeptide comprises an amino acid sequence set forth in any one of SEQ ID NOs: 324, 325, 326, 327, 328, 329, 330, and 331.

In some embodiments, the MND U3 promoter comprises the polynucleotide sequence set forth in SEQ ID NO: 320.

In certain embodiments, the EF1α promoter comprises the polynucleotide sequence set forth in SEQ ID NO: 319.

In particular embodiments, the anti-BCMA CAR comprises an anti-BCMA scFv comprising an amino acid sequence selected from the group consisting of: 19, 20, 29, 30, 39, 40, 49, 50, 59, 60, 69, 70, 79, 80, 89, 90, 99, and 100.

In additional embodiments, the anti-BCMA CAR comprises an anti-BCMA scFv comprising an amino acid sequence selected from the group consisting of: 20, 30, 39, 50, 59, 70, 80, 90, and 100.

In particular embodiments, the anti-BCMA CAR comprises an anti-BCMA scFv comprising an amino acid sequence selected from the group consisting of: 39, 59, 70, and 90.

In particular embodiments, the anti-BCMA CAR comprises an anti-BCMA VHH comprising an amino acid sequence selected from the group consisting of: 101, 105, 109, 113, 117, 121, 125, 129, 133, 137, and 141.

In some embodiments, the anti-BCMA CAR comprises an anti-BCMA VHH comprising an amino acid sequence selected from the group consisting of: 101 and 117.

In further embodiments, the anti-BCMA CAR comprises the amino acid sequence set forth in any one of SEQ ID NOs: 259, 263, 266, 270, 273 and 277, preferably SEQ ID NO: 266.

In certain embodiments, the polynucleotide encoding the anti-BCMA CAR comprises the polynucleotide sequence set forth in any one of SEQ ID NOs: 297, 299, 300, 302, 304, and 308.

In particular embodiments, the polynucleotide encoding the anti-BCMA CAR further comprises a polynucleotide sequence encoding a signal peptide.

In additional embodiments, the polynucleotide encoding the anti-BCMA CAR further comprises a polynucleotide sequence encoding a signal peptide isolated from a polypeptide selected from the group consisting of: CD8α, mIgGY, hIgGk, CD33, tPA, SEAP, hGM-CSF, CSF2R, and B2M.

In certain embodiments, the polynucleotide encoding the anti-BCMA CAR further comprises a polynucleotide sequence encoding a signal peptide comprising an amino acid sequence set forth in any one of SEQ ID NOs: 245, 246, 247, 248, 249, 250, 251, 252, 253, and 254.

In particular embodiments, the polynucleotide encoding the signal peptide comprises the polynucleotide sequence set forth in SEQ ID NO: 294.

In further embodiments, the lentiviral vector further comprises a WPRE operably linked to the 3′ end of the polynucleotide encoding the anti-BCMA CAR.

In some embodiments, the lentiviral vector further comprises a WPRE that comprises, consists essentially of, or consists of a polynucleotide sequence set forth in any one of SEQ ID NOs: 315, 316, and 317.

In various embodiments, the disclosure contemplates, in part, a recombinant lentiviral particle comprising: (a) a viral envelope comprising (i) a mutated viral envelope glycoprotein comprising an amino acid sequence set forth in any one of SEQ ID NOs: 332, 333, 334, and 335 and (ii) a non-viral membrane-bound tropism polypeptide comprising an amino acid sequence set forth in any one of SEQ ID NOs: 324, 325, 326, 327, 328, 329, 330, and 331; and (b) a recombinant lentiviral vector comprising a 5′ long terminal repeat (LTR) comprising R and U5 regions; a Psi (Ψ) packaging signal, a cPPT/FLAP, a rev response element (RRE); a polynucleotide encoding an MND promoter or an EF1α promoter operably linked to a polynucleotide encoding an anti-BCMA chimeric antigen receptor (CAR) comprising an amino acid sequence set forth in any one of SEQ ID NOs: 259, 263, 266, 270, 273, and 277 or an amino acid sequence at least 95%, at least 96%, at least 97%, at least 98%, at least 99% identical thereto; optionally a WPRE; a 3′ LTR comprising U3 and R regions; a polyadenylation signal and a poly(A) tail.

In particular embodiments, the mutated viral envelope glycoprotein comprises an amino acid sequence set forth in SEQ ID NO: 332.

In some embodiments, the mutated viral envelope glycoprotein comprises an amino acid sequence set forth in SEQ ID NO: 333.

In certain embodiments, the mutated viral envelope glycoprotein comprises an amino acid sequence set forth in SEQ ID NO: 334.

In additional embodiments, the mutated viral envelope glycoprotein comprises an amino acid sequence set forth in SEQ ID NO: 335.

In various embodiments, the disclosure contemplates, in part, a recombinant lentiviral particle comprising: (a) a viral envelope comprising (i) a mutated viral envelope glycoprotein comprising an amino acid sequence set forth in any one of SEQ ID NOs: 336, 337, 338, and 339 and (ii) a non-viral membrane-bound tropism polypeptide comprising an amino acid sequence set forth in any one of SEQ ID NOs: 324, 325, 326, 327, 328, 329, 330, and 331; and (b) a recombinant lentiviral vector comprising a 5′ long terminal repeat (LTR) comprising R and U5 regions; a Psi (Ψ) packaging signal, a cPPT/FLAP, a rev response element (RRE); a polynucleotide encoding an MND promoter or an EF1α promoter operably linked to a polynucleotide encoding an anti-BCMA chimeric antigen receptor (CAR) comprising an amino acid sequence set forth in any one of SEQ ID NOs: 259, 263, 266, 270, 273, and 277 or an amino acid sequence at least 95%, at least 96%, at least 97%, at least 98%, at least 99% identical thereto; optionally a WPRE; a 3′ LTR comprising U3 and R regions; a polyadenylation signal and a poly(A) tail.

In some embodiments, the mutated viral envelope glycoprotein comprises an amino acid sequence set forth in SEQ ID NO: 336.

In further embodiments, the mutated viral envelope glycoprotein comprises an amino acid sequence set forth in SEQ ID NO: 337.

In certain embodiments, the mutated viral envelope glycoprotein comprises an amino acid sequence set forth in SEQ ID NO: 338.

In some embodiments, the mutated viral envelope glycoprotein comprises an amino acid sequence set forth in SEQ ID NO: 339.

In particular embodiments, the non-viral membrane-bound tropism polypeptide comprises an amino acid sequence set forth in SEQ ID NO: 324.

In additional embodiments, the non-viral membrane-bound tropism polypeptide comprises an amino acid sequence set forth in SEQ ID NO: 325.

In certain embodiments, the non-viral membrane-bound tropism polypeptide comprises an amino acid sequence set forth in SEQ ID NO: 326.

In particular embodiments, the non-viral membrane-bound tropism polypeptide comprises an amino acid sequence set forth in SEQ ID NO: 327.

In further embodiments, the non-viral membrane-bound tropism polypeptide comprises an amino acid sequence set forth in SEQ ID NO: 328.

In some embodiments, the non-viral membrane-bound tropism polypeptide comprises an amino acid sequence set forth in SEQ ID NO: 329.

In additional embodiments, the non-viral membrane-bound tropism polypeptide comprises an amino acid sequence set forth in SEQ ID NO: 330.

In particular embodiments, the non-viral membrane-bound tropism polypeptide comprises an amino acid sequence set forth in SEQ ID NO: 331.

In particular embodiments, the recombinant lentiviral vector is derived from HIV-1 or HIV-2.

In additional embodiments, the MND U3 promoter comprises the polynucleotide sequence set forth in SEQ ID NO: 320.

In some embodiments, the EF1α promoter comprises the polynucleotide sequence set forth in SEQ ID NO: 319.

In particular embodiments, the polynucleotide encoding the anti-BCMA CAR comprises the polynucleotide sequence set forth in any one of SEQ ID NOs: 297, 299, 300, 302, 304, and 308.

In some embodiments, the polynucleotide encoding the anti-BCMA CAR further comprises a polynucleotide sequence encoding a signal peptide.

In further embodiments, the polynucleotide encoding the anti-BCMA CAR further comprises a polynucleotide sequence encoding a signal peptide isolated from a polypeptide selected from the group consisting of: CD8α, mIgGx, hIgGk, CD33, tPA, SEAP, hGM-CSF, CSF2R, and B2M.

In particular embodiments, the polynucleotide encoding the anti-BCMA CAR further comprises a polynucleotide sequence encoding a signal peptide comprising an amino acid sequence set forth in any one of SEQ ID NOs: 245, 246, 247, 248, 249, 250, 251, 252, 253, and 254.

In certain embodiments, the lentiviral vector further comprises a WPRE operably linked to the 3′ end of the polynucleotide encoding the anti-BCMA CAR.

In some embodiments, the lentiviral vector further comprises a WPRE operably linked to the 3′ end of the polynucleotide encoding the anti-BCMA CAR, wherein the WPRE comprises, consists essentially of, or consists of the polynucleotide sequence set forth in any one of SEQ ID NOs: 315, 316, and 317.

In various embodiments, the disclosure contemplates, in part, a cell transduced with a particle contemplated herein.

In further embodiments, the cell is an immune effector cell.

In additional embodiments, the cell is a T cell or a natural killer T (NKT) cell.

In various embodiments, the disclosure contemplates, in part, a composition comprising a particle or a cell contemplated herein.

In various embodiments, the disclosure contemplates, in part, a pharmaceutical composition comprising a pharmaceutically acceptable carrier and a particle, a cell, or a composition contemplated herein.

In various embodiments, the disclosure contemplates, in part, a method of treating, preventing, or ameliorating at least one symptom of a disease, disorder or condition associated therewith in a subject, comprising administering to the subject an effective amount of a particle, a cell, a composition, or a pharmaceutical composition contemplated herein.

In some embodiments, the disease, disorder, or condition is a cancer.

In certain embodiments, the cancer is a multiple myeloma (MM).

In particular embodiments, the cancer is MM selected from the group consisting of: active multiple myeloma, smoldering multiple myeloma, light chain myeloma, non-secretory myeloma, IgD myeloma, IgE myeloma, osteosclerotic myeloma, solitary plasmacytoma of bone, and extramedullary plasmacytoma.

In further embodiments, the cancer is relapsed and/or refractory.

In various embodiments, the disclosure contemplates, in part, a method of treating a subject that has, or has been diagnosed with, a multiple myeloma, comprising administering the subject an effective amount of a particle, a cell, a composition, or a pharmaceutical composition contemplated herein.

In some embodiments, the administration is parenteral administration.

In particular embodiments, the administration is intravenous.

In various embodiments, the disclosure contemplates, in part, a method of transducing an immune effector cell in vivo, comprising administering to a subject a pharmaceutical composition comprising a pharmaceutically acceptable carrier and an effective amount of a particle, a cell, a composition, or a pharmaceutical composition contemplated herein.

In various embodiments, the disclosure contemplates, in part, a method of making a recombinant lentivirus comprising (a) transfecting a host cell with four polynucleotides: a first polynucleotide that encodes lentiviral gag-pol, a second polynucleotide that encodes lentiviral rev, a third polynucleotide that encodes a mutated viral envelope glycoprotein and a non-viral membrane-bound tropism polypeptide contemplated herein, and a fourth polynucleotide that is a transfer plasmid encoding the recombinant lentiviral vector contemplated herein; and b) culturing the transduced cell for about 1 to 3 days to produce the recombinant lentivirus.

In various embodiments, the disclosure contemplates, in part, a kit comprising the particle contemplated herein, a pharmaceutically acceptable carrier, and instructions for use.

### BRIEF DESCRIPTION OF SEVERAL VIEWS OF THE DRAWINGS

The patent or application file contains at least one drawing executed in color. Copies of this patent or patent application publication with color drawings will be provided by the Office upon request and payment of the necessary fee.

FIG. 1 is a cartoon of a recombinant lentiviral particle comprising a viral envelope expressing a mutated viral envelope glycoprotein and non-viral membrane-bound tropism polypeptide and two copies of a lentiviral vector encoding a promoter operably linked to a polynucleotide encoding an anti-BCMA CAR and an optional post-transcriptional response element (PRE) operably linked to the 3′ prime end of the polynucleotide encoding the anti-BCMA CAR.

FIG. 2A shows Jurkat cell titer of recombinant lentiviruses comprising a viral envelope expressing a mutated vesicular stomatitis Indiana virus envelope glycoprotein G (VSIV-G) and a non-viral membrane-bound tropism polypeptide that binds CD3 and a lentiviral vector encoding an MNDU3 promoter operably linked to a polynucleotide encoding a CD8α signal peptide and an anti-BCMA CAR and a WPRE operably linked to the 3′ prime end of the polynucleotide encoding the anti-BCMA CAR (18 anti-BCMA CARs were evaluated).

FIG. 2B shows anti-BCMA CAR expression on PBMCs transduced with recombinant lentiviral particles comprising a viral envelope expressing a mutated VSIV-G and a non-viral membrane-bound tropism polypeptide that binds CD3 and a lentiviral vector encoding an MNDU3 promoter operably linked to a polynucleotide encoding a CD8α signal peptide and an anti-BCMA CAR and a WPRE operably linked to the 3′ prime end of the polynucleotide encoding the anti-BCMA CAR (18 anti-BCMA CARs were evaluated).

FIG. 2C shows the vector copy number (VCN) in PBMCs transduced with recombinant lentiviral particles comprising a viral envelope expressing a mutated VSIV-G and a non-viral membrane-bound tropism polypeptide that binds CD3 and a lentiviral vector encoding an MNDU3 promoter operably linked to a polynucleotide encoding a CD8α signal peptide and an anti-BCMA CAR and a WPRE operably linked to the 3′ prime end of the polynucleotide encoding the anti-BCMA CAR (18 anti-BCMA CARs were evaluated).

FIG. 2D shows the anti-BCMA CAR activity measured as the amount of IFNγ produced in a co-culture assay. PBMCs transduced with recombinant lentiviral particles comprising a viral envelope expressing a mutated VSIV-G and a non-viral membrane-bound tropism polypeptide that binds CD3 and a lentiviral vector encoding an MNDU3 promoter operably linked to a polynucleotide encoding a CD8α signal peptide and an anti-BCMA CAR and a WPRE operably linked to the 3′ prime end of the polynucleotide encoding the anti-BCMA CAR (18 anti-BCMA CARs were evaluated) were co-cultured with Daudi cells (low BCMA expression) or RPMI-8226 cells (high BCMA expression) for 24 hours. After 24 hours, IFNγ levels in co-culture supernatant were measured and plotted as a function of % anti-BCMA CAR positive cells in the co-culture.

FIG. 3A shows Jurkat cell functional titer of recombinant lentivirus comprising a viral envelope expressing a mutated VSIV-G and a non-viral membrane-bound tropism polypeptide that binds CD3 and a lentiviral vector encoding either an MNDU3 promoter, an SFFV promoter, or an EF1α promoter operably linked to a polynucleotide encoding a CD8α signal peptide and an anti-BCMA CAR (6 anti-BCMA CARs were evaluated) and either no PRE or a wild-type or mutated WPRE operably linked to the 3′ prime end of the polynucleotide encoding the anti-BCMA CAR.

FIG. 3B shows the VCN in transduced PBMCs as a function of the percentage of PBMCs expressing an anti-BCMA CAR. Human PBMCs were transduced with a recombinant lentivirus comprising a viral envelope expressing a mutated VSIV-G and a non-viral membrane-bound tropism polypeptide that binds CD3 and a lentiviral vector encoding either an MNDU3 promoter, an SFFV promoter, or an EF1α promoter operably linked to a polynucleotide encoding a CD8α signal peptide and an anti-BCMA CAR (6 anti-BCMA CARs were evaluated) and either no PRE or a wild-type or mutated WPRE operably linked to the 3′ prime end of the polynucleotide encoding the anti-BCMA CAR.

FIG. 3C shows the amount of IFNγ secreted from PBMCs expressing an anti-BCMA CAR co-cultured with RPMI-8226 cells (BCMA expressing cells) for 24 hours as a function of the CAR-expressing cells in the co-culture.

FIG. 3D shows the amount of IL-2 secreted from PBMCs expressing an anti-BCMA CAR co-cultured with RPMI-8226 cells (BCMA expressing cells) for 24 hours as a function of the CAR-expressing cells in the co-culture.

FIG. 3E shows the amount of IFNγ secreted from PBMCs expressing an anti-BCMA CAR in the absence of target cells. Human PBMCs were transduced with a recombinant lentivirus comprising a viral envelope expressing a mutated VSIV-G and a non-viral membrane-bound tropism polypeptide that binds CD3 and a lentiviral vector comprising one of the following lentiviral vector architectures, MNDU3 promoter and wild-type WPRE, MNDU3 promoter and a mutated WPRE, SFFV promoter and a mutated WPRE, and EF1α promoter and no WPRE and encoding an anti-BCMA CAR (6 anti-BCMA CARs were evaluated).

FIG. 3F shows the levels of off-target transduction in BCMA expressing cells (RPMI-8226 and KMS-11) of recombinant lentiviruses comprising a viral envelope expressing a mutated VSIV-G and a non-viral membrane-bound tropism polypeptide that binds CD3 and a lentiviral vector comprising one of the following lentiviral vector architectures, MNDU3 promoter and wild-type WPRE, MNDU3 promoter and a mutated WPRE, SFFV promoter and a mutated WPRE, and EF1α promoter and no WPRE and encoding an anti-BCMA CAR (6 anti-BCMA CARs were evaluated). Transduction was normalized to VCN in cells transduced with a recombinant lentivirus encoding GFP in place of an anti-BCMA CAR.

FIG. 4A shows the results from an in vivo Daudi mouse model. Mice were administered a recombinant lentivirus comprising a viral envelope expressing a mutated VSIV-G and a non-viral membrane-bound tropism polypeptide that binds CD3 and a lentiviral vector encoding either an MNDU3 promoter operably linked to a polynucleotide encoding a CD8α signal peptide and an anti-BCMA CAR (4 anti-BCMA CARs were evaluated) and a wild-type WPRE operably linked to the 3′ prime end of the polynucleotide encoding the anti-BCMA CAR; ex vivo CAR T cells, or vehicle.

FIG. 4B shows the results from an in vivo Daudi mouse model. Mice were administered a recombinant lentivirus comprising a viral envelope expressing a mutated VSIV-G and a non-viral membrane-bound tropism polypeptide that binds CD3 and a lentiviral vector comprising one of the following lentiviral vector architectures, MNDU3 promoter and wild-type WPRE, MNDU3 promoter and a mutated WPRE, SFFV promoter and a mutated WPRE, and EF1α promoter and no WPRE and encoding an anti-BCMA CAR (5 anti-BCMA CARs were evaluated); ex vivo CAR T cells, or vehicle.

FIG. 4C shows the results from an in vivo Daudi mouse model. Mice were administered a recombinant lentivirus comprising a viral envelope expressing a mutated VSIV-G and a non-viral membrane-bound tropism polypeptide that binds CD3 and a lentiviral vector comprising one of the following lentiviral vector architectures, MNDU3 promoter and a mutated WPRE, SFFV promoter and a mutated WPRE, and EF1α promoter and no WPRE and encoding an anti-BCMA CAR (4 anti-BCMA CARs were evaluated); ex vivo CAR T cells, or vehicle.

FIG. 4D shows the results from an in vivo RPMI-8226 mouse model. Mice were administered a recombinant lentivirus comprising a viral envelope expressing a mutated VSIV-G and a non-viral membrane-bound tropism polypeptide that binds CD3 and a lentiviral vector comprising one of the following lentiviral vector architectures, MNDU3 promoter and a mutated WPRE, or EF1α promoter and no WPRE and encoding an anti-BCMA CAR (2 anti-BCMA CARs were evaluated); ex vivo CAR T cells, or vehicle.

FIG. 4E shows the results from an in vivo RPMI-8226 mouse model. Mice were administered three doses (1.25×10⁷ IU, 5.0×10⁷ IU, or 1.25×10⁸ IU) of a recombinant lentivirus comprising a viral envelope expressing a mutated VSIV-G and a non-viral membrane-bound tropism polypeptide that binds CD3 and a lentiviral vector comprising an EF1α promoter operably linked to a polynucleotide encoding an anti-BCMA CAR without a PRE; ex vivo CAR T cells, or vehicle.

FIG. 4F shows the results from an in vivo RPMI-8226 mouse model. Mice were administered two doses (5.0×10⁷ IU or 1.25×10⁸ IU) of a recombinant lentivirus comprising a viral envelope expressing a mutated VSIV-G and a non-viral membrane-bound tropism polypeptide that binds CD3 and a lentiviral vector comprising an EF1α promoter operably linked to a polynucleotide encoding an anti-BCMA CAR without a PRE; ex vivo CAR T cells, or vehicle.

FIG. 4G shows the results from an in vivo Daudi mouse model. Mice were administered a recombinant lentivirus comprising a viral envelope expressing a mutated VSIV-G and a non-viral membrane-bound tropism polypeptide that binds CD3 and a lentiviral vector and encoding an anti-BCMA CAR and comprising one of the following lentiviral vector architectures, MNDU3 promoter and a mutated WPRE or MNDU3 promoter and a mutated WPRE (at 1.25×10⁸ IU) or an EF1α promoter and no WPRE (5.6×10⁷ IU); ex vivo CAR T cells, or vehicle.

FIG. 5A shows the results from a Daudi mouse model interrogated with in vivo lentivirus. Mice were administered vehicle control or recombinant lentivirus comprising a viral envelope expressing a mutated VSIV-G and a non-viral membrane-bound tropism polypeptide that binds CD3 and a lentiviral vector encoding one of three anti-BCMA CARs or a GFP control.

FIG. 5B shows the results from a Daudi mouse model interrogated with ex vivo manufactured CAR T cells. Mice were administered vehicle control, untransduced PBMCS, or PBMCs transduced with a recombinant lentivirus comprising a viral envelope expressing a mutated VSIV-G and a non-viral membrane-bound tropism polypeptide that binds CD3 and a lentiviral vector and encoding one of three anti-BCMA CARs.

### BRIEF DESCRIPTION OF THE SEQUENCE IDENTIFIERS

SEQ ID NOs: 1-9 set forth amino acid sequences of fusogens.

SEQ ID NO: 10 sets forth an amino acid sequence of BCMA.

SEQ ID NOs: 11-224 set forth amino acid sequences of antibodies.

SEQ ID NOs: 225-230 set forth amino acid sequences of spacer/hinge domains.

SEQ ID NOs: 231-237 set forth amino acid sequences of transmembrane domains.

SEQ ID NOs: 238-244 set forth amino acid sequences of intracellular domains.

SEQ ID NOs: 245-254 set forth amino acid sequences of signal peptides.

SEQ ID NOs: 255-283 set forth amino acid sequences of chimeric antigen receptors.

SEQ ID NOs: 284-287 set forth nucleic acid sequences encoding spacer/hinge domains.

SEQ ID NOs: 288-289 set forth nucleic acid sequences encoding transmembrane domains.

SEQ ID NOs: 290-293 set forth nucleic acid sequences encoding intracellular signaling domains.

SEQ ID NO: 294 sets forth the nucleic acid sequence encoding a signal peptide.

SEQ ID NOs: 295-314 set forth nucleic acid sequences encoding chimeric antigen receptors without a signal peptide.

SEQ ID NOs: 315-317 set forth nucleic acid sequences of post-transcriptional response elements.

SEQ ID NOs: 318-323 set forth nucleic acid sequences of promoters.

SEQ ID NOs: 324-331 set forth amino acid sequences of non-viral membrane bound tropism polypeptides.

SEQ ID NOs: 332-339 set forth amino acid sequences of fusogens.

SEQ ID NOs: 340-341 set forth amino acid sequences of anti-BCMA CARs.

SEQ ID NOs: 342-354 set forth amino acid sequences of polypeptide linkers.

SEQ ID NOs: 355-374 set forth amino acid sequences of viral self-cleaving peptides.

In the foregoing sequences, X, if present, refers to any amino acid, a specified group of amino acids or the absence of an amino acid.

Throughout the disclosure, the amino acid positions of a fusogen are with reference to the fusogen lacking a signal sequence (i.e., the amino acid sequence after the signal peptide has been cleaved).

### DETAILED DESCRIPTION

### A. Overview

The field of ex vivo gene therapy is not new and has been evolving for decades. Despite huge potential, ex vivo gene therapy has been met with limited success. Moreover, substantial obstacles still plague the field of ex vivo gene therapy, obstacles including limited precision and lack of commercial viability are likely among the reasons that it has yet to see widespread adoption in a clinical setting.

Recently, ex vivo CAR T cell therapies that target B cell maturation antigen (BCMA) have been used to treat relapsed and refractory multiple myeloma. Although many multiple myeloma patients that have been treated with ex vivo anti-BCMA CAR T cell therapies experience partial or complete remissions, most relapse and succumb to the disease. There is a significant unmet need for a durable, one-time, and potentially curative treatment for multiple myeloma.

Recombinant lentiviral particles that enable delivery of vectors encoding chimeric antigen receptors to immune effector cells in vivo are new and offer the potential to deliver life-altering therapies on an unprecedented scale. In vivo CAR T cell therapy solves the commercial viability issues associated with the astronomical costs associated with ex vivo CAR T cell manufacturing. But in vivo CAR T cell therapy also comes with its own set of challenges related to the delivery mechanism, including potential off-target toxicity, low efficacy, and immunogenicity.

The present disclosure offers solutions to foregoing challenges and others that exist in the field of using recombinant lentiviral particles to efficiently and safely provide in vivo CAR T cell therapy.

The present disclosure generally relates to, in part, to an engineered cell-targeting particle (e.g., a fusosome; an extracellular vesicle, including a microvesicle, an apoptotic body, and an exosome; a lipid nanoparticle; a virus-like particle (VLPs); or a viral particle) that has a surface that expresses a non-viral tropism polypeptide engineered to bind an immune effector cell and a mutated viral glycoprotein that promotes fusion of the particle and the immune effector cell; and one or more copies of a vector that encodes or comprises a promoter operably linked to a polynucleotide encoding a chimeric antigen receptor that binds B cell maturation antigen (BCMA).

The disclosure contemplates, in part, recombinant lentiviral particles engineered to bind and transduce immune effector cells with a vector encoding an anti-BCMA CAR, in vivo. In various embodiments, a recombinant lentiviral particle comprises an envelope engineered to express a non-viral tropism polypeptide that bind an immune effector cell and a mutated vesiculovirus glycoprotein that does not bind its cognate receptor, e.g., low density lipoprotein receptor (LDLR), but that promotes fusion of the particle and the immune effector cell; and one or more copies of a lentiviral vector that encodes or comprises a promoter operably linked to a polynucleotide encoding an anti-BCMA CAR. The recombinant lentiviral particles may be used for ex vivo CAR T cell therapy but provide substantial advantages for use in in vivo CAR T cell therapy.

The disclosure further contemplates, in part, methods of making the recombinant lentiviral particles contemplated herein, along with methods of using the particles for treating a subject in need thereof.

In particular embodiments, the disclosure contemplates, methods of using a recombinant lentiviral particle contemplated herein to generate anti-BCMA immune effector cells, e.g., T cells, in vivo, to treat a disorder, disease, condition or symptoms associated therewith, preferably to treat cancer, and more preferably, to treat a multiple myeloma, e.g., relapsed refractory multiple myeloma.

Compositions, pharmaceutical compositions, and kits comprising one or more recombinant lentiviral particles contemplated herein and methods of making and using the same are also provided in particular embodiments.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 4th ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience (2002); Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid the Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells and Enzymes (IRL Press, 1986); the treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods in Cell and Molecular Biology (Mayer and Walker, eds., Academic Press, London, 1987); Handbook of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination of the recited alternatives.

The term “and/or” should be understood to mean either one of, or both of, the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length. In particular embodiments, “substantially lacks cell binding or cell attachment activity and/or cognate receptor binding activity” or “substantially abates cell binding or cell attachment activity and/or cognate receptor binding activity” refers to the negligible or undetectable or absent cell binding activity or cell attachment activity of a modified membrane-bound viral glycoprotein contemplated herein to bind or attach to its cognate receptor on the surface of a cell compared to the cell binding activity or cell attachment activity of the unmodified membrane-bound viral glycoprotein to bind or attach to its cognate receptor on the surface of the cell.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory and that no other elements may be present. The phrase “consisting essentially of” means including any elements listed after the phrase and other elements that do not interfere with or contribute to the activity or action specified in the disclosure for the listed elements. Thus, the phrase “consisting essentially of” indicates that the listed elements are required or mandatory but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The terms “spacer,” “spacer domain,” or “spacer polypeptide” are used interchangeably and refer to a polypeptide domain or sequence of amino acids in a non-viral membrane-bound tropism polypeptide disposed between an extracellular antigen targeting domain and a transmembrane domain. A spacer positions the extracellular antigen targeting domain away from the particle surface to enable proper particle/target cell contact, attachment, or binding. A spacer may be derived either from a natural, synthetic, semi-synthetic, or recombinant source. Illustrative examples of spacer domains include but are not limited to hinge or stalk domains derived, obtained, or isolated from CD8α, CD28, and CD45 isoforms, and polypeptide linkers of similar amino acid composition, rigidity, flexibility and/or length.

A “hinge domain,” is a type of spacer domain that plays a role in positioning the antigen binding domain away from the effector cell surface to enable proper cell/cell contact, antigen binding and activation. A hinge domain is placed between a binding domain and a transmembrane domain (TM) of a non-viral membrane-bound tropism polypeptide or between an anti-BCMA antibody or antigen binding fragment thereof and a TM domain of a chimeric antigen receptor. A hinge domain may be derived either from a natural, synthetic, semi-synthetic, or recombinant source. A hinge domain may be altered by substituting one or more cysteine and/or proline residues in a naturally occurring immunoglobulin hinge domain with one or more other amino acid residues (e.g., one or more serine residues).

A “transmembrane domain” or “TM domain” refers to a hydrophobic portion of polypeptide that anchors the polypeptide to the plasma membrane of the cell. The TM domain may be derived either from a natural, synthetic, semi-synthetic, or recombinant source.

An “intracellular signaling domain” refers to a polypeptide domain that participates in transducing the message of effective binding of a target antigen by a chimeric antigen receptor expressed on an immune effector cell to the immune effector cell's interior to elicit one or more effector functions (an “effector function” refers to a specialized function of an immune effector cell), e.g., activation, cytokine production, proliferation and cytotoxic activity, including the release of cytotoxic factors, or other cellular responses elicited with antigen binding to the receptor expressed on the immune effector cell. “Intracellular signaling domains” include a polypeptide domain or functional fragment thereof, which transduces an effector function signal and that directs a cell to perform a specialized function. The term intracellular signaling domain is meant to include any truncated portion of an intracellular signaling domain sufficient to transduce effector function signal.

T cell activation can be said to be mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal.

A “primary signaling domain” refers to a signaling domain that regulates the primary activation of a TCR complex either in a stimulatory way, or in an inhibitory way. Primary signaling domains that act in a stimulatory manner may contain one or more signaling motifs which are known as immunoreceptor tyrosine-based activation motifs or ITAMs.

A “costimulatory signaling domain” or “costimulatory domain” refers to an intracellular signaling domain of a costimulatory molecule. Costimulatory molecules are cell surface molecules other than antigen receptors or Fc receptors that provide a second signal required for efficient activation and function of T lymphocytes upon binding to antigen.

“Linker,” “peptide linker,” and “polypeptide linker” are used interchangeably and refer to a plurality of amino acid residues between various polypeptide domains added for appropriate spacing, conformation, and function. A polypeptide linker sequence may be employed to separate any two or more polypeptide components by a distance sufficient to ensure that each polypeptide folds into its appropriate secondary and tertiary structures so as to allow the polypeptide domains to exert their desired functions. Linkers include a “variable domain linking sequence,” an amino acid sequence that connects two or more domains of an antibody or antigen binding fragments thereof and provides a spacer function compatible with interaction of the two sub-binding domains so that the resulting polypeptide retains a specific binding affinity to the same target molecule as an antibody that comprises the same light and/or heavy chain variable domains. A linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more amino acids long. Illustrative examples of linkers include, but are not limited to the following amino acid sequences: TGEKP (SEQ ID NO: 342); (GGGGS)n wherein n=1, 2, 3, 4 or 5 (SEQ ID NOs: 343-347); EGKSSGSGSESKVD (SEQ ID NO: 348); KESGSVSSEQLAQFRSLD (SEQ ID NO: 349); LRQRDGERP (SEQ ID NO: 350); LRQKDGGGSERP (SEQ ID NO: 351); LRQKD(GGGS)₂ERP (SEQ ID NO: 352), GEGTSTGSGGSGGSGGAD (SEQ ID NO: 353), and GSTSGSGKPGSGEGSTKG (SEQ ID NO: 354).

The terms “individual” and “subject” are often used interchangeably and refer to any animal that exhibits a symptom of a disease, disorder, or condition, e.g., cancer, that can be treated with the recombinant particles, e.g., recombinant lentiviral particles contemplated elsewhere herein. Suitable subjects (e.g., patients) include laboratory animals (e.g., mouse, rat, rabbit, or guinea pig), farm animals, and domestic animals or pets (e.g., cat or dog). Non-human primates and, preferably, human patients, are preferred subjects.

A “patient” refers to a subject that has been diagnosed with a particular disease, disorder, or condition that can be treated with the recombinant particles disclosed elsewhere herein.

“Treatment” or “treating,” as used herein includes any beneficial or desirable effect on the symptoms or pathology of a disease or pathological condition and may include even minimal reductions in one or more measurable markers of the disease or condition being treated. Optionally, treatment can include reducing the disease burden or delaying disease progression. “Treatment” may, but does not necessarily, indicate complete eradication or cure of the disease or condition, or associated symptoms thereof.

“Prevent,” “prevention,” “preventing” and the like, as used herein, indicate an approach for preventing, inhibiting, or reducing the likelihood of occurrence or recurrence of a disease or condition. It also refers to delaying the onset or recurrence of a disease or condition or delaying the occurrence or recurrence of the symptoms of a disease or condition. Prevention includes reducing the intensity, effect, symptoms and/or burden of a disease or condition prior to onset or recurrence of the disease or condition.

“Ameliorating at least one symptom of” as used herein, refers to decreasing one or more symptoms of the disease or condition for which a subject is being treated. In particular embodiments, the disease or condition being treated is a cancer, and the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

Additional definitions are set forth throughout this disclosure.

### C. Recombinant Particles

Various particles may be used as gene delivery vehicles, e.g., a fusosome; an extracellular vesicle, including a microvesicle, an apoptotic body, and an exosome; a lipid nanoparticle; a virus-like particle (VLPs); or a recombinant viral particle. The present disclosure contemplates, in part, a recombinant lentiviral particle engineered to bind to an immune effector cell and transduce the cell with a vector encoding an anti-BCMA CAR.

Recombinant retroviral particles have been used as a gene delivery platform for treatments of severe genetic diseases and cancer. A “lentivirus” refers to a complex retrovirus. Among retroviruses, lentiviruses are the most efficient at transducing resting or growth-arrested cells. In preferred embodiments, a recombinant particle is a recombinant lentiviral particle or recombinant lentivirus. The terms “recombinant lentiviral particle” and “recombinant lentivirus” are used interchangeably.

Lentiviruses suitable for deriving or engineering recombinant lentiviruses contemplated in particular embodiments herein include but are not limited to human immunodeficiency virus (HIV), including HIV type 1 (HIV-1) and HIV type 2 (HIV-2); visna-maedi virus (VMV); caprine arthritis-encephalitis virus (CAEV); equine infectious anemia virus (EIAV); feline immunodeficiency virus (FIV); bovine immune deficiency virus (BIV); and simian immunodeficiency virus (SIV).

In particular embodiments, a recombinant lentiviral particle is derived or engineered from an HIV-1 or HIV-2 lentivirus.

In particular embodiments, a recombinant lentiviral particle comprises (i) a particle surface comprising (a) a mutated viral envelope glycoprotein and (b) a non-viral membrane-bound tropism polypeptide that binds an immune effector cell; and (ii) two copies of a lentiviral vector comprising or encoding a promoter operably linked to a polynucleotide encoding an anti-BCMA CAR.

### D. Particle Surface

Recombinant particles are engineered to bind to an immune effector cell, e.g., a CD3+ cell and deliver one or more copies of a vector encoding an anti-BCMA chimeric antigen receptor to the cell.

In particular embodiments, a recombinant lentiviral particle comprises a viral envelope comprising a mutated vesiculovirus envelope glycoprotein that does not bind its cognate receptor, e.g., LDLR, but that mediates virus-cell fusion and a non-viral membrane-bound tropism polypeptide that redirects the particle to immune effector cells that express CD3; and a lentiviral vector encoding or comprising a promoter operably linked to a polynucleotide encoding an anti-BCMA CAR. The foregoing engineering strategy enables highly efficient on-target delivery of vectors encoding CARs to immune effector cells while minimizing, reducing and/or eliminating delivery to undesired cell types.

### 1. Viral Envelope Glycoproteins

In particular embodiments, a recombinant lentiviral particle comprises a mutated vesiculovirus envelope glycoprotein in which the native or a heterologous signal peptide has been cleaved. Thus, amino acid positions identified in mutated vesiculovirus envelope glycoproteins are with reference to the mature polypeptide, in which the signal peptide has been cleaved.

In particular embodiments, a vesiculovirus is vesicular stomatitis Indiana virus (VSIV). In particular embodiments, a mutated viral envelope glycoprotein is derived from a VSIV envelope glycoprotein (VSIV-G) set forth in Table 1.

TABLE 1
VSIV-G polypeptides

| SEQ | |
|---|---|
| ID | |
| NO: | AMINO ACID SEQUENCE |
| 1 | KFTIVFPHNQKGNWKNVPSNYHYCPSSSDLNWHNDLIGTALQVKMPKSHKAIQADGWMCHASKWVTT |
| | CDFRWYGPKYITHSIRSFTPSVEQCKESIEQTKQGTWLNPGFPPQSCGYATVIDAEAVIVQVTPHHV |
| | LVDEYTGEWVDSQFINGKCSNYICPTVHNSTTWHSDYKVKGLCDSNLISMDITFFSEDGELSSLGKE |
| | GTGFRSNYFAYETGGKACKMQYCKHWGVRLPSGVWFEMADKDLFAAARFPECPEGSSISAPSQTSVD |
| | VSLIQDVERILDYSLCQETWSKIRAGLPISPVDLSYLAPKNPGTGPAFTIINGTLKYFETRYIRVDI |
| | AAPILSRMVGMISGTTTERELWDDWAPYEDVEIGPNGVLRTSSGYKFPLYMIGHGMLDSDLHLSSKA |
| | QVFEHPHIQDAASQLPDDESLFFGDTGLSKNPIELVEGWFSSWKSSIASFFFIIGLIIGLELVLRVG |
| | IHLCIKLKHTKKRQIYTDIEMNRLGK |
| 2 | KFTIVFPHNQKGNWKNVPSNYHYCPSSSDLNWHNDLIGTAIQVKMPKSHKAIQADGWMCHASKWVTT |
| | CDFRWYGPKYITQSIRSFTPSVEQCKESIEQTKQGTWLNPGFPPQSCGYATVIDAEAVIVQVTPHHV |
| | LVDEYTGEWVDSQFINGKCSNYICPTVHNSTTWHSDYKVKGLCDSNLISMDITFFSEDGELSSLGKE |
| | GTGFRSNYFAYETGGKACKMQYCKHWGVRLPSGVWFEMADKDLFAAARFPECPEGSSISAPSQTSVD |
| | VSLIQDVERILDYSLCQETWSKIRAGLPISPVDLSYLAPKNPGTGPAFTIINGTLKYFETRYIRVDI |
| | AAPILSRMVGMISGTTTERELWDDWAPYEDVEIGPNGVLRTSSGYKFPLYMIGHGMLDSDLHLSSKA |
| | QVFEHPHIQDAASQLPDDESLFFGDTGLSKNPIELVEGWFSSWKSSIASFFFIIGLIIGLFLVLRVG |
| | IHLCIKLKHTKKRQIYTDIEMNRLGK |
| 3 | KFTIVFPHNQKGNWKNVPSNYHYCPSSSDLNWHNDLIGTAIQVKMPKSHKAIQADGWMCHASKWVTT |
| | CDFRWYGPKYITHSIRSFTPSVEQCKESIEQTKQGTWLNPGFPPQSCGYATVIDAEAVIVQVTPHHV |
| | LVDEYTGEWVDSQFINGKCSNYICPTVHNSTTWHSDYKVKGLCDSNLISMDITFFSEDGELSSLGKE |
| | GTGFRSNYFAYETGGKACKMQYCKHWGVRLPSGVWFEMADKDLFAAARFPECPEGSSISAPSQTSVD |
| | VSLIQDVERILDYSLCQETWSKIRAGLPISPVDLSYLAPKNPGTGPAFTIINGTLKYFETRYIRVDI |
| | AAPILSRMVGMISGTTTERELWDDWAPYEDVEIGPNGVLRTSSGYKFPLYMIGHGMLDSDLHLSSKA |
| | QVFEHPHIQDAASQLPDDESLFFGDTGLSKNPIELVEGWFSSWKSSIASFFFIIGLIIGLFLVLRVG |
| | IHLCIKLKHTKKRQIYTDIEMNRLGK |
| 4 | KFTIVFPHNQKGNWKNVPSNYHYCPSSSDLNWHNDLIGTALQVKMPKSHKAIQADGWMCHASKWVTT |
| | CDFRWYGPKYITQSIRSFTPSVEQCKESIEQTKQGTWLNPGFPPQSCGYATVIDAEAVIVQVTPHHV |
| | LVDEYTGEWVDSQFINGKCSNYICPTVHNSTTWHSDYKVKGLCDSNLISMDITFFSEDGELSSLGKE |
| | GTGFRSNYFAYETGGKACKMQYCKHWGVRLPSGVWFEMADKDLFAAARFPECPEGSSISAPSQTSVD |
| | VSLIQDVERILDYSLCQETWSKIRAGLPISPVDLSYLAPKNPGTGPAFTIINGTLKYFETRYIRVDI |
| | AAPILSRMVGMISGTTTERELWDDWAPYEDVEIGPNGVLRTSSGYKFPLYMIGHGMLDSDLHLSSKA |
| | QVFEHPHIQDAASQLPDDESLFFGDTGLSKNPIELVEGWFSSWKSSIASFFFIIGLIIGLFLVLRVG |
| | IHLCIKLKHTKKRQIYTDIEMNRLGK |
| 5 | KFTIVFPHNQKGNWKNVPSNYHYCPSSSDLNWHNDLIGTALQVKMPKSHKAIQADGWMCHASKWITT |
| | CDFRWYGPKYITHSIQSFTPSVEQCKESIEQTKQGTWLNPGFPPQSCGYATVTDAEAVIVQVTPHHV |
| | LVDEYTGEWVDSQFINGKCSNDICLTVHNSTTWHSDYKVKGLCDSNLISMDITFFSEDGELSSLGKA |
| | GTGFRSNYFAYETGDKACKMQYCKHWGVRLPSGVWFEMADKDLFAAAKFPECPEGSSISAPSQTSVD |
| | VSLIQDVERILDYSLCQETWSKIRAGLPISPVDLSYLAPKNPGTGPAFTIINGTLKYFETRYIRVDI |
| | AAPILSRMVGMISGTNTERELWEDWAPYEDVEIGPNGVLRTSSGYKFPLYMIGHGMLDSDLHLSSKV |
| | QVFEHPHIQDAASQLPDDETLFFGDTGLSKNPIELVEGWFSGWKSSIASFFFIIGLIIGLFLVLRVG |
| | IYLCIKLKHTRKRKIYADIEMNRLGK |
| 6 | KFTTVFPHNKKGDWKNVPSNYHYCPSSSDLNWHNDLIGTALQVKMPKSHKAIQADGWMCHASKWVTT |
| | CDFRWYGPKYITHSIRSFTPSVEQCKESIEQTKQGTWLNPGFPPQSCGYATVIDAEAVIVQVTPHHV |
| | LVDEYTGEWVDSQFINGKCSDDICPTVHNSTTWHSDYKVKGLCDSNLISMDITFFSEDGELSSLGKE |
| | GTGFRSNYFAYETGDKACKMQYCKHWGVRLPSGVWFEMADKNLFAAAKFPECPEGSSISAPSQTSVD |
| | VSLIQDVERILDYSLCQETWSKIRAGLPISPVDLSYLAPKNPGTGPAFTIINGTLKYFETRYIRVDI |
| | AAPILSRMVGMISGTTTERELWEDWAPYEDVEIGPNGVLRTSSGYKFPLYMIGHGMLDSDLHLSSKA |
| | QVFEHPHIPDATSQLPDDETLFFGDTGLSKDPIELVEGWFSGWKSSIASFFFIIGLIIGLFFVLRIG |
| | VYLCIKLKHTNKRQIYTDIEMNRLGK |
| 7 | KFTIVFPHNQKGTWKNVPSNYHYCPSSSDLNWHNDLIGTALQVKMPKSHKAIQADGWMCHASKWVTT |
| | CDFRWYGPKYITHSIRSFTPSVEQCRESIEQTKQGTWLNPGFPPQSCGYATVIDAEAVIVQVTPHHV |
| | LVDEYTGEWVDSQFINGKCSNDICPTVHNSTTWHSDYKVKGLCDSNLISMDITFFSEDGELSSLGKE |
| | GTGFRSNHFAYETGDKACKMQYCKHWGVRLPSGVWFEMADQDLFAAARFPECPEGSSISAPSQTSVD |
| | VSLIQDVERILDYSLCQETWSKIGAGLPISPVDLSYLAPKNPGTGPAFTIINGTLKYFETRYIRVDI |
| | AAPILSRMVGMISGTTTERELWDDWAPYEDVEIGPNGVLRTSSGYKFPLYMIGHGMLDSDLHLSSKA |
| | QVFEHPHIQDAASQLPDDETLFFGDTGLSKNPIELVEGWFSGWKSSIASFFFIIGLIIGLFLVLRVG |
| | IYLCIKLKHTKKRQIYTDIEMNRLGK |
| 8 | KFTIVFPHNQKGNWKNVPSNYHYCPSSSDLNWHNDLIGTGLQVKMPKSHKAIQADGWMCHASKWVTT |
| | CDFRWYGPKYITHSIRSFTPSVEQCKESIEQTKQGTWLNPGFPPQSCGYATVTDAEAVIVQVTPHHV |
| | LVDEYTGEWVDSQFINGKCSNDICPTVHNSTTWHSDYKVKGLCDSNLISTDITFFSEDRELSSLGKE |
| | GTGFRSNYFAYETGDKACKMQYCKHWGVRLPSGVWFEMADKDLFAAARFPECPEGSSISAPSQTSVD |
| | VSLIQDVERILDYSLCQETWSKIRAGLPISPVDLSYLAPKNPGTGPAFTIINGTLKYFETRYIRVDI |
| | AAPILSRMVGMISGTTTERELWDDWAPYEDVEIGPNGVLRTSSGYKFPLYMIGHGMLDSGLHLSSKA |
| | QVFEHPHIQDAASQLPDDEILFFGDTGLSKNPIDFVEGWFSSWKSSIASFFFIIGLIIGLFLVLRVG |
| | IYLYIKLKHTKKRQIYTDIEMNRLGR |

In particular embodiments, a mutated viral envelope glycoprotein is derived from a VSIV envelope glycoprotein (VSIV-G) comprising an amino acid sequence set forth in any one of SEQ ID NOs: 1-8 or an amino acid sequence at least 95%, 96%, 97%, 98%, or 99% identical thereto.

In particular embodiments, a mutated VSIV-G polypeptide comprises one or more amino acid substitutions at K47, I182, and/or R354 (substitution with any amino acid; a conservation substitution; a disruptive substitution; substitution with D, E, A, G, F, or Q; or substitution with A, G, F, or Q). In particular embodiments, a mutated VSIV-G polypeptide comprises amino acid substitutions at K47, I182, or R354; K47 and I182; K47 and R354; I182 and R354; or at K47, I182, and R354 of any one of SEQ ID NOs: 1-8.

In particular embodiments, a mutated VSIV-G polypeptide comprises one or more of the following amino acid substitutions: K47A, K47Q, I182E, I182D, R354A, and/or R354Q. In particular embodiments, a mutated VSIV-G polypeptide comprises the following amino acid substitutions: K47A, K47Q, I182E, I182D, R354A, or R354Q; K47A and I182E; K47A and I182D; K47Q and I182E; K47Q and I182D; I182E and R354A; I182E and R354Q; I182D and R354A; I182D and R354Q; K47A and R354A; K47A and R354Q; K47Q and R354A; K47Q and R354Q; K47A, I182E, and R354A; K47A, I182D, and R354A; K47Q, I182E, and R354A; K47Q, I182D, and R354A; K47A, I182E, and R354Q; K47A, I182D, and R354Q; K47Q, I182E, and R354Q; or K47Q, I182D, and R354Q of any one of SEQ ID NOs: 1-8. In a preferred embodiment, a VSIV-G polypeptide comprises the amino acid substitutions K47Q and R354A of any one of SEQ ID NOs: 1, 2, 3, 4, 5, 6, 7, or 8.

In particular embodiments, a mutated VSIV-G polypeptide comprises an amino acid sequence set forth in Table 2 (SEQ ID NOs: 332, 333, 334, or 335) or an amino acid sequence at least 95%, 96%, 97%, 98%, or 99% identical thereto that mediates fusion of the lentiviral particle and an immune effector cell but that substantially ablates or ablates the polypeptide's ability to bind its cognate receptor expressed on a cell, e.g., LDL-R.

TABLE 2

| SEQ | |
|---|---|
| ID | |
| NO: | AMINO ACID SEQUENCE |
| 332 | KFTIVFPHNQKGNWKNVPSNYHYCPSSSDLNWHNDLIGTAIQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 333 | KFTIVFPHNQKGNWKNVPSNYHYCPSSSDLNWHNDLIGTALQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 334 | KFTIVFPHNQKGNWKNVPSNYHYCPSSSDLNWHNDLIGTAIQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 335 | KFTIVFPHNQKGNWKNVPSNYHYCPSSSDLNWHNDLIGTALQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |

In particular embodiments, a vesiculovirus is cocal virus (COCV). In particular embodiments, a mutated viral envelope glycoprotein is derived from a COCV envelope glycoprotein (COCV-G) set forth in Table 3.

TABLE 3
COCV-G polypeptide

| SEQ | |
|---|---|
| ID | |
| NO | AMINO ACID SEQUENCE |
| 9 | KFSIVFPQSQKGNWKNVPSSYHYCPSSSDQNWHNDLLGITMKVKM |
| | PKTHKAIQADGWMCHAAKWITTCDFRWYGPKYITHSIHSIQPTSE |
| | QCKESIKQTKQGTWMSPGFPPQNCGYATVTDSVAVVVQATPHHVL |
| | VDEYTGEWIDSQFPNGKCETEECETVHNSTVWYSDYKVTGLCDAT |
| | LVDTEITFFSEDGKKESIGKPNTGYRSNYFAYEKGDKVCKMNYCK |
| | HAGVRLPSGVWFEFVDQDVYAAAKLPECPVGATISAPTQTSVDVS |
| | LILDVERILDYSLCQETWSKIRSKQPVSPVDLSYLAPKNPGTGPA |
| | FTIINGTLKYFETRYIRIDIDNPIISKMVGKISGSQTERELWTEW |
| | FPYEGVEIGPNGILKTPTGYKFPLFMIGHGMLDSDLHKTSQAEVF |
| | EHPHLAEAPKQLPEEETLFFGDTGISKNPVELIEGWFSSWKSTVV |
| | TFFFAIGVFILLYVVARIVIAVRYRYQGSNNKRIYNDIEMSRFRK |

In particular embodiments, a mutated viral envelope glycoprotein is derived from a COCV envelope glycoprotein (COCV-G) comprising an amino acid sequence set forth in SEQ ID NO: 9 or an amino acid sequence at least 95%, 96%, 97%, 98%, or 99% identical thereto.

In particular embodiments, a mutated COCV-G polypeptide comprises one or more amino acid substitutions at K47, V182, and/or R354 (substitution with any amino acid; a conservation substitution; a disruptive substitution; substitution with D, E, A, G, F, or Q; or substitution with A, G, F, or Q). In particular embodiments, a mutated COCV-G polypeptide comprises amino acid substitutions at K47, V182, or R354; K47 and V182; K47 and R354; V182 and R354; or at K47, V182, and R354 of SEQ ID NO: 9.

In particular embodiments, a mutated COCV-G polypeptide comprises one or more of the following amino acid substitutions: K47A, K47Q, V182E, V182D, R354A, and/or R354Q. In particular embodiments, a mutated VSIV-G polypeptide comprises the following amino acid substitutions: K47A, K47Q, V182E, V182D, R354A, or R354Q; K47A and V182E; K47A and V182D; K47Q and V182E; K47Q and V182D; V182E and R354A; V182E and R354Q; V182D and R354A; V182D and R354Q; K47A and R354A; K47A and R354Q; K47Q and R354A; K47Q and R354Q; K47A, V182E, and R354A; K47A, V182D, and R354A; K47Q, V182E, and R354A; K47Q, V182D, and R354A; K47A, V182E, and R354Q; K47A, V182D, and R354Q; K47Q, V182E, and R354Q; or K47Q, V182D, and R354Q of SEQ ID NO: 9. In a preferred embodiment, a COCV-G polypeptide comprises the amino acid substitutions K47Q and R354A of SEQ ID NO: 9.

In particular embodiments, a mutated COCV-G polypeptide comprises an amino acid sequence set forth in Table 4 (SEQ ID NOs: 336, 337, 338, and 339) or an amino acid sequence at least 95%, 96%, 97%, 98%, or 99% identical thereto that mediates fusion of the lentiviral particle and an immune effector cell but that substantially ablates or ablates the polypeptide's ability to bind its cognate receptor expressed on a cell, e.g., LDLR.

TABLE 4

| SEQ | |
|---|---|
| ID | |
| NO: | AMINO ACID SEQUENCE |
| 336 | KFSIVFPQSQKGNWKNVPSSYHYCPSSSDQNWHNDLLGITMKVKMPATHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEAELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 337 | KFSIVFPQSQKGNWKNVPSSYHYCPSSSDQNWHNDLLGITMKVKMPATHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEQELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 338 | KFSIVFPQSQKGNWKNVPSSYHYCPSSSDQNWHNDLLGITMKVKMPQTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEAELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 339 | KFSIVFPQSQKGNWKNVPSSYHYCPSSSDQNWHNDLLGITMKVKMPQTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEQELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |

### 2. Tropism Polypeptides

Recombinant particles contemplated herein comprise a tropism polypeptide that governs targeting the particle to an immune effector cell. A “tropism polypeptide” is a polypeptide that binds one or more antigens on a desired cell type. A “non-viral membrane bound tropism polypeptide” is a polypeptide that binds one or more antigens on a desired cell type; that is not native to, or derived from, either in whole or in part, a virus.

In particular embodiments, a recombinant lentiviral particle comprises an envelope comprising or expressing a mutated VSIV-G polypeptide comprising one or more amino acid substitutions at positions K47, I182, and/or R354 or a mutated COCV-G polypeptide comprising one or more amino acid substitutions at positions K47, V182, and/or R354; a non-viral membrane-bound tropism polypeptide that binds an antigen expressed on an immune effector cell, e.g., CD3; and one or more copies of a lentiviral vector encoding or comprising a promoter operably linked to a polynucleotide encoding an anti-BCMA CAR. In particular embodiments, a recombinant lentiviral particle comprises an envelope that further comprises a secondary non-viral membrane-bound tropism polypeptide that an antigen specifically expressed on an immune effector cell, e.g., CD80, CD86, OX40L, 4-1BBL, and ICOSL.

Non-viral membrane-bound tropism polypeptides contemplated herein comprise, consist essentially of, or consist of an extracellular antigen targeting domain, a spacer and a transmembrane domain. As used herein, an “extracellular antigen targeting domain” (also referred to as an “extracellular targeting domain,” “antigen targeting domain,” or “targeting domain”) refers to any naturally occurring, synthetic, semi-synthetic, or recombinantly produced binding partner that binds a biological molecule or target antigen expressed or displayed on the surface of a cell. In preferred embodiments, an extracellular antigen targeting domain binds an antigen, e.g., CD3ε, expressed on an immune effector cell.

In particular embodiments, a non-viral membrane-bound tropism polypeptide comprises an extracellular antigen targeting domain comprising an antibody or antigen binding fragment thereof that binds CD3R. In particular embodiments, an extracellular antigen targeting domain comprises an anti-CD3ε scFv isolated from an antibody selected from the group consisting of: OKT3, UCHT1, YTH12.5, TR66, and variants thereof.

In certain embodiments, a non-viral membrane-bound tropism polypeptide comprises an extracellular antigen targeting domain comprising an anti-CD3p scFv comprising an amino acid sequence set forth in Table 5.

TABLE 5
anti-CD3 antibodies

| | SEQ ID | | |
|---|---|---|---|
| Ab ID | NO | ID | AMINO ACID SEQUENCE |
| CD3.9 | 145 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDHYCLDYWGQGTTVTVSS |
| | 146 | CDRH1 | RYTMH |
| | 147 | CDRH2 | YINPSRGYTNYADSVKG |
| | 148 | CDRH3 | YYDDHYCLDY |
| | 149 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 150 | CDRL1 | RASQSVSYMN |
| | 151 | CDRL2 | DTSKVAS |
| | 152 | CDRL3 | QQWSSNPLT |
| | 153 | scFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDHYCLDYWGQGTTVTVSSSGGGGSGGGGSGGGGS |
| | | | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 154 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYYDDH |
| | | | YCLDYWGQGTTVTVSS |
| CD3.10 | 155 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDPYCLDYWGQGTTVTVSS |
| | 156 | CDRH1 | RYTMH |
| | 157 | CDRH2 | YINPSRGYTNYADSVKG |
| | 158 | CDRH3 | YYDDPYCLDY |
| | 159 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 160 | CDRL1 | RASQSVSYMN |
| | 161 | CDRL2 | DTSKVAS |
| | 162 | CDRL3 | QQWSSNPLT |
| | 163 | scFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDPYCLDYWGQGTTVTVSSSGGGGSGGGGSGGGGS |
| | | | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 164 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYYDDP |
| | | | YCLDYWGQGTTVTVSS |
| CD3.11 | 165 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYNDQYCLDYWGQGTTVTVSSS |
| | 166 | CDRH1 | RYTMH |
| | 167 | CDRH2 | YINPSRGYTNYADSVKG |
| | 168 | CDRH3 | YYNDQYCLDY |
| | 169 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 170 | CDRL1 | RASQSVSYMN |
| | 171 | CDRL2 | DTSKVAS |
| | 172 | CDRL3 | QQWSSNPLT |
| | 173 | scFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYNDQYCLDYWGQGTTVTVSSSSGGGGSGGGGSGGGG |
| | | | SDIVLTQSPATLSLSPGERATLSCRASQSVSYMNWYQQKPGKAPK |
| | | | RWIYDTSKVASGVPARFSGSGSGTDYSLTINSLEAEDAATYYCQQ |
| | | | WSSNPLTFGGGTKVEIK |
| | 174 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYYNDQ |
| | | | YCLDYWGQGTTVTVSSS |
| CD3.12 | 175 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDAHNCLDYWGQGTTVTVSS |
| | 176 | CDRH1 | RYTMH |
| | 177 | CDRH2 | YINPSRGYTNYADSVKG |
| | 178 | CDRH3 | YYDAHNCLDY |
| | 179 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 180 | CDRL1 | RASQSVSYMN |
| | 181 | CDRL2 | DTSKVAS |
| | 182 | CDRL3 | QQWSSNPLT |
| | 183 | scFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDAHNCLDYWGQGTTVTVSSSGGGGSGGGGSGGGGS |
| | | | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 184 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYYDAH |
| | | | NCLDYWGQGTTVTVSS |
| CD3.13 | 185 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYSDDHYCLDYWGQGTTVTVSS |
| | 186 | CDRH1 | RYTMH |
| | 187 | CDRH2 | YINPSRGYTNYADSVKG |
| | 188 | CDRH3 | YSDDHYCLDY |
| | 189 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 190 | CDRL1 | RASQSVSYMN |
| | 191 | CDRL2 | DTSKVAS |
| | 192 | CDRL3 | QQWSSNPLT |
| | 193 | scFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYSDDHYCLDYWGQGTTVTVSSSGGGGSGGGGSGGGGS |
| | | | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 194 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYSDDH |
| | | | YCLDYWGQGTTVTVSS |
| CD3.14 | 195 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYSDDRYCLDYWGQGTTVTVSS |
| | 196 | CDRH1 | RYTMH |
| | 197 | CDRH2 | YINPSRGYTNYADSVKG |
| | 198 | CDRH3 | YSDDRYCLDY |
| | 199 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 200 | CDRL1 | RASQSVSYMN |
| | 201 | CDRL2 | DTSKVAS |
| | 202 | CDRL3 | QQWSSNPLT |
| | 203 | scFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYSDDRYCLDYWGQGTTVTVSSSGGGGSGGGGSGGGGS |
| | | | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 204 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYSDDR |
| | | | YCLDYWGQGTTVTVSS |
| CD3.15 | 205 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDNYCLDYWGQGTTVTVSSS |
| | 206 | CDRH1 | RYTMH |
| | 207 | CDRH2 | YINPSRGYTNYADSVKG |
| | 208 | CDRH3 | YYDDNYCLDY |
| | 209 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 210 | CDRL1 | RASQSVSYMN |
| | 211 | CDRL2 | DTSKVAS |
| | 212 | CDRL3 | QQWSSNPLT |
| | 213 | scFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDNYCLDYWGQGTTVTVSSSSGGGGSGGGGSGGGG |
| | | | SDIVLTQSPATLSLSPGERATLSCRASQSVSYMNWYQQKPGKAPK |
| | | | RWIYDTSKVASGVPARFSGSGSGTDYSLTINSLEAEDAATYYCQQ |
| | | | WSSNPLTFGGGTKVEIK |
| | 214 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYYDDN |
| | | | YCLDYWGQGTTVTVSSS |
| CD3.16 | 215 | VH | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDQYCLDYWGQGTTVTVSS |
| | 216 | CDRH1 | RYTMH |
| | 217 | CDRH2 | YINPSRGYTNYADSVKG |
| | 218 | CDRH3 | YYDDQYCLDY |
| | 219 | VL | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 220 | CDRL1 | RASQSVSYMN |
| | 221 | CDRL2 | DTSKVAS |
| | 222 | CDRL3 | QQWSSNPLT |
| | 223 | SCFv | DVQLVQSGAEVKKPGASVKVSCKASGYTFTRYTMHWVRQAPGQGL |
| | | | EWIGYINPSRGYTNYADSVKGRFTITTDKSTSTAYMELSSLRSED |
| | | | TATYYCARYYDDQYCLDYWGQGTTVTVSSSGGGGSGGGGSGGGGS |
| | | | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIK |
| | 224 | scFv | DIVLTQSPATLSLSPGERATLSCRASQSVSYMNWYQQKPGKAPKR |
| | | | WIYDTSKVASGVPARFSGSGSGTDYSLTINSLEAEDAATYYCQQW |
| | | | SSNPLTFGGGTKVEIKSGGGGSGGGGSGGGGSDVQLVQSGAEVKK |
| | | | PGASVKVSCKASGYTFTRYTMHWVRQAPGQGLEWIGYINPSRGYT |
| | | | NYADSVKGRFTITTDKSTSTAYMELSSLRSEDTATYYCARYYDDQ |
| | | | YCLDYWGQGTTVTVSS |

In particular embodiments, a non-viral membrane-bound tropism polypeptide comprises an extracellular antigen targeting domain comprising an anti-CD3p scFv that comprises an amino acid sequence set forth in any one of SEQ ID NOs: 153, 154, 163, 164, 173, 174, 183, 184, 193, 194, 203, 204, 213, 214, 223, and 224. In particular preferred embodiments, the anti-CD3p scFv comprises an amino acid sequence set forth in SEQ ID NO: 153 or 154. In particular embodiments, the anti-CD3p scFv comprises an amino acid sequence set forth in SEQ ID NO: 163 or 164. In particular embodiments, the anti-CD3p scFv comprises an amino acid sequence set forth in SEQ ID NO: 173 or 174. In particular embodiments, the anti-CD3p scFv comprises an amino acid sequence set forth in SEQ ID NO: 183 or 184. In particular embodiments, the anti-CD3p scFv comprises an amino acid sequence set forth in SEQ ID NO: 193 or 194. In particular embodiments, the anti-CD3p scFv comprises an amino acid sequence set forth in SEQ ID NO: 203 or 204. In particular embodiments, the anti-CD3p scFv comprises an amino acid sequence set forth in SEQ ID NO: 213 or 214. In particular embodiments, the anti-CD3p scFv comprises an amino acid sequence set forth in SEQ ID NO: 223 or 224.

Non-viral membrane-bound tropism polypeptides contemplated herein comprise, consist essentially of, or consist of an anti-CD3p scFv comprising an amino acid sequence set forth in any one of SEQ ID NOs: 153, 154, 163, 164, 173, 174, 183, 184, 193, 194, 203, 204, 213, 214, 223, and 224, a spacer domain comprising a hinge domain isolated from a polypeptide selected from the group consisting of CD4, CD8α, CD28, IgG4, or IgG1 and a transmembrane domain.

Non-viral membrane-bound tropism polypeptides contemplated herein comprise, consist essentially of, or consist of an anti-CD3ε scFv comprising an amino acid sequence set forth in any one of SEQ ID NOs: 153, 154, 163, 164, 173, 174, 183, 184, 193, 194, 203, 204, 213, 214, 223, and 224, a spacer domain comprising an amino acid sequence set forth in Table 6 and a transmembrane domain.

TABLE 6
Spacer Domains

| SEQ | |
|---|---|
| ID | |
| NO: | AMINO ACID SEQUENCE |
| 225 | TTTPAPRPPTPAPTIASQPLSLRPEACRPAAGGAVHTRGLDFACD |
| 226 | IEVMYPPPYLDNEKSNGTIIHVKGKHLCPSPLFPGPSKP |
| 227 | SGQVLLESNIKVLPTWSTPVQP |
| 228 | ESKYGPPCPPCP |
| 229 | ESKYGPPCPPCPAPPVAGPSVFLFPPKPKDTLMISRTPEVTCVVV |
| | DVSQEDPEVQFNWYVDGVEVHNAKTKPREEQFQSTYRVVSVLTVL |
| | HQDWLNGKEYKCKVSNKGLPSSIEKTISKAKGQPREPQVYTLPPS |
| | QEEMTKNQVSLTCLVKGFYPSDIAVEWESNGQPENNYKTTPPVLD |
| | SDGSFFLYSRLTVDKSRWQEGNVFSCSVMHEALHNHYTQKSLSLS |
| | LGK |
| 230 | LEPKSCDKTHTCPPCP |

Non-viral membrane-bound tropism polypeptides contemplated herein comprise, consist essentially of, or consist of an anti-CD3ε scFv comprising an amino acid sequence set forth in any one of SEQ ID NOs: 153, 154, 163, 164, 173, 174, 183, 184, 193, 194, 203, 204, 213, 214, 223, and 224, a spacer domain comprising an amino acid sequence set forth in any one of SEQ ID NOs: 225, 226, 227, 228, 229, and 230 and a transmembrane domain.

Non-viral membrane-bound tropism polypeptides contemplated herein comprise, consist essentially of, or consist of an anti-CD3ε scFv comprising an amino acid sequence set forth in any one of SEQ ID NOs: 153, 154, 163, 164, 173, 174, 183, 184, 193, 194, 203, 204, 213, 214, 223, and 224, a spacer domain comprising an amino acid sequence set forth in any one of SEQ ID NOs: 225, 226, 227, 228, 229, and 230 and a transmembrane domain isolated from a polypeptide selected from the group consisting of CD3c, CD4, CD8α, CD28, CD134, CD137, and CD278.

Non-viral membrane-bound tropism polypeptides contemplated herein comprise, consist essentially of, or consist of an anti-CD3ε scFv comprising an amino acid sequence set forth in any one of SEQ ID NOs: 153, 154, 163, 164, 173, 174, 183, 184, 193, 194, 203, 204, 213, 214, 223, and 224, a spacer domain comprising an amino acid sequence set forth in any one of SEQ ID NOs: 225, 226, 227, 228, 229, and 230 and a transmembrane domain comprising an amino acid sequence set forth in Table 7.

TABLE 7
Transmembrane Domains

| SEQ | |
|---|---|
| ID | |
| NO: | AMINO ACID SEQUENCE |
| 231 | IYIWAPLAGTCGVLLLSLVITLYC |
| 232 | IISFFLALTSTALLFLLFFLTLRFSVV |
| 233 | FWVLVVVGGVLACYSLLVTVAFIIFWV |
| 234 | VAAILGLGLVLGLLGPLAILL |
| 235 | WLPIGCAAFVVVCILGCILICWL |
| 236 | VMSVATIVIVDICITGGLLLLVYYWS |
| 237 | MALIVLGGVAGLLLFIGLGIFF |

Non-viral membrane-bound tropism polypeptides contemplated herein comprise, consist essentially of, or consist of an anti-CD3ε scFv comprising an amino acid sequence set forth in any one of SEQ ID NOs: 153, 154, 163, 164, 173, 174, 183, 184, 193, 194, 203, 204, 213, 214, 223, and 224, a spacer domain comprising an amino acid sequence set forth in any one of SEQ ID NOs: 225, 226, 227, 228, 229, and 230 and a transmembrane domain comprising an amino acid sequence set forth in any one of SEQ ID NOs: 231, 232, 2333, 234, 235, 236, and 237.

In particular embodiments, a non-viral membrane bound tropism polypeptide comprises, consists essentially of, or consists of an amino acid sequence set forth in Table 8.

TABLE 8
Non-viral membrane bound tropism polypeptides

| SEQ | |
|---|---|
| ID | |
| NO: | AMINO ACID SEQUENCE |
| 324 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYYDDHYCLDYWGQGTTVTVSSSGGGGSG |
| | GGGSGGGGSDIVLTQSPATLSLSPGERATLSCRASQSVSYMNWYQQKPGKAPKRWIYDTSKVA |
| | SGVPARFSGSGSGTDYSLTINSLEAEDAATYYCQQWSSNPLTFGGGTKVEIKTTTPAPRPPTP |
| | APTIASQPLSLRPEACRPAAGGAVHTRGLDFACDIYIWAPLAGTCGVLLLSLVITLYC |
| 325 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYYDDPYCLDYWGQGTTVTVSSSGGGGSG |
| | GGGSGGGGSDIVLTQSPATLSLSPGERATLSCRASQSVSYMNWYQQKPGKAPKRWIYDTSKVA |
| | SGVPARFSGSGSGTDYSLTINSLEAEDAATYYCQQWSSNPLTFGGGTKVEIKTTTPAPRPPTP |
| | APTIASQPLSLRPEACRPAAGGAVHTRGLDFACDIYIWAPLAGTCGVLLLSLVITLYC |
| 326 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYYNDQYCLDYWGQGTTVTVSSSSGGGGS |
| | GGGGSGGGGSDIVLTQSPATLSLSPGERATLSCRASQSVSYMNWYQQKPGKAPKRWIYDTSKV |
| | ASGVPARFSGSGSGTDYSLTINSLEAEDAATYYCQQWSSNPLTFGGGTKVEIKTTTPAPRPPT |
| | PAPTIASQPLSLRPEACRPAAGGAVHTRGLDFACDIYIWAPLAGTCGVLLLSLVITLYC |
| 327 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYYDAHNCLDYWGQGTTVTVSSSGGGGSG |
| | GGGSGGGGSDIVLTQSPATLSLSPGERATLSCRASQSVSYMNWYQQKPGKAPKRWIYDTSKVA |
| | SGVPARFSGSGSGTDYSLTINSLEAEDAATYYCQQWSSNPLTFGGGTKVEIKTTTPAPRPPTP |
| | APTIASQPLSLRPEACRPAAGGAVHTRGLDFACDIYIWAPLAGTCGVLLLSLVITLYC |
| 328 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYSDDHYCLDYWGQGTTVTVSSSGGGGSG |
| | GGGSGGGGSDIVLTQSPATLSLSPGERATLSCRASQSVSYMNWYQQKPGKAPKRWIYDTSKVA |
| | SGVPARFSGSGSGTDYSLTINSLEAEDAATYYCQQWSSNPLTFGGGTKVEIKTTTPAPRPPTP |
| | APTIASQPLSLRPEACRPAAGGAVHTRGLDFACDIYIWAPLAGTCGVLLLSLVITLYC |
| 329 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYSDDRYCLDYWGQGTTVTVSSSGGGGSG |
| | GGGSGGGGSDIVLTQSPATLSLSPGERATLSCRASQSVSYMNWYQQKPGKAPKRWIYDTSKVA |
| | SGVPARFSGSGSGTDYSLTINSLEAEDAATYYCQQWSSNPLTFGGGTKVEIKTTTPAPRPPTP |
| | APTIASQPLSLRPEACRPAAGGAVHTRGLDFACDIYIWAPLAGTCGVLLLSLVITLYC |
| 330 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYYDDNYCLDYWGQGTTVTVSSSSGGGGS |
| | GGGGSGGGGSDIVLTQSPATLSLSPGERATLSCRASQSVSYMNWYQQKPGKAPKRWIYDTSKV |
| | ASGVPARFSGSGSGTDYSLTINSLEAEDAATYYCQQWSSNPLTFGGGTKVEIKTTTPAPRPPT |
| | PAPTIASQPLSLRPEACRPAAGGAVHTRGLDFACDIYIWAPLAGTCGVLLLSLVITLYC |
| 331 | DVQLVQSGAEVKKPGASVKVSCKASGYTFTRYTMHWVRQAPGQGLEWIGYINPSRGYTNYADS |
| | VKGRFTITTDKSTSTAYMELSSLRSEDTATYYCARYYDDQYCLDYWGQGTTVTVSSSGGGGSG |
| | GGGSGGGGSDIVLTQSPATLSLSPGERATLSCRASQSVSYMNWYQQKPGKAPKRWIYDTSKVA |
| | SGVPARFSGSGSGTDYSLTINSLEAEDAATYYCQQWSSNPLTFGGGTKVEIKTTTPAPRPPTP |
| | APTIASQPLSLRPEACRPAAGGAVHTRGLDFACDIYIWAPLAGTCGVLLLSLVITLYC |

In particular embodiments, a non-viral membrane bound tropism polypeptide comprises an amino acid sequence set forth in any one of SEQ ID NOs: 324, 325, 326, 327, 328, 329, 330, and 331, preferably SEQ ID NO: 324.

In particular embodiments, a non-viral membrane bound tropism polypeptide consists essentially of an amino acid sequence set forth in any one of SEQ ID NOs: 324, 325, 326, 327, 328, 329, 330, and 331.

In particular embodiments, a non-viral membrane bound tropism polypeptide consists of an amino acid sequence set forth in any one of SEQ ID NOs: 324, 325, 326, 327, 328, 329, 330, and 331.

### E. Chimeric Antigen Receptors

Recombinant particles contemplated herein are engineered to efficiently deliver one or more copies of a vector encoding or comprising a promotor operably linked to a polynucleotide encoding an anti-BCMA chimeric antigen receptor. Chimeric antigen receptors (CARs) contemplated herein are fusion polypeptides that exploit antibody-based specificity for BCMA to redirect immune effector cell specificity thereby triggering proliferation, cytokine production, phagocytosis or production of molecules that can mediate cell death of BCMA-expressing cells in a major histocompatibility (MHC) independent manner. As used herein, the term “chimeric” refers to a molecule that is composed of two or more polypeptides, or polynucleotides, of different origins.

The present disclosure contemplates improved anti-BCMA CARs that are suitable for in vivo modification, or ex vivo manufacture, of immune effector cells to redirect cytotoxicity toward BCMA-expressing cells (e.g., B cells, plasma cells).

In various embodiments, a CAR comprises an anti-BCMA scFv or VHH; a hinge domain; a transmembrane domain; one or more costimulatory signaling domains; and a primary signaling domain.

In particular embodiments, a CAR comprises an extracellular antigen binding domain that comprises an antibody or antigen binding fragment thereof that specifically binds to a human BCMA polypeptide. The term “binding domain” or “extracellular antigen binding domain” are used interchangeably and refer to one or more antibodies or antigen binding fragments thereof that bind BCMA. The binding domain may be derived either from a natural, synthetic, semi-synthetic, or recombinant source.

B cell maturation antigen (BCMA) is a member of the tumor necrosis factor receptor superfamily 17 (TNFRSF17) and is highly expressed on the plasma cells of multiple myeloma (MM) patients. The restricted expression of BCMA makes it a suitable therapeutic target for treating multiple myeloma. The present disclosure contemplates antibodies and antigen binding fragments thereof that bind BCMA. An “antibody” refers to a polypeptide or antigen binding fragment thereof that comprises at least a light chain immunoglobulin variable region and/or a heavy chain immunoglobulin variable region, which specifically recognizes and binds one or more epitopes of a BCMA polypeptide, e.g., SEQ ID NO: 10

(MLQMAGQCSQNEYFDSLLHACIPCQLRCSSNTPPLTCQRYCNASVTNSV
KGTNAILWTCLGLSLIISLAVFVLMFLLRKINSEPLKDEFKNTGSGLLGM
ANIDLEKSRTGDEIILPRGLEYTVEECTCEDCIKSKPKVDSDHCFPLPAM
EEGATILVTTKTNDYCKSLPAALSATEIEKSISAR).

In various embodiments, a CAR comprises an anti-BCMA antibody or antigen binding fragment thereof comprising an amino acid sequence set forth in Table 9; a hinge domain; a transmembrane domain; one or more costimulatory signaling domains; and a primary signaling domain.

TABLE 9
anti-BCMA antibodies

| | SEQ | | |
|---|---|---|---|
| | ID | | |
| AB ID | NO | ID | AMINO ACID SEQUENCE |
| BCMA.1 | 11 | VH | QITLKESGPTLVKPTQTLTLTCTFSGFSLSTSGVGVGWIRQPPG |
| | | | KALEWLALIYWNDEKRYSPSLKSRLTITKDTSKNQVVLTMTNMD |
| | | | PVDTAVYYCARDEYGGFDIWGQGTMVTVSS |
| | 12 | CDRH1 | TSGVGVG |
| | 13 | CDRH2 | LIYWNDEKRYSPSLKS |
| | 14 | CDRH3 | DEYGGFDI |
| | 15 | VL | EIVLTQSPATLSLSPGERATLSCRASQSVSSYLAWYQQKPGQAP |
| | | | RLLIYDASNRATGIPARFSGSGSGTDFTLTISSLEPEDFAVYYC |
| | | | QQRVVYPITFGGGTKVEIK |
| | 16 | CDRL1 | RASQSVSSYLA |
| | 17 | CDRL2 | DASNRAT |
| | 18 | CDRL3 | QQRVVYPIT |
| | 19 | scFv | QITLKESGPTLVKPTQTLTLTCTFSGFSLSTSGVGVGWIRQPPG |
| | | | KALEWLALIYWNDEKRYSPSLKSRLTITKDTSKNQVVLTMTNMD |
| | | | PVDTAVYYCARDEYGGFDIWGQGTMVTVSSGGGGSGGGGSGGGG |
| | | | SGGGGSEIVLTQSPATLSLSPGERATLSCRASQSVSSYLAWYQQ |
| | | | KPGQAPRLLIYDASNRATGIPARFSGSGSGTDFTLTISSLEPED |
| | | | FAVYYCQQRVVYPITFGGGTKVEIK |
| | 20 | SCFv | EIVLTQSPATLSLSPGERATLSCRASQSVSSYLAWYQQKPGQAP |
| | | | RLLIYDASNRATGIPARFSGSGSGTDFTLTISSLEPEDFAVYYC |
| | | | QQRVVYPITFGGGTKVEIKGGGGSGGGGSGGGGSGGGGSQITLK |
| | | | ESGPTLVKPTQTLTLTCTFSGFSLSTSGVGVGWIRQPPGKALEW |
| | | | LALIYWNDEKRYSPSLKSRLTITKDTSKNQVVLTMTNMDPVDTA |
| | | | VYYCARDEYGGFDIWGQGTMVTVSS |
| BCMA.2 | 21 | VH | QITLKESGPTLVKPTQTLTLTCTFSGFSLSTSGVGVGWIRQPPG |
| | | | KALEWLALIYWNDDKRYSPSLKSRLTITKDTSKNQVVLTMTNMD |
| | | | PVDTAVYYCARDEYGGFDIWGQGTMVTVSS |
| | 22 | CDRH1 | TSGVGVG |
| | 23 | CDRH2 | LIYWNDDKRYSPSLKS |
| | 24 | CDRH3 | DEYGGFDI |
| | 25 | VL | EIVLTQSPATLSLSPGERATLSCRASQSVSSYLAWYQQKPGQAP |
| | | | RLLIYDASNRATGIPARFSGSGSGTDFTLTISSLEPEDFAVYYC |
| | | | QQRFDYPITFGGGTKVEIK |
| | 26 | CDRL1 | RASQSVSSYLA |
| | 27 | CDRL2 | DASNRAT |
| | 28 | CDRL3 | QQRFDYPIT |
| | 29 | scFv | QITLKESGPTLVKPTQTLTLTCTFSGFSLSTSGVGVGWIRQPPG |
| | | | KALEWLALIYWNDDKRYSPSLKSRLTITKDTSKNQVVLTMTNMD |
| | | | PVDTAVYYCARDEYGGFDIWGQGTMVTVSSGGGGSGGGGSGGGG |
| | | | SGGGGSEIVLTQSPATLSLSPGERATLSCRASQSVSSYLAWYQQ |
| | | | KPGQAPRLLIYDASNRATGIPARFSGSGSGTDFTLTISSLEPED |
| | | | FAVYYCQQRFDYPITFGGGTKVEIK |
| | 30 | scFv | EIVLTQSPATLSLSPGERATLSCRASQSVSSYLAWYQQKPGQAP |
| | | | RLLIYDASNRATGIPARFSGSGSGTDFTLTISSLEPEDFAVYYC |
| | | | QQRFDYPITFGGGTKVEIKGGGGSGGGGSGGGGSGGGGSQITLK |
| | | | ESGPTLVKPTQTLTLTCTFSGFSLSTSGVGVGWIRQPPGKALEW |
| | | | LALIYWNDDKRYSPSLKSRLTITKDTSKNQVVLTMTNMDPVDTA |
| | | | VYYCARDEYGGFDIWGQGTMVTVSS |
| BCMA.3 | 31 | VH | QVQLVESGGGVVQPGRSLRLSCAASGFTFSSYGMHWVRQAPGKG |
| | | | LEWVAVISYEGSNKYYADSVKGRFTISRDNSKNTLYLQMNSLRA |
| | | | EDTAVYYCARELGDGMDVWGQGTTVTVSS |
| | 32 | CDRH1 | SYGMH |
| | 33 | CDRH2 | VISYEGSNKYYADSVKG |
| | 34 | CDRH3 | ELGDGMDV |
| | 35 | VL | EIVLTQSPATLSLSPGERATLSCRASQSVSSYLAWFQQKPGQAP |
| | | | RLLIYDASNRATGIPARFSGSGSGTDFTLTISSLEPEDFAVYYC |
| | | | QQRVDLWTFGGGTKVEIK |
| | 36 | CDRL1 | RASQSVSSYLA |
| | 37 | CDRL2 | DASNRAT |
| | 38 | CDRL3 | QQRVDLWT |
| | 39 | scFv | QVQLVESGGGVVQPGRSLRLSCAASGFTFSSYGMHWVRQAPGKG |
| | | | LEWVAVISYEGSNKYYADSVKGRFTISRDNSKNTLYLQMNSLRA |
| | | | EDTAVYYCARELGDGMDVWGQGTTVTVSSGGGGSGGGGSGGGGS |
| | | | GGGGSEIVLTQSPATLSLSPGERATLSCRASQSVSSYLAWFQQK |
| | | | PGQAPRLLIYDASNRATGIPARFSGSGSGTDFTLTISSLEPEDF |
| | | | AVYYCQQRVDLWTFGGGTKVEIK |
| | 40 | scFv | EIVLTQSPATLSLSPGERATLSCRASQSVSSYLAWFQQKPGQAP |
| | | | RLLIYDASNRATGIPARFSGSGSGTDFTLTISSLEPEDFAVYYC |
| | | | QQRVDLWTFGGGTKVEIKGGGGSGGGGSGGGGSGGGGSQVQLVE |
| | | | SGGGVVQPGRSLRLSCAASGFTFSSYGMHWVRQAPGKGLEWVAV |
| | | | ISYEGSNKYYADSVKGRFTISRDNSKNTLYLQMNSLRAEDTAVY |
| | | | YCARELGDGMDVWGQGTTVTVSS |
| BCMA.4 | 41 | VH | EVQLVESGGGLVKPGGSLRLSCAASGFTFSDYYMSWIRQAPGKG |
| | | | LEWVSYISSSGSTIYYADSVKGRFTISRDNAKNSLYLQMNSLRA |
| | | | EDTAVYYCARDQGNYGVDVWGQGTTVTVSS |
| | 42 | CDRH1 | DYYMS |
| | 43 | CDRH2 | YISSSGSTIYYADSVKG |
| | 44 | CDRH3 | DQGNYGVDV |
| | 45 | VL | DIQMTQSPSTLSASVGDRVTITCRASQSISSWLAWYQQKPGKAP |
| | | | KLLIYDASSLESGVPSRFSGSGSGTEFTLTISSLQPDDFATYYC |
| | | | QQVSSLPPTFGGGTKVEIK |
| | 46 | CDRL1 | RASQSISSWLA |
| | 47 | CDRL2 | DASSLES |
| | 48 | CDRL3 | QQVSSLPPT |
| | 49 | scFv | EVQLVESGGGLVKPGGSLRLSCAASGFTFSDYYMSWIRQAPGKG |
| | | | LEWVSYISSSGSTIYYADSVKGRFTISRDNAKNSLYLQMNSLRA |
| | | | EDTAVYYCARDQGNYGVDVWGQGTTVTVSSGGGGSGGGGSGGGG |
| | | | SGGGGSDIQMTQSPSTLSASVGDRVTITCRASQSISSWLAWYQQ |
| | | | KPGKAPKLLIYDASSLESGVPSRFSGSGSGTEFTLTISSLQPDD |
| | | | FATYYCQQVSSLPPTFGGGTKVEIK |
| | 50 | scFv | DIQMTQSPSTLSASVGDRVTITCRASQSISSWLAWYQQKPGKAP |
| | | | KLLIYDASSLESGVPSRFSGSGSGTEFTLTISSLQPDDFATYYC |
| | | | QQVSSLPPTFGGGTKVEIKGGGGSGGGGSGGGGSGGGGSEVQLV |
| | | | ESGGGLVKPGGSLRLSCAASGFTFSDYYMSWIRQAPGKGLEWVS |
| | | | YISSSGSTIYYADSVKGRFTISRDNAKNSLYLQMNSLRAEDTAV |
| | | | YYCARDQGNYGVDVWGQGTTVTVSS |
| BCMA.5 | 51 | VH | QVQLVESGGGLVKPGGSLRLSCAASGFTFSDYYMSWIRQAPGKG |
| | | | LEWVSYISSSGSTIYYADSVKGRFTISRDNAKNSLYLQMNSLRA |
| | | | EDTAVYYCARDQGNYGVDVWGQGTTVTVSS |
| | 52 | CDRH1 | DYYMS |
| | 53 | CDRH2 | YISSSGSTIYYADSVKG |
| | 54 | CDRH3 | DQGNYGVDV |
| | 55 | VL | DIQMTQSPSTLSASVGDRVTITCRASQSISSWLAWYQQKPGKAP |
| | | | KLLIYEASSLESGVPSRFSGSGSGTEFTLTISSLQPDDFATYYC |
| | | | QQSDSHPITFGGGTKVEIK |
| | 56 | CDRL1 | RASQSISSWLA |
| | 57 | CDRL2 | EASSLES |
| | 58 | CDRL3 | QQSDSHPIT |
| | 59 | scFv | QVQLVESGGGLVKPGGSLRLSCAASGFTFSDYYMSWIRQAPGKG |
| | | | LEWVSYISSSGSTIYYADSVKGRFTISRDNAKNSLYLQMNSLRA |
| | | | EDTAVYYCARDQGNYGVDVWGQGTTVTVSSGGGGSGGGGSGGGG |
| | | | SGGGGSDIQMTQSPSTLSASVGDRVTITCRASQSISSWLAWYQQ |
| | | | KPGKAPKLLIYEASSLESGVPSRFSGSGSGTEFTLTISSLQPDD |
| | | | FATYYCQQSDSHPITFGGGTKVEIK |
| | 60 | scFv | DIQMTQSPSTLSASVGDRVTITCRASQSISSWLAWYQQKPGKAP |
| | | | KLLIYEASSLESGVPSRFSGSGSGTEFTLTISSLQPDDFATYYC |
| | | | QQSDSHPITFGGGTKVEIKGGGGSGGGGSGGGGSGGGGSQVQLV |
| | | | ESGGGLVKPGGSLRLSCAASGFTFSDYYMSWIRQAPGKGLEWVS |
| | | | YISSSGSTIYYADSVKGRFTISRDNAKNSLYLQMNSLRAEDTAV |
| | | | YYCARDQGNYGVDVWGQGTTVTVSS |
| BCMA.6 | 61 | VH | EVQLVESGGGLVKPGGSLRLSCAASGFTFSDYYMSWIRQAPGKG |
| | | | LEWVSYISSSGSTIYYADSVKGRFTISRDNAKNSLYLQMNSLRA |
| | | | EDTAVYYCARDQGNYGVDVWGQGTTVTVSS |
| | 62 | CDRH1 | DYYMS |
| | 63 | CDRH2 | YISSSGSTIYYADSVKG |
| | 64 | CDRH3 | DQGNYGVDV |
| | 65 | VL | DIQMTQSPSTLSASVGDRVTITCRASQSISSWLAWYQQKPGKAP |
| | | | KLLIYEASSLESGVPSRFSGSGSGTEFTLTISSLQPDDFATYYC |
| | | | QQANSHPITFGGGTKVEIK |
| | 66 | CDRL1 | RASQSISSWLA |
| | 67 | CDRL2 | EASSLES |
| | 68 | CDRL3 | QQANSHPIT |
| | 69 | scFv | EVQLVESGGGLVKPGGSLRLSCAASGFTFSDYYMSWIRQAPGKG |
| | | | LEWVSYISSSGSTIYYADSVKGRFTISRDNAKNSLYLQMNSLRA |
| | | | EDTAVYYCARDQGNYGVDVWGQGTTVTVSSGGGGSGGGGSGGGG |
| | | | SGGGGSDIQMTQSPSTLSASVGDRVTITCRASQSISSWLAWYQQ |
| | | | KPGKAPKLLIYEASSLESGVPSRFSGSGSGTEFTLTISSLQPDD |
| | | | FATYYCQQANSHPITFGGGTKVEIK |
| | 70 | scFv | DIQMTQSPSTLSASVGDRVTITCRASQSISSWLAWYQQKPGKAP |
| | | | KLLIYEASSLESGVPSRFSGSGSGTEFTLTISSLQPDDFATYYC |
| | | | QQANSHPITFGGGTKVEIKGGGGSGGGGSGGGGSGGGGSEVQLV |
| | | | ESGGGLVKPGGSLRLSCAASGFTFSDYYMSWIRQAPGKGLEWVS |
| | | | YISSSGSTIYYADSVKGRFTISRDNAKNSLYLQMNSLRAEDTAV |
| | | | YYCARDQGNYGVDVWGQGTTVTVSS |
| BCMA.7 | 71 | VH | EVQLLESGGGLVQPGGSLRLSCAASGFTFSNYAMSWVRQAPGKG |
| | | | LEWVSAISGSGGSTYYADSVKGRFTISRDNSKNTLYLQMNSLRA |
| | | | EDTAVYYCARPGDGYYEGVYFDYWGQGTLVTVSS |
| | 72 | CDRH1 | NYAMS |
| | 73 | CDRH2 | AISGSGGSTYYADSVKG |
| | 74 | CDRH3 | PGDGYYEGVYFDY |
| | 75 | VL | DIQMTQSPSSLSASVGDRVTITCRASQSISSYLNWYQQKPGKAP |
| | | | KLLIYAASSLQSGVPSRFSGSGSGTDFTLTISSLQPEDFATYYC |
| | | | QQAHSSPITFGGGTKVEIK |
| | 76 | CDRL1 | RASQSISSYLN |
| | 77 | CDRL2 | AASSLQS |
| | 78 | CDRL3 | QQAHSSPIT |
| | 79 | scFv | EVQLLESGGGLVQPGGSLRLSCAASGFTFSNYAMSWVRQAPGKG |
| | | | LEWVSAISGSGGSTYYADSVKGRFTISRDNSKNTLYLQMNSLRA |
| | | | EDTAVYYCARPGDGYYEGVYFDYWGQGTLVTVSSGGGGSGGGGS |
| | | | GGGGSGGGGSDIQMTQSPSSLSASVGDRVTITCRASQSISSYLN |
| | | | WYQQKPGKAPKLLIYAASSLQSGVPSRFSGSGSGTDFTLTISSL |
| | | | QPEDFATYYCQQAHSSPITFGGGTKVEIK |
| | 80 | scFv | DIQMTQSPSSLSASVGDRVTITCRASQSISSYLNWYQQKPGKAP |
| | | | KLLIYAASSLQSGVPSRFSGSGSGTDFTLTISSLQPEDFATYYC |
| | | | QQAHSSPITFGGGTKVEIKGGGGSGGGGSGGGGSGGGGSEVQLL |
| | | | ESGGGLVQPGGSLRLSCAASGFTFSNYAMSWVRQAPGKGLEWVS |
| | | | AISGSGGSTYYADSVKGRFTISRDNSKNTLYLQMNSLRAEDTAV |
| | | | YYCARPGDGYYEGVYFDYWGQGTLVTVSS |
| BCMA.8 | 81 | VH | QITLKESGPTLVKPTQTLTLTCTFSGFSLSTSGVGVGWIRQPPG |
| | | | KALEWLALIYWNDEKRYSPSLKSRLTITKDTSKNQVVLTMTNMD |
| | | | PVDTAVYYCAREGSHDYKSSNWEDPWGQGTLVTVSS |
| | 82 | CDRH1 | TSGVGVG |
| | 83 | CDRH2 | LIYWNDEKRYSPSLKS |
| | 84 | CDRH3 | EGSHDYKSSNWFDP |
| | 85 | VL | DIQMTQSPSSLSASVGDRVTITCQASQDIANYLNWYQQKPGKAP |
| | | | KLLIYDASNLETGVPSRFSGSGSGTDFTFTISSLQPEDIATYYC |
| | | | QQHFNLPLTFGGGTKVEIK |
| | 86 | CDRL1 | QASQDIANYLN |
| | 87 | CDRL2 | DASNLET |
| | 88 | CDRL3 | QQHFNLPLTF |
| | 89 | scFv | QITLKESGPTLVKPTQTLTLTCTFSGFSLSTSGVGVGWIRQPPG |
| | | | KALEWLALIYWNDEKRYSPSLKSRLTITKDTSKNQVVLTMTNMD |
| | | | PVDTAVYYCAREGSHDYKSSNWFDPWGQGTLVTVSSGGGGSGGG |
| | | | GSGGGGSGGGGSDIQMTQSPSSLSASVGDRVTITCQASQDIANY |
| | | | LNWYQQKPGKAPKLLIYDASNLETGVPSRFSGSGSGTDFTFTIS |
| | | | SLQPEDIATYYCQQHFNLPLTFGGGTKVEIK |
| | 90 | scFv | DIQMTQSPSSLSASVGDRVTITCQASQDIANYLNWYQQKPGKAP |
| | | | KLLIYDASNLETGVPSRFSGSGSGTDFTFTISSLQPEDIATYYC |
| | | | QQHFNLPLTFGGGTKVEIKGGGGSGGGGSGGGGSGGGGSQITLK |
| | | | ESGPTLVKPTQTLTLTCTFSGFSLSTSGVGVGWIRQPPGKALEW |
| | | | LALIYWNDEKRYSPSLKSRLTITKDTSKNQVVLTMTNMDPVDTA |
| | | | VYYCAREGSHDYKSSNWFDPWGQGTLVTVSS |
| BCMA.9 | 91 | VH | EVQLVESGGGLVKPGGSLRLSCAASGFTFSSYSMNWVRQAPGKG |
| | | | LEWVSSISSSSSYIYYADSVKGRFTISRDNAKNSLYLQMNSLRA |
| | | | EDTAVYYCARAGDTYSAADYYYMDVWGKGTTVTVSS |
| | 92 | CDRH1 | SYSMN |
| | 93 | CDRH2 | SISSSSSYIYYADSVKG |
| | 94 | CDRH3 | AGDTYSAADYYYMDV |
| | 95 | VL | DIVMTQSPLSLPVTPGEPASISCRSSQSLLHSNGYNYLDWYLQK |
| | | | PGQSPQLLIYLGSNRASGVPDRFSGSGSGTDFTLKISRVEAEDV |
| | | | GVYYCMQALGLITFGGGTKVEIK |
| | 96 | CDRL1 | RSSQSLLHSNGYNYLD |
| | 97 | CDRL2 | LGSNRAS |
| | 98 | CDRL3 | MQALGLIT |
| | 99 | scFv | EVQLVESGGGLVKPGGSLRLSCAASGFTFSSYSMNWVRQAPGKG |
| | | | LEWVSSISSSSSYIYYADSVKGRFTISRDNAKNSLYLQMNSLRA |
| | | | EDTAVYYCARAGDTYSAADYYYMDVWGKGTTVTVSSGGGGSGGG |
| | | | GSGGGGSGGGGSDIVMTQSPLSLPVTPGEPASISCRSSQSLLHS |
| | | | NGYNYLDWYLQKPGQSPQLLIYLGSNRASGVPDRFSGSGSGTDF |
| | | | TLKISRVEAEDVGVYYCMQALGLITFGGGTKVEIK |
| | 100 | scFv | DIVMTQSPLSLPVTPGEPASISCRSSQSLLHSNGYNYLDWYLQK |
| | | | PGQSPQLLIYLGSNRASGVPDRFSGSGSGTDFTLKISRVEAEDV |
| | | | GVYYCMQALGLITFGGGTKVEIKGGGGSGGGGSGGGGSGGGGSE |
| | | | VQLVESGGGLVKPGGSLRLSCAASGFTFSSYSMNWVRQAPGKGL |
| | | | EWVSSISSSSSYIYYADSVKGRFTISRDNAKNSLYLQMNSLRAE |
| | | | DTAVYYCARAGDTYSAADYYYMDVWGKGTTVTVSS |
| BCMA.10 | 101 | VHH | EVQLLESGGGLVQPGGSLRLSCAASGFTFGSEAMSWVRQAPGKE |
| | | | RELVSAISGSGEVTYYADSVKGRFTISRDNSKNTLYLQMNSLRA |
| | | | EDTAVYYCQRLVEAKRHWGQGTQVTVSS |
| | 102 | CDRH1 | SEAMS |
| | 103 | CDRH2 | AISGSGEVTYYADSVKG |
| | 104 | CDRH3 | LVEAKRH |
| BCMA.11 | 105 | VHH | EVQLLESGGGLVQPGGSLRLSCAASGFTFESEAMSWYRQAPGKE |
| | | | RELVSVITSEGSTYYADSVKGRFTISRDNSKNTLYLQMNSLRAE |
| | | | DTAVYYCAHIEWETRLNWGQGTQVTVSS |
| | 106 | CDRH1 | SEAMS |
| | 107 | CDRH2 | VITSEGSTYYADSVKG |
| | 108 | CDRH3 | IEWETRLN |
| BCMA.12 | 109 | VHH | EVQLLESGGGLVQPGGSLRLSCAASGFTFDEYTMHWFRQAPGKE |
| | | | REFVSAISGGGSETYYADSVKGRFTISRDNSKNTLYLQMNSLRA |
| | | | EDTAVYYCAAGGEEAGVGYWGQGTQVTVSS |
| | 110 | CDRH1 | EYTMH |
| | 111 | CDRH2 | AISGGGSETYYADSVKG |
| | 112 | CDRH3 | GGEEAGVGY |
| BCMA.13 | 113 | VHH | EVQLLESGGGLVQPGGSLRLSCAASGFTFEDYAMSWFRQAPGKE |
| | | | REGVSAISGKGGSTYYADSVKGRFTISRDNSKNTLYLQMNSLRA |
| | | | EDTAVYYCAVLDEEAGAEGGYWGQGTQVTVSS |
| | 114 | CDRH1 | DYAMS |
| | 115 | CDRH2 | AISGKGGSTYYADSVKG |
| | 116 | CDRH3 | LDEEAGAEGGY |
| BCMA.14 | 117 | VHH | EVQLLESGGGLVQPGGSLRLSCAASGFTFDRYAMSWFRQAPGKE |
| | | | REGVSAISTSGDSTYYADSVKGRFTISRDNSKNTLYLQMNSLRA |
| | | | EDTAVYYCAVLDEEAGAEGGYWGQGTQVTVSS |
| | 118 | CDRH1 | RYAMS |
| | 119 | CDRH2 | AISTSGDSTYYADSVKG |
| | 120 | CDRH3 | LDEEAGAEGGY |
| BCMA.15 | 121 | VHH | EVQLLESGGGLVQPGGSLRLSCAASGFTFASDAMSWYRQAPGKE |
| | | | RELVSAISGSGGSTYYADSVKGRFTISRDNSKNTLYLQMNSLRA |
| | | | EDTAVYYCAAHDSGEAYLAFDYWGQGTQVTVSS |
| | 122 | CDRH1 | SDAMS |
| | 123 | CDRH2 | AISGSGGSTYYADSVKG |
| | 124 | CDRH3 | HDSGEAYLAFDY |
| BCMA.16 | 125 | VHH | EVQLLESGGGLVQPGGSLRLSCAASGFTFDSYTMSWYRQAPGKE |
| | | | RELVSAISGHGDSTYYADSVKGRFTISRDNSKNTLYLQMNSLRA |
| | | | EDTAVYYCTRISITTEWLAGDYWGQGTQVTVSS |
| | 126 | CDRH1 | SYTMS |
| | 127 | CDRH2 | AISGHGDSTYYADSVKG |
| | 128 | CDRH3 | ISITTEWLAGDY |
| BCMA.17 | 129 | VHH | EVQLLESGGGLVQPGGSLRLSCAASGFTFSSYAMSWFRQAPGKE |
| | | | REFVSFISGSGDSTYYADSVKGRFTISRDNSKNTLYLQMNSLRA |
| | | | EDTAVYYCTRWPYDFEEPSEPGVYWGQGTQVTVSS |
| | 130 | CDRH1 | SYAMS |
| | 131 | CDRH2 | FISGSGDSTYYADSVKG |
| | 132 | CDRH3 | WPYDFEEPSEPGVY |
| BCMA.18 | 133 | VHH | EVQLLESGGGLVQPGGSLRLSCAASGFTFTDYDMSWYRQAPGKE |
| | | | RELVSVIHSGGSTYYADSVKGRFTISRDNSKNTLYLQMNSLRAE |
| | | | DTAVYYCAPGYYSDLSFDYYNFDYWGQGTQVTVSS |
| | 134 | CDRH1 | DYDMS |
| | 135 | CDRH2 | VIHSGGSTYYADSVKG |
| | 136 | CDRH3 | GYYSDLSFDYYNFDY |
| BCMA.19 | 137 | VHH | EVQLLESGGGLVQPGGSLRLSCAASGFTFSDYAMHWFRQAPGKE |
| | | | RVLVSSIDSGGSTYYADSVKGRFTISRDNSKNTLYLQMNSLRAE |
| | | | DTAVYYCNAGFKGDHPHPKDAFDIWGQGTQVTVSS |
| | 138 | CDRH1 | DYAMH |
| | 139 | CDRH2 | SIDSGGSTYYADSVKG |
| | 140 | CDRH3 | GFKGDHPHPKDAFDI |
| BCMA.20 | 141 | VHH | EVQLLESGGGLVQPGGSLRLSCAASGFTFSSEGMSWVRQAPGKE |
| | | | RELVSAISGSGDHTYYADSVRGRFTISRDNSKNTLYLQMNSLRA |
| | | | EDTAVYYCNALEGGPTTAIQPGGPDYWGQGTQVTVSS |
| | 142 | CDRH1 | SEGMS |
| | 143 | CDRH2 | AISGSGDHTYYADSVRG |
| | 144 | CDRH3 | LEGGPTTAIQPGGPDY |

In particular embodiments, a CAR comprises a binding domain that comprises an anti-BCMA scFv comprising an amino acid sequence set forth in any one of SEQ ID NOs: 19, 20, 29, 30, 39, 40, 49, 50, 59, 60, 69, 70, 79, 80, 89, 90, 99, and 100; a hinge domain; a transmembrane domain; one or more costimulatory signaling domains; and a primary signaling domain. In particular embodiments, a CAR comprises a binding domain that comprises an anti-BCMA scFv comprising an amino acid sequence set forth in any one of SEQ ID NOs: 20, 30, 39, 50, 59, 70, 80, 90, and 100; a hinge domain; a transmembrane domain; one or more costimulatory signaling domains; and a primary signaling domain. In particular embodiments, a CAR comprises a binding domain that comprises an anti-BCMA scFv comprising an amino acid sequence set forth in any one of SEQ ID NOs: 39, 59, 70, and 90; a hinge domain; a transmembrane domain; one or more costimulatory signaling domains; and a primary signaling domain.

In particular embodiments, a CAR comprises a binding domain that comprises an anti-BCMA VHH comprising an amino acid sequence set forth in any one of SEQ ID NOs: 101, 105, 109, 113, 117, 121, 125, 129, 133, 137, and 141; a hinge domain; a transmembrane domain; one or more costimulatory signaling domains; and a primary signaling domain. In particular embodiments, a CAR comprises a binding domain that comprises an anti-BCMA VHH comprising an amino acid sequence set forth in SEQ ID NO: 101 or 117; a hinge domain; a transmembrane domain; one or more costimulatory signaling domains; and a primary signaling domain.

Chimeric antigen receptors contemplated herein comprise a hinge domain disposed between the extracellular antigen binding domain and the transmembrane domain of a CAR. A hinge domain plays a role in positioning the extracellular antigen binding domain away from the effector cell surface to enable proper cell/cell contact, antigen binding and activation. A hinge domain may be derived from a naturally occurring polypeptide or from a synthetic, semi-synthetic, or recombinant source.

In particular embodiments, a CAR comprises a binding domain that comprises an anti-BCMA scFv comprising an amino acid sequence set forth in any one of SEQ ID NOs: 19, 20, 29, 30, 39, 40, 49, 50, 59, 60, 69, 70, 79, 80, 89, 90, 99, and 100; a hinge domain isolated from CD4, CD8α, CD28, IgG1, and IgG4; a transmembrane domain; one or more costimulatory signaling domains; and a primary signaling domain. In particular embodiments, a CAR comprises a binding domain that comprises an anti-BCMA scFv comprising an amino acid sequence set forth in any one of SEQ ID NOs: 39, 59, 70, and 90; a hinge domain comprising an amino acid sequence set forth in any one of SEQ ID NOs: 225, 226, 227, 228, 229 and 230; a transmembrane domain; one or more costimulatory signaling domains; and a primary signaling domain.

In particular embodiments, a CAR comprises a binding domain that comprises an anti-BCMA VHH comprising an amino acid sequence set forth in any one of SEQ ID NOs: 101, 105, 109, 113, 117, 121, 125, 129, 133, 137, and 141; a hinge domain isolated from CD4, CD8α, CD28, IgG1, and IgG4; a transmembrane domain; one or more costimulatory signaling domains; and a primary signaling domain. In particular embodiments, a CAR comprises a binding domain that comprises an anti-BCMA VHH comprising an amino acid sequence set forth in SEQ ID NO: 101 or 117; a hinge domain comprising an amino acid sequence set forth in any one of SEQ ID NOs: 225, 226, 227, 228, 229 and 230; a transmembrane domain; one or more costimulatory signaling domains; and a primary signaling domain.

Chimeric antigen receptors contemplated herein comprise a transmembrane domain. The transmembrane domain is a hydrophobic domain that fuses the extracellular and intracellular portions of the CAR and anchors the CAR to the plasma membrane of the immune effector cell. The transmembrane domain may be derived either from a natural, synthetic, semi-synthetic, or recombinant source. In particular embodiments, the CAR further comprises a short oligo- or polypeptide linker, preferably between 1, 2, 3, 4, 5, 6, 7, 8, 9, or 10 amino acids in length disposed between the transmembrane domain and the intracellular domains of the CAR.

In particular embodiments, a CAR comprises a binding domain that comprises an anti-BCMA scFv comprising an amino acid sequence set forth in any one of SEQ ID NOs: 19, 20, 29, 30, 39, 40, 49, 50, 59, 60, 69, 70, 79, 80, 89, 90, 99, and 100; a hinge domain isolated from CD4, CD8α, CD28, IgG1, and IgG4; a transmembrane domain isolated or derived from a polypeptide selected from the group consisting of CD3ε, CD4, CD8α, CD28, CD33, CD134, CD137, and CD278; one or more costimulatory signaling domains; and a primary signaling domain. In particular embodiments, a CAR comprises a binding domain that comprises an anti-BCMA scFv comprising an amino acid sequence set forth in any one of SEQ ID NOs: 39, 59, 70, and 90; a hinge domain comprising an amino acid sequence set forth in any one of SEQ ID NOs: 225, 227, 228, 229, and 230; a transmembrane domain comprising an amino acid sequence set forth in any one of SEQ ID NOs: 231, 323, 233, 234, 235, 236, and 237; one or more costimulatory signaling domains; and a primary signaling domain.

In particular embodiments, a CAR comprises a binding domain that comprises an anti-BCMA VHH comprising an amino acid sequence set forth in any one of SEQ ID NOs: 101, 105, 109, 113, 117, 121, 125, 129, 133, 137, and 141; a hinge domain isolated from CD4, CD8α, CD28, IgG1, and IgG4; a transmembrane domain isolated or derived from a polypeptide selected from the group consisting of CD3ε, CD4, CD8α, CD28, CD33, CD134, CD137, and CD278; one or more costimulatory signaling domains; and a primary signaling domain. In particular embodiments, a CAR comprises a binding domain that comprises an anti-BCMA VHH comprising an amino acid sequence set forth in SEQ ID NO: 101 or 117; a hinge domain comprising an amino acid sequence set forth in any one of SEQ ID NOs: 225, 227, 228, 229, and 230; a transmembrane domain comprising an amino acid sequence set forth in any one of SEQ ID NOs: 231, 323, 233, 234, 235, 236, and 237; one or more costimulatory signaling domains; and a primary signaling domain.

Chimeric antigen receptors contemplated herein comprise on or more intracellular signaling domains that function to transduce a signal of extracellular antigen recognition to the interior of the immune effector cell and elicit one or more effector cell functions including but not limited to activation, cytokine production, proliferation and cytotoxic activity. T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. The intracellular primary signaling and costimulatory signaling domains may be linked in any order in tandem to the carboxyl terminus of the transmembrane domain.

In particular embodiments, a CAR comprises a binding domain that comprises an anti-BCMA scFv comprising an amino acid sequence set forth in any one of SEQ ID NOs: 19, 20, 29, 30, 39, 40, 49, 50, 59, 60, 69, 70, 79, 80, 89, 90, 99, and 100; a hinge domain isolated from CD4, CD8α, CD28, IgG1, and IgG4; a transmembrane domain isolated or derived from a polypeptide selected from the group consisting of CD3ε, CD4, CD8α, CD28, CD33, CD134, CD137, and CD278; a costimulatory signaling domain isolated or derived from a polypeptide selected from the group consisting of CD27, CD28, CD134, CD137, CD278, and TNRF2; and a CD3 ζ primary signaling domain. In particular embodiments, a CAR comprises a binding domain that comprises an anti-BCMA scFv comprising an amino acid sequence set forth in any one of SEQ ID NOs: 39, 59, 70, and 90; a hinge domain comprising an amino acid sequence set forth in any one of SEQ ID NOs: 225, 227, 228, 229, and 230; a transmembrane domain comprising an amino acid sequence set forth in any one of SEQ ID NOs: 231, 323, 233, 234, 235, 236, and 237; a costimulatory signaling domain comprising an amino acid sequence set forth in any one of SEQ ID NOs: 239, 240, 241, 242, 243, and 244; and a primary signaling domain comprising an amino acid sequence set forth in SEQ ID NO: 238.

In particular embodiments, a CAR comprises a binding domain that comprises an anti-BCMA VHH comprising an amino acid sequence set forth in any one of SEQ ID NOs: 101, 105, 109, 113, 117, 121, 125, 129, 133, 137, and 141; a hinge domain isolated from CD4, CD8α, CD28, IgG1, and IgG4; a transmembrane domain isolated or derived from a polypeptide selected from the group consisting of CD3ε, CD4, CD8α, CD28, CD33, CD134, CD137, and CD278; a costimulatory signaling domain isolated or derived from a polypeptide selected from the group consisting of CD27, CD28, CD134, CD137, CD278, and TNRF2; and a CD3ζ primary signaling domain. In particular embodiments, a CAR comprises a binding domain that comprises an anti-BCMA VHH comprising an amino acid sequence set forth in SEQ ID NO: 101 or 117; a hinge domain comprising an amino acid sequence set forth in any one of SEQ ID NOs: 225, 227, 228, 229, and 230; a transmembrane domain comprising an amino acid sequence set forth in any one of SEQ ID NOs: 231, 323, 233, 234, 235, 236, and 237; a costimulatory signaling domain comprising an amino acid sequence set forth in any one of SEQ ID NOs: 239, 240, 241, 242, 243, and 244; and a primary signaling domain comprising an amino acid sequence set forth in SEQ ID NO: 238.

TABLE 10

| SEQ ID | | |
|---|---|---|
| NO: | DOMAIN | AMINO ACID SEQUENCE |
| 238 | PRIMARY | RVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATK |
| | | DTYDALHMQALPPR |
| 239 | COSTIM | KRGRKKLLYIFKQPFMRPVQTTQEEDGCSCRFPEEEEGGCEL |
| 240 | COSTIM | RSKRSRLLHSDYMNMTPRRPGPTRKHYQPYAPPRDFAAYRS |
| 241 | COSTIM | ALYLLRRDQRLPPDAHKPPGGGSFRTPIQEEQADAHSTLAKI |
| 242 | COSTIM | TKKKYSSSVHDPNGEYMFMRAVNTAKKSRLTDVTL |
| 243 | COSTIM | QRRKYRSNKGESPVEPAEPCHYSCPREEEGSTIPIQEDYRKPEPACSP |
| 244 | COSTIM | KKKPLCLQREAKVPHLPADKARGTQGPEQQHLLITAPSSSSSSLESSAS |
| | | ALDRRAPTRNQPQAPGVEASGAGEARASTGSSDSSPGGHGTQVNVTCIV |
| | | NVCSSSDHSSQCSSQASSTMGDTDSSPSESPKDEQVPFSKEECAFRSQL |
| | | ETPETLLGSTEEKPLPLGVPDAGMKPS |

In particular embodiments, a CAR comprises an scFv or VHH comprising an amino acid sequence set forth in Table 9; a CD8α hinge, a CD8α transmembrane domain; a CD137 costimulatory signaling domain; and a CD3ζ primary signaling domain.

In particular embodiments, a CAR comprises a binding domain that comprises an anti-BCMA scFv comprising an amino acid sequence set forth in any one of SEQ ID NOs: 19, 20, 29, 30, 39, 40, 49, 50, 59, 60, 69, 70, 79, 80, 89, 90, 99, and 100; a CD8α hinge, a CD8α transmembrane domain; a CD137 costimulatory signaling domain; and a CD3 primary signaling domain. In particular embodiments, a CAR comprises a binding domain that comprises an anti-BCMA scFv comprising an amino acid sequence set forth in any one of SEQ ID NOs: 39, 59, 70, and 90; a CD8α hinge, a CD8α transmembrane domain; a CD137 costimulatory signaling domain; and a CD3 ζ primary signaling domain.

In particular embodiments, a CAR comprises a binding domain that comprises an anti-BCMA VHH comprising an amino acid sequence set forth in any one of SEQ ID NOs: 101, 105, 109, 113, 117, 121, 125, 129, 133, 137, and 141; a CD8α hinge, a CD8α transmembrane domain; a CD137 costimulatory signaling domain; and a CD3 ζ primary signaling domain. In particular embodiments, a CAR comprises a binding domain that comprises an anti-BCMA VHH comprising an amino acid sequence set forth in SEQ ID NO: 101 or 117; a CD8α hinge, a CD8α transmembrane domain; a CD137 costimulatory signaling domain; and a CD3ζ primary signaling domain.

In some embodiments, a CAR comprises an amino acid sequence set forth in Table 11.

TABLE 11
Chimeric antigen receptors

| SEQ | |
|---|---|
| ID | |
| NO. | AMINO ACID SEQUENCE |
| 255 | QITLKESGPTLVKPTQTLTLTCTFSGFSLSTSGVGVGWIRQPPGKALEWLALIYWNDEKRYSPS |
| | LKSRLTITKDTSKNQVVLTMTNMDPVDTAVYYCARDEYGGFDIWGQGTMVTVSSGGGGSGGGGS |
| | GGGGSGGGGSEIVLTQSPATLSLSPGERATLSCRASQSVSSYLAWYQQKPGQAPRLLIYDASNR |
| | ATGIPARFSGSGSGTDFTLTISSLEPEDFAVYYCQQRVVYPITFGGGTKVEIKTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 256 | EIVLTQSPATLSLSPGERATLSCRASQSVSSYLAWYQQKPGQAPRLLIYDASNRATGIPARFSG |
| | SGSGTDFTLTISSLEPEDFAVYYCQQRVVYPITFGGGTKVEIKGGGGSGGGGSGGGGSGGGGSQ |
| | ITLKESGPTLVKPTQTLTLTCTFSGFSLSTSGVGVGWIRQPPGKALEWLALIYWNDEKRYSPSL |
| | KSRLTITKDTSKNQVVLTMTNMDPVDTAVYYCARDEYGGFDIWGQGTMVTVSSTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 257 | QITLKESGPTLVKPTQTLTLTCTFSGFSLSTSGVGVGWIRQPPGKALEWLALIYWNDDKRYSPS |
| | LKSRLTITKDTSKNQVVLTMTNMDPVDTAVYYCARDEYGGFDIWGQGTMVTVSSGGGGSGGGGS |
| | GGGGSGGGGSEIVLTQSPATLSLSPGERATLSCRASQSVSSYLAWYQQKPGQAPRLLIYDASNR |
| | ATGIPARFSGSGSGTDFTLTISSLEPEDFAVYYCQQRFDYPITFGGGTKVEIKTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 258 | EIVLTQSPATLSLSPGERATLSCRASQSVSSYLAWYQQKPGQAPRLLIYDASNRATGIPARFSG |
| | SGSGTDFTLTISSLEPEDFAVYYCQQRFDYPITFGGGTKVEIKGGGGSGGGGSGGGGSGGGGSQ |
| | ITLKESGPTLVKPTQTLTLTCTFSGFSLSTSGVGVGWIRQPPGKALEWLALIYWNDDKRYSPSL |
| | KSRLTITKDTSKNQVVLTMTNMDPVDTAVYYCARDEYGGFDIWGQGTMVTVSSTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 259 | QVQLVESGGGVVQPGRSLRLSCAASGFTFSSYGMHWVRQAPGKGLEWVAVISYEGSNKYYADSV |
| | KGRFTISRDNSKNTLYLQMNSLRAEDTAVYYCARELGDGMDVWGQGTTVTVSSGGGGSGGGGSG |
| | GGGSGGGGSEIVLTQSPATLSLSPGERATLSCRASQSVSSYLAWFQQKPGQAPRLLIYDASNRA |
| | TGIPARFSGSGSGTDFTLTISSLEPEDFAVYYCQQRVDLWTFGGGTKVEIKTTTPAPRPPTPAP |
| | TIASQPLSLRPEACRPAAGGAVHTRGLDFACDIYIWAPLAGTCGVLLLSLVITLYCKRGRKKLL |
| | YIFKQPFMRPVQTTQEEDGCSCRFPEEEEGGCELRVKFSRSADAPAYQQGQNQLYNELNLGRRE |
| | EYDVLDKRRGRDPEMGGKPRRKNPQEGLYNELQKDKMAEAYSEIGMKGERRRGKGHDGLYQGLS |
| | TATKDTYDALHMQALPPR |
| 260 | EIVLTQSPATLSLSPGERATLSCRASQSVSSYLAWFQQKPGQAPRLLIYDASNRATGIPARFSG |
| | SGSGTDFTLTISSLEPEDFAVYYCQQRVDLWTFGGGTKVEIKGGGGSGGGGSGGGGSGGGGSQV |
| | QLVESGGGVVQPGRSLRLSCAASGFTFSSYGMHWVRQAPGKGLEWVAVISYEGSNKYYADSVKG |
| | RFTISRDNSKNTLYLQMNSLRAEDTAVYYCARELGDGMDVWGQGTTVTVSSTTTPAPRPPTPAP |
| | TIASQPLSLRPEACRPAAGGAVHTRGLDFACDIYIWAPLAGTCGVLLLSLVITLYCKRGRKKLL |
| | YIFKQPFMRPVQTTQEEDGCSCRFPEEEEGGCELRVKFSRSADAPAYQQGQNQLYNELNLGRRE |
| | EYDVLDKRRGRDPEMGGKPRRKNPQEGLYNELQKDKMAEAYSEIGMKGERRRGKGHDGLYQGLS |
| | TATKDTYDALHMQALPPR |
| 261 | EVQLVESGGGLVKPGGSLRLSCAASGFTFSDYYMSWIRQAPGKGLEWVSYISSSGSTIYYADSV |
| | KGRFTISRDNAKNSLYLQMNSLRAEDTAVYYCARDQGNYGVDVWGQGTTVTVSSGGGGSGGGGS |
| | GGGGSGGGGSDIQMTQSPSTLSASVGDRVTITCRASQSISSWLAWYQQKPGKAPKLLIYDASSL |
| | ESGVPSRFSGSGSGTEFTLTISSLQPDDFATYYCQQVSSLPPTFGGGTKVEIKTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 262 | DIQMTQSPSTLSASVGDRVTITCRASQSISSWLAWYQQKPGKAPKLLIYDASSLESGVPSRFSG |
| | SGSGTEFTLTISSLQPDDFATYYCQQVSSLPPTFGGGTKVEIKGGGGSGGGGSGGGGSGGGGSE |
| | VQLVESGGGLVKPGGSLRLSCAASGFTFSDYYMSWIRQAPGKGLEWVSYISSSGSTIYYADSVK |
| | GRFTISRDNAKNSLYLQMNSLRAEDTAVYYCARDQGNYGVDVWGQGTTVTVSSTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 263 | QVQLVESGGGLVKPGGSLRLSCAASGFTFSDYYMSWIRQAPGKGLEWVSYISSSGSTIYYADSV |
| | KGRFTISRDNAKNSLYLQMNSLRAEDTAVYYCARDQGNYGVDVWGQGTTVTVSSGGGGSGGGGS |
| | GGGGSGGGGSDIQMTQSPSTLSASVGDRVTITCRASQSISSWLAWYQQKPGKAPKLLIYEASSL |
| | ESGVPSRFSGSGSGTEFTLTISSLQPDDFATYYCQQSDSHPITFGGGTKVEIKTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 264 | DIQMTQSPSTLSASVGDRVTITCRASQSISSWLAWYQQKPGKAPKLLIYEASSLESGVPSRFSG |
| | SGSGTEFTLTISSLQPDDFATYYCQQSDSHPITFGGGTKVEIKGGGGSGGGGSGGGGSGGGGSQ |
| | VQLVESGGGLVKPGGSLRLSCAASGFTFSDYYMSWIRQAPGKGLEWVSYISSSGSTIYYADSVK |
| | GRFTISRDNAKNSLYLQMNSLRAEDTAVYYCARDQGNYGVDVWGQGTTVTVSSTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 265 | EVQLVESGGGLVKPGGSLRLSCAASGFTFSDYYMSWIRQAPGKGLEWVSYISSSGSTIYYADSV |
| | KGRFTISRDNAKNSLYLQMNSLRAEDTAVYYCARDQGNYGVDVWGQGTTVTVSSGGGGSGGGGS |
| | GGGGSGGGGSDIQMTQSPSTLSASVGDRVTITCRASQSISSWLAWYQQKPGKAPKLLIYEASSL |
| | ESGVPSRFSGSGSGTEFTLTISSLQPDDFATYYCQQANSHPITFGGGTKVEIKTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 266 | DIQMTQSPSTLSASVGDRVTITCRASQSISSWLAWYQQKPGKAPKLLIYEASSLESGVPSRFSG |
| | SGSGTEFTLTISSLQPDDFATYYCQQANSHPITFGGGTKVEIKGGGGSGGGGSGGGGSGGGGSE |
| | VQLVESGGGLVKPGGSLRLSCAASGFTFSDYYMSWIRQAPGKGLEWVSYISSSGSTIYYADSVK |
| | GRFTISRDNAKNSLYLQMNSLRAEDTAVYYCARDQGNYGVDVWGQGTTVTVSSTTTPAPRPPTP |
| | APTIASQPLSLRPEACRPAAGGAVHTRGLDFACDIYIWAPLAGTCGVLLLSLVITLYCKRGRKK |
| | LLYIFKQPFMRPVQTTQEEDGCSCRFPEEEEGGCELRVKFSRSADAPAYQQGQNQLYNELNLGR |
| | REEYDVLDKRRGRDPEMGGKPRRKNPQEGLYNELQKDKMAEAYSEIGMKGERRRGKGHDGLYQG |
| | LSTATKDTYDALHMQALPPR |
| 267 | EVQLLESGGGLVQPGGSLRLSCAASGFTFSNYAMSWVRQAPGKGLEWVSAISGSGGSTYYADSV |
| | KGRFTISRDNSKNTLYLQMNSLRAEDTAVYYCARPGDGYYEGVYFDYWGQGTLVTVSSGGGGSG |
| | GGGSGGGGSGGGGSDIQMTQSPSSLSASVGDRVTITCRASQSISSYLNWYQQKPGKAPKLLIYA |
| | ASSLQSGVPSRFSGSGSGTDFTLTISSLQPEDFATYYCQQAHSSPITFGGGTKVEIKTTTPAPR |
| | PPTPAPTIASQPLSLRPEACRPAAGGAVHTRGLDFACDIYIWAPLAGTCGVLLLSLVITLYCKR |
| | GRKKLLYIFKQPFMRPVQTTQEEDGCSCRFPEEEEGGCELRVKFSRSADAPAYQQGQNQLYNEL |
| | NLGRREEYDVLDKRRGRDPEMGGKPRRKNPQEGLYNELQKDKMAEAYSEIGMKGERRRGKGHDG |
| | LYQGLSTATKDTYDALHMQALPPR |
| 268 | DIQMTQSPSSLSASVGDRVTITCRASQSISSYLNWYQQKPGKAPKLLIYAASSLQSGVPSRESG |
| | SGSGTDFTLTISSLQPEDFATYYCQQAHSSPITFGGGTKVEIKGGGGSGGGGSGGGGSGGGGSE |
| | VQLLESGGGLVQPGGSLRLSCAASGFTFSNYAMSWVRQAPGKGLEWVSAISGSGGSTYYADSVK |
| | GRFTISRDNSKNTLYLQMNSLRAEDTAVYYCARPGDGYYEGVYFDYWGQGTLVTVSSTTTPAPR |
| | PPTPAPTIASQPLSLRPEACRPAAGGAVHTRGLDFACDIYIWAPLAGTCGVLLLSLVITLYCKR |
| | GRKKLLYIFKQPFMRPVQTTQEEDGCSCRFPEEEEGGCELRVKFSRSADAPAYQQGQNQLYNEL |
| | NLGRREEYDVLDKRRGRDPEMGGKPRRKNPQEGLYNELQKDKMAEAYSEIGMKGERRRGKGHDG |
| | LYQGLSTATKDTYDALHMQALPPR |
| 269 | QITLKESGPTLVKPTQTLTLTCTFSGFSLSTSGVGVGWIRQPPGKALEWLALIYWNDEKRYSPS |
| | LKSRLTITKDTSKNQVVLTMTNMDPVDTAVYYCAREGSHDYKSSNWFDPWGQGTLVTVSSGGGG |
| | SGGGGSGGGGSGGGGSDIQMTQSPSSLSASVGDRVTITCQASQDIANYLNWYQQKPGKAPKLLI |
| | YDASNLETGVPSRFSGSGSGTDFTFTISSLQPEDIATYYCQQHFNLPLTFGGGTKVEIKTTTPA |
| | PRPPTPAPTIASQPLSLRPEACRPAAGGAVHTRGLDFACDIYIWAPLAGTCGVLLLSLVITLYC |
| | KRGRKKLLYIFKQPFMRPVQTTQEEDGCSCRFPEEEEGGCELRVKFSRSADAPAYQQGQNQLYN |
| | ELNLGRREEYDVLDKRRGRDPEMGGKPRRKNPQEGLYNELQKDKMAEAYSEIGMKGERRRGKGH |
| | DGLYQGLSTATKDTYDALHMQALPPR |
| 270 | DIQMTQSPSSLSASVGDRVTITCQASQDIANYLNWYQQKPGKAPKLLIYDASNLETGVPSRFSG |
| | SGSGTDFTFTISSLQPEDIATYYCQQHFNLPLTFGGGTKVEIKGGGGSGGGGSGGGGSGGGGSQ |
| | ITLKESGPTLVKPTQTLTLTCTFSGFSLSTSGVGVGWIRQPPGKALEWLALIYWNDEKRYSPSL |
| | KSRLTITKDTSKNQVVLTMTNMDPVDTAVYYCAREGSHDYKSSNWFDPWGQGTLVTVSSTTTPA |
| | PRPPTPAPTIASQPLSLRPEACRPAAGGAVHTRGLDFACDIYIWAPLAGTCGVLLLSLVITLYC |
| | KRGRKKLLYIFKQPFMRPVQTTQEEDGCSCRFPEEEEGGCELRVKFSRSADAPAYQQGQNQLYN |
| | ELNLGRREEYDVLDKRRGRDPEMGGKPRRKNPQEGLYNELQKDKMAEAYSEIGMKGERRRGKGH |
| | DGLYQGLSTATKDTYDALHMQALPPR |
| 271 | EVQLVESGGGLVKPGGSLRLSCAASGFTFSSYSMNWVRQAPGKGLEWVSSISSSSSYIYYADSV |
| | KGRFTISRDNAKNSLYLQMNSLRAEDTAVYYCARAGDTYSAADYYYMDVWGKGTTVTVSSGGGG |
| | SGGGGSGGGGSGGGGSDIVMTQSPLSLPVTPGEPASISCRSSQSLLHSNGYNYLDWYLQKPGQS |
| | PQLLIYLGSNRASGVPDRFSGSGSGTDFTLKISRVEAEDVGVYYCMQALGLITFGGGTKVEIKT |
| | TTPAPRPPTPAPTIASQPLSLRPEACRPAAGGAVHTRGLDFACDIYIWAPLAGTCGVLLLSLVI |
| | TLYCKRGRKKLLYIFKQPFMRPVQTTQEEDGCSCRFPEEEEGGCELRVKFSRSADAPAYQQGQN |
| | QLYNELNLGRREEYDVLDKRRGRDPEMGGKPRRKNPQEGLYNELQKDKMAEAYSEIGMKGERRR |
| | GKGHDGLYQGLSTATKDTYDALHMQALPPR |
| 272 | DIVMTQSPLSLPVTPGEPASISCRSSQSLLHSNGYNYLDWYLQKPGQSPQLLIYLGSNRASGVP |
| | DRFSGSGSGTDFTLKISRVEAEDVGVYYCMQALGLITFGGGTKVEIKGGGGSGGGGSGGGGSGG |
| | GGSEVQLVESGGGLVKPGGSLRLSCAASGFTFSSYSMNWVRQAPGKGLEWVSSISSSSSYIYYA |
| | DSVKGRFTISRDNAKNSLYLQMNSLRAEDTAVYYCARAGDTYSAADYYYMDVWGKGTTVTVSST |
| | TTPAPRPPTPAPTIASQPLSLRPEACRPAAGGAVHTRGLDFACDIYIWAPLAGTCGVLLLSLVI |
| | TLYCKRGRKKLLYIFKQPFMRPVQTTQEEDGCSCRFPEEEEGGCELRVKFSRSADAPAYQQGQN |
| | QLYNELNLGRREEYDVLDKRRGRDPEMGGKPRRKNPQEGLYNELQKDKMAEAYSEIGMKGERRR |
| | GKGHDGLYQGLSTATKDTYDALHMQALPPR |
| 273 | EVQLLESGGGLVQPGGSLRLSCAASGFTFGSEAMSWVRQAPGKERELVSAISGSGEVTYYADSV |
| | KGRFTISRDNSKNTLYLQMNSLRAEDTAVYYCQRLVEAKRHWGQGTQVTVSSTTTPAPRPPTPA |
| | PTIASQPLSLRPEACRPAAGGAVHTRGLDFACDIYIWAPLAGTCGVLLLSLVITLYCKRGRKKL |
| | LYIFKQPFMRPVQTTQEEDGCSCRFPEEEEGGCELRVKFSRSADAPAYQQGQNQLYNELNLGRR |
| | EEYDVLDKRRGRDPEMGGKPRRKNPQEGLYNELQKDKMAEAYSEIGMKGERRRGKGHDGLYQGL |
| | STATKDTYDALHMQALPPR |
| 274 | EVQLLESGGGLVQPGGSLRLSCAASGFTFESEAMSWYRQAPGKERELVSVITSEGSTYYADSVK |
| | GRFTISRDNSKNTLYLQMNSLRAEDTAVYYCAHIEWETRLNWGQGTQVTVSSTTTPAPRPPTPA |
| | PTIASQPLSLRPEACRPAAGGAVHTRGLDFACDIYIWAPLAGTCGVLLLSLVITLYCKRGRKKL |
| | LYIFKQPFMRPVQTTQEEDGCSCRFPEEEEGGCELRVKFSRSADAPAYQQGQNQLYNELNLGRR |
| | EEYDVLDKRRGRDPEMGGKPRRKNPQEGLYNELQKDKMAEAYSEIGMKGERRRGKGHDGLYQGL |
| | STATKDTYDALHMQALPPR |
| 275 | EVQLLESGGGLVQPGGSLRLSCAASGFTFDEYTMHWFRQAPGKEREFVSAISGGGSETYYADSV |
| | KGRFTISRDNSKNTLYLQMNSLRAEDTAVYYCAAGGEEAGVGYWGQGTQVTVSSTTTPAPRPPT |
| | PAPTIASQPLSLRPEACRPAAGGAVHTRGLDFACDIYIWAPLAGTCGVLLLSLVITLYCKRGRK |
| | KLLYIFKQPFMRPVQTTQEEDGCSCRFPEEEEGGCELRVKFSRSADAPAYQQGQNQLYNELNLG |
| | RREEYDVLDKRRGRDPEMGGKPRRKNPQEGLYNELQKDKMAEAYSEIGMKGERRRGKGHDGLYQ |
| | GLSTATKDTYDALHMQALPPR |
| 276 | EVQLLESGGGLVQPGGSLRLSCAASGFTFEDYAMSWFRQAPGKEREGVSAISGKGGSTYYADSV |
| | KGRFTISRDNSKNTLYLQMNSLRAEDTAVYYCAVLDEEAGAEGGYWGQGTQVTVSSTTTPAPRP |
| | PTPAPTIASQPLSLRPEACRPAAGGAVHTRGLDFACDIYIWAPLAGTCGVLLLSLVITLYCKRG |
| | RKKLLYIFKQPFMRPVQTTQEEDGCSCRFPEEEEGGCELRVKFSRSADAPAYQQGQNQLYNELN |
| | LGRREEYDVLDKRRGRDPEMGGKPRRKNPQEGLYNELQKDKMAEAYSEIGMKGERRRGKGHDGL |
| | YQGLSTATKDTYDALHMQALPPR |
| 277 | EVQLLESGGGLVQPGGSLRLSCAASGFTFDRYAMSWFRQAPGKEREGVSAISTSGDSTYYADSV |
| | KGRFTISRDNSKNTLYLQMNSLRAEDTAVYYCAVLDEEAGAEGGYWGQGTQVTVSSTTTPAPRP |
| | PTPAPTIASQPLSLRPEACRPAAGGAVHTRGLDFACDIYIWAPLAGTCGVLLLSLVITLYCKRG |
| | RKKLLYIFKQPFMRPVQTTQEEDGCSCRFPEEEEGGCELRVKFSRSADAPAYQQGQNQLYNELN |
| | LGRREEYDVLDKRRGRDPEMGGKPRRKNPQEGLYNELQKDKMAEAYSEIGMKGERRRGKGHDGL |
| | YQGLSTATKDTYDALHMQALPPR |
| 278 | EVQLLESGGGLVQPGGSLRLSCAASGFTFASDAMSWYRQAPGKERELVSAISGSGGSTYYADSV |
| | KGRFTISRDNSKNTLYLQMNSLRAEDTAVYYCAAHDSGEAYLAFDYWGQGTQVTVSSTTTPAPR |
| | PPTPAPTIASQPLSLRPEACRPAAGGAVHTRGLDFACDIYIWAPLAGTCGVLLLSLVITLYCKR |
| | GRKKLLYIFKQPFMRPVQTTQEEDGCSCRFPEEEEGGCELRVKFSRSADAPAYQQGQNQLYNEL |
| | NLGRREEYDVLDKRRGRDPEMGGKPRRKNPQEGLYNELQKDKMAEAYSEIGMKGERRRGKGHDG |
| | LYQGLSTATKDTYDALHMQALPPR |
| 279 | EVQLLESGGGLVQPGGSLRLSCAASGFTFDSYTMSWYRQAPGKERELVSAISGHGDSTYYADSV |
| | KGRFTISRDNSKNTLYLQMNSLRAEDTAVYYCTRISITTEWLAGDYWGQGTQVTVSSTTTPAPR |
| | PPTPAPTIASQPLSLRPEACRPAAGGAVHTRGLDFACDIYIWAPLAGTCGVLLLSLVITLYCKR |
| | GRKKLLYIFKQPFMRPVQTTQEEDGCSCRFPEEEEGGCELRVKFSRSADAPAYQQGQNQLYNEL |
| | NLGRREEYDVLDKRRGRDPEMGGKPRRKNPQEGLYNELQKDKMAEAYSEIGMKGERRRGKGHDG |
| | LYQGLSTATKDTYDALHMQALPPR |
| 280 | EVQLLESGGGLVQPGGSLRLSCAASGFTFSSYAMSWFRQAPGKEREFVSFISGSGDSTYYADSV |
| | KGRFTISRDNSKNTLYLQMNSLRAEDTAVYYCTRWPYDFEEPSEPGVYWGQGTQVTVSSTTTPA |
| | PRPPTPAPTIASQPLSLRPEACRPAAGGAVHTRGLDFACDIYIWAPLAGTCGVLLLSLVITLYC |
| | KRGRKKLLYIFKQPFMRPVQTTQEEDGCSCRFPEEEEGGCELRVKFSRSADAPAYQQGQNQLYN |
| | ELNLGRREEYDVLDKRRGRDPEMGGKPRRKNPQEGLYNELQKDKMAEAYSEIGMKGERRRGKGH |
| | DGLYQGLSTATKDTYDALHMQALPPR |
| 281 | EVQLLESGGGLVQPGGSLRLSCAASGFTFTDYDMSWYRQAPGKERELVSVIHSGGSTYYADSVK |
| | GRFTISRDNSKNTLYLQMNSLRAEDTAVYYCAPGYYSDLSFDYYNFDYWGQGTQVTVSSTTTPA |
| | PRPPTPAPTIASQPLSLRPEACRPAAGGAVHTRGLDFACDIYIWAPLAGTCGVLLLSLVITLYC |
| | KRGRKKLLYIFKQPFMRPVQTTQEEDGCSCRFPEEEEGGCELRVKFSRSADAPAYQQGQNQLYN |
| | ELNLGRREEYDVLDKRRGRDPEMGGKPRRKNPQEGLYNELQKDKMAEAYSEIGMKGERRRGKGH |
| | DGLYQGLSTATKDTYDALHMQALPPR |
| 282 | EVQLLESGGGLVQPGGSLRLSCAASGFTFSDYAMHWFRQAPGKERVLVSSIDSGGSTYYADSVK |
| | GRFTISRDNSKNTLYLQMNSLRAEDTAVYYCNAGFKGDHPHPKDAFDIWGQGTQVTVSSTTTPA |
| | PRPPTPAPTIASQPLSLRPEACRPAAGGAVHTRGLDFACDIYIWAPLAGTCGVLLLSLVITLYC |
| | KRGRKKLLYIFKQPFMRPVQTTQEEDGCSCRFPEEEEGGCELRVKFSRSADAPAYQQGQNQLYN |
| | ELNLGRREEYDVLDKRRGRDPEMGGKPRRKNPQEGLYNELQKDKMAEAYSEIGMKGERRRGKGH |
| | DGLYQGLSTATKDTYDALHMQALPPR |
| 283 | EVQLLESGGGLVQPGGSLRLSCAASGFTFSSEGMSWVRQAPGKERELVSAISGSGDHTYYADSV |
| | RGRFTISRDNSKNTLYLQMNSLRAEDTAVYYCNALEGGPTTAIQPGGPDYWGQGTQVTVSSTTT |
| | PAPRPPTPAPTIASQPLSLRPEACRPAAGGAVHTRGLDFACDIYIWAPLAGTCGVLLLSLVITL |
| | YCKRGRKKLLYIFKQPFMRPVQTTQEEDGCSCRFPEEEEGGCELRVKFSRSADAPAYQQGQNQL |
| | YNELNLGRREEYDVLDKRRGRDPEMGGKPRRKNPQEGLYNELQKDKMAEAYSEIGMKGERRRGK |
| | GHDGLYQGLSTATKDTYDALHMQALPPR |

In particular embodiments, a CAR comprises an amino acid sequence set forth in any one of SEQ ID NOs: 255, 256, 257, 258, 259, 260, 261, 262, 263, 264, 265, 266, 267, 268, 269, 270, 271, 272, 273, 274, 275, 276, 277, 278, 279, 280, 281, 282, and 283.

In particular embodiments, a CAR comprises an amino acid sequence set forth in any one of SEQ ID NOs: 256, 258, 259, 262, 263, 266, 268, 270, and 272.

In particular embodiments, a CAR comprises an amino acid sequence set forth in any one of SEQ ID NOs: 259, 263, 266, and 270.

In particular embodiments, a CAR comprises an amino acid sequence set forth in SEQ ID NO: 273 or 277.

### F. Polypeptides

Polypeptides, fusion polypeptides, and polypeptide variants are contemplated in particular embodiments. Exemplary polypeptides contemplated herein include but not limited to fusion polypeptides, fusogens, tropism polypeptides, chimeric antigen receptors (CARs) and components thereof, and variants and/or fragments thereof, e.g., SEQ ID NOs: 1-283 and 324-374. Polypeptides contemplated herein also include those encoded by polynucleotide sequences set forth in any one of SEQ ID NOs: 284-323.

Polypeptide,” “polypeptide,” “peptide,” and “protein” are used interchangeably, unless specified to the contrary, and according to conventional meaning, i.e., as a sequence of amino acids. In particular embodiments, a “polypeptide” is a fusion polypeptide or polypeptide variant. Polypeptides can be prepared using any of a variety of well-known recombinant and/or synthetic techniques. Polypeptides are not limited to a specific length, e.g., they may comprise a full-length protein sequence, a fragment of a full-length protein, or a fusion protein, and may include post-translational modifications, e.g., glycosylations, acetylations, phosphorylations and the like, as well as other modifications known in the art, both naturally occurring and non-naturally occurring.

An “isolated peptide,” “isolated protein” or an “isolated polypeptide” as used herein, refers to isolation, separation, and/or purification of a polypeptide molecule from a cellular environment, and from association with other components of the cell, i.e., it is not significantly associated with in vivo substances.

Polypeptides include “polypeptide variants.” In particular embodiments, a polypeptide variant is referred to as a “modified polypeptide.” Polypeptide variants may differ from a naturally occurring polypeptide in one or more amino acid substitutions, deletions, additions and/or insertions. For example, in particular embodiments, it may be desirable to modulate one or more biological activities of a chimeric antigen receptor by introducing one or more amino acid substitutions, deletions, additions and/or insertions into the polypeptide. Such variants may be naturally occurring or may be synthetically generated. In particular embodiments, polypeptides include polypeptide variants having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any reference sequence contemplated herein, typically where the variant maintains at least one biological activity of the reference sequence.

Polypeptides variants include “polypeptide fragments.” Illustrative examples of polypeptide fragments include but are not limited to anti-BCMA antibodies or antigen binding fragments thereof, anti-CD3 antibodies or antigen binding fragments thereof, spacer domains, hinges, transmembrane domains, intracellular signaling domains, and the like. In particular embodiments, a polypeptide fragment is a biologically active polypeptide fragment. As used herein, the term “biologically active polypeptide fragment” refers to a polypeptide fragment that retains at least 100%, at least 95%, at least 90%, at least 85%, at least 80%, at least 75%, at least 70%, at least 65%, at least 60%, at least 55%, or at least 50% of the naturally occurring polypeptide activity. In certain embodiments, a polypeptide fragment comprises an amino acid sequence at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, or 500 or more amino acids long.

In particular embodiments, polypeptides contemplated herein may comprise one or more amino acids denoted as “X” or “Xₙ” wherein n is an integer that denotes the particular X amino acid. “X” if present in an amino acid SEQ ID NO, refers to any one or more amino acids or particular amino acids if disclosed.

In particular embodiments, a polypeptide comprises one or more amino acid substitutions, deletions, truncations, or insertions using methods that are well known in the art. See, for example, Kunkel (Proc. Natl. Acad. Sci. USA. 82: 488-492. (1985)), Kunkel et al., (Methods in Enzymol, 154: 367-382. (1987)), U.S. Pat. No. 4,873,192, Watson, J. D. et al., (Molecular Biology of the Gene, Fourth Edition, Benjamin/Cummings, Menlo Park, Calif. (1987)) and the references cited therein. Guidance as to appropriate amino acid substitutions that do not affect biological activity of the protein of interest may be found in the model of Dayhoff et al., Atlas of Protein Sequence and Structure (Natl. Biomed. Res. Found., Washington, D.C. (1978)).

In certain embodiments, a polypeptide variant comprises one or more conservative substitutions or disruptive substitutions. A “conservative substitution” is one in which an amino acid is substituted for another amino acid that has similar properties. In particular embodiments, polypeptide variants contemplated herein comprise one or more conservative amino acid changes compared to a reference polypeptide. In particular embodiments, a conservative amino acid substitution involves substituting an amino acid with an amino acid having a related side chain. A “disruptive substitution” is one in which an amino acid is substituted for another amino acid that has different properties, e.g., polar vs. non-polar, bulky vs. non-bulky, charged vs. uncharged, acidic vs. basic. In particular embodiments, polypeptide variants contemplated herein comprise one or more disruptive amino acid changes compared to a reference polypeptide. In particular embodiments, a disruptive amino acid substitution involves substituting an amino acid with an amino acid having an unrelated side chain or side change with a different chemical property. Guidance in determining which amino acid residues can be substituted, inserted, or deleted can be found using computer programs well known in the art, such as DNASTAR, DNA Strider, Geneious, Mac Vector, or Vector NTI software.

Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In particular embodiments, a conservative amino acid substitution refers to substituting amino acids within the same group or family.

Those of skill in this art recognize that, in general, conservative single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224), whereas disruptive single amino acid substitutions may.

In particular embodiments, a conservative amino acid substitution refers to substituting amino acids having a similar hydropathic index or score. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In particular embodiments, a conservative amino acid substitution refers to substituting amino acids having a similar hydropathic index or score. In particular embodiments, substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

In particular embodiments, a conservative amino acid substitution refers to substituting amino acids having a similar hydrophilic index or score. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In particular embodiments, a conservative amino acid substitution refers to substituting amino acids having a similar hydrophilic index or score. In particular embodiments, substitution of amino acids whose hydrophilic indices are substitution of amino acids whose hydrophilicity values are within ±2 is preferred, those within ±1 are particularly preferred, and those within ±0.5 are even more particularly preferred.

In particular embodiments, a conservative amino acid substitution may be based on the relative similarity of the amino acid side-chain substituents, for example, their hydrophobicity, hydrophilicity, charge, size, and the like. In particular embodiments, a disruptive amino acid substitution may be based on the relative dissimilarity of the amino acid side-chain substituents, for example, their hydrophobicity, hydrophilicity, charge, size, and the like.

In particular embodiments, a polypeptide comprises a vesiculovirus envelope glycoprotein G comprising one or more amino acid substitutions at positions 47 and/or 354 of the mature polypeptide lacking a signal peptide. In particular embodiments, a polypeptide comprises a mutated cocal virus envelope glycoprotein (COCV-G) or a mutated vesicular stomatitis Indiana virus envelope glycoprotein (VSIV-G), wherein the COCV-G or VSIV-G comprises amino acid substitutions at positions 47 and 354.

Polypeptides contemplated in particular embodiments include fusion polypeptides. In particular embodiments, fusion polypeptides and polynucleotides encoding fusion polypeptides are provided. Fusion polypeptides can include one or more polypeptide domains or segments including but not limited to signal peptides, antibodies or antigen binding fragments thereof, polypeptide linkers, spacer domains, transmembrane domains, intracellular signaling domains, and polypeptide cleavage signals. Fusion proteins and polypeptides are typically linked C-terminus to N-terminus, although they can also be linked C-terminus to C-terminus, N-terminus to N-terminus, or N-terminus to C-terminus. Fusion polypeptides and fusion proteins refer to a polypeptide having at least two, three, four, five, six, seven, eight, nine, or ten polypeptide segments.

Fusion polypeptides may optionally comprise a polypeptide linker contemplated elsewhere herein that can be used to link one or more polypeptides or domains within a polypeptide.

In particular embodiments, a polypeptide or fusion polypeptide comprises a non-viral membrane bound tropism polypeptide that binds an antigen expressed on an immune effector cell. In particular embodiments, a polypeptide comprises a non-viral membrane-bound tropism polypeptide comprising an anti-CD3ε scFv and a human CD8α hinge and transmembrane domain.

In particular embodiments, a polypeptide or fusion polypeptide comprises an anti-BCMA chimeric antigen receptor comprising an anti-BCMA antibody or antigen binding fragment thereof comprising an amino acid sequence set forth in any one of SEQ ID NOs: 39, 59, 70, 90, 101 and 117; a CD8α hinge, a CD8α transmembrane domain; a CD137 costimulatory signaling domain; and a CD3 ζ primary signaling domain.

In particular embodiments, a polypeptide comprises signal peptide set forth in any one of SEQ ID NOs: 245, 246, 247, 248, 249, 250, 251, 252, 253, and 254 that is subsequently cleaved from the polypeptide. Signal peptides are short 16 to 30 amino acid N-terminal sequences of nascently synthesized polypeptide chains that mediate protein targeting to the membrane of the endoplasmic reticulum (ER). Typically, signal peptides are cleaved cotranslationally by signal peptidase, a heterooligomeric polypeptide complex. In particular embodiments, a polypeptide comprises a signal peptide. In preferred embodiments, a polynucleotide encoding a polypeptide comprises a polynucleotide encoding a signal polypeptide; and the translated polypeptide does not comprise a signal peptide. Exemplary signal peptides are set forth in Table 12.

TABLE 12
Exemplary Signal Peptides

| SEQ ID NO | AMINO ACID SEQUENCE |
|---|---|
| 245 | MALPVTALLLPLALLLHAARP |
| 246 | METDTLLLWVLLLWVPGSTG |
| 247 | MDMRVPAQLLGLLLLWLRGARC |
| 248 | MPLLLLLPLLWAGALA |
| 249 | MDAMKRGLCCVLLLCGAVFVSPS |
| 250 | MLLLLLLLGLRLQLSLG |
| 251 | MWLQSLLLLGTVACSIS |
| 252 | MLLLVTSLLLCELPHPAFLLIP |
| 253 | MSRSVALAVLALLSLSGLEA |
| 254 | MLLLLLLLLLLALALA |

In particular embodiments, a polypeptide comprises a signal peptide set forth in any one of SEQ ID NOs: 245, 246, 247, 248, 249, 250, 251, 252, 253, and 254 and a chimeric antigen receptor comprising an amino acid sequence set forth in any one of SEQ ID NOs: 255, 256, 257, 258, 259, 260, 261, 262, 263, 264, 265, 266, 267, 268, 269, 270, 271, 272, 273, 274, 275, 276, 277, 278, 279, 280, 281, 282, and 283.

In particular embodiments, a polypeptide comprises a signal peptide set forth in any one of SEQ ID NOs: 245, 246, 247, 248, 249, 250, 251, 252, 253, and 254 and a chimeric antigen receptor encoded by a polynucleotide sequence set forth in any one of SEQ ID NOs: 295, 296, 297, 298, 299, 300, 301, 302, 303, 304, 305, 306, 307, 308, 309, 310, 311, 312, 313 and 314.

In particular embodiments, two or more polypeptides can be expressed as a fusion polypeptide that comprises one or more polypeptide cleavage signals disposed between the two or more polypeptides.

Exemplary polypeptide cleavage signals include, but are not limited to, protease cleavage sites, nuclease cleavage sites and ribosomal skipping polypeptide or self-cleaving viral polypeptides (see, e.g., in Ryan et al., 1997. J. Gener. Virol. 78, 699-722; deFelipe and Ryan, 2004. Traffic, 5(8); 616-26; and Scymczak et al. (2004) Nature Biotech. 5, 589-594).

Exemplary protease cleavage sites include, but are not limited to the cleavage sites of potyvirus NIa proteases (e.g, tobacco etch virus protease), poty virus HC proteases, potyvirus PI (P35) proteases, byovirus NIa proteases, byovirus RNA-2-encoded proteases, aphthovirus L proteases, enterovirus 2A proteases, rhinovirus 2 A proteases, picoma 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase.

Illustrative examples of ribosomal skipping polypeptides include but are not limited to: a viral 2A peptide or sequence (Donnelly et al, 2001. J. Gen. Virol. 82: 1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of viral 2A sequences include, but are not limited to:

(SEQ ID NO: 355)
GSGATNFSLLKQAGDVEENPGP;
(SEQ ID NO: 356)
ATNFSLLKQAGDVEENPGP;
(SEQ ID NO: 357)
LLKQAGDVEENPGP;
(SEQ ID NO: 358)
GSGEGRGSLLTCGDVEENPGP;
(SEQ ID NO: 359)
EGRGSLLTCGDVEENPGP;
(SEQ ID NO: 360)
LLTCGDVEENPGP;
(SEQ ID NO: 361)
GSGQCTNYALLKLAGDVESNPGP;
(SEQ ID NO: 362)
QCTNYALLKLAGDVESNPGP;
(SEQ ID NO: 363)
LLKLAGDVESNPGP;
(SEQ ID NO: 364)
GSGVKQTLNFDLLKLAGDVESNPGP;
(SEQ ID NO: 365)
VKQTLNFDLLKLAGDVESNPGP;
(SEQ ID NO: 366)
LLNFDLLKLAGDVESNPGP;
(SEQ ID NO: 367)
TLNFDLLKLAGDVESNPGP;
(SEQ ID NO: 368)
NFDLLKLAGDVESNPGP;
(SEQ ID NO: 369)
QLLNFDLLKLAGDVESNPGP;
(SEQ ID NO: 370)
APVKQTLNFDLLKLAGDVESNPGP;
(SEQ ID NO: 371)
VTELLYRMKRAETYCPRPLLAIHPTEARHKQKIVAPVKQT;
(SEQ ID NO: 372)
LNFDLLKLAGDVESNPGP;
(SEQ ID NO: 373)
LLAIHPTEARHKQKIVAPVKQTLNFDLLKLAGDVESNPGP;
and
(SEQ ID NO: 374)
EARHKQKIVAPVKQTLNFDLLKLAGDVESNPGP.

### G. Polynucleotides

Polynucleotides comprising or encoding fusogens, tropism polypeptides, chimeric antigen receptors (CARs) and components thereof, and variants and/or fragments thereof, vectors, promoters, enhancers, Kozak sequences, polyadenylation signals, untranslated regions, and posttranscriptional response elements as well as other polynucleotides are contemplated in various embodiments.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include but are not limited to: pre-messenger RNA (pre-mRNA), messenger RNA (mRNA), RNA, circular RNA (circRNA), synthetic RNA, short interfering RNA (siRNA), short hairpin RNA (shRNA), microRNA (miRNA), ribozymes, genomic RNA (gRNA), viral genomic RNA, plus strand RNA (RNA(+)), minus strand RNA (RNA(−)), tracrRNA, crRNA, single guide RNA (sgRNA), Doggybone DNA (dbDNA), linear DNA, circular DNA, PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc., 151, 152, 153, etc., 201, 202, 203, etc.

As used herein, “isolated polynucleotide” refers to a polynucleotide that has been isolated from or purified from the sequences which flank it in a naturally-occurring state. In particular embodiments, an isolated polynucleotide is a synthetic polynucleotide, a semi-synthetic polynucleotide, or a polynucleotide obtained or derived from a recombinant source, or other polynucleotide that does not exist in nature and that has been made by the hand of man.

In particular embodiments, polynucleotides contemplated herein are polynucleotide variants. As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of one or more nucleotides. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide or wherein the function or activity of the altered polynucleotide is modulated. In particular embodiments, polynucleotides or polynucleotide variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

In particular embodiments, a polynucleotide variant includes a polynucleotide fragment that encodes biologically active polypeptide fragments or variants. As used herein, the term “polynucleotide fragment” refers to a polynucleotide fragment at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more nucleotides in length that encodes a polypeptide variant that retains at least 100%, at least 90%, at least 80%, at least 70%, at least 60%, at least 50%, at least 40%, at least 30%, at least 20%, at least 10%, or at least 5% of the naturally occurring polypeptide activity. Polynucleotide fragments refer to a polynucleotide that encodes a polypeptide that has an amino-terminal deletion, a carboxyl-terminal deletion, and/or an internal deletion or substitution of one or more amino acids of a naturally occurring or recombinantly-produced polypeptide.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 5′ strand is designated the “sense,” “plus,” or “coding” strand because its sequence is identical to the sequence of the RNA [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

As used herein, the phrases “sequence identity” or, for example, comprising a “sequence 50% identical to,” refer to the extent that sequences are identical on a nucleotide-by-nucleotide basis or an amino acid-by-amino acid basis over a window of comparison. A “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. In particular embodiments, polynucleotides and polypeptides comprise at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences described herein, e.g., SEQ ID NOs: 1-339.

Illustrative examples of polynucleotides include, but are not limited to, polynucleotide sequences set forth in any one of SEQ ID NOs: 284-323 and polynucleotides encoding polypeptides set forth in SEQ ID NOs: 1-283 and 324-374.

In various embodiments, a polynucleotide encodes a polypeptide comprising an amino acid sequence set forth in any one of SEQ ID NOs: 1-283 and 324-374.

In particular embodiments, a polynucleotide encodes a chimeric antigen receptor comprising an amino acid sequence set forth in any one of SEQ ID NOs: 255, 256, 257, 258, 259, 260, 261, 262, 263, 264, 265, 266, 267, 268, 269, 270, 271, 272, 273, 274, 275, 276, 277, 278, 279, 280, 281, 282, and 283. In particular embodiments, a polynucleotide encodes a chimeric antigen receptor comprising an amino acid sequence set forth in any one of SEQ ID NOs: 256, 258, 259, 262, 263, 266, 268, 270, and 272. In particular embodiments, a polynucleotide encodes a chimeric antigen receptor comprising an amino acid sequence set forth in any one of SEQ ID NOs: 259, 263, 266, and 270. In particular embodiments, a polynucleotide encodes a chimeric antigen receptor comprising an amino acid sequence set forth in any one of SEQ ID NO: 273 or 277. In particular embodiments, a polynucleotide encoding a chimeric antigen receptor comprises a polynucleotide sequence set forth in any one of SEQ ID NOs: 295, 296, 297, 298, 299, 300, 301, 302, 303, 304, 305, 306, 307, 308, 309, 310, 311, 312, 313, and 314.

Table 13 sets forth the SEQ ID NOs. and associated nucleic acid sequences encoding chimeric antigen receptor components and chimeric antigen receptors and the corresponding amino acid SEQ ID NO (AA SEQ ID NO.) encoded by the nucleic acid sequence.

TABLE 13

| SEQ | | |
|---|---|---|
| ID | AA SEQ | |
| NO. | ID NO. | NUCLEIC ACID SEQUENCE |
| 284 | 225 | ACCACAACACCTGCTCCAAGGCCCCCCACACCCGCTCCAACTATAGCCAGCCAA |
| | | CCATTGAGCCTCAGACCTGAAGCTTGCAGGCCCGCAGCAGGAGGCGCCGTCCAT |
| | | ACGCGAGGCCTGGACTTCGCGTGTGAT |
| 285 | 226 | ATTGAAGTTATGTATCCTCCTCCTTACCTAGACAATGAGAAGAGCAATGGAACC |
| | | ATTATCCATGTGAAAGGGAAACACCTTTGTCCAAGTCCCCTATTTCCCGGACCT |
| | | TCTAAGCCC |
| 286 | 228 | GAGTCCAAATATGGTCCCCCGTGCCCACCATGCCCA |
| 287 | 230 | CTTGAGCCCAAATCTTGTGACAAAACTCACACATGCCCACCGTGCCCA |
| 288 | 231 | ATTTATATTTGGGCACCTTTGGCCGGAACATGTGGGGTGTTGCTTCTCTCCCTT |
| | | GTGATCACTCTGTATTGT |
| 289 | 233 | TTTTGGGTGCTGGTGGTGGTTGGTGGAGTCCTGGCTTGCTATAGCTTGCTAGTA |
| | | ACAGTGGCCTTTATTATTTTCTGGGTG |
| 290 | 239 | AAGCGCGGGAGAAAGAAGCTCCTGTACATCTTCAAGCAGCCTTTTATGCGACCT |
| | | GTGCAAACCACTCAGGAAGAAGATGGGTGTTCATGCCGCTTCCCCGAGGAGGAA |
| | | GAAGGAGGGTGTGAACTG |
| 291 | 240 | AGGAGTAAGAGGAGCAGGCTCCTGCACAGTGACTACATGAACATGACTCCCCGC |
| | | CGCCCCGGGCCCACCCGCAAGCATTACCAGCCCTATGCCCCACCACGCGACTTC |
| | | GCAGCCTATCGCTCC |
| 292 | 242 | ACAAAAAAGAAGTATTCATCCAGTGTGCACGACCCTAACGGTGAATACATGTTC |
| | | ATGAGAGCAGTGAACACAGCCAAAAAATCTAGACTCACAGATGTGACCCTA |
| 293 | 238 | AGGGTGAAATTTTCTAGAAGCGCCGATGCTCCCGCATATCAGCAGGGTCAGAAT |
| | | CAGCTCTACAATGAATTGAATCTCGGCAGGCGAGAAGAGTACGATGTTCTGGAC |
| | | AAAAGACGGGGCAGGGATCCCGAGATGGGGGGAAAGCCCCGGAGAAAAAATCCT |
| | | CAGGAGGGGTTGTACAATGAGCTGCAGAAGGACAAGATGGCTGAAGCCTATAGC |
| | | GAGATCGGAATGAAAGGCGAAAGACGCAGAGGCAAGGGGCATGACGGTCTGTAC |
| | | CAGGGTCTCTCTACAGCCACCAAGGACACTTATGATGCGTTGCATATGCAAGCC |
| | | TTGCCACCCCGC |
| 294 | 245 | ATGGCTCTTCCCGTAACAGCCCTTTTGTTGCCCCTTGCACTCCTTCTGCATGCA |
| | | GCACGACCG |
| 295 | 256 | GAGATCGTGCTGACACAGTCTCCCGCCACACTGTCACTGTCTCCAGGCGAAAGA |
| | | GCCACACTGAGCTGTAGAGCCAGCCAGAGCGTGTCCTCTTACCTGGCCTGGTAT |
| | | CAGCAGAAGCCTGGACAGGCTCCCCGGCTGCTGATCTACGATGCCAGCAATAGA |
| | | GCCACAGGCATCCCCGCCAGATTTTCTGGCAGCGGCTCTGGCACCGATTTCACC |
| | | CTGACCATAAGCAGCCTGGAACCTGAGGACTTCGCCGTGTACTACTGCCAGCAG |
| | | AGAGTGGTGTACCCCATCACCTTTGGCGGAGGCACCAAGGTGGAAATCAAAGGC |
| | | GGCGGAGGAAGCGGAGGCGGAGGATCTGGTGGTGGTGGATCTGGCGGAGGTGGC |
| | | AGCCAGATCACACTGAAAGAGTCTGGCCCCACACTGGTCAAGCCCACACAGACC |
| | | CTGACACTGACCTGCACCTTCAGCGGCTTTAGCCTGAGCACATCTGGCGTCGGC |
| | | GTTGGCTGGATTAGACAGCCTCCTGGAAAGGCCCTGGAATGGCTGGCCCTGATC |
| | | TACTGGAACGACGAGAAGAGATACAGCCCCAGCCTGAAGTCCCGGCTGACCATC |
| | | ACCAAGGACACCAGCAAGAACCAGGTGGTGCTGACCATGACAAACATGGACCCC |
| | | GTGGACACCGCCGTGTATTATTGCGCCAGAGATGAGTACGGCGGCTTCGACATT |
| | | TGGGGCCAGGGCACAATGGTCACCGTGTCTAGTACCACAACACCTGCTCCAAGG |
| | | CCCCCCACACCCGCTCCAACTATAGCCAGCCAACCATTGAGCCTCAGACCTGAA |
| | | GCTTGCAGGCCCGCAGCAGGAGGCGCCGTCCATACGCGAGGCCTGGACTTCGCG |
| | | TGTGATATTTATATTTGGGCACCTTTGGCCGGAACATGTGGGGTGTTGCTTCTC |
| | | TCCCTTGTGATCACTCTGTATTGTAAGCGCGGGAGAAAGAAGCTCCTGTACATC |
| | | TTCAAGCAGCCTTTTATGCGACCTGTGCAAACCACTCAGGAAGAAGATGGGTGT |
| | | TCATGCCGCTTCCCCGAGGAGGAAGAAGGAGGGTGTGAACTGAGGGTGAAATTT |
| | | TCTAGAAGCGCCGATGCTCCCGCATATCAGCAGGGTCAGAATCAGCTCTACAAT |
| | | GAATTGAATCTCGGCAGGCGAGAAGAGTACGATGTTCTGGACAAAAGACGGGGC |
| | | AGGGATCCCGAGATGGGGGGAAAGCCCCGGAGAAAAAATCCTCAGGAGGGGTTG |
| | | TACAATGAGCTGCAGAAGGACAAGATGGCTGAAGCCTATAGCGAGATCGGAATG |
| | | AAAGGCGAAAGACGCAGAGGCAAGGGGCATGACGGTCTGTACCAGGGTCTCTCT |
| | | ACAGCCACCAAGGACACTTATGATGCGTTGCATATGCAAGCCTTGCCACCCCGC |
| 296 | 258 | GAGATCGTGCTGACCCAGTCCCCTGCTACCCTGAGCCTGTCTCCAGGCGAGCGG |
| | | GCCACACTGAGCTGTAGAGCTTCTCAGAGCGTGTCCAGCTACCTGGCCTGGTAT |
| | | CAGCAGAAACCTGGCCAGGCCCCTAGACTGCTGATCTACGACGCCAGCAACCGG |
| | | GCCACCGGCATCCCCGCCAGATTCAGCGGATCTGGCAGCGGCACAGATTTTACC |
| | | CTCACCATCAGCAGCCTGGAACCTGAGGACTTCGCCGTCTACTACTGCCAGCAA |
| | | AGATTCGACTACCCCATCACCTTCGGCGGCGGAACAAAGGTGGAAATTAAGGGT |
| | | GGTGGGGGCAGCGGTGGAGGTGGGAGCGGAGGCGGGGGTAGCGGAGGCGGGGGT |
| | | AGCCAAATCACACTGAAAGAGAGCGGCCCTACACTCGTGAAACCTACCCAGACC |
| | | CTGACACTGACATGTACCTTCAGCGGCTTCTCCCTGAGCACCTCTGGCGTCGGC |
| | | GTTGGATGGATCAGACAGCCTCCAGGCAAGGCCCTGGAATGGCTGGCTCTGATC |
| | | TATTGGAACGACGACAAGCGGTACAGCCCCAGCCTGAAGTCTAGACTGACCATC |
| | | ACAAAGGACACCAGCAAGAACCAGGTGGTGCTGACCATGACAAATATGGACCCC |
| | | GTGGACACCGCCGTGTACTACTGCGCCAGAGATGAGTACGGCGGATTTGATATC |
| | | TGGGGCCAGGGCACCATGGTGACCGTGTCCAGCACCACAACACCTGCTCCAAGG |
| | | CCCCCCACACCCGCTCCAACTATAGCCAGCCAACCATTGAGCCTCAGACCTGAA |
| | | GCTTGCAGGCCCGCAGCAGGAGGCGCCGTCCATACGCGAGGCCTGGACTTCGCG |
| | | TGTGATATTTATATTTGGGCACCTTTGGCCGGAACATGTGGGGTGTTGCTTCTC |
| | | TCCCTTGTGATCACTCTGTATTGTAAGCGCGGGAGAAAGAAGCTCCTGTACATC |
| | | TTCAAGCAGCCTTTTATGCGACCTGTGCAAACCACTCAGGAAGAAGATGGGTGT |
| | | TCATGCCGCTTCCCCGAGGAGGAAGAAGGAGGGTGTGAACTGAGGGTGAAATTT |
| | | TCTAGAAGCGCCGATGCTCCCGCATATCAGCAGGGTCAGAATCAGCTCTACAAT |
| | | GAATTGAATCTCGGCAGGCGAGAAGAGTACGATGTTCTGGACAAAAGACGGGGC |
| | | AGGGATCCCGAGATGGGGGGAAAGCCCCGGAGAAAAAATCCTCAGGAGGGGTTG |
| | | TACAATGAGCTGCAGAAGGACAAGATGGCTGAAGCCTATAGCGAGATCGGAATG |
| | | AAAGGCGAAAGACGCAGAGGCAAGGGGCATGACGGTCTGTACCAGGGTCTCTCT |
| | | ACAGCCACCAAGGACACTTATGATGCGTTGCATATGCAAGCCTTGCCACCCCGC |
| 297 | 259 | CAAGTGCAGCTCGTGGAAAGCGGCGGCGGAGTGGTGCAGCCCGGCCGGAGCCTG |
| | | AGACTGTCCTGCGCCGCTTCTGGATTTACCTTCAGCAGCTACGGCATGCACTGG |
| | | GTCAGACAGGCCCCTGGCAAAGGCCTGGAGTGGGTGGCCGTTATCAGCTACGAG |
| | | GGCAGCAACAAGTATTACGCCGACAGCGTGAAGGGCCGCTTCACAATCTCTAGA |
| | | GATAATAGCAAGAACACCCTGTACCTGCAGATGAACAGCCTGCGGGCCGAAGAT |
| | | ACCGCCGTGTACTACTGTGCTAGAGAGCTGGGCGACGGCATGGACGTGTGGGGA |
| | | CAGGGCACAACCGTGACCGTGTCCTCTGGTGGTGGGGGCAGCGGTGGAGGTGGG |
| | | AGCGGAGGCGGGGGTAGCGGAGGCGGGGGTAGCGAGATCGTGCTGACCCAGTCC |
| | | CCTGCTACACTGAGCCTGTCTCCAGGCGAGCGGGCCACACTGAGCTGTAGAGCT |
| | | TCTCAGAGCGTGTCCAGCTATCTGGCCTGGTTCCAGCAGAAACCTGGCCAGGCC |
| | | CCTAGACTGCTGATCTACGACGCCAGCAACCGGGCCACCGGCATCCCCGCCAGA |
| | | TTCAGCGGCTCTGGCAGCGGCACCGACTTCACCCTCACCATCAGCAGCCTGGAA |
| | | CCCGAGGATTTTGCCGTCTACTACTGCCAGCAAAGAGTGGACCTGTGGACCTTC |
| | | GGCGGAGGAACAAAGGTGGAAATCAAGACCACAACACCTGCTCCAAGGCCCCCC |
| | | ACACCCGCTCCAACTATAGCCAGCCAACCATTGAGCCTCAGACCTGAAGCTTGC |
| | | AGGCCCGCAGCAGGAGGCGCCGTCCATACGCGAGGCCTGGACTTCGCGTGTGAT |
| | | ATTTATATTTGGGCACCTTTGGCCGGAACATGTGGGGTGTTGCTTCTCTCCCTT |
| | | GTGATCACTCTGTATTGTAAGCGCGGGAGAAAGAAGCTCCTGTACATCTTCAAG |
| | | CAGCCTTTTATGCGACCTGTGCAAACCACTCAGGAAGAAGATGGGTGTTCATGC |
| | | CGCTTCCCCGAGGAGGAAGAAGGAGGGTGTGAACTGAGGGTGAAATTTTCTAGA |
| | | AGCGCCGATGCTCCCGCATATCAGCAGGGTCAGAATCAGCTCTACAATGAATTG |
| | | AATCTCGGCAGGCGAGAAGAGTACGATGTTCTGGACAAAAGACGGGGCAGGGAT |
| | | CCCGAGATGGGGGGAAAGCCCCGGAGAAAAAATCCTCAGGAGGGGTTGTACAAT |
| | | GAGCTGCAGAAGGACAAGATGGCTGAAGCCTATAGCGAGATCGGAATGAAAGGC |
| | | GAAAGACGCAGAGGCAAGGGGCATGACGGTCTGTACCAGGGTCTCTCTACAGCC |
| | | ACCAAGGACACTTATGATGCGTTGCATATGCAAGCCTTGCCACCCCGC |
| 298 | 262 | GACATCCAGATGACCCAGAGCCCTTCGACCCTATCCGCTTCCGTGGGTGACCGT |
| | | GTGACCATCACCTGTCGCGCGTCGCAGAGCATCTCCTCCTGGCTCGCGTGGTAC |
| | | CAACAGAAGCCTGGCAAGGCCCCCAAGCTGCTGATTTACGACGCCAGTTCCCTG |
| | | GAGTCTGGCGTGCCATCCCGCTTCTCCGGCAGCGGCAGCGGTACCGAGTTCACC |
| | | CTGACGATCAGCTCCCTGCAGCCGGATGACTTTGCTACCTACTACTGTCAGCAG |
| | | GTCTCCTCCCTCCCCCCCACCTTCGGTGGCGGTACCAAGGTGGAGATCAAGGGC |
| | | GGCGGCGGCTCTGGTGGCGGAGGTTCTGGCGGGGGAGGTTCGGGGGGGGGAGGC |
| | | TCCGAGGTGCAACTGGTAGAGAGCGGCGGGGGACTGGTAAAACCCGGCGGCTCC |
| | | CTGCGGCTGTCATGCGCTGCTAGCGGCTTCACGTTCAGCGATTACTACATGAGT |
| | | TGGATCCGCCAGGCCCCCGGGAAGGGTTTGGAGTGGGTCTCGTATATCTCTTCC |
| | | AGCGGATCTACCATTTACTATGCGGACAGCGTGAAGGGGCGCTTCACCATATCT |
| | | CGGGACAACGCCAAGAACTCCCTGTACCTGCAGATGAATTCCCTGCGTGCCGAG |
| | | GACACGGCCGTGTATTACTGTGCCCGCGACCAGGGCAACTACGGCGTCGACGTG |
| | | TGGGGCCAGGGTACAACCGTCACCGTGTCCAGTACCACAACACCTGCTCCAAGG |
| | | CCCCCCACACCCGCTCCAACTATAGCCAGCCAACCATTGAGCCTCAGACCTGAA |
| | | GCTTGCAGGCCCGCAGCAGGAGGCGCCGTCCATACGCGAGGCCTGGACTTCGCG |
| | | TGTGATATTTATATTTGGGCACCTTTGGCCGGAACATGTGGGGTGTTGCTTCTC |
| | | TCCCTTGTGATCACTCTGTATTGTAAGCGCGGGAGAAAGAAGCTCCTGTACATC |
| | | TTCAAGCAGCCTTTTATGCGACCTGTGCAAACCACTCAGGAAGAAGATGGGTGT |
| | | TCATGCCGCTTCCCCGAGGAGGAAGAAGGAGGGTGTGAACTGAGGGTGAAATTT |
| | | TCTAGAAGCGCCGATGCTCCCGCATATCAGCAGGGTCAGAATCAGCTCTACAAT |
| | | GAATTGAATCTCGGCAGGCGAGAAGAGTACGATGTTCTGGACAAAAGACGGGGC |
| | | AGGGATCCCGAGATGGGGGGAAAGCCCCGGAGAAAAAATCCTCAGGAGGGGTTG |
| | | TACAATGAGCTGCAGAAGGACAAGATGGCTGAAGCCTATAGCGAGATCGGAATG |
| | | AAAGGCGAAAGACGCAGAGGCAAGGGGCATGACGGTCTGTACCAGGGTCTCTCT |
| | | ACAGCCACCAAGGACACTTATGATGCGTTGCATATGCAAGCCTTGCCACCCCGC |
| 299 | 263 | CAAGTGCAGCTGGTCGAGAGCGGAGGAGGCCTGGTTAAGCCCGGCGGATCTCTC |
| | | AGACTGAGCTGCGCCGCTAGCGGCTTTACATTCAGCGACTACTACATGAGCTGG |
| | | ATCCGGCAGGCCCCTGGCAAGGGCCTGGAATGGGTGTCCTACATCAGCTCCTCC |
| | | GGCAGCACCATCTACTACGCCGACAGCGTGAAAGGCAGATTCACAATCTCTAGA |
| | | GATAATGCCAAGAACAGCCTGTACCTGCAGATGAACAGCCTGCGGGCCGAGGAC |
| | | ACCGCCGTGTACTATTGTGCTAGAGATCAGGGCAACTACGGCGTGGACGTGTGG |
| | | GGCCAGGGCACCACCGTGACCGTGTCTAGCGGTGGTGGGGGCAGCGGTGGAGGT |
| | | GGGAGCGGAGGCGGGGGTAGCGGAGGCGGGGGTAGCGATATCCAGATGACCCAG |
| | | TCCCCATCTACACTGAGCGCCTCTGTGGGCGACCGGGTGACCATTACATGTAGA |
| | | GCCAGCCAGAGCATCAGCAGCTGGCTGGCTTGGTATCAGCAGAAACCTGGCAAG |
| | | GCCCCTAAGCTGCTGATCTACGAGGCCAGCAGCCTGGAAAGCGGCGTCCCCAGC |
| | | AGATTCAGCGGCAGCGGCTCTGGAACAGAGTTCACCCTGACCATCTCCTCCCTG |
| | | CAGCCTGACGACTTCGCCACCTACTACTGCCAGCAATCTGATAGCCACCCCATC |
| | | ACCTTTGGCGGAGGCACCAAGGTGGAAATCAAGACCACAACACCTGCTCCAAGG |
| | | CCCCCCACACCCGCTCCAACTATAGCCAGCCAACCATTGAGCCTCAGACCTGAA |
| | | GCTTGCAGGCCCGCAGCAGGAGGCGCCGTCCATACGCGAGGCCTGGACTTCGCG |
| | | TGTGATATTTATATTTGGGCACCTTTGGCCGGAACATGTGGGGTGTTGCTTCTC |
| | | TCCCTTGTGATCACTCTGTATTGTAAGCGCGGGAGAAAGAAGCTCCTGTACATC |
| | | TTCAAGCAGCCTTTTATGCGACCTGTGCAAACCACTCAGGAAGAAGATGGGTGT |
| | | TCATGCCGCTTCCCCGAGGAGGAAGAAGGAGGGTGTGAACTGAGGGTGAAATTT |
| | | TCTAGAAGCGCCGATGCTCCCGCATATCAGCAGGGTCAGAATCAGCTCTACAAT |
| | | GAATTGAATCTCGGCAGGCGAGAAGAGTACGATGTTCTGGACAAAAGACGGGGC |
| | | AGGGATCCCGAGATGGGGGGAAAGCCCCGGAGAAAAAATCCTCAGGAGGGGTTG |
| | | TACAATGAGCTGCAGAAGGACAAGATGGCTGAAGCCTATAGCGAGATCGGAATG |
| | | AAAGGCGAAAGACGCAGAGGCAAGGGGCATGACGGTCTGTACCAGGGTCTCTCT |
| | | ACAGCCACCAAGGACACTTATGATGCGTTGCATATGCAAGCCTTGCCACCCCGC |
| 300 | 266 | GATATCCAGATGACCCAGTCCCCATCTACACTGAGCGCCTCTGTGGGCGACCGG |
| | | GTGACAATTACCTGTAGAGCTAGCCAGAGCATCTCCTCCTGGCTGGCTTGGTAC |
| | | CAGCAAAAACCTGGCAAGGCCCCTAAGCTGCTGATCTACGAGGCCAGCAGCCTG |
| | | GAAAGCGGCGTCCCCTCTAGATTCAGCGGCAGCGGCTCTGGAACCGAGTTCACC |
| | | CTGACAATCAGCAGCCTGCAGCCTGACGACTTCGCCACCTATTACTGCCAGCAG |
| | | GCCAACAGCCACCCCATCACCTTTGGCGGAGGCACCAAGGTGGAAATCAAGGGT |
| | | GGTGGGGGCAGCGGTGGAGGTGGGAGCGGAGGCGGGGGTAGCGGAGGCGGGGGT |
| | | AGCGAGGTGCAGCTGGTGGAAAGCGGCGGAGGACTCGTTAAGCCCGGCGGCAGC |
| | | CTGAGACTGAGCTGCGCCGCTAGCGGATTTACCTTCAGCGACTACTACATGAGC |
| | | TGGATCCGGCAGGCCCCTGGCAAGGGCCTGGAATGGGTCAGCTACATCAGCTCC |
| | | TCTGGCTCTACAATCTACTACGCCGACAGCGTGAAAGGCAGATTCACCATCTCT |
| | | AGAGATAATGCCAAGAACAGCCTGTACCTGCAAATGAACAGCCTGCGGGCCGAG |
| | | GACACCGCCGTGTACTATTGTGCTAGAGATCAGGGCAACTACGGCGTGGACGTG |
| | | TGGGGCCAGGGCACCACCGTGACAGTGTCCTCCACCACAACACCTGCTCCAAGG |
| | | CCCCCCACACCCGCTCCAACTATAGCCAGCCAACCATTGAGCCTCAGACCTGAA |
| | | GCTTGCAGGCCCGCAGCAGGAGGCGCCGTCCATACGCGAGGCCTGGACTTCGCG |
| | | TGTGATATTTATATTTGGGCACCTTTGGCCGGAACATGTGGGGTGTTGCTTCTC |
| | | TCCCTTGTGATCACTCTGTATTGTAAGCGCGGGAGAAAGAAGCTCCTGTACATC |
| | | TTCAAGCAGCCTTTTATGCGACCTGTGCAAACCACTCAGGAAGAAGATGGGTGT |
| | | TCATGCCGCTTCCCCGAGGAGGAAGAAGGAGGGTGTGAACTGAGGGTGAAATTT |
| | | TCTAGAAGCGCCGATGCTCCCGCATATCAGCAGGGTCAGAATCAGCTCTACAAT |
| | | GAATTGAATCTCGGCAGGCGAGAAGAGTACGATGTTCTGGACAAAAGACGGGGC |
| | | AGGGATCCCGAGATGGGGGGAAAGCCCCGGAGAAAAAATCCTCAGGAGGGGTTG |
| | | TACAATGAGCTGCAGAAGGACAAGATGGCTGAAGCCTATAGCGAGATCGGAATG |
| | | AAAGGCGAAAGACGCAGAGGCAAGGGGCATGACGGTCTGTACCAGGGTCTCTCT |
| | | ACAGCCACCAAGGACACTTATGATGCGTTGCATATGCAAGCCTTGCCACCCCGC |
| 301 | 268 | GACATCCAGATGACCCAGAGCCCTAGCTCCCTGAGCGCCAGCGTGGGCGATAGA |
| | | GTGACCATTACCTGTAGAGCCTCTCAGAGCATCTCCTCCTACCTGAACTGGTAT |
| | | CAGCAGAAACCCGGCAAGGCCCCTAAGCTGCTGATCTACGCCGCTAGCAGCCTG |
| | | CAGTCTGGCGTCCCCAGCCGGTTCAGCGGCAGCGGATCTGGCACCGACTTCACC |
| | | CTGACAATCAGCAGCCTGCAACCTGAGGACTTTGCTACATACTACTGCCAGCAG |
| | | GCCCACAGCTCTCCAATCACCTTCGGCGGCGGAACAAAGGTGGAAATCAAGGGT |
| | | GGTGGGGGCAGCGGTGGAGGTGGGAGCGGAGGCGGGGGTAGCGGAGGCGGGGGT |
| | | AGCGAGGTGCAGCTGCTGGAAAGCGGAGGCGGACTCGTTCAACCTGGCGGCAGC |
| | | CTGAGACTGAGCTGCGCCGCTTCTGGATTTACCTTCAGCAACTACGCCATGAGC |
| | | TGGGTGCGGCAGGCCCCTGGCAAAGGCCTGGAATGGGTCTCCGCCATCAGCGGC |
| | | TCTGGCGGCTCCACCTACTACGCCGACAGCGTGAAGGGCAGATTCACCATCTCT |
| | | AGAGATAATAGCAAGAACACCCTGTACCTGCAGATGAACAGCCTGCGGGCCGAG |
| | | GACACCGCCGTGTACTATTGTGCTAGACCCGGAGATGGCTACTACGAGGGCGTG |
| | | TACTTCGACTACTGGGGCCAGGGCACACTGGTGACAGTGTCCAGCACCACAACA |
| | | CCTGCTCCAAGGCCCCCCACACCCGCTCCAACTATAGCCAGCCAACCATTGAGC |
| | | CTCAGACCTGAAGCTTGCAGGCCCGCAGCAGGAGGCGCCGTCCATACGCGAGGC |
| | | CTGGACTTCGCGTGTGATATTTATATTTGGGCACCTTTGGCCGGAACATGTGGG |
| | | GTGTTGCTTCTCTCCCTTGTGATCACTCTGTATTGTAAGCGCGGGAGAAAGAAG |
| | | CTCCTGTACATCTTCAAGCAGCCTTTTATGCGACCTGTGCAAACCACTCAGGAA |
| | | GAAGATGGGTGTTCATGCCGCTTCCCCGAGGAGGAAGAAGGAGGGTGTGAACTG |
| | | AGGGTGAAATTTTCTAGAAGCGCCGATGCTCCCGCATATCAGCAGGGTCAGAAT |
| | | CAGCTCTACAATGAATTGAATCTCGGCAGGCGAGAAGAGTACGATGTTCTGGAC |
| | | AAAAGACGGGGCAGGGATCCCGAGATGGGGGGAAAGCCCCGGAGAAAAAATCCT |
| | | CAGGAGGGGTTGTACAATGAGCTGCAGAAGGACAAGATGGCTGAAGCCTATAGC |
| | | GAGATCGGAATGAAAGGCGAAAGACGCAGAGGCAAGGGGCATGACGGTCTGTAC |
| | | CAGGGTCTCTCTACAGCCACCAAGGACACTTATGATGCGTTGCATATGCAAGCC |
| | | TTGCCACCCCGC |
| 302 | 270 | GATATTCAGATGACCCAGAGCCCATCTAGCCTGAGCGCCAGCGTGGGCGATAGA |
| | | GTGACCATCACCTGTCAGGCCTCTCAGGACATCGCTAATTACCTGAACTGGTAT |
| | | CAGCAGAAACCCGGCAAGGCCCCTAAGCTGCTGATCTACGACGCCTCCAACCTG |
| | | GAAACCGGCGTGCCCAGCCGGTTCAGCGGCAGCGGATCTGGCACAGACTTCACC |
| | | TTTACCATCAGCTCCCTCCAGCCTGAGGACATCGCCACATACTACTGCCAGCAA |
| | | CACTTCAACCTGCCTCTGACCTTCGGCGGCGGAACAAAGGTCGAGATCAAGGGT |
| | | GGTGGGGGCAGCGGTGGAGGTGGGAGCGGAGGCGGGGGTAGCGGAGGCGGGGGT |
| | | AGCCAAATCACCCTGAAAGAGAGCGGACCTACACTGGTCAAGCCTACCCAGACA |
| | | CTGACCCTCACATGTACATTCAGCGGCTTTAGCCTGAGCACCTCCGGCGTGGGA |
| | | GTGGGCTGGATCAGACAGCCCCCCGGCAAGGCCCTGGAATGGCTGGCTCTGATC |
| | | TATTGGAATGACGAGAAGCGGTACAGCCCTAGCCTGAAATCTAGACTGACAATC |
| | | ACCAAGGACACCAGCAAGAACCAGGTGGTGCTGACCATGACCAACATGGATCCT |
| | | GTGGATACCGCCGTGTACTACTGCGCCAGAGAAGGCTCTCACGACTACAAGAGC |
| | | TCCAACTGGTTCGACCCATGGGGCCAGGGCACCCTGGTTACAGTGTCTAGCACC |
| | | ACAACACCTGCTCCAAGGCCCCCCACACCCGCTCCAACTATAGCCAGCCAACCA |
| | | TTGAGCCTCAGACCTGAAGCTTGCAGGCCCGCAGCAGGAGGCGCCGTCCATACG |
| | | CGAGGCCTGGACTTCGCGTGTGATATTTATATTTGGGCACCTTTGGCCGGAACA |
| | | TGTGGGGTGTTGCTTCTCTCCCTTGTGATCACTCTGTATTGTAAGCGCGGGAGA |
| | | AAGAAGCTCCTGTACATCTTCAAGCAGCCTTTTATGCGACCTGTGCAAACCACT |
| | | CAGGAAGAAGATGGGTGTTCATGCCGCTTCCCCGAGGAGGAAGAAGGAGGGTGT |
| | | GAACTGAGGGTGAAATTTTCTAGAAGCGCCGATGCTCCCGCATATCAGCAGGGT |
| | | CAGAATCAGCTCTACAATGAATTGAATCTCGGCAGGCGAGAAGAGTACGATGTT |
| | | CTGGACAAAAGACGGGGCAGGGATCCCGAGATGGGGGGAAAGCCCCGGAGAAAA |
| | | AATCCTCAGGAGGGGTTGTACAATGAGCTGCAGAAGGACAAGATGGCTGAAGCC |
| | | TATAGCGAGATCGGAATGAAAGGCGAAAGACGCAGAGGCAAGGGGCATGACGGT |
| | | CTGTACCAGGGTCTCTCTACAGCCACCAAGGACACTTATGATGCGTTGCATATG |
| | | CAAGCCTTGCCACCCCGC |
| 303 | 272 | GATATCGTGATGACCCAATCTCCACTGAGCCTGCCTGTGACACCTGGCGAGCCT |
| | | GCTTCTATCAGCTGTAGAAGCAGCCAGTCCCTGCTGCACAGCAACGGCTACAAC |
| | | TACCTGGACTGGTATCTGCAGAAACCCGGCCAGAGCCCCCAGCTGCTGATCTAC |
| | | CTCGGCTCTAATCGGGCCAGCGGAGTGCCTGATAGATTCAGCGGAAGCGGCTCC |
| | | GGCACCGACTTCACCCTGAAGATCAGCAGAGTGGAAGCCGAGGACGTGGGCGTC |
| | | TACTACTGCATGCAGGCCCTGGGCCTGATTACATTTGGCGGCGGAACCAAGGTG |
| | | GAAATCAAGGGTGGTGGGGGCAGCGGTGGAGGTGGGAGCGGAGGCGGGGGTAGC |
| | | GGAGGCGGGGGTAGCGAAGTGCAGCTGGTTGAGAGCGGCGGCGGACTGGTGAAG |
| | | CCCGGAGGCAGCCTCAGACTGAGCTGTGCTGCTTCTGGCTTTACCTTCAGCTCT |
| | | TATAGCATGAACTGGGTGCGGCAGGCCCCTGGCAAGGGCCTGGAATGGGTCAGC |
| | | TCCATCAGCTCTTCTAGCAGCTACATCTACTACGCCGACAGCGTGAAGGGCAGA |
| | | TTCACCATCAGCAGAGATAACGCCAAGAACAGCCTGTACCTGCAGATGAATAGC |
| | | CTGCGGGCCGAGGACACCGCCGTGTACTACTGCGCCAGAGCCGGCGACACCTAC |
| | | AGCGCCGCCGATTACTACTACATGGACGTGTGGGGCAAAGGAACAACCGTGACA |
| | | GTGTCCTCCACCACAACACCTGCTCCAAGGCCCCCCACACCCGCTCCAACTATA |
| | | GCCAGCCAACCATTGAGCCTCAGACCTGAAGCTTGCAGGCCCGCAGCAGGAGGC |
| | | GCCGTCCATACGCGAGGCCTGGACTTCGCGTGTGATATTTATATTTGGGCACCT |
| | | TTGGCCGGAACATGTGGGGTGTTGCTTCTCTCCCTTGTGATCACTCTGTATTGT |
| | | AAGCGCGGGAGAAAGAAGCTCCTGTACATCTTCAAGCAGCCTTTTATGCGACCT |
| | | GTGCAAACCACTCAGGAAGAAGATGGGTGTTCATGCCGCTTCCCCGAGGAGGAA |
| | | GAAGGAGGGTGTGAACTGAGGGTGAAATTTTCTAGAAGCGCCGATGCTCCCGCA |
| | | TATCAGCAGGGTCAGAATCAGCTCTACAATGAATTGAATCTCGGCAGGCGAGAA |
| | | GAGTACGATGTTCTGGACAAAAGACGGGGCAGGGATCCCGAGATGGGGGGAAAG |
| | | CCCCGGAGAAAAAATCCTCAGGAGGGGTTGTACAATGAGCTGCAGAAGGACAAG |
| | | ATGGCTGAAGCCTATAGCGAGATCGGAATGAAAGGCGAAAGACGCAGAGGCAAG |
| | | GGGCATGACGGTCTGTACCAGGGTCTCTCTACAGCCACCAAGGACACTTATGAT |
| | | GCGTTGCATATGCAAGCCTTGCCACCCCGC |
| 304 | 273 | GAAGTGCAACTGCTGGAAAGCGGCGGAGGCCTGGTCCAGCCCGGCGGCTCTCTG |
| | | CGGCTCAGCTGCGCCGCTTCTGGATTTACCTTCGGCAGCGAGGCTATGAGCTGG |
| | | GTGCGGCAGGCCCCTGGAAAAGAGAGAGAGCTGGTGTCCGCCATCAGCGGCAGC |
| | | GGCGAGGTGACCTACTACGCCGACAGCGTGAAGGGCAGATTCACCATCTCTAGA |
| | | GATAATAGCAAGAACACCCTGTACCTGCAGATGAACAGCCTGAGAGCCGAGGAC |
| | | ACCGCCGTGTACTATTGTCAGAGACTGGTGGAAGCCAAGCGGCACTGGGGCCAG |
| | | GGCACACAGGTTACAGTGTCCAGCACCACAACACCTGCTCCAAGGCCCCCCACA |
| | | CCCGCTCCAACTATAGCCAGCCAACCATTGAGCCTCAGACCTGAAGCTTGCAGG |
| | | CCCGCAGCAGGAGGCGCCGTCCATACGCGAGGCCTGGACTTCGCGTGTGATATT |
| | | TATATTTGGGCACCTTTGGCCGGAACATGTGGGGTGTTGCTTCTCTCCCTTGTG |
| | | ATCACTCTGTATTGTAAGCGCGGGAGAAAGAAGCTCCTGTACATCTTCAAGCAG |
| | | CCTTTTATGCGACCTGTGCAAACCACTCAGGAAGAAGATGGGTGTTCATGCCGC |
| | | TTCCCCGAGGAGGAAGAAGGAGGGTGTGAACTGAGGGTGAAATTTTCTAGAAGC |
| | | GCCGATGCTCCCGCATATCAGCAGGGTCAGAATCAGCTCTACAATGAATTGAAT |
| | | CTCGGCAGGCGAGAAGAGTACGATGTTCTGGACAAAAGACGGGGCAGGGATCCC |
| | | GAGATGGGGGGAAAGCCCCGGAGAAAAAATCCTCAGGAGGGGTTGTACAATGAG |
| | | CTGCAGAAGGACAAGATGGCTGAAGCCTATAGCGAGATCGGAATGAAAGGCGAA |
| | | AGACGCAGAGGCAAGGGGCATGACGGTCTGTACCAGGGTCTCTCTACAGCCACC |
| | | AAGGACACTTATGATGCGTTGCATATGCAAGCCTTGCCACCCCGC |
| 305 | 274 | GAAGTGCAACTGCTGGAATCTGGCGGAGGACTGGTGCAGCCCGGCGGCAGCCTG |
| | | CGGCTGAGCTGTGCTGCTTCTGGCTTTACCTTCGAGTCTGAGGCCATGAGCTGG |
| | | TATAGACAGGCCCCTGGCAAGGAAAGAGAGCTGGTCAGCGTGATCACCAGCGAG |
| | | GGCTCCACCTACTACGCCGACAGCGTGAAAGGCAGATTCACAATCAGCCGGGAC |
| | | AATAGCAAGAACACCCTGTACCTGCAGATGAACAGCCTGCGCGCCGAAGATACA |
| | | GCCGTGTACTACTGCGCCCACATCGAGTGGGAGACAAGACTCAACTGGGGCCAG |
| | | GGCACCCAGGTGACCGTGTCCAGCACCACAACACCTGCTCCAAGGCCCCCCACA |
| | | CCCGCTCCAACTATAGCCAGCCAACCATTGAGCCTCAGACCTGAAGCTTGCAGG |
| | | CCCGCAGCAGGAGGCGCCGTCCATACGCGAGGCCTGGACTTCGCGTGTGATATT |
| | | TATATTTGGGCACCTTTGGCCGGAACATGTGGGGTGTTGCTTCTCTCCCTTGTG |
| | | ATCACTCTGTATTGTAAGCGCGGGAGAAAGAAGCTCCTGTACATCTTCAAGCAG |
| | | CCTTTTATGCGACCTGTGCAAACCACTCAGGAAGAAGATGGGTGTTCATGCCGC |
| | | TTCCCCGAGGAGGAAGAAGGAGGGTGTGAACTGAGGGTGAAATTTTCTAGAAGC |
| | | GCCGATGCTCCCGCATATCAGCAGGGTCAGAATCAGCTCTACAATGAATTGAAT |
| | | CTCGGCAGGCGAGAAGAGTACGATGTTCTGGACAAAAGACGGGGCAGGGATCCC |
| | | GAGATGGGGGGAAAGCCCCGGAGAAAAAATCCTCAGGAGGGGTTGTACAATGAG |
| | | CTGCAGAAGGACAAGATGGCTGAAGCCTATAGCGAGATCGGAATGAAAGGCGAA |
| | | AGACGCAGAGGCAAGGGGCATGACGGTCTGTACCAGGGTCTCTCTACAGCCACC |
| | | AAGGACACTTATGATGCGTTGCATATGCAAGCCTTGCCACCCCGC |
| 306 | 275 | GAGGTGCAGCTGCTGGAAAGCGGAGGGGGCCTGGTCCAACCCGGCGGGTCTCTT |
| | | CGCCTAAGCTGTGCCGCTTCTGGCTTCACCTTCGACGAGTACACCATGCACTGG |
| | | TTCAGACAGGCCCCCGGCAAGGAGCGCGAGTTCGTCAGTGCAATCAGCGGAGGC |
| | | GGTAGCGAGACTTATTACGCGGACTCCGTGAAGGGCCGCTTCACCATTAGCCGC |
| | | GACAACTCCAAGAACACGCTGTACCTGCAGATGAATTCGCTGCGCGCCGAAGAT |
| | | ACGGCCGTGTACTACTGTGCCGCTGGTGGGGAGGAGGCTGGCGTGGGCTATTGG |
| | | GGCCAGGGCACCCAGGTCACCGTGTCGTCCACCACAACACCTGCTCCAAGGCCC |
| | | CCCACACCCGCTCCAACTATAGCCAGCCAACCATTGAGCCTCAGACCTGAAGCT |
| | | TGCAGGCCCGCAGCAGGAGGCGCCGTCCATACGCGAGGCCTGGACTTCGCGTGT |
| | | GATATTTATATTTGGGCACCTTTGGCCGGAACATGTGGGGTGTTGCTTCTCTCC |
| | | CTTGTGATCACTCTGTATTGTAAGCGCGGGAGAAAGAAGCTCCTGTACATCTTC |
| | | AAGCAGCCTTTTATGCGACCTGTGCAAACCACTCAGGAAGAAGATGGGTGTTCA |
| | | TGCCGCTTCCCCGAGGAGGAAGAAGGAGGGTGTGAACTGAGGGTGAAATTTTCT |
| | | AGAAGCGCCGATGCTCCCGCATATCAGCAGGGTCAGAATCAGCTCTACAATGAA |
| | | TTGAATCTCGGCAGGCGAGAAGAGTACGATGTTCTGGACAAAAGACGGGGCAGG |
| | | GATCCCGAGATGGGGGGAAAGCCCCGGAGAAAAAATCCTCAGGAGGGGTTGTAC |
| | | AATGAGCTGCAGAAGGACAAGATGGCTGAAGCCTATAGCGAGATCGGAATGAAA |
| | | GGCGAAAGACGCAGAGGCAAGGGGCATGACGGTCTGTACCAGGGTCTCTCTACA |
| | | GCCACCAAGGACACTTATGATGCGTTGCATATGCAAGCCTTGCCACCCCGC |
| 307 | 276 | GAGGTGCAGCTGCTGGAGAGCGGAGGCGGCCTCGTGCAGCCAGGAGGTTCCCTA |
| | | CGACTCTCCTGTGCCGCCAGCGGCTTCACCTTCGAGGACTACGCCATGAGTTGG |
| | | TTCCGCCAGGCCCCGGGGAAGGAGCGCGAGGGCGTGAGCGCGATTTCTGGAAAG |
| | | GGCGGCTCCACCTATTACGCGGACTCCGTGAAGGGTCGCTTTACCATCTCTCGC |
| | | GACAACTCCAAGAACACGCTGTACCTGCAGATGAATAGCCTGCGCGCTGAGGAC |
| | | ACTGCCGTGTACTACTGTGCTGTCTTGGACGAGGAGGCCGGCGCAGAGGGCGGC |
| | | TATTGGGGCCAGGGTACCCAGGTCACCGTGTCGTCCACCACAACACCTGCTCCA |
| | | AGGCCCCCCACACCCGCTCCAACTATAGCCAGCCAACCATTGAGCCTCAGACCT |
| | | GAAGCTTGCAGGCCCGCAGCAGGAGGCGCCGTCCATACGCGAGGCCTGGACTTC |
| | | GCGTGTGATATTTATATTTGGGCACCTTTGGCCGGAACATGTGGGGTGTTGCTT |
| | | CTCTCCCTTGTGATCACTCTGTATTGTAAGCGCGGGAGAAAGAAGCTCCTGTAC |
| | | ATCTTCAAGCAGCCTTTTATGCGACCTGTGCAAACCACTCAGGAAGAAGATGGG |
| | | TGTTCATGCCGCTTCCCCGAGGAGGAAGAAGGAGGGTGTGAACTGAGGGTGAAA |
| | | TTTTCTAGAAGCGCCGATGCTCCCGCATATCAGCAGGGTCAGAATCAGCTCTAC |
| | | AATGAATTGAATCTCGGCAGGCGAGAAGAGTACGATGTTCTGGACAAAAGACGG |
| | | GGCAGGGATCCCGAGATGGGGGGAAAGCCCCGGAGAAAAAATCCTCAGGAGGGG |
| | | TTGTACAATGAGCTGCAGAAGGACAAGATGGCTGAAGCCTATAGCGAGATCGGA |
| | | ATGAAAGGCGAAAGACGCAGAGGCAAGGGGCATGACGGTCTGTACCAGGGTCTC |
| | | TCTACAGCCACCAAGGACACTTATGATGCGTTGCATATGCAAGCCTTGCCACCC |
| | | CGC |
| 308 | 277 | GAGGTGCAACTGCTGGAAAGCGGCGGTGGACTGGTGCAGCCCGGCGGCAGCCTG |
| | | AGACTGTCTTGTGCTGCTTCTGGATTTACATTCGACAGATACGCCATGAGCTGG |
| | | TTCCGCCAGGCCCCTGGCAAAGAGCGGGAAGGCGTGTCCGCCATCTCCACAAGC |
| | | GGAGATAGCACATACTATGCCGACAGCGTGAAGGGCAGATTCACCATCAGCAGA |
| | | GATAATAGCAAGAACACCCTGTACCTGCAGATGAACAGCCTCCGGGCCGAGGAC |
| | | ACCGCCGTCTACTACTGCGCCGTGCTGGACGAGGAAGCCGGCGCCGAGGGCGGC |
| | | TACTGGGGCCAGGGCACCCAGGTGACCGTGTCTAGCACCACAACACCTGCTCCA |
| | | AGGCCCCCCACACCCGCTCCAACTATAGCCAGCCAACCATTGAGCCTCAGACCT |
| | | GAAGCTTGCAGGCCCGCAGCAGGAGGCGCCGTCCATACGCGAGGCCTGGACTTC |
| | | GCGTGTGATATTTATATTTGGGCACCTTTGGCCGGAACATGTGGGGTGTTGCTT |
| | | CTCTCCCTTGTGATCACTCTGTATTGTAAGCGCGGGAGAAAGAAGCTCCTGTAC |
| | | ATCTTCAAGCAGCCTTTTATGCGACCTGTGCAAACCACTCAGGAAGAAGATGGG |
| | | TGTTCATGCCGCTTCCCCGAGGAGGAAGAAGGAGGGTGTGAACTGAGGGTGAAA |
| | | TTTTCTAGAAGCGCCGATGCTCCCGCATATCAGCAGGGTCAGAATCAGCTCTAC |
| | | AATGAATTGAATCTCGGCAGGCGAGAAGAGTACGATGTTCTGGACAAAAGACGG |
| | | GGCAGGGATCCCGAGATGGGGGGAAAGCCCCGGAGAAAAAATCCTCAGGAGGGG |
| | | TTGTACAATGAGCTGCAGAAGGACAAGATGGCTGAAGCCTATAGCGAGATCGGA |
| | | ATGAAAGGCGAAAGACGCAGAGGCAAGGGGCATGACGGTCTGTACCAGGGTCTC |
| | | TCTACAGCCACCAAGGACACTTATGATGCGTTGCATATGCAAGCCTTGCCACCC |
| | | CGC |
| 309 | 278 | GAGGTGCAACTGCTGGAAAGCGGCGGAGGACTCGTCCAGCCCGGCGGCAGCCTG |
| | | CGGCTGAGCTGTGCTGCTTCTGGATTTACCTTCGCCAGCGACGCCATGAGCTGG |
| | | TATAGACAGGCCCCTGGCAAAGAGCGGGAACTGGTGTCCGCCATCAGCGGCTCT |
| | | GGCGGCTCCACCTACTACGCCGATAGCGTGAAGGGCAGATTCACAATCTCTAGA |
| | | GATAATAGCAAGAACACCCTGTACCTGCAGATGAACAGCCTGAGAGCCGAGGAC |
| | | ACCGCCGTGTACTACTGCGCCGCTCACGACAGCGGCGAGGCCTACCTGGCCTTC |
| | | GACTACTGGGGCCAGGGCACACAGGTGACCGTGTCTAGCACCACAACACCTGCT |
| | | CCAAGGCCCCCCACACCCGCTCCAACTATAGCCAGCCAACCATTGAGCCTCAGA |
| | | CCTGAAGCTTGCAGGCCCGCAGCAGGAGGCGCCGTCCATACGCGAGGCCTGGAC |
| | | TTCGCGTGTGATATTTATATTTGGGCACCTTTGGCCGGAACATGTGGGGTGTTG |
| | | CTTCTCTCCCTTGTGATCACTCTGTATTGTAAGCGCGGGAGAAAGAAGCTCCTG |
| | | TACATCTTCAAGCAGCCTTTTATGCGACCTGTGCAAACCACTCAGGAAGAAGAT |
| | | GGGTGTTCATGCCGCTTCCCCGAGGAGGAAGAAGGAGGGTGTGAACTGAGGGTG |
| | | AAATTTTCTAGAAGCGCCGATGCTCCCGCATATCAGCAGGGTCAGAATCAGCTC |
| | | TACAATGAATTGAATCTCGGCAGGCGAGAAGAGTACGATGTTCTGGACAAAAGA |
| | | CGGGGCAGGGATCCCGAGATGGGGGGAAAGCCCCGGAGAAAAAATCCTCAGGAG |
| | | GGGTTGTACAATGAGCTGCAGAAGGACAAGATGGCTGAAGCCTATAGCGAGATC |
| | | GGAATGAAAGGCGAAAGACGCAGAGGCAAGGGGCATGACGGTCTGTACCAGGGT |
| | | CTCTCTACAGCCACCAAGGACACTTATGATGCGTTGCATATGCAAGCCTTGCCA |
| | | CCCCGC |
| 310 | 279 | GAGGTGCAACTGCTGGAAAGCGGCGGAGGACTCGTCCAGCCCGGCGGCAGCCTG |
| | | AGGCTGAGCTGTGCTGCTTCTGGCTTTACCTTCGACTCCTACACAATGAGCTGG |
| | | TATAGACAGGCCCCTGGCAAGGAGCGGGAACTGGTGTCCGCCATCAGCGGCCAC |
| | | GGCGACTCTACATACTACGCCGACAGCGTGAAAGGCAGATTCACAATCTCTAGA |
| | | GATAATAGCAAGAACACCCTGTACCTGCAGATGAACAGCCTGCGGGCCGAGGAC |
| | | ACCGCCGTGTACTACTGCACCAGAATCAGCATCACCACCGAGTGGCTGGCCGGA |
| | | GATTACTGGGGCCAGGGCACCCAGGTGACAGTGTCCAGCACCACAACACCTGCT |
| | | CCAAGGCCCCCCACACCCGCTCCAACTATAGCCAGCCAACCATTGAGCCTCAGA |
| | | CCTGAAGCTTGCAGGCCCGCAGCAGGAGGCGCCGTCCATACGCGAGGCCTGGAC |
| | | TTCGCGTGTGATATTTATATTTGGGCACCTTTGGCCGGAACATGTGGGGTGTTG |
| | | CTTCTCTCCCTTGTGATCACTCTGTATTGTAAGCGCGGGAGAAAGAAGCTCCTG |
| | | TACATCTTCAAGCAGCCTTTTATGCGACCTGTGCAAACCACTCAGGAAGAAGAT |
| | | GGGTGTTCATGCCGCTTCCCCGAGGAGGAAGAAGGAGGGTGTGAACTGAGGGTG |
| | | AAATTTTCTAGAAGCGCCGATGCTCCCGCATATCAGCAGGGTCAGAATCAGCTC |
| | | TACAATGAATTGAATCTCGGCAGGCGAGAAGAGTACGATGTTCTGGACAAAAGA |
| | | CGGGGCAGGGATCCCGAGATGGGGGGAAAGCCCCGGAGAAAAAATCCTCAGGAG |
| | | GGGTTGTACAATGAGCTGCAGAAGGACAAGATGGCTGAAGCCTATAGCGAGATC |
| | | GGAATGAAAGGCGAAAGACGCAGAGGCAAGGGGCATGACGGTCTGTACCAGGGT |
| | | CTCTCTACAGCCACCAAGGACACTTATGATGCGTTGCATATGCAAGCCTTGCCA |
| | | CCCCGC |
| 311 | 280 | GAGGTGCAGCTGCTGGAAAGCGGAGGAGGCCTGGTCCAACCTGGCGGCAGCCTG |
| | | CGGCTGAGCTGCGCCGCTTCTGGCTTCACCTTCAGCAGCTACGCCATGAGCTGG |
| | | TTCCGGCAGGCCCCTGGCAAGGAAAGAGAGTTCGTGTCTTTTATCAGCGGATCT |
| | | GGCGACTCCACCTACTACGCTGATAGCGTGAAAGGCAGATTTACCATCTCTAGA |
| | | GATAATAGCAAGAACACCCTGTACCTCCAGATGAACAGCCTGCGCGCCGAGGAC |
| | | ACAGCCGTGTACTATTGTACCAGATGGCCTTACGACTTCGAGGAACCAAGCGAG |
| | | CCCGGCGTGTACTGGGGCCAGGGCACACAGGTGACAGTGTCCTCCACCACAACA |
| | | CCTGCTCCAAGGCCCCCCACACCCGCTCCAACTATAGCCAGCCAACCATTGAGC |
| | | CTCAGACCTGAAGCTTGCAGGCCCGCAGCAGGAGGCGCCGTCCATACGCGAGGC |
| | | CTGGACTTCGCGTGTGATATTTATATTTGGGCACCTTTGGCCGGAACATGTGGG |
| | | GTGTTGCTTCTCTCCCTTGTGATCACTCTGTATTGTAAGCGCGGGAGAAAGAAG |
| | | CTCCTGTACATCTTCAAGCAGCCTTTTATGCGACCTGTGCAAACCACTCAGGAA |
| | | GAAGATGGGTGTTCATGCCGCTTCCCCGAGGAGGAAGAAGGAGGGTGTGAACTG |
| | | AGGGTGAAATTTTCTAGAAGCGCCGATGCTCCCGCATATCAGCAGGGTCAGAAT |
| | | CAGCTCTACAATGAATTGAATCTCGGCAGGCGAGAAGAGTACGATGTTCTGGAC |
| | | AAAAGACGGGGCAGGGATCCCGAGATGGGGGGAAAGCCCCGGAGAAAAAATCCT |
| | | CAGGAGGGGTTGTACAATGAGCTGCAGAAGGACAAGATGGCTGAAGCCTATAGC |
| | | GAGATCGGAATGAAAGGCGAAAGACGCAGAGGCAAGGGGCATGACGGTCTGTAC |
| | | CAGGGTCTCTCTACAGCCACCAAGGACACTTATGATGCGTTGCATATGCAAGCC |
| | | TTGCCACCCCGC |
| 312 | 281 | GAGGTGCAGCTGCTGGAAAGCGGCGGAGGCCTGGTGCAACCTGGCGGATCTCTC |
| | | AGACTGAGCTGTGCTGCTTCTGGCTTCACATTCACCGACTACGACATGAGCTGG |
| | | TATAGACAGGCCCCTGGAAAAGAGCGGGAACTGGTCTCCGTGATCCACAGCGGC |
| | | GGCTCCACCTACTACGCCGATAGCGTGAAGGGCAGATTCACCATCAGCAGAGAT |
| | | AATAGCAAGAACACCCTGTACCTGCAGATGAACAGCCTGCGGGCCGAGGACACC |
| | | GCCGTGTACTACTGCGCCCCCGGCTACTACAGCGACCTGTCTTTTGATTATTAC |
| | | AACTTCGACTACTGGGGCCAGGGCACACAGGTGACAGTGTCCAGCACCACAACA |
| | | CCTGCTCCAAGGCCCCCCACACCCGCTCCAACTATAGCCAGCCAACCATTGAGC |
| | | CTCAGACCTGAAGCTTGCAGGCCCGCAGCAGGAGGCGCCGTCCATACGCGAGGC |
| | | CTGGACTTCGCGTGTGATATTTATATTTGGGCACCTTTGGCCGGAACATGTGGG |
| | | GTGTTGCTTCTCTCCCTTGTGATCACTCTGTATTGTAAGCGCGGGAGAAAGAAG |
| | | CTCCTGTACATCTTCAAGCAGCCTTTTATGCGACCTGTGCAAACCACTCAGGAA |
| | | GAAGATGGGTGTTCATGCCGCTTCCCCGAGGAGGAAGAAGGAGGGTGTGAACTG |
| | | AGGGTGAAATTTTCTAGAAGCGCCGATGCTCCCGCATATCAGCAGGGTCAGAAT |
| | | CAGCTCTACAATGAATTGAATCTCGGCAGGCGAGAAGAGTACGATGTTCTGGAC |
| | | AAAAGACGGGGCAGGGATCCCGAGATGGGGGGAAAGCCCCGGAGAAAAAATCCT |
| | | CAGGAGGGGTTGTACAATGAGCTGCAGAAGGACAAGATGGCTGAAGCCTATAGC |
| | | GAGATCGGAATGAAAGGCGAAAGACGCAGAGGCAAGGGGCATGACGGTCTGTAC |
| | | CAGGGTCTCTCTACAGCCACCAAGGACACTTATGATGCGTTGCATATGCAAGCC |
| | | TTGCCACCCCGC |
| 313 | 282 | GAGGTGCAGCTGCTGGAGAGCGGTGGAGGGTTGGTGCAGCCCGGGGGTAGCCTG |
| | | CGTCTGTCGTGCGCCGCTTCCGGCTTCACGTTCTCTGATTACGCCATGCACTGG |
| | | TTCCGGCAGGCCCCCGGTAAGGAGCGCGTGCTGGTGTCGTCTATTGACTCCGGC |
| | | GGCTCCACTTACTACGCAGACAGTGTCAAGGGCCGTTTCACCATCAGCCGCGAC |
| | | AACAGCAAGAACACGCTGTACCTGCAGATGAACTCCCTTCGAGCAGAGGACACC |
| | | GCGGTGTACTACTGTAATGCGGGCTTCAAGGGCGATCACCCCCACCCCAAGGAT |
| | | GCCTTCGACATTTGGGGCCAGGGCACCCAGGTCACCGTGTCGTCCACCACAACA |
| | | CCTGCTCCAAGGCCCCCCACACCCGCTCCAACTATAGCCAGCCAACCATTGAGC |
| | | CTCAGACCTGAAGCTTGCAGGCCCGCAGCAGGAGGCGCCGTCCATACGCGAGGC |
| | | CTGGACTTCGCGTGTGATATTTATATTTGGGCACCTTTGGCCGGAACATGTGGG |
| | | GTGTTGCTTCTCTCCCTTGTGATCACTCTGTATTGTAAGCGCGGGAGAAAGAAG |
| | | CTCCTGTACATCTTCAAGCAGCCTTTTATGCGACCTGTGCAAACCACTCAGGAA |
| | | GAAGATGGGTGTTCATGCCGCTTCCCCGAGGAGGAAGAAGGAGGGTGTGAACTG |
| | | AGGGTGAAATTTTCTAGAAGCGCCGATGCTCCCGCATATCAGCAGGGTCAGAAT |
| | | CAGCTCTACAATGAATTGAATCTCGGCAGGCGAGAAGAGTACGATGTTCTGGAC |
| | | AAAAGACGGGGCAGGGATCCCGAGATGGGGGGAAAGCCCCGGAGAAAAAATCCT |
| | | CAGGAGGGGTTGTACAATGAGCTGCAGAAGGACAAGATGGCTGAAGCCTATAGC |
| | | GAGATCGGAATGAAAGGCGAAAGACGCAGAGGCAAGGGGCATGACGGTCTGTAC |
| | | CAGGGTCTCTCTACAGCCACCAAGGACACTTATGATGCGTTGCATATGCAAGCC |
| | | TTGCCACCCCGC |
| 314 | 283 | GAGGTGCAACTGCTGGAATCCGGCGGAGGCCTGGTGCAGCCCGGCGGCAGCCTC |
| | | AGACTGAGCTGTGCCGCTTCTGGCTTTACCTTCAGCAGCGAGGGCATGAGCTGG |
| | | GTGCGGCAGGCCCCTGGCAAGGAAAGAGAGCTGGTCTCCGCCATCAGCGGATCT |
| | | GGCGACCACACCTACTATGCCGATAGCGTGCGCGGAAGATTCACAATCTCTAGA |
| | | GATAATAGCAAGAACACCCTGTACCTGCAGATGAACAGCCTGCGGGCCGAGGAC |
| | | ACCGCCGTGTACTACTGCAACGCCCTGGAAGGCGGCCCTACAACAGCTATCCAG |
| | | CCAGGAGGCCCTGACTACTGGGGCCAGGGCACCCAGGTGACCGTGTCCAGCACC |
| | | ACAACACCTGCTCCAAGGCCCCCCACACCCGCTCCAACTATAGCCAGCCAACCA |
| | | TTGAGCCTCAGACCTGAAGCTTGCAGGCCCGCAGCAGGAGGCGCCGTCCATACG |
| | | CGAGGCCTGGACTTCGCGTGTGATATTTATATTTGGGCACCTTTGGCCGGAACA |
| | | TGTGGGGTGTTGCTTCTCTCCCTTGTGATCACTCTGTATTGTAAGCGCGGGAGA |
| | | AAGAAGCTCCTGTACATCTTCAAGCAGCCTTTTATGCGACCTGTGCAAACCACT |
| | | CAGGAAGAAGATGGGTGTTCATGCCGCTTCCCCGAGGAGGAAGAAGGAGGGTGT |
| | | GAACTGAGGGTGAAATTTTCTAGAAGCGCCGATGCTCCCGCATATCAGCAGGGT |
| | | CAGAATCAGCTCTACAATGAATTGAATCTCGGCAGGCGAGAAGAGTACGATGTT |
| | | CTGGACAAAAGACGGGGCAGGGATCCCGAGATGGGGGGAAAGCCCCGGAGAAAA |
| | | AATCCTCAGGAGGGGTTGTACAATGAGCTGCAGAAGGACAAGATGGCTGAAGCC |
| | | TATAGCGAGATCGGAATGAAAGGCGAAAGACGCAGAGGCAAGGGGCATGACGGT |
| | | CTGTACCAGGGTCTCTCTACAGCCACCAAGGACACTTATGATGCGTTGCATATG |
| | | CAAGCCTTGCCACCCCGC |

In particular embodiments, polynucleotides encoding a chimeric antigen receptor may be codon-optimized. As used herein, the term “codon-optimized” refers to substituting codons in a polynucleotide encoding a polypeptide in order to modulate polypeptide expression, stability and/or activity. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

A “nucleic acid cassette,” “expression cassette” or “nucleic acid expression cassette” refers to polynucleotide sequences sufficient to transcribe an RNA, which is ultimately translated to a polypeptide. In particular embodiments, a nucleic acid cassette comprises a polynucleotide-of-interest, a polynucleotide that encodes a polypeptide, e.g., a CAR. Nucleic acid expression cassettes contemplated in particular embodiments comprise one or more expression control sequences, e.g., a promoter, enhancer, poly(A) sequence, and one or more polynucleotide(s)-of-interest. In particular embodiments, a vector contemplated herein comprises one or more nucleic acid cassettes. In particular embodiments, a nucleic acid cassette is oriented in a vector to enable transcription of a polynucleotide-of-interest.

In particular embodiments, a polynucleotide encoding a polypeptide may be combined with other polynucleotide sequences, such as expression control sequences, promoters and/or enhancers, untranslated regions (UTRs), polynucleotides encoding signal peptides, Kozak sequences, polyadenylation signals, restriction enzyme sites, multiple cloning sites, internal ribosomal entry sites (IRES), recombinase recognition sites, termination codons, transcriptional termination signals, and polynucleotides encoding self-cleaving polypeptides or epitope tags, as disclosed elsewhere herein or as known in the art.

Polynucleotides can be prepared, manipulated, expressed and/or delivered using any of a variety of well-established techniques known and available in the art. In order to express a desired polypeptide, a nucleotide sequence encoding the polypeptide, can be inserted into an appropriate vector, e.g., a lentiviral vector.

In particular embodiments, a vector comprises a polynucleotide comprising or encoding one or more exogenous, endogenous, or heterologous expression control sequences operably linked to a polynucleotide encoding one or more polynucleotides and/or polypeptides contemplated herein.

“Expression control sequences,” “control elements,” or “regulatory sequences” contemplated in particular embodiments include but not limited to promoters, enhancers, translation initiation signals (Shine Dalgamo sequence or Kozak sequence), introns, polyadenylation signals, 5′ and 3′ untranslated regions, all of which may interact with host cell proteins to carry out transcription and translation.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between an expression control sequence (such as a promoter, and/or enhancer) and a second polynucleotide sequence encoding a polypeptide, wherein the expression control sequence directs transcription of the nucleic acid corresponding to the second sequence.

Illustrative ubiquitous expression control sequences suitable for use in particular embodiments include, but are not limited to, a 3-actin promoter, a cytomegalovirus (CMV) immediate early promoter, a simian virus 40 (SV40) (e.g., early or late) promoter, a Moloney murine leukemia virus (MoMLV) promoter, a Rous sarcoma virus (RSV) promoter, a herpes simplex virus (HSV) (thymidine kinase) promoter, an SV40/CD43 promoter, a spleen focus forming virus (SFFV) promoter, an elongation factor 1-alpha (EF1α) short promoter (intronless), an EF1α long promoter containing an intron, a Ubiquitin C (UBC) promoter, a phosphoglycerate kinase-1 (PGK) promoter, a cytomegalovirus enhancer/chicken β-actin (CAG) promoter, and a myeloproliferative sarcoma virus enhancer, negative control region deleted, dl587rev primer-binding site substituted (MND) U3 promoter (Haas et al., Journal of Virology. 2003; 77(17): 9439-9450).

Illustrative examples of ubiquitous expression control sequences suitable for use in particular embodiments contemplated herein include those comprising polynucleotide sequences set forth in Table 14.

TABLE 14

| SEQ | |
|---|---|
| ID | |
| NO: | NUCLEIC ACID SEQUENCE |
| 318 | GCGTTACATAACTTACGGTAAATGGCCCGCCTGGCTGACCGCCCAACGACCCCCGCCCATTGAC |
| | GTCAATAATGACGTATGTTCCCATAGTAACGCCAATAGGGACTTTCCATTGACGTCAATGGGTG |
| | GAGTATTTACGGTAAACTGCCCACTTGGCAGTACATCAAGTGTATCATATGCCAAGTACGCCCC |
| | CTATTGACGTCAATGACGGTAAATGGCCCGCCTGGCATTATGCCCAGTACATGACCTTATGGGA |
| | CTTTCCTACTTGGCAGTACATCTACGTATTAGTCATCGCTATTACCATGGTCGAGGTGAGCCCC |
| | ACGTTCTGCTTCACTCTCCCCATCTCCCCCCCCTCCCCACCCCCAATTTTGTATTTATTTATTT |
| | TTTAATTATTTTGTGCAGCGATGGGGGCGGGGGGGGGGGGGGGGCGCGCGCCAGGCGGGGCGGG |
| | GCGGGGCGAGGGGCGGGGCGGGGCGAGGCGGAGAGGTGCGGCGGCAGCCAATCAGAGCGGCGCG |
| | CTCCGAAAGTTTCCTTTTATGGCGAGGCGGCGGCGGCGGCGGCCCTATAAAAAGCGAAGCGCGC |
| | GGCGGGCG |
| 319 | CGTGAGGCTCCGGTGCCCGTCAGTGGGCAGAGCGCACATCGCCCACAGTCCCCGAGAAGTTGGG |
| | GGGAGGGGTCGGCAATTGAACCGGTGCCTAGAGAAGGTGGCGCGGGGTAAACTGGGAAAGTGAT |
| | GTCGTGTACTGGCTCCGCCTTTTTCCCGAGGGTGGGGGAGAACCGTATATAAGTGCAGTAGTCG |
| | CCGTGAACGTTCTTTTTCGCAACGGGTTTGCCGCCAGAACACAGGTAAGTGCCGTGTGTGGTTC |
| | CCGCGGGCCTGGCCTCTTTACGGGTTATGGCCCTTGCGTGCCTTGAATTACTTCCACCTGGCTG |
| | CAGTACGTGATTCTTGATCCCGAGCTTCGGGTTGGAAGTGGGTGGGAGAGTTCGAGGCCTTGCG |
| | CTTAAGGAGCCCCTTCGCCTCGTGCTTGAGTTGAGGCCTGGCCTGGGCGCTGGGGCCGCCGCGT |
| | GCGAATCTGGTGGCACCTTCGCGCCTGTCTCGCTGCTTTCGATAAGTCTCTAGCCATTTAAAAT |
| | TTTTGATGACCTGCTGCGACGCTTTTTTTCTGGCAAGATAGTCTTGTAAATGCGGGCCAAGATC |
| | TGCACACTGGTATTTCGGTTTTTGGGGCCGCGGGCGGCGACGGGGCCCGTGCGTCCCAGCGCAC |
| | ATGTTCGGCGAGGCGGGGCCTGCGAGCGCGGCCACCGAGAATCGGACGGGGGTAGTCTCAAGCT |
| | GGCCGGCCTGCTCTGGTGCCTGGCCTCGCGCCGCCGTGTATCGCCCCGCCCTGGGCGGCAAGGC |
| | TGGCCCGGTCGGCACCAGTTGCGTGAGCGGAAAGATGGCCGCTTCCCGGCCCTGCTGCAGGGAG |
| | CTCAAAATGGAGGACGCGGCGCTCGGGAGAGCGGGCGGGTGAGTCACCCACACAAAGGAAAAGG |
| | GCCTTTCCGTCCTCAGCCGTCGCTTCATGTGACTCCACGGAGTACCGGGCGCCGTCCAGGCACC |
| | TCGATTAGTTCTCGAGCTTTTGGAGTACGTCGTCTTTAGGTTGGGGGGAGGGGTTTTATGCGAT |
| | GGAGTTTCCCCACACTGAGTGGGTGGAGACTGAAGTTAGGCCAGCTTGGCACTTGATGTAATTC |
| | TCCTTGGAATTTGCCCTTTTTGAGTTTGGATCTTGGTTCATTCTCAAGCCTCAGACAGTGGTTC |
| | AAAGTTTTTTTCTTCCATTTCAGGTGTCGTGA |
| 320 | AATGAAAGACCCCACCTGTAGGTTTGGCAAGCTAGGATCAAGGTTAGGAACAGAGAGACAGCAG |
| | AATATGGGCCAAACAGGATATCTGTGGTAAGCAGTTCCTGCCCCGGCTCAGGGCCAAGAACAGT |
| | TGGAACAGCAGAATATGGGCCAAACAGGATATCTGTGGTAAGCAGTTCCTGCCCCGGCTCAGGG |
| | CCAAGAACAGATGGTCCCCAGATGCGGTCCCGCCCTCAGCAGTTTCTAGAGAACCATCAGATGT |
| | TTCCAGGGTGCCCCAAGGACCTGAAATGACCCTGTGCCTTATTTGAACTAACCAATCAGTTCGC |
| | TTCTCGCTTCTGTTCGCGCGCTTCTGCTCCCCGAGCTCAATAAAAGAGCCCACAACCCCTCACT |
| | CGGC |
| 321 | GGGTAGGGGAGGCGCTTTTCCCAAGGCAGTCTGGAGCATGCGCTTTAGCAGCCCCGCTGGGCAC |
| | TTGGCGCTACACAAGTGGCCTCTGGCCTCGCACACATTCCACATCCACCGGTAGGCGCCAACCG |
| | GCTCCGTTCTTTGGTGGCCCCTTCGCGCCACCTTCTACTCCTCCCCTAGTCAGGAAGTTCCCCC |
| | CCGCCCCGCAGCTCGCGTCGTGCAGGACGTGACAAATGGAAGTAGCACGTCTCACTAGTCTCGT |
| | GCAGATGGACAGCACCGCTGAGCAATGGAAGCGGGTAGGCCTTTGGGGCAGCGGCCAATAGCAG |
| | CTTTGCTCCTTCGCTTTCTGGGCTCAGAGGCTGGGAAGGGGTGGGTCCGGGGGCGGGCTCAGGG |
| | GCGGGCTCAGGGGCGGGGCGGGCGCCCGAAGGTCCTCCGGAGGCCCGGCATTCTGCACGCTTCA |
| | AAAGCGCACGTCTGCCGCGCTGTTCTCCTCTTCCTCATCTCCGGGCCTTTCG |
| 322 | TGAAAGACCCCACCTGTAGGTTTGGCAAGATAGCTGCAGTAACGCCATTTTGCAAGGCATGGAA |
| | AAATACCAAACCAAGAATAGAGAAGTTCAGATCAAGGGCGGGTACATGAAAATAGCTAACGTTG |
| | GGCCAAACAGGATATCTGCGGTGAGCAGTTTCGGCCCCGGCCCGGGGCCAAGAACAGATGGTCA |
| | CCGCAGTTTCGGCCCCGGCCCGAGGCCAAGAACAGATGGTCCCCAGATATGGCCCAACCCTCAG |
| | CAGTTTCTTAAGACCCATCAGATGTTTCCAGGCTCCCCCAAGGACCTGAAATGACCCTGCGCCT |
| | TATTTGAATTAACCAATCAGCCTGCTTCTCGCTTCTGTTCGCGCGCTTCTGCTTCCCGAGCTCT |
| | ATAAAAGAGCTCACAACCCCTCACTCGGCGCGCCAGTCCTCCGATTGACTGAGTCGCCC |
| 323 | GGCCTCCGCGCCGGGTTTTGGCGCCTCCCGCGGGCGCCCCCCTCCTCACGGCGAGCGCTGCCAC |
| | GTCAGACGAAGGGCGCAGCGAGCGTCCTGATCCTTCCGCCCGGACGCTCAGGACAGCGGCCCGC |
| | TGCTCATAAGACTCGGCCTTAGAACCCCAGTATCAGCAGAAGGACATTTTAGGACGGGACTTGG |
| | GTGACTCTAGGGCACTGGTTTTCTTTCCAGAGAGCGGAACAGGCGAGGAAAAGTAGTCCCTTCT |
| | CGGCGATTCTGCGGAGGGATCTCCGTGGGGCGGTGAACGCCGATGATTATATAAGGACGCGCCG |
| | GGTGTGGCACAGCTAGTTCCGTCGCAGCCGGGATTTGGGTCGCGGTTCTTGTTTGTGGATCGCT |
| | GTGATCGTCACTTGGTGAGTAGCGGGCTGCTGGGCTGGCCGGGGCTTTCGTGGCCGCCGGGCCG |
| | CTCGGTGGGACGGAAGCGTGTGGAGAGACCGCCAAGGGCTGTAGTCTGGGTCCGCGAGCAAGGT |
| | TGCCCTGAACTGGGGGTTGGGGGGAGCGCAGCAAAATGGCGGCTGTTCCCGAGTCTTGAATGGA |
| | AGACGCTTGTGAGGCGGGCTGTGAGGTCGTTGAAACAAGGTGGGGGGCATGGTGGGCGGCAAGA |
| | ACCCAAGGTCTTGAGGCCTTCGCTAATGCGGGAAAGCTCTTATTCGGGTGAGATGGGCTGGGGC |
| | ACCATCTGGGGACCCTGACGTGAAGTTTGTCACTGACTGGAGAACTCGGTTTGTCGTCTGTTGC |
| | GGGGGCGGCAGTTATGGCGGTGCCGTTGGGCAGTGCACCCGTACCTTTGGGAGCGCGCGCCCTC |
| | GTCGTGTCGTGACGTCACCCGTTCTGTTGGCTTATAATGCAGGGTGGGGCCACCTGCCGGTAGG |
| | TGTGCGGTAGGCTTTTCTCCGTCGCAGGACGCAGGGTTCGGGCCTAGGGTAGGCTCTCCTGAAT |
| | CGACAGGCGCCGGACCTCTGGTGAGGGGAGGGATAAGTGAGGCGTCAGTTTCTTTGGTCGGTTT |
| | TATGTACCTATCTTCTTAAGTAGCTGAAGCTCCGGTTTTGAACTATGCGCTCGGGGTTGGCGAG |
| | TGTGTTTTGTGAAGTTTTTTAGGCACCTTTTGAAATGTAATCATTTGGGTCAATATGTAATTTT |
| | CAGTGTTAGACTAGTAAATTGTCCGCTAAATTCTGGCCGTTTTTGGCTTTTTTGTTAGAC |

In particular embodiments, a vector comprises a promoter comprising a polynucleotide sequence set forth in any one of SEQ ID NOs: 318, 319, 320, 321, 322, and 323 operably linked to a polynucleotide sequence encoding a CAR set forth in any one of SEQ ID NOs: 255, 256, 257, 258, 259, 260, 261, 262, 263, 264, 265, 266, 267, 268, 269, 270, 271, 272, 273, 274, 275, 276, 277, 278, 279, 280, 281, 282, and 283. In particular embodiments, a vector comprises a promoter comprising a polynucleotide sequence set forth in any one of SEQ ID NOs: 318, 319, 320, 321, 322, and 323 operably linked to a polynucleotide sequence encoding a signal peptide set forth in SEQ ID NO: 245 and a polynucleotide encoding a CAR set forth in any one of SEQ ID NOs: 259, 263, 266, 270, 273, and 277. In particular embodiments, a vector comprises a promoter comprising a polynucleotide sequence set forth in any one of SEQ ID NOs: 318, 319, 320, 321, 322, and 323 operably linked to a polynucleotide comprising a polynucleotide sequence set forth in any one of SEQ ID NOs: 295, 296, 297, 298, 299, 300, 301, 302, 303, 304, 305, 306, 307, 308, 309, 310, 311, 312, 313, and 314. In particular embodiments, a vector comprises a promoter comprising a polynucleotide sequence set forth in any one of SEQ ID NOs: 318, 319, 320, 321, 322, and 323 operably linked to a polynucleotide sequence set forth in SEQ ID NO: 294 and a polynucleotide sequence set forth in any one of SEQ ID NOs: 295, 296, 297, 298, 299, 300, 301, 302, 303, 304, 305, 306, 307, 308, 309, 310, 311, 312, 313, and 314.

In particular embodiments, a vector comprises a promoter comprising a polynucleotide sequence set forth in SEQ ID NO: 319 operably linked to a polynucleotide sequence encoding a CAR set forth in any one of SEQ ID NOs: 259, 263, 266, 270, 273 or 277. In particular embodiments, a vector comprises a promoter comprising a polynucleotide sequence set forth in SEQ ID NO: 319 operably linked to a polynucleotide comprising a polynucleotide sequence set forth in any one of SEQ ID NOs: 297, 299, 300, 302, 304, and 308. In particular embodiments, a vector comprises a promoter comprising a polynucleotide sequence set forth in SEQ ID NO: 319 operably linked to a polynucleotide sequence set forth in SEQ ID NO: 294 and a polynucleotide sequence set forth in any one of SEQ ID NOs: 297, 299, 300, 302, 304, and 308.

In particular embodiments, a vector comprises a promoter comprising a polynucleotide sequence set forth in SEQ ID NO: 320 operably linked to a polynucleotide sequence encoding a CAR set forth in any one of SEQ ID NOs: 259, 263, 266, 270, 273 or 277. In particular embodiments, a vector comprises a promoter comprising a polynucleotide sequence set forth in any one of SEQ ID NO: 320 operably linked to a polynucleotide comprising a polynucleotide sequence set forth in any one of SEQ ID NOs: 297, 299, 300, 302, 304, and 308. In particular embodiments, a vector comprises a promoter comprising a polynucleotide sequence set forth in SEQ ID NO: 320 operably linked to a polynucleotide sequence set forth in SEQ ID NO: 294 and a polynucleotide sequence set forth in any one of SEQ ID NOs: 297, 299, 300, 302, 304, and 308.

In particular embodiments, expression of polynucleotide sequences may be modulated by incorporating posttranscriptional regulatory elements into vectors. A variety of posttranscriptional regulatory elements may increase expression of a heterologous nucleic acid, e.g., woodchuck hepatitis virus posttranscriptional regulatory element (WPRE; Zufferey et al., 1999, J. Virol., 73: 2886); the posttranscriptional regulatory element present in hepatitis B vims (HPRE) (Huang et al., Mol. Cell. Biol., 5:3864); and the like (Liu et al., 1995, Genes Dev., 9:1766).

Illustrative examples of posttranscriptional control sequences suitable for use in particular embodiments contemplated herein include those comprising polynucleotide sequences set forth in Table 15.

TABLE 15

| SEQ | |
|---|---|
| ID | |
| NO: | NUCLEIC ACID SEQUENCE |
| 315 | AATCAACCTCTGGATTACAAAATTTGTGAAAGATTGACTGGTATTCTTAACTATGTTGCTC |
| | CTTTTACGCTATGTGGATACGCTGCTTTAATGCCTTTGTATCATGCTATTGCTTCCCGTAT |
| | GGCTTTCATTTTCTCCTCCTTGTATAAATCCTGGTTGCTGTCTCTTTATGAGGAGTTGTGG |
| | CCCGTTGTCAGGCAACGTGGCGTGGTGTGCACTGTGTTTGCTGACGCAACCCCCACTGGTT |
| | GGGGCATTGCCACCACCTGTCAGCTCCTTTCCGGGACTTTCGCTTTCCCCCTCCCTATTGC |
| | CACGGCGGAACTCATCGCCGCCTGCCTTGCCCGCTGCTGGACAGGGGCTCGGCTGTTGGGC |
| | ACTGACAATTCCGTGGTGTTGTCGGGGAAGCTGACGTCCTTTCCATGGCTGCTCGCCTGTG |
| | TTGCCACCTGGATTCTGCGCGGGACGTCCTTCTGCTACGTCCCTTCGGCCCTCAATCCAGC |
| | GGACCTTCCTTCCCGCGGCCTGCTGCCGGCTCTGCGGCCTCTTCCGCGTCTTCGCCTTCGC |
| | CCTCAGACGAGTCGGATCTCCCTTTGGGCCGCCTCCCCGCCTG |
| 316 | AATCAACCTCTGGATTACAAAATTTGTGAAAGATTGACTGGTATTCTTAACTATGTTGCTC |
| | CTTTTACGCTATGTGGATACGCTGCTTTAATGCCTTTGTATCATGCTATTGCTTCCCGTAT |
| | GGCTTTCATTTTCTCCTCCTTGTATAAATCCTGGTTGCTGTCTCTTTATGAGGAGTTGTGG |
| | CCCGTTGTCAGGCAACGTGGCGTGGTGTGCACTGTGTTTGCTGACGCAACCCCCACTGGTT |
| | GGGGCATTGCCACCACCTGTCAGCTCCTTTCCGGGACTTTCGCTTTCCCCCTCCCTATTGC |
| | CACGGCGGAACTCATCGCCGCCTGCCTTGCCCGCTGCTGGACAGGGGCTCGGCTGTTGGGC |
| | ACTGACAATTCCGTGGTGTTGTCGGGGAAGGTCTGCTGAGACTCGGGGCTGCTCGCCTGTG |
| | TTGCCACCTGGATTCTGCGCGGGACGTCCTTCTGCTACGTCCCTTCGGCCCTCAATCCAGC |
| | GGACCTTCCTTCCCGCGGCCTGCTGCCGGCTCTGCGGCCTCTTCCGCGTCTTCGCCTTCGC |
| | CCTCAGACGAGTCGGATCTCCCTTTGGGCCGCCTCCCCGCCTG |
| 317 | AATCAACCTCTGGATTACAAAATTTGTGAAAGATTGACTGATATTCTTAACTATGTTGCTC |
| | CTTTTACGCTGTGTGGATATGCTGCTTTAATGCCTCTGTATCATGCTATTGCTTCCCGTAC |
| | GGCTTTCGTTTTCTCCTCCTTGTATAAATCCTGGTTGCTGTCTCTTTATGAGGAGTTGTGG |
| | CCCGTTGTCCGTCAACGTGGCGTGGTGTGCTCTGTGTTTGCTGACGCAACCCCCACTGGCT |
| | GGGGCATTGCCACCACCTGTCAACTCCTTTCTGGGACTTTCGCTTTCCCCCTCCCGATCGC |
| | CACGGCAGAACTCATCGCCGCCTGCCTTGCCCGCTGCTGGACAGGGGCTAGGTTGCTGGGC |
| | ACTGATAATTCCGTGGTGTTGTC |

In particular embodiments, a vector comprises or encodes (in the case of an RNA vector, e.g., a retroviral vector) an MNDU3 promoter (e.g., SEQ ID NO: 320) operably linked to a polynucleotide encoding a chimeric antigen receptor comprising an amino acid set forth in any one of SEQ ID NOs: 259, 263, 266, 270, 273 or 277, and a polynucleotide comprising a posttranscriptional regulatory element set forth in any one of SEQ ID NOs: 315, 316, and 317. In particular embodiments, a vector comprises or encodes an MNDU3 promoter (e.g., SEQ ID NO: 320) operably linked to a polynucleotide sequence encoding a signal peptide set forth in SEQ ID NO: 245 and a polynucleotide encoding a chimeric antigen receptor comprising an amino acid set forth in any one of SEQ ID NOs: 259, 263, 266, 270, 273 or 277, and a polynucleotide comprising a posttranscriptional regulatory element set forth in any one of SEQ ID NOs: 315, 316, and 317. In particular embodiments, a vector comprises or encodes an MNDU3 promoter (e.g., SEQ ID NO: 320) operably linked to a polynucleotide sequence set forth in SEQ ID NO: 294, a polynucleotide encoding a chimeric antigen receptor comprising an amino acid set forth in any one of SEQ ID NOs: 259, 263, 266, 270, 273 or 277, and a polynucleotide comprising a posttranscriptional regulatory element set forth in any one of SEQ ID NOs: 315, 316, and 317.

In particular embodiments, a vector comprises or encodes an MNDU3 promoter (e.g., SEQ ID NO: 320) operably linked to a polynucleotide encoding a chimeric antigen receptor comprising a polynucleotide sequence set forth in any one of SEQ ID NOs: 297, 299, 300, 302, 304, and 308, and a polynucleotide comprising a posttranscriptional regulatory element set forth in any one of SEQ ID NOs: 315, 316, and 317. In particular embodiments, a vector comprises or encodes an MNDU3 promoter (e.g., SEQ ID NO: 320) operably linked to a polynucleotide sequence set forth in SEQ ID NO: 294, a polynucleotide sequence set forth in any one of SEQ ID NOs: 297, 299, 300, 302, 304, and 308, and a polynucleotide comprising a posttranscriptional regulatory element set forth in any one of SEQ ID NOs: 315, 316, and 317.

In particular embodiments, a vector comprises or encodes (in the case of an RNA vector, e.g., a retroviral vector) an EF1α promoter (e.g., SEQ ID NO: 319) operably linked to a polynucleotide encoding a chimeric antigen receptor comprising an amino acid set forth in any one of SEQ ID NOs: 259, 263, 266, 270, 273 or 277, and a polynucleotide comprising a posttranscriptional regulatory element set forth in any one of SEQ ID NOs: 315, 316, and 317. In particular embodiments, a vector comprises or encodes an EF1α promoter (e.g., SEQ ID NO: 319) operably linked to a polynucleotide sequence encoding a signal peptide set forth in SEQ ID NO: 245 and a polynucleotide encoding a chimeric antigen receptor comprising an amino acid set forth in any one of SEQ ID NOs: 259, 263, 266, 270, 273 or 277, and a polynucleotide comprising a posttranscriptional regulatory element set forth in any one of SEQ ID NOs: 315, 316, and 317. In particular embodiments, a vector comprises or encodes an EF1α promoter (e.g., SEQ ID NO: 319) operably linked to a polynucleotide sequence set forth in SEQ ID NO: 294, a polynucleotide encoding a chimeric antigen receptor comprising an amino acid set forth in any one of SEQ ID NOs: 259, 263, 266, 270, 273 or 277, and a polynucleotide comprising a posttranscriptional regulatory element set forth in any one of SEQ ID NOs: 315, 316, and 317.

In particular embodiments, a vector comprises or encodes an EF1α promoter (e.g., SEQ ID NO: 319) operably linked to a polynucleotide encoding a chimeric antigen receptor comprising a polynucleotide sequence set forth in any one of SEQ ID NOs: 297, 299, 300, 302, 304, and 308, and a polynucleotide comprising a posttranscriptional regulatory element set forth in any one of SEQ ID NOs: 315, 316, and 317. In particular embodiments, a vector comprises or encodes an EF1α promoter (e.g., SEQ ID NO: 319) operably linked to a polynucleotide sequence set forth in SEQ ID NO: 294, a polynucleotide sequence set forth in any one of SEQ ID NOs: 297, 299, 300, 302, 304, and 308, and a polynucleotide comprising a posttranscriptional regulatory element set forth in any one of SEQ ID NOs: 315, 316, and 317.

Efficient expression of polynucleotides can also be increased in some embodiments, by using sequences that increase translational efficiency, e.g., through an increase in mRNA ribosomal binding or an increase in mRNA stability. In certain embodiments, polynucleotides encoding a chimeric antigen receptor comprise a short recognition sequence, i.e., a Kozak sequence, that greatly facilitates the initial binding of mRNA to the small subunit of the ribosome and increases translation. The consensus Kozak sequence is (GCC)RCCATGG, where R is a purine (A or G) (Kozak, Cell. 44:283-92 (1986), and Kozak, Nucleic Acids Res. 15:8125-48 (1987)).

Elements directing the efficient termination and polyadenylation of heterologous nucleic acid transcripts may also increase heterologous gene expression. Transcription termination signals are generally found downstream of the polyadenylation signal. In particular embodiments, vectors comprise a polyadenylation sequence 3′ to a sequence to be transcribed and/or expressed. The term “polyadenylation (or poly(A)) signal” refers to a DNA sequence which directs both the termination and polyadenylation of the nascent RNA transcript by RNA polymerase II. Polyadenylation signals can promote mRNA stability by addition of a poly(A) tail to the 3′ end of the coding sequence and thus, contribute to increased translational efficiency. Cleavage and polyadenylation are directed by a poly(A) signal in the RNA. The core poly(A) signal for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) signal is an ideal poly(A) signal (e.g., AATAAA, ATTAAA, AGTAAA). In particular embodiments, the poly(A) signal is an SV40 poly(A) signal, a bovine growth hormone poly(A) signal (BGHpA), a rabbit β-globin poly(A) signal (rpgpA), variants thereof, or another suitable heterologous or endogenous poly(A) signal known in the art. In particular embodiments, the poly(A) signal is synthetic.

In particular embodiments, a polynucleotide comprises or encodes a promoter operably a polynucleotide sequence encoding a chimeric antigen receptor comprising a signal peptide isolated from a polypeptide selected from the group consisting of CD8α, murine IgGκ, human IgGk, CD33, tPA, SEAP, hGM-CSF, gaussian luciferase, CSF2R, B2M, and CD80, wherein the signal peptide is subsequently cleaved from the translated chimeric antigen receptor. In particular embodiments, a polynucleotide comprises or encodes a promoter operably linked to a polynucleotide sequence encoding a chimeric antigen receptor comprising a signal peptide comprising an amino acid sequence set forth in any one of SEQ ID NOs: 245, 246, 247, 248, 249, 250, 251, 252, 253, and 254. An illustrative example of a polynucleotide encoding a signal peptide is set forth in SEQ ID NO: 294 (5′ ATGGCTCTTCCCGTAACAGCCCTTTTGTTGCCCCTTGCACTCCTTCTGCATGCA GCACGACCG 3′).

### H. Vectors

Recombinant lentiviral particles contemplated in particular embodiments comprise (i) a viral envelope comprising (a) a mutated vesiculovirus envelope glycoprotein, e.g., VSIV-G, COCV-G, that does not bind its cognate receptor, e.g., LDLR, and (b) a non-viral membrane-bound tropism polypeptide that redirects the particle to immune effector cells; and (ii) a lentiviral vector comprising a polynucleotide encoding a promoter operably linked to a polynucleotide encoding an anti-BCMA chimeric antigen receptor comprising an amino acid sequence set forth in any one of SEQ ID NOs: 255, 256, 257, 258, 259, 260, 261, 262, 263, 264, 265, 266, 267, 268, 269, 270, 271, 272, 273, 274, 275, 276, 277, 278, 279, 280, 281, 282, and 283.

In various embodiments, a lentiviral vector (lentivector) is engineered or derived from a lentivirus genome selected from the group consisting of HIV-1, HIV-2, VMV, CAEV, EIAV, FIV, BIV, and SIV. In particular embodiments, lentiviral vectors are derived from HIV viral genomes, preferably HIV-1 or HIV-2 viral genomes and more preferably, HIV-1 viral genomes (i.e., HIV-1 cis-acting sequence elements are preferred).

In various embodiments, a recombinant lentiviral particle comprises (i) a viral envelope comprising (a) a mutated vesiculovirus envelope glycoprotein, e.g., VSIV-G, COCV-G, that does not bind its cognate receptor, e.g., LDLR, and (b) a non-viral membrane-bound tropism polypeptide that redirects the particle to immune effector cells that express CD3; and (ii) two copies of a lentiviral vector-based RNA genome comprising a 5′ long terminal repeat (LTR) comprising R and U5 regions; a Psi (Ψ) packaging signal; a cPPT/FLAP, an export element; a polynucleotide comprising or encoding a promoter operably linked to a polynucleotide encoding an anti-BCMA CAR; optionally a WPRE or HPRE; a 3′ LTR comprising U3 and R regions; a polyadenylation signal and a poly(A) tail.

In various embodiments, a recombinant lentiviral particle comprises (i) a viral envelope comprising (a) a mutated vesiculovirus envelope glycoprotein, e.g., VSIV-G, COCV-G, that does not bind its cognate receptor, e.g., LDLR, and (b) a non-viral membrane-bound tropism polypeptide that redirects the particle to immune effector cells that express CD3; and (ii) two copies of a lentiviral vector-based RNA genome comprising a 5′ long terminal repeat (LTR) comprising R and U5 regions; a Psi (Ψ) packaging signal; a cPPT/FLAP, an export element; a polynucleotide comprising or encoding a promoter operably linked to a polynucleotide encoding an anti-BCMA CAR comprising an amino acid sequence set forth in any one of SEQ ID NOs: 259, 263, 266, 270, 273, and 277; optionally a WPRE or HPRE; a 3′ LTR comprising U3 and R regions; a polyadenylation signal and a poly(A) tail.

The term “long terminal repeat (LTR),” as used herein, refers to elements located at the ends of lentiviral polynucleotides which, in their natural sequence context, are direct repeats and contain U3, R and U5 regions. LTRs generally provide functions fundamental to the expression of lentiviral genes (e.g., promotion, initiation and polyadenylation of gene transcripts) and to lentiviral replication. The LTR contains numerous regulatory signals including transcriptional control elements, polyadenylation signals and sequences needed for replication and integration of the lentiviral genome. The lentiviral LTR is divided into three regions called U3, R and U5. The U3 region contains the enhancer and promoter elements. The U5 region is the sequence between the primer binding site and the R region and contains the polyadenylation signal. The R (repeat) region is flanked by the U3 and U5 regions. A transfer plasmid, which is used to package a lentiviral vector genome comprises a 5′ LTR comprising U3, R and/or U5 regions and a 3′ LTR comprising U3, R and/or U5 regions. Adjacent to the 5′ LTR are sequences necessary for reverse transcription of the genome (the tRNA primer binding site) and for efficient packaging of lentiviral RNA into particles (the Psi “Ψ” site). A lentiviral vector-based genome packaged in a particle comprises a 5′ LTR comprising R and U5 regions and a 3′ LTR comprising U3 and R regions. The lentiviral vector-based genome is reverse transcribed and integrated into the host cell genome as a provector. Through reverse transcription and second strand synthesis of the lentiviral vector genome, provectors comprise two copies of the 3′ LTR, one copy that replaces the 5′ LTR and the 3′ LTR.

A “TAR” element as used herein, refers to the “trans-activation response” genetic element located in the R region of lentiviral vector LTRs. This element interacts with the lentiviral trans-activator (tat) genetic element to enhance lentiviral vector genome replication. In third generation lentiviral vectors, this element is not usually present because lentiviral vector transfer plasmids comprise a 5′ LTR U3 region replaced by a heterologous promoter.

An “R region,” as used herein, refers to the region within LTRs beginning at the start of the capping group (i.e., the start of transcription) and ending immediately prior to the start of the polyA signal. The R region is also defined as being flanked by the U3 and U5 regions. The R region plays a role during reverse transcription in permitting the transfer of nascent DNA from one end of the genome to the other.

As used herein, a “packaging signal” or “packaging sequence” refers to sequences located within the lentiviral genome which are required for insertion of the lentiviral RNA into the lentiviral capsid or particle, see e.g., Clever et al., 1995. J. of Virology, Vol. 69, No. 4; pp. 2101-2109. Several lentiviral vectors use the minimal packaging signal (also referred to as the psi [Ψ] or [Ψ+] sequence) needed for encapsidation of the lentiviral genome. Thus, as used herein, the terms “packaging sequence,” “packaging signal,” “psi” and the symbol “Ψ,” are used in reference to the non-coding sequence required for encapsidation of lentiviral RNA strands during lentiviral particle formation.

A “FLAP element” or “cPPT/FLAP,” as used herein refers to a nucleic acid whose sequence includes the central polypurine tract and central termination sequences (cPPT and CTS) of a lentivirus, e.g., HIV-1 or HIV-2. “FLAP element” and “cPPT/FLAP” may used interchangeably to refer to the foregoing FLAP element. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any particular theory, the DNA flap may act as a cis-active determinant of lentiviral vector nuclear import and/or may increase lentiviral titer.

As used herein, an “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), the woodchuck hepatitis virus posttranscriptional regulatory element (WPRE), and the hepatitis B virus post-transcriptional regulatory element (HPRE).

Lentiviral vectors may contain one or more safety enhancements to reduce the risk of replication, insertional mutagenesis, and off-target transduction and/or expression. In particular embodiments, a lentiviral vector comprises one or more or the following safety enhancements: one or more modifications of the 5′ and 3′ LTRs, cell or tissue specific expression control sequences, e.g., promoters, enhancers. A “modified LTR,” as used herein, refers to one or more nucleotide additions, deletions or substitutions in the native HIV-1 5′ LTR and/or 3′ LTR. The skilled artisan would be able to determine whether an LTR is modified by comparison to a reference LTR.

“Self-inactivating” (SIN) vectors, as used herein, refer to replication-defective vectors, e.g., lentiviral vectors, in which the right (3′) LTR enhancer-promoter region, known as the U3 region, has been modified (e.g., by deletion or substitution) to prevent viral transcription beyond the first round of lentiviral replication. Self-inactivation is achieved through a deletion in the U3 region of the 3′ LTR of the lentiviral vector transfer plasmid that removes the LTR TATA box (e.g., deletions from −418 to −18), without significant reductions in titers.

An additional safety enhancement is provided by replacing the U3 region of the 5′ LTR of the lentiviral vector transfer plasmid with a heterologous promoter to drive transcription of the lentiviral genome during production of recombinant lentiviral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

In particular embodiments, a lentiviral vector is engineered to integrate into an immune effector cell genome.

In certain embodiments, a lentiviral vector is engineered to be integration defective, episomal, and not integrate in the cell genome. As used herein, the term “integration defective lentivirus” or “IDLV” refers to a lentivirus having an integrase that lacks the capacity to integrate the viral vector into the host cell genome. Integration-incompetent lentiviral vectors have been described in patent application WO 2006/010834, which is herein incorporated by reference in its entirety. Illustrative mutations in HIV-1 integrase suitable to reduce integrase activity include, but are not limited to: H12N, H12C, H16C, H16V, S81R, D41A, K42A, H51A, Q53C, D55V, D64E, D64V, E69A, K71A, E85A, E87A, D116N, D116I, D116A, N120G, N120I, N120E, E152G, E152A, K156E, K156A, E157A, K159E, K159A, K160A, R166A, D167A, E170A, H171A, K173A, K186Q, K186T, K188T, E198A, R199C, R199T, R199A, D202A, K211A, Q214L, Q216L, Q221L, W235F, W235E, K236S, K236A, K246A, G247W, D253A, R262A, R263A and K264H. In particular embodiments, an HIV-1 integration deficient integrase comprises a D64V, D161I, D116A, E152G, or E152A mutation; D64V, D116A, and E152G mutations; D64V, D116A, and E152A mutations; or a D64V mutation.

In particular embodiments, a recombinant lentiviral particle comprises (i) a viral envelope comprising (a) a mutated VSIV-G or COCV-G polypeptide comprising amino acid substitutions at positions 47 and 354 of the mature polypeptide and (b) a non-viral membrane-bound tropism polypeptide comprising an anti-CD3 scFv and a CD8α hinge and transmembrane domain; and (ii) two copies of an HIV-1 lentiviral vector-based RNA genome comprising a 5′ LTR comprising R and U5 regions; a Psi (Ψ) packaging signal; a cPPT/FLAP, an RRE export element; a polynucleotide comprising or encoding an MNDU3 or EF1α promoter operably linked to a polynucleotide encoding a signal peptide, an anti-BCMA CAR, optionally a WPRE or HPRE; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 332, 333, 334, and 335 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 320 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 332, 333, 334, and 335 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 320 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, and 270, and optionally, a polynucleotide comprising a posttranscriptional regulatory element set forth in any one of SEQ ID NOs: 315, 316, and 317; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 332, 333, 334, and 335 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 320 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 332, 333, 334, and 335 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 319 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 332, 333, 334, and 335 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 319 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, and 270, and optionally, a polynucleotide comprising a posttranscriptional regulatory element set forth in any one of SEQ ID NOs: 315, 316, and 317; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 332, 333, 334, and 335 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 319 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 336, 337, 338, and 339 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 320 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and a polynucleotide comprising a posttranscriptional regulatory element set forth in any one of SEQ ID NOs: 315, 316, and 317; a 3′ LTR comprising U3 and R regions; a polyadenylation signal and a poly(A) tail.

In particular embodiments, a recombinant lentiviral particle comprises (i) a viral envelope comprising (a) a fusogen comprising an amino acid sequence set forth in any one of SEQ ID NOs: 336, 337, 338, and 339 and (b) a non-viral membrane-bound tropism polypeptide comprising an amino acid sequence set forth in any one of SEQ ID NOs: 324, 325, 326, 327, 328, 329, 330, and 331; and (ii) two copies of an HIV-1 lentiviral vector-based RNA genome comprising a 5′ LTR comprising R and U5 regions; a Psi (Ψ) packaging signal; a cPPT/FLAP, an RRE export element; a promoter comprising or encoding a polynucleotide sequence set forth in SEQ ID NO: 319 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and, a polynucleotide comprising a posttranscriptional regulatory element set forth in any one of SEQ ID NOs: 315, 316, and 317; a 3′ LTR comprising U3 and R regions; a polyadenylation signal and a poly(A) tail.

### I. Cells

Recombinant particles contemplated herein are engineered to bind and transduce an immune effector cell. In particular embodiments, a recombinant lentiviral particle engineered to bind and transduce and immune effector cell comprises a viral envelope comprising a mutated COCV-G polypeptide or a mutated VSIV-G polypeptide, wherein the mutated COCV-G or VSIV-G polypeptide comprises amino acid substitutions at positions 47 and 354; a non-viral membrane-bound tropism polypeptide comprising an anti-CD3ε scFv and a human CD8α hinge and transmembrane domain; and a recombinant lentiviral vector comprising a polynucleotide encoding a myeloproliferative sarcoma virus enhancer, negative control region deleted, dl587rev primer-binding site substituted (MND) U3 promoter or an EF1α promoter operably linked to a polynucleotide encoding an anti-BCMA chimeric antigen receptor comprising an anti-BCMA scFv or anti-BCMA VHH, a CD8α hinge and transmembrane domain, a CD137 costimulatory domain, a CD3ζ primary signaling domain.

An “immune effector cell” is any cell of the immune system that has one or more effector functions (e.g., cytotoxic cell killing activity, secretion of cytokines, induction of ADCC and/or CDC). Illustrative types of immune effector cells contemplated in particular embodiments include, without limitation, T lymphocytes, dendritic cells (DC), Treg cells, natural killer (NK) cells, natural killer T (NKT) cells, and macrophages. The terms “T cell” or “T lymphocyte” are art-recognized and are intended, in particular embodiments, to include thymocytes, immature T lymphocytes, mature T lymphocytes, resting T lymphocytes, and/or activated T lymphocytes. Illustrative examples of T lymphocytes suitable for use in particular embodiments, include but not limited to cytotoxic T cells (CTLs; CD8⁺ T cells), TILs, helper T cells (HTLs; CD4⁺ T cells), CD4⁺CD8⁺ T cells, CD4⁻ CD8⁻ T cells, or any other subset of T cells that has an effector function. In a particular embodiment, the cells comprise at T cells. In a particular embodiment, the cells comprise 76 T cells.

### J. Compositions and Formulations

Compositions contemplated herein comprise a recombinant particle and/or an immune effector cell modified ex vivo formulated with a pharmaceutically acceptable or physiologically-acceptable carrier for administration to a cell, tissue, organ, or an animal, either alone, or in combination with one or more other modalities of therapy.

In particular embodiments, a composition comprises a recombinant lentiviral particle comprising a viral envelope comprising a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain; and one or more copies of a lentiviral vector comprising a polynucleotide comprising or encoding an MNDU3 or EF1α promoter operably linked to a polynucleotide encoding an anti-BCMA CAR, and optionally a WPRE.

In particular embodiments, a composition comprises a recombinant lentiviral particle comprising a viral envelope comprising a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain; and one or more copies of a recombinant lentiviral vector comprising a polynucleotide comprising or encoding an MNDU3 or EF1α promoter operably linked to a polynucleotide encoding a signal peptide, an anti-BCMA chimeric antigen receptor comprising an anti-BCMA scFv or anti-BCMA VHH, a CD8α hinge and transmembrane domain, a CD137 costimulatory domain, a CD3ζ primary signaling domain, and optionally a WPRE.

In particular embodiments, a composition comprises a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 320 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317.

In particular embodiments, a composition comprises a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 320 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317.

In particular embodiments, a composition comprises a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 319 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317.

In particular embodiments, a composition comprises a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 319 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317.

In particular embodiments, the composition is a pharmaceutical composition. A “pharmaceutical composition” refers to a composition formulated in a pharmaceutically-acceptable or physiologically-acceptable solution for administration to a cell or a subject, either alone, or in combination with one or more other modalities of therapy.

“Pharmaceutically acceptable” refers to molecular entities and compositions that do not produce excessive toxicity, irritation, allergic response, or other problems or complications, commensurate with a reasonable benefit/risk ratio when administered to a human.

In particular embodiments, a composition comprises a pharmaceutically acceptable carrier and a recombinant particle contemplated herein. The term “pharmaceutically acceptable carrier” refers to a diluent, adjuvant, excipient, vehicle and the like with which a recombinant particle, e.g., a recombinant retroviral or lentiviral particle, is physiologically compatible with administration to a human, including but not limited to pharmaceutically acceptable cell culture media, Dulbecco's phosphate buffered saline (PBS), Ringer's solution, 5% dextrose in water (D5W), and normal/physiologic saline (0.9% NaCl).

In particular embodiments, a composition comprises a pharmaceutically acceptable carrier and a recombinant lentiviral particle comprising a viral envelope comprising a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain; and one or more copies of a lentiviral vector comprising a polynucleotide comprising or encoding an MNDU3 or EF1α promoter operably linked to a polynucleotide encoding an anti-BCMA CAR, and optionally a WPRE.

In particular embodiments, a composition comprises a pharmaceutically acceptable carrier and a recombinant lentiviral particle comprising a viral envelope comprising a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain; and one or more copies of a recombinant lentiviral vector comprising a polynucleotide comprising or encoding an MNDU3 or EF1α promoter operably linked to a polynucleotide encoding a signal peptide, an anti-BCMA chimeric antigen receptor comprising an anti-BCMA scFv or anti-BCMA VHH, a CD8α hinge and transmembrane domain, a CD137 costimulatory domain, a CD3ζ primary signaling domain, and optionally a WPRE.

In particular embodiments, a composition comprises a pharmaceutically acceptable carrier and a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 320 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317.

In particular embodiments, a composition comprises a pharmaceutically acceptable carrier and a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 320 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317.

In particular embodiments, a composition comprises a pharmaceutically acceptable carrier and a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 319 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317.

In particular embodiments, a composition comprises a pharmaceutically acceptable carrier and a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 319 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317.

In particular embodiments, a composition comprises a recombinant particle and a pharmaceutically acceptable carrier suitable for enteral or parenteral, e.g., intravascular (intravenous or intraarterial), intraosseous, intraperitoneal, intraventricular, intracerebral, intracranial, intraspinal, intrathecal, intramuscular, and intramedullary, administration and formulation.

In particular embodiments, a composition is substantially free of mycoplasma, endotoxin, and microbial contamination. By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells. In particular embodiments, compositions contemplated herein contain about 0.5 EU/mL to about 5.0 EU/mL, or about 0.5 EU/mL, 1.0 EU/mL, 1.5 EU/mL, 2.0 EU/mL, 2.5 EU/mL, 3.0 EU/mL, 3.5 EU/mL, 4.0 EU/mL, 4.5 EU/mL, or 5.0 EU/mL.

In particular embodiments, compositions contemplated herein are used in the treatment of a cancer. In particular embodiments, a composition comprises a recombinant particle contemplated herein and one or more cytokines, growth factors, steroids, NSAIDs, DMARDs, anti-inflammatories, chemotherapeutics, radiotherapeutics, therapeutic antibodies, or other active and ancillary agents, either alone or in combination.

It would be understood by the skilled artisan that particular embodiments contemplated herein may comprise other formulations, such as those that are well known in the pharmaceutical art, and are described, for example, in Remington: The Science and Practice of Pharmacy, Volume I and Volume II. 23^{rd} Edition. Edited by Adeboye Adejare. Academic Press, 2020, which is incorporated by reference herein, in its entirety.

### K. Methods of Making

The manufacturing processes contemplated herein comprise an upstream process that produces a recombinant lentiviral particle and a downstream process that purifies the recombinant lentiviral particle. Methods of manufacturing lentiviral particles are described in WO2023/003844, which is hereby incorporated by reference in its entirety. See, also Kutner et al., BMC Biotechnol. 2009; 9:10. doi: 10.1186/1472-6750-9-10 and Kutner et al. Nat. Protoc. 2009; 4(4):495-505. doi: 10.1038/nprot.2009.22.

In particular embodiments, a method of manufacturing recombinant lentiviral particles comprises transfecting a host cell culture with packaging plasmids and a transfer plasmid, culturing transfected host cells to produce lentiviral particles; and collecting and processing the culture supernatant that contains the crude lentiviral particles to remove impurities and concentrate and formulate the particles for clinical use.

In particular embodiments, lentiviral particles are manufactured by transfecting host cells with a multi-plasmid system comprising (i) an envelope plasmid encoding a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain, (ii) a packaging plasmid encoding lentiviral gag-pol, (iii) a packaging plasmid encoding lentiviral rev, and (iv) a transfer plasmid comprising a polynucleotide encoding a lentiviral vector contemplated herein.

In particular embodiments, lentiviral particles are manufactured by transfecting host cells with a multi-plasmid system comprising (i) an envelope plasmid encoding a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain, (ii) a packaging plasmid encoding lentiviral gag-pol, (iii) a packaging plasmid encoding lentiviral rev, and (iv) a transfer plasmid comprising a polynucleotide encoding a lentiviral vector comprising or encoding an MNDU3 or EF1α promoter operably linked to a polynucleotide encoding a signal peptide, an anti-BCMA chimeric antigen receptor comprising an anti-BCMA scFv or anti-BCMA VHH, a CD8α hinge and transmembrane domain, a CD137 costimulatory domain, a CD3ζ primary signaling domain, and optionally a WPRE.

In particular embodiments, lentiviral particles are manufactured by transfecting host cells with a multi-plasmid system comprising (i) an envelope plasmid encoding a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain, (ii) a packaging plasmid encoding lentiviral gag-pol, (iii) a packaging plasmid encoding lentiviral rev, and (iv) a transfer plasmid comprising a polynucleotide encoding a lentiviral vector comprising a promoter comprising or encoding a polynucleotide sequence set forth in SEQ ID NO: 320 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317.

In particular embodiments, lentiviral particles are manufactured by transfecting host cells with a multi-plasmid system comprising (i) an envelope plasmid encoding a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain, (ii) a packaging plasmid encoding lentiviral gag-pol, (iii) a packaging plasmid encoding lentiviral rev, and (iv) a transfer plasmid comprising a polynucleotide encoding a lentiviral vector comprising a promoter comprising or encoding a polynucleotide sequence set forth in SEQ ID NO: 320 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317.

In particular embodiments, lentiviral particles are manufactured by transfecting host cells with a multi-plasmid system comprising (i) an envelope plasmid encoding a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain, (ii) a packaging plasmid encoding lentiviral gag-pol, (iii) a packaging plasmid encoding lentiviral rev, and (iv) a transfer plasmid comprising a polynucleotide encoding a lentiviral vector comprising a promoter comprising or encoding a polynucleotide sequence set forth in SEQ ID NO: 319 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317.

In particular embodiments, lentiviral particles are manufactured by transfecting host cells with a multi-plasmid system comprising (i) an envelope plasmid encoding a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain, (ii) a packaging plasmid encoding lentiviral gag-pol, (iii) a packaging plasmid encoding lentiviral rev, and (iv) a transfer plasmid comprising a polynucleotide encoding a lentiviral vector comprising a promoter comprising or encoding a polynucleotide sequence set forth in SEQ ID NO: 319 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317.

A “transfer plasmid” or “transfer vector” encodes a lentiviral genomic RNA modified to package a lentiviral vector that comprises a polynucleotide sequence delivered by recombinant lentiviral particle to a cell. In particular embodiments, a transfer plasmid comprises one or more polynucleotide sequences of interest flanked by LTR sequences, which facilitate packaging, reverse transcription and integration of the lentiviral vector and associated polynucleotide sequences into the host genome. Lentiviral vectors contemplated herein are replication incompetent, i.e., lack the genetic elements necessary for generation of infective particles in the host cell. For example, a lentiviral vector may comprise a deletion of the 3′ LTR, rendering the virus “self-inactivating” (SIN).

Illustrative examples of host cells suitable for transfection with the aforementioned plasmid systems include but are not limited to HEK293 cells, HEK293S cells, HEK293T cells adapted for suspension culture (HEK293Ts), HEK293F cells, HEK293FT cells, HEK293FTM cells, and HEK293E cells.

Once host cells are transfected and produce lentiviral particles, the cell culture is subjected to a downstream process that yields particles sufficient for clinical use, In particular embodiments, a downstream process comprises treating the contents of the bioreactor with a DNA endonuclease, e.g., benzonase; harvesting and clarifying the suspension culture supernatant by filtration; capturing and concentrating the lentiviral particles in the resultant filtrate using affinity chromatography or cation exchange chromatography; filtering the eluate comprising the lentiviral particles; ultrafiltering and diafiltering the lentiviral particles using tangential flow filtration (TFF); and formulating the lentiviral particles in a physiologically acceptable diluent to produce a formulated bulk lentiviral particles. In one embodiment, the formulated bulk lentiviral particles are sterile filtered, filled, and frozen; and subsequently thawed, sterile filtered, subjected to a final fill finish, and frozen. In another embodiment, the bulk lentiviral particles are sterile filtered, subjected to a final fillfinish, and frozen.

### L. Methods of Use

Recombinant particles contemplated herein are engineered to modify an immune cell that expresses CD3 in vivo to express an anti-BCMA CAR, which redirects the CD3-expressing immune cell to a target cell expressing BCMA, thereby preventing, treating, or ameliorating at least one symptom associated with a disease, disorder, or condition associated therewith.

In particular embodiments, a method of preventing, treating, or ameliorating at least one symptom of a cancer comprises administering the subject an amount of recombinant particle contemplated herein. The term “amount” as used herein, refers to “an amount effective” or “an effective amount” of a recombinant particle contemplated herein comprising a payload contemplated herein, etc., to achieve a beneficial or desired prophylactic or therapeutic result, including clinical results. A “prophylactically effective amount” refers to an amount of recombinant particle contemplated herein comprising payload contemplated herein, effective to achieve the desired prophylactic result. A “therapeutically effective amount” refers to an amount of recombinant particle contemplated herein comprising a lentiviral vector encoding an anti-BCMA CAR, that is effective to “treat” a subject (e.g., a patient). When a therapeutic amount is indicated, the precise amount of the compositions to be administered can be determined by a physician with consideration of individual differences in age, weight, tumor size, extent of infection or metastasis, and condition of the patient (subject).

In particular embodiments, a recombinant lentiviral particle comprising a viral envelope comprising a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain; and one or more copies of a lentiviral vector comprising a polynucleotide comprising or encoding an MNDU3 or EF1α promoter operably linked to a polynucleotide encoding an anti-BCMA CAR, and optionally a WPRE, is administered to a subject to treat, prevent, or ameliorate at least one symptom of multiple myeloma in the subject.

In particular embodiments, a recombinant lentiviral particle comprising a viral envelope comprising a mutated VSIV-G or COCV-G polypeptide comprising amino acid substitutions at positions 47 and 354 of the mature polypeptide and a non-viral membrane-bound tropism polypeptide comprising an anti-CD3 scFv and a CD8α hinge and transmembrane domain; and one or more copies of a recombinant lentiviral vector comprising a polynucleotide comprising or encoding an MNDU3 or EF1α promoter operably linked to a polynucleotide encoding a signal peptide, an anti-BCMA chimeric antigen receptor comprising an anti-BCMA scFv or anti-BCMA VHH, a CD8α hinge and transmembrane domain, a CD137 costimulatory domain, a CD3ζ primary signaling domain, and optionally a WPRE, is administered to a subject to treat, prevent, or ameliorate at least one symptom of multiple myeloma in the subject.

In particular embodiments, a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 320 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317, is administered to a subject to treat, prevent, or ameliorate at least one symptom of multiple myeloma in the subject.

In particular embodiments, a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 320 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317, is administered to a subject to treat, prevent, or ameliorate at least one symptom of multiple myeloma in the subject.

In particular embodiments, a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 319 operably linked to a polynucleotide encoding a signal peptide comprising an amino acid sequence set forth in SEQ ID NO: 245 and an anti-BCMA CAR comprising an amino acid sequence set forth in any one of SEQ ID NOs: 259, 263, 266, 270, 273, and 277, and optionally, a polynucleotide comprising a posttranscriptional regulatory element set forth in any one of SEQ ID NOs: 315, 316, and 317, is administered to a subject to treat, prevent, or ameliorate at least one symptom of multiple myeloma in the subject.

In particular embodiments, a recombinant lentiviral particle comprising a viral envelope comprising a fusogen comprising an amino acid sequence set forth in any one of SEQ ID NOs: 332, 333, 334, and 335 and a non-viral membrane-bound tropism polypeptide comprising an amino acid sequence set forth in any one of SEQ ID NOs: 324, 325, 326, 327, 328, 329, 330, and 331; and two copies of a lentiviral vector comprising a promoter comprising or encoding a polynucleotide sequence set forth in SEQ ID NO: 319 operably linked to a polynucleotide encoding a signal peptide encoded by a polynucleotide sequence set forth in SEQ ID NO: 294 and an anti-BCMA CAR encoded by a polynucleotide sequence set forth in any one of SEQ ID NOs: 297, 299, 300, 302, 304, and 308, and optionally, a polynucleotide comprising a posttranscriptional regulatory element set forth in any one of SEQ ID NOs: 315, 316, and 317, is administered to a subject to treat, prevent, or ameliorate at least one symptom of multiple myeloma in the subject.

In particular embodiments, a recombinant lentiviral particle contemplated herein is administered to a subject to treat, prevent, or ameliorate at least one symptom of a multiple myeloma or precursor thereof selected from the group consisting of: monoclonal gammopathy of undetermined significance (MGUS), active multiple myeloma, smoldering multiple myeloma, light chain myeloma, non-secretory myeloma, IgD myeloma, IgE myeloma, osteosclerotic myeloma, solitary plasmacytoma of bone, and extramedullary plasmacytoma.

In particular embodiments, a recombinant lentiviral particle is administered to a subject in combination with one or more anti-cancer therapies including, but not limited to, an autologous stem cell transplant (ASCT), radiation, surgery, a chemotherapeutic agent, an immunomodulatory agent and a targeted cancer therapy.

In particular embodiments, the one or more anti-cancer therapies is selected from the group consisting of 6-mercaptopurine, abiraterone, alemtuzumab, all-trans retinoic acid, anastrozole, aprepitant, arsenic trioxide, atezolizumab, avelumab, azacytidine, bafetinib, bavituximab, bevacizumab, bivatuzumab, bleomycin, blinatumomab, bortezomib, bosutinib, cabazitaxel, capecitabine, carboplatin, carfilzomib, cetuximab, cisplatin, cladribine, conatumumab, corticosteroid, crizotinib, cyclophosphamide, cytarabine, dacetuzumab, dalotuzumab, daratumumab, dasatinib, daunorubicin, danusertib, decitabine, denosumab, dexamethasone, docetaxel, doxorubicin, duligotumab, durvalumab, elotozumab, eribulin, erlotinib, etoposide, everolimus, exemestane, filgrastim, fludarabine, fluorouracil, fulvestrant, gemcitabine, gemtuzumab, hydroxyurea, ibritumomab, idarubicin, imatinib, imiquimod, indatuximab, inotuzumab, ipilimumab, ixabepilone, ixazomib, lapatinib, lenalidomide, letrozole, leuprolide, lorvotuzumab, lucatumumab, melphalan, methotrexate, milatuzumab, mitoxantrone, moxetumomab, nilotinib, nivolumab, ocaratuzumab, ofatumumab, oxaliplatin, paclitaxel, palonosetron, pembrolizumab, pemetrexed, pomalidomide, ponatinib, prednisone, radium-223, rituximab, saracatinib, siltuximab, sipuleucel-T, sorafenib, sunitinib, tamoxifen, temozolomide, temsirolimus, teprotumumab, thalidomide, tinorelbine, topotecan, tozasertib, trastuzumab, ublituximab, vincristine, and zoledronic acid.

### M. Enumerated Embodiments

Embodiment 1: A recombinant lentiviral particle comprising:
(a) a viral envelope comprising (i) a mutated cocal virus envelope glycoprotein (COCV-G) or a mutated vesicular stomatitis Indiana virus envelope glycoprotein (VSIV-G), wherein the mutated COCV-G or VSIV-G comprises amino acid substitutions at positions 47 and 354; and (ii) a non-viral membrane-bound tropism polypeptide comprising an anti-CD3ε scFv and a human CD8α hinge and transmembrane domain; and
(b) a recombinant lentiviral vector comprising a polynucleotide encoding a myeloproliferative sarcoma virus enhancer, negative control region deleted, dl587rev primer-binding site substituted (MND) U3 promoter or an EF1α promoter operably linked to a polynucleotide encoding an anti-BCMA chimeric antigen receptor comprising an anti-BCMA scFv or anti-BCMA VHH, a CD8α hinge and transmembrane domain, a CD137 costimulatory domain, a CD3ζ primary signaling domain.

Embodiment 2: The particle of embodiment 1, wherein the mutated COCV-G or the mutated VSIV-G comprises amino acid substitutions selected from the group consisting of: K47A and R354A; K47A and R354Q; K47Q and R354A; and K47Q and R354Q.

Embodiment 3: The particle of embodiment 1 or embodiment 2, wherein the mutated COCV-G or the mutated VSIV-G comprises the amino acid substitutions K47A and R354A.

Embodiment 4: The particle of embodiment 1 or embodiment 2, wherein the mutated COCV-G or the mutated VSIV-G comprises the amino acid substitutions K47A and R354Q.

Embodiment 5: The particle of embodiment 1 or embodiment 2, wherein the mutated COCV-G or the mutated VSIV-G comprises the amino acid substitutions K47Q and R354A.

Embodiment 6: The particle of embodiment 1 or embodiment 2, wherein the mutated COCV-G or the mutated VSIV-G comprises the amino acid substitutions K47Q and R354Q.

Embodiment 7: The particle of any one of embodiments 1 to 6, wherein the mutated COCV-G or the mutated VSIV-G comprises the amino acid sequence set forth in any one of SEQ ID NOs: 332, 333, 334, 335, 336, 337, 338, and 339.

Embodiment 8: The particle of any one of embodiments 1 to 7, wherein the mutated COCV-G or the mutated VSIV-G comprises the amino acid sequence set forth in any one of SEQ ID NOs: 332, 333, 334, and 335.

Embodiment 9: The particle of any one of embodiments 1 to 7, wherein the mutated COCV-G or the mutated VSIV-G comprises the amino acid sequence set forth in any one of SEQ ID NOs: 336, 337, 338, and 339.

Embodiment 10: The particle of any one of embodiments 1 to 9, wherein the anti-CD3ε scFv is isolated from an antibody selected from the group consisting of: OKT3, UCHT1, YTH12.5, TR66, and variants thereof.

Embodiment 11: The particle of any one of embodiments 1 to 10, wherein the anti-CD3ε scFv is isolated from OKT3.

Embodiment 12: The particle of any one of embodiments 1 to 10, wherein the anti-CD3ε scFv is isolated from UCHT1.

Embodiment 13: The particle of any one of embodiments 1 to 10, wherein the anti-CD3ε scFv is isolated from YTH12.5.

Embodiment 14: The particle of any one of embodiments 1 to 10, wherein the anti-CD3ε scFv is isolated from TR66.

Embodiment 15: The particle of any one of embodiments 1 to 10, wherein the anti-CD3ε scFv comprises an amino acid sequence set forth in any one of SEQ ID NOs: 153, 154, 163, 164, 173, 174, 183, 184, 193, 194, 203, 204, 213, 214, 223, and 224.

Embodiment 16: The particle of any one of embodiments 1 to 10, wherein the non-viral membrane-bound tropism polypeptide comprises an amino acid sequence set forth in any one of SEQ ID NOs: 324, 325, 326, 327, 328, 329, 330, and 331.

Embodiment 17: The particle of any one of embodiments 1 to 16, wherein the MND U3 promoter comprises the polynucleotide sequence set forth in SEQ ID NO: 320.

Embodiment 18: The particle of any one of embodiments 1 to 16, wherein the EF1α promoter comprises the polynucleotide sequence set forth in SEQ ID NO: 319.

Embodiment 19: The particle of any one of embodiments 1 to 18, wherein the anti-BCMA CAR comprises an anti-BCMA scFv comprising an amino acid sequence selected from the group consisting of: 19, 20, 29, 30, 39, 40, 49, 50, 59, 60, 69, 70, 79, 80, 89, 90, 99, and 100.

Embodiment 20: The particle of any one of embodiments 1 to 19, wherein the anti-BCMA CAR comprises an anti-BCMA scFv comprising an amino acid sequence selected from the group consisting of: 20, 30, 39, 50, 59, 70, 80, 90, and 100.

Embodiment 21: The particle of any one of embodiments 1 to 20, wherein the anti-BCMA CAR comprises an anti-BCMA scFv comprising an amino acid sequence selected from the group consisting of: 39, 59, 70, and 90.

Embodiment 22: The particle of any one of embodiments 1 to 18, wherein the anti-BCMA CAR comprises an anti-BCMA VHH comprising an amino acid sequence selected from the group consisting of: 101, 105, 109, 113, 117, 121, 125, 129, 133, 137, and 141.

Embodiment 23: The particle of any one of embodiments 1 to 18, wherein the anti-BCMA CAR comprises an anti-BCMA VHH comprising an amino acid sequence selected from the group consisting of: 101 and 117.

Embodiment 24: The particle of any one of embodiments 1 to 23, wherein the anti-BCMA CAR comprises the amino acid sequence set forth in any one of SEQ ID NOs: 259, 263, 266, 270, 273 and 277.

Embodiment 25: The particle of any one of embodiments 1 to 24, wherein the polynucleotide encoding the anti-BCMA CAR comprises the polynucleotide sequence set forth in any one of SEQ ID NOs: 297, 299, 300, 302, 304, and 308.

Embodiment 26: The particle of any one of embodiments 1 to 25, wherein the polynucleotide encoding the anti-BCMA CAR further comprises a polynucleotide sequence encoding a signal peptide.

Embodiment 27: The particle of any one of embodiments 1 to 25, wherein the polynucleotide encoding the anti-BCMA CAR further comprises a polynucleotide sequence encoding a signal peptide isolated from a polypeptide selected from the group consisting of: CD8α, mIgGx, hIgGk, CD33, tPA, SEAP, hGM-CSF, CSF2R, and B2M.

Embodiment 28: The particle of any one of embodiments 1 to 27, wherein the polynucleotide encoding the anti-BCMA CAR further comprises a polynucleotide sequence encoding a signal peptide comprising an amino acid sequence set forth in any one of SEQ ID NOs: 245, 246, 247, 248, 249, 250, 251, 252, 253, and 254.

Embodiment 29: The particle of any one of embodiments 21 to 23, wherein the polynucleotide encoding the signal peptide comprises the polynucleotide sequence set forth in SEQ ID NO: 294.

Embodiment 30: The particle of any one of embodiments 1 to 24, wherein the lentiviral vector further comprises a WPRE operably linked to the 3′ end of the polynucleotide encoding the anti-BCMA CAR.

Embodiment 31: The particle of any one of embodiments 1 to 25, wherein the lentiviral vector further comprises a WPRE that comprises, consists essentially of, or consists of a polynucleotide sequence set forth in any one of SEQ ID NOs: 315, 316, and 317.

Embodiment 32: A recombinant lentiviral particle comprising:
(a) a viral envelope comprising (i) a mutated viral envelope glycoprotein comprising an amino acid sequence set forth in any one of SEQ ID NOs: 332, 333, 334, and 335 and (ii) a non-viral membrane-bound tropism polypeptide comprising an amino acid sequence set forth in any one of SEQ ID NOs: 324, 325, 326, 327, 328, 329, 330, and 331; and
(b) a recombinant lentiviral vector comprising a 5′ long terminal repeat (LTR) comprising R and U5 regions; a Psi (Ψ) packaging signal, a cPPT/FLAP, a rev response element (RRE); a polynucleotide encoding an MND promoter or an EF1α promoter operably linked to a polynucleotide encoding an anti-BCMA chimeric antigen receptor (CAR) comprising an amino acid sequence set forth in any one of SEQ ID NOs: 259, 263, 266, 270, 273, and 277 or an amino acid sequence at least 95%, at least 96%, at least 97%, at least 98%, at least 99% identical thereto; optionally a WPRE; a 3′ LTR comprising U3 and R regions; a polyadenylation signal and a poly(A) tail.

Embodiment 33: The particle of embodiment 32, wherein the mutated viral envelope glycoprotein comprises an amino acid sequence set forth in SEQ ID NO: 332.

Embodiment 34: The particle of embodiment 32, wherein the mutated viral envelope glycoprotein comprises an amino acid sequence set forth in SEQ ID NO: 333.

Embodiment 35: The particle of embodiment 32, wherein the mutated viral envelope glycoprotein comprises an amino acid sequence set forth in SEQ ID NO: 334.

Embodiment 36: The particle of embodiment 32, wherein the mutated viral envelope glycoprotein comprises an amino acid sequence set forth in SEQ ID NO: 335.

Embodiment 37: A recombinant lentiviral particle comprising:
(a) a viral envelope comprising (i) a mutated viral envelope glycoprotein comprising an amino acid sequence set forth in any one of SEQ ID NOs: 336, 337, 338, and 339 and (ii) a non-viral membrane-bound tropism polypeptide comprising an amino acid sequence set forth in any one of SEQ ID NOs: 324, 325, 326, 327, 328, 329, 330, and 331; and
(b) a recombinant lentiviral vector comprising a 5′ long terminal repeat (LTR) comprising R and U5 regions; a Psi (Ψ) packaging signal, a cPPT/FLAP, a rev response element (RRE); a polynucleotide encoding an MND promoter or an EF1α promoter operably linked to a polynucleotide encoding an anti-BCMA chimeric antigen receptor (CAR) comprising an amino acid sequence set forth in any one of SEQ ID NOs: 259, 263, 266, 270, 273, and 277 or an amino acid sequence at least 95%, at least 96%, at least 97%, at least 98%, at least 99% identical thereto; optionally a WPRE; a 3′ LTR comprising U3 and R regions; a polyadenylation signal and a poly(A) tail.

Embodiment 38: The particle of embodiment 37, wherein the mutated viral envelope glycoprotein comprises an amino acid sequence set forth in SEQ ID NO: 336.

Embodiment 39: The particle of embodiment 37, wherein the mutated viral envelope glycoprotein comprises an amino acid sequence set forth in SEQ ID NO: 337.

Embodiment 40: The particle of embodiment 37, wherein the mutated viral envelope glycoprotein comprises an amino acid sequence set forth in SEQ ID NO: 338.

Embodiment 41: The particle of embodiment 37, wherein the mutated viral envelope glycoprotein comprises an amino acid sequence set forth in SEQ ID NO: 339.

Embodiment 42: The particle of any one of embodiments 32 to 41, wherein the non-viral membrane-bound tropism polypeptide comprises an amino acid sequence set forth in SEQ ID NO: 324.

Embodiment 43: The particle of any one of embodiments 32 to 41, wherein the non-viral membrane-bound tropism polypeptide comprises an amino acid sequence set forth in SEQ ID NO: 325.

Embodiment 44: The particle of any one of embodiments 32 to 41, wherein the non-viral membrane-bound tropism polypeptide comprises an amino acid sequence set forth in SEQ ID NO: 326.

Embodiment 45: The particle of any one of embodiments 32 to 41, wherein the non-viral membrane-bound tropism polypeptide comprises an amino acid sequence set forth in SEQ ID NO: 327.

Embodiment 46: The particle of any one of embodiments 32 to 41, wherein the non-viral membrane-bound tropism polypeptide comprises an amino acid sequence set forth in SEQ ID NO: 328.

Embodiment 47: The particle of any one of embodiments 32 to 41, wherein the non-viral membrane-bound tropism polypeptide comprises an amino acid sequence set forth in SEQ ID NO: 329.

Embodiment 48: The particle of any one of embodiments 32 to 41, wherein the non-viral membrane-bound tropism polypeptide comprises an amino acid sequence set forth in SEQ ID NO: 330.

Embodiment 49: The particle of any one of embodiments 32 to 41, wherein the non-viral membrane-bound tropism polypeptide comprises an amino acid sequence set forth in SEQ ID NO: 331.

Embodiment 50: The particle of any one of embodiments 32 to 49, wherein the recombinant lentiviral vector is derived from HIV-1 or HIV-2.

Embodiment 51: The particle of any one of embodiments 32 to 50, wherein the MND U3 promoter comprises the polynucleotide sequence set forth in SEQ ID NO: 320.

Embodiment 52: The particle of any one of embodiments 32 to 50, wherein the EF1α promoter comprises the polynucleotide sequence set forth in SEQ ID NO: 319.

Embodiment 53: The particle of any one of embodiments 32 to 52, wherein the polynucleotide encoding the anti-BCMA CAR comprises the polynucleotide sequence set forth in any one of SEQ ID NOs: 297, 299, 300, 302, 304, and 308.

Embodiment 54: The particle of any one of embodiments 32 to 53, wherein the polynucleotide encoding the anti-BCMA CAR further comprises a polynucleotide sequence encoding a signal peptide.

Embodiment 55: The particle of any one of embodiments 32 to 54, wherein the polynucleotide encoding the anti-BCMA CAR further comprises a polynucleotide sequence encoding a signal peptide isolated from a polypeptide selected from the group consisting of: CD8α, mIgGx, hIgGk, CD33, tPA, SEAP, hGM-CSF, CSF2R, and B2M.

Embodiment 56: The particle of any one of embodiments 32 to 55, wherein the polynucleotide encoding the anti-BCMA CAR further comprises a polynucleotide sequence encoding a signal peptide comprising an amino acid sequence set forth in any one of SEQ ID NOs: 245, 246, 247, 248, 249, 250, 251, 252, 253, and 254.

Embodiment 57: The particle of any one of embodiments 32 to 56, wherein the lentiviral vector further comprises a WPRE operably linked to the 3′ end of the polynucleotide encoding the anti-BCMA CAR.

Embodiment 58: The particle of any one of embodiments 32 to 56, wherein the lentiviral vector further comprises a WPRE operably linked to the 3′ end of the polynucleotide encoding the anti-BCMA CAR, wherein the WPRE comprises, consists essentially of, or consists of the polynucleotide sequence set forth in any one of SEQ ID NOs: 315, 316, and 317.

Embodiment 59: A recombinant lentiviral particle comprising:
(a) a viral envelope comprising (i) a mutated viral envelope glycoprotein comprising an amino acid sequence set forth in SEQ ID NO: 335 and (ii) a non-viral membrane-bound tropism polypeptide comprising an amino acid sequence set forth in SEQ ID NO: 324; and (b) a recombinant lentiviral vector comprising a 5′ long terminal repeat (LTR) comprising R and U5 regions; a Psi (Ψ) packaging signal, a cPPT/FLAP, a rev response element (RRE); a polynucleotide encoding an EF1α promoter operably linked to a polynucleotide encoding an anti-BCMA chimeric antigen receptor (CAR) comprising an amino acid sequence set forth in SEQ ID NO: 266 or an amino acid sequence at least 95%, at least 96%, at least 97%, at least 98%, at least 99% identical thereto; optionally a WPRE; a 3′ LTR comprising U3 and R regions; a polyadenylation signal and a poly(A) tail.

Embodiment 60: A cell transduced with the particle of any one of embodiments 1 to 59.

Embodiment 61: The cell of embodiment 60, wherein the cell is an immune effector cell.

Embodiment 62: The cell of embodiment 60 or embodiment 61, wherein the cell is a T cell or a natural killer T (NKT) cell.

Embodiment 63: A composition comprising the particle of any one of embodiments 1 to 58 or the cell of any one of embodiments 60 to 62.

Embodiment 64: A pharmaceutical composition comprising a pharmaceutically acceptable carrier and the particle of any one of embodiments 1 to 59, the cell of any one of embodiments 60 to 62, or the composition of embodiment 63.

Embodiment 65: A method of treating, preventing, or ameliorating at least one symptom of a disease, disorder or condition associated therewith in a subject, comprising administering to the subject an effective amount of the particle of any one of embodiments 1 to 59, the cell of any one of embodiments 60 to 62, the composition of embodiment 63, or the pharmaceutical composition of embodiment 64.

Embodiment 66: The method of embodiment 65, wherein the disease, disorder, or condition is a cancer.

Embodiment 67: The method of embodiment 65 or embodiment 66, wherein the cancer is a multiple myeloma (MM).

Embodiment 68: The method of embodiment 66 or embodiment 67, wherein the cancer is MM selected from the group consisting of: active multiple myeloma, smoldering multiple myeloma, light chain myeloma, non-secretory myeloma, IgD myeloma, IgE myeloma, osteosclerotic myeloma, solitary plasmacytoma of bone, and extramedullary plasmacytoma.

Embodiment 69: The method of any one of embodiments 66 to 68, wherein the cancer is relapsed and/or refractory.

Embodiment 70: A method of treating a subject that has, or has been diagnosed with, a multiple myeloma, comprising administering the subject an effective amount of the particle of any one of embodiments 1 to 59, the cell of any one of embodiments 60 to 62, the composition of embodiment 63, or the pharmaceutical composition of embodiment 64.

Embodiment 71: The method of embodiment 70, wherein the administration is parenteral administration.

Embodiment 72: The methods of embodiment 70 or embodiment 71, wherein the administration is intravenous.

Embodiment 73: A method of transducing an immune effector cell in vivo, comprising administering to a subject a pharmaceutical composition comprising a pharmaceutically acceptable carrier and an effective amount of the particle of any one of embodiments 1 to 59, the cell of any one of embodiments 60 to 62, the composition of embodiment 63, or the pharmaceutical composition of embodiment 64.

Embodiment 74: A method of making a recombinant lentivirus comprising (a) transfecting a host cell with four polynucleotides: a first polynucleotide that encodes lentiviral gag-pol, a second polynucleotide that encodes lentiviral rev, a third polynucleotide that encodes the mutated viral envelope glycoprotein set forth in any one of embodiments 1 to 59 and the non-viral membrane-bound tropism polypeptide set forth in any one of embodiments 1 to 59, and a fourth polynucleotide that is a transfer plasmid encoding the recombinant lentiviral vector of any one of embodiments 1 to 59; and b) culturing the transduced cell for about 1 to 3 days to produce the recombinant lentivirus.

Embodiment 75: A kit comprising the particle of any one of embodiments 1 to 59, a pharmaceutically acceptable carrier, and instructions for use.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### N. Examples

### Example 1

### Recombinant Lentivirus Delivers Functional Anti-BCMA CARs to T Cells

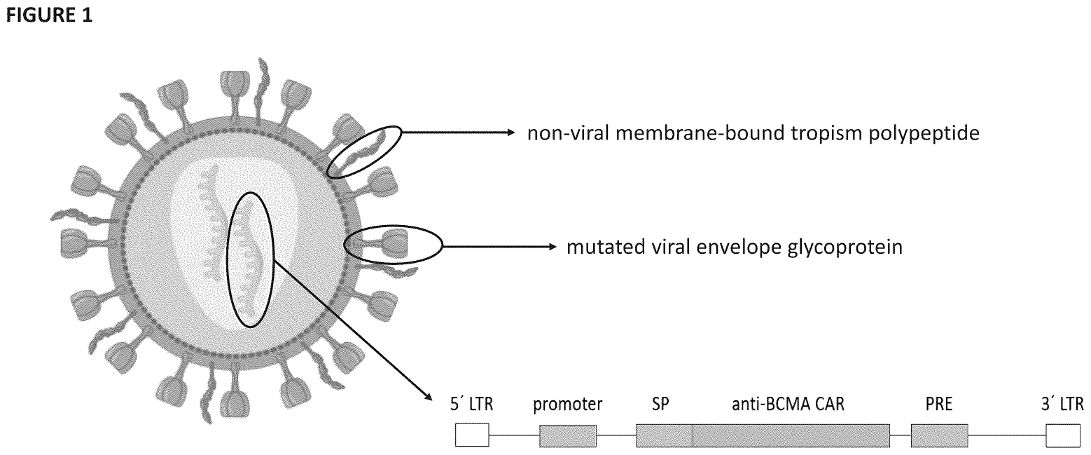

Recombinant T cell specific lentiviral particles with a viral envelope expressing a mutated viral envelope glycoprotein(fusogen) and a non-viral membrane bound tropism molecule and harboring a lentiviral vector encoding an anti-BCMA CAR were generated. FIG. 1.

HEK293T cells were transfected with plasmids encoding a non-viral membrane bound tropism molecule comprising an anti-CD3 scFv fused to a CD8α hinge and transmembrane domain (e.g., SEQ ID NO: 324); a mutant VSIV-G fusogen (e.g., SEQ ID NOs: 335); lentiviral GAG/POL; lentiviral REV; and a transfer plasmid encoding a lentiviral vector comprising an MNDU3 promoter operably linked to a polynucleotide encoding a CD8α signal peptide and an anti-BCMA CAR and a WPRE element operably linked to the 3′ end of the polynucleotide encoding the anti-BCMA CAR.

Table 16 lists the recombinant lentivirus (LV) reference number and the corresponding SEQ ID NOs of the amino acid sequences of the anti-BCMA CARs and their CARchitectures.

TABLE 16

| | SEQ | anti-BCMA | | | | |
|---|---|---|---|---|---|---|
| LV Ref. | ID NO. | binding domain | Hinge | TM | Costim | Primary |
| LV 1 | 256 | scFv | CD8α | CD8α | CD137 | CD3ζ |
| LV 2 | 258 | scFv | CD8α | CD8α | CD137 | CD3ζ |
| LV 3 | 259 | scFv | CD8α | CD8α | CD137 | CD3ζ |
| LV 4 | 262 | scFv | CD8α | CD8α | CD137 | CD3ζ |
| LV 5 | 263 | scFv | CD8α | CD8α | CD137 | CD3ζ |
| LV 6 | 266 | scFv | CD8α | CD8α | CD137 | CD3ζ |
| LV 7 | 268 | scFv | CD8α | CD8α | CD137 | CD3ζ |
| LV 8 | 270 | scFv | CD8α | CD8α | CD137 | CD3ζ |
| LV 9 | 273 | VHH | CD8α | CD8α | CD137 | CD3ζ |
| LV 10 | 274 | VHH | CD8α | CD8α | CD137 | CD3ζ |
| LV 11 | 275 | VHH | CD8α | CD8α | CD137 | CD3ζ |
| LV 12 | 276 | VHH | CD8α | CD8α | CD137 | CD3ζ |
| LV 13 | 277 | VHH | CD8α | CD8α | CD137 | CD3ζ |
| LV 14 | 278 | VHH | CD8α | CD8α | CD137 | CD3ζ |
| LV 15 | 279 | VHH | CD8α | CD8α | CD137 | CD3ζ |
| LV 16 | 281 | VHH | CD8α | CD8α | CD137 | CD3ζ |
| LV 17 | 282 | VHH | CD8α | CD8α | CD137 | CD3ζ |
| LV 18 | 283 | VHH | CD8α | CD8α | CD137 | CD3ζ |
| LV 19 | NA | scFv | CD8α | CD8α | CD137 | CD3ζ |

### Jurkat Functional Titer

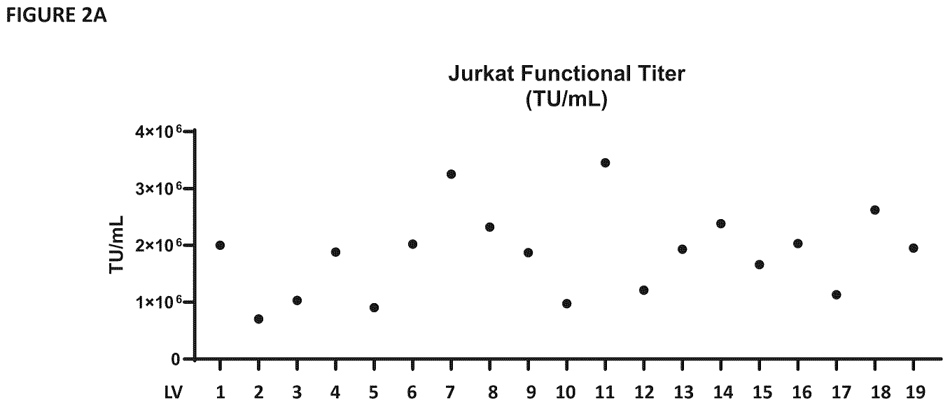

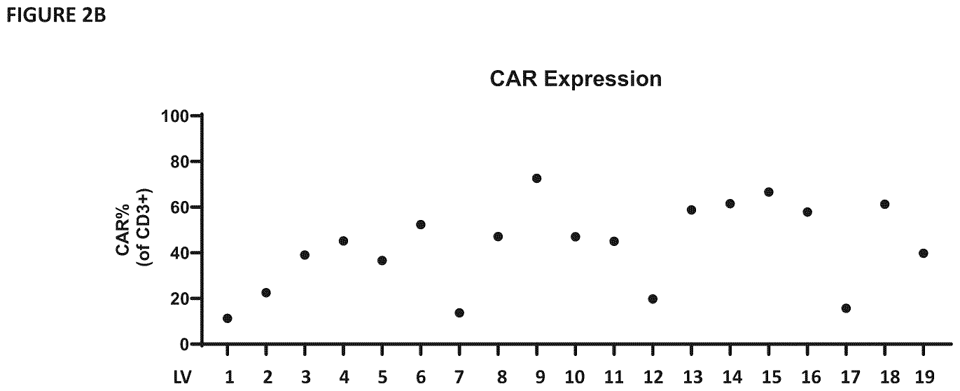

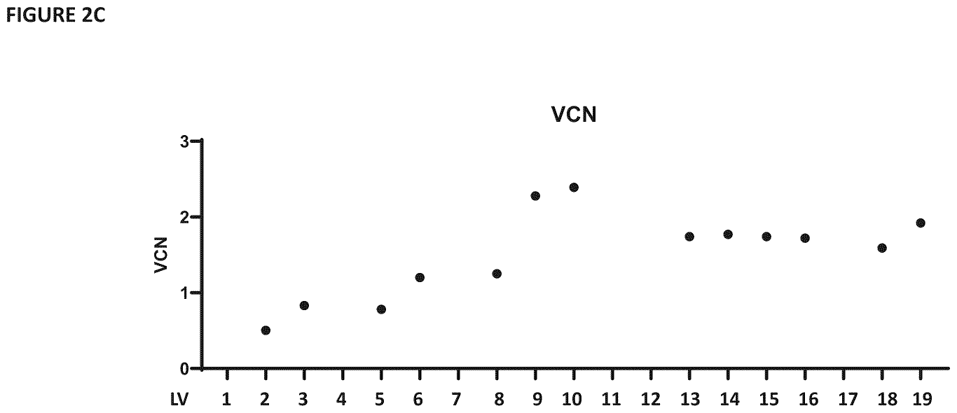

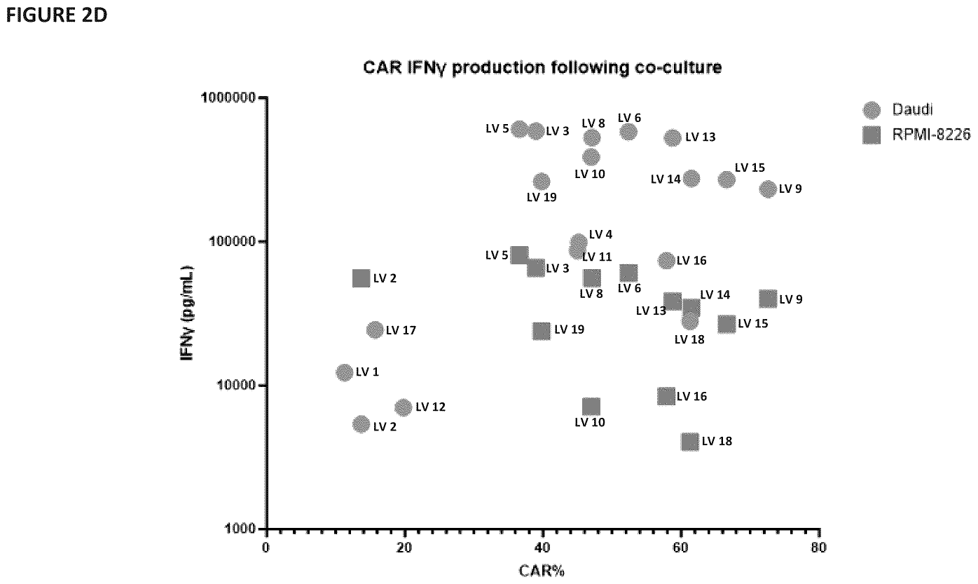

1×10⁵ Jurkat cells were plated in each well of a 96-well plate. Cells were transduced with recombinant lentiviruses LV 1 to LV 18 that harbor novel anti-BCMA CARs and LV 19, which harbors a control anti-BCMA CAR obtained from the literature. Seven days post-transduction, Jurkat cells were harvested and stained with a recombinant, phycoerythrin (PE) labeled, BCMA extracellular domain-FC fusion protein (BCMA-PE) and analyzed by flow cytometry. Functional titer, expressed as the number of transducing units (TU) per mL, was determined by measuring the number of transduced Jurkat cells. FIG. 2A.

### Anti-BCMA CAR Expression

5×10⁵ human PBMCs were plated in each well of a 24-well plate. Cells were transduced with recombinant lentiviruses LV 1 to LV 19 at a MOI 2 based on the Jurkat functional titer, or a 0.5 mL volumetric transduction if MOI 2 was not achievable. Seven days post-transduction, PBMCs were harvested and stained with BCMA-PE and analyzed by flow cytometry to assess the percentage of anti-BCMA CAR expressing cells. FIG. 2B.

### Anti-BCMA CAR Activity

5×10⁴ PBMCs transduced with recombinant lentiviruses LV 1 to LV 19 were co-cultured with 5×10⁴ high BCMA-expressing tumor cells (RPMI-8226) or 5×10⁴ low BCMA-expressing tumor cells (Daudi) for 24 hours. Anti-BCMA CAR activity was assessed by harvesting co-culture supernatants and measuring IFNγ levels using a Meso Scale Discovery (MSD®) assay. The percentage of anti-BCMA CAR positive cells was plotted against IFNγ levels produced in co-culture. FIG. 2D.

### Summary

These data indicate that the recombinant T cell specific lentiviral particles harboring anti-BCMA CARs (LV 1 to LV 18) are able to transduce CD3 expressing cells, that anti-BCMA CARs are expressed on PBMCs transduced with LV 1 to LV 18 and that the transduced PBMCs express anti-BCMA CARs that recognize high or low BCMA-expressing cells and produce IFNγ in response to binding antigen.

### Example 2

### Lentiviral Vector Architecture and Anti-BCMA CAR Expression and Function

Recombinant T cell specific lentiviral particles with a viral envelope expressing a mutated viral envelope glycoprotein(fusogen) and a non-viral membrane bound tropism molecule and harboring a lentiviral vector encoding various promoters, anti-BCMA CARs, and either no posttranscriptional response element (PRE) or a wild-type WPRE, or a mutated WPRE.

HEK293T cells were transfected with plasmids encoding a non-viral membrane bound tropism molecule comprising an anti-CD3 scFv fused to a CD8α hinge and transmembrane domain (e.g., SEQ ID NO: 324); a mutant VSIV-G fusogen (e.g., SEQ ID NOs: 335); lentiviral GAG/POL; lentiviral REV; and a transfer plasmid encoding a lentiviral vector comprising either an MNDU3 promoter (SEQ ID NO: 319), an SFFV promoter (SEQ ID NO: 322), or an EF1α promoter (SEQ ID NO: 320) operably linked to a polynucleotide encoding a CD8α signal peptide and an anti-BCMA CAR and either no posttranscriptional response element or a wild-type WPRE (SEQ ID NO: 315) or a mutated WPRE (SEQ ID NO: 316) operably linked to the 3′ end of the polynucleotide encoding the anti-BCMA CAR.

Table 17 lists the recombinant lentivirus reference number and the corresponding SEQ ID NOs of the amino acid sequences of the anti-BCMA CARs and the different lentiviral vector architectures.

TABLE 17

| Ref. | SEQ ID NO. | Promoter | WPRE |
|---|---|---|---|
| LV 3.1 | 259 | MNDU3 | wild-type |
| LV 3.2 | 259 | MNDU3 | no PRE |
| LV 3.3 | 259 | MNDU3 | mutant WPRE |
| LV 3.4 | 259 | SFFV | wild-type |
| LV 3.5 | 259 | SFFV | no PRE |
| LV 3.6 | 259 | SFFV | mutant WPRE |
| LV 3.7 | 259 | EF1α | wild-type |
| LV 3.8 | 259 | EF1α | no PRE |
| LV 3.9 | 259 | EF1α | mutant WPRE |
| LV 5.1 | 263 | MNDU3 | wild-type |
| LV 5.2 | 263 | MNDU3 | no PRE |
| LV 5.3 | 263 | MNDU3 | mutant WPRE |
| LV 5.4 | 263 | SFFV | wild-type |
| LV 5.5 | 263 | SFFV | no PRE |
| LV 5.6 | 263 | SFFV | mutant WPRE |
| LV 5.7 | 263 | EF1α | wild-type |
| LV 5.8 | 263 | EF1α | no PRE |
| LV 5.9 | 263 | EF1α | mutant WPRE |
| LV 6.1 | 266 | MNDU3 | wild-type |
| LV 6.2 | 266 | MNDU3 | no PRE |
| LV 6.3 | 266 | MNDU3 | mutant WPRE |
| LV 6.4 | 266 | SFFV | wild-type |
| LV 6.5 | 266 | SFFV | no PRE |
| LV 6.6 | 266 | SFFV | mutant WPRE |
| LV 6.7 | 266 | EF1α | wild-type |
| LV 6.8 | 266 | EF1α | no PRE |
| LV 6.9 | 266 | EF1α | mutant WPRE |
| LV 8.1 | 270 | MNDU3 | wild-type |
| LV 8.2 | 270 | MNDU3 | no PRE |
| LV 8.3 | 270 | MNDU3 | mutant WPRE |
| LV 8.4 | 270 | SFFV | wild-type |
| LV 8.5 | 270 | SFFV | no PRE |
| LV 8.6 | 270 | SFFV | mutant WPRE |
| LV 8.7 | 270 | EF1α | wild-type |
| LV 8.8 | 270 | EF1α | no PRE |
| LV 8.9 | 270 | EF1α | mutant WPRE |
| LV 9.1 | 273 | MNDU3 | wild-type |
| LV 9.2 | 273 | MNDU3 | no PRE |
| LV 9.3 | 273 | MNDU3 | mutant WPRE |
| LV 9.4 | 273 | SFFV | wild-type |
| LV 9.5 | 273 | SFFV | no PRE |
| LV 9.6 | 273 | SFFV | mutant WPRE |
| LV 9.7 | 273 | EF1α | wild-type |
| LV 9.8 | 273 | EF1α | no PRE |
| LV 9.9 | 273 | EF1α | mutant WPRE |
| LV 13.1 | 277 | MNDU3 | wild-type |
| LV 13.2 | 277 | MNDU3 | no PRE |
| LV 13.3 | 277 | MNDU3 | mutant WPRE |
| LV 13.4 | 277 | SFFV | wild-type |
| LV 13.5 | 277 | SFFV | no PRE |
| LV 13.6 | 277 | SFFV | mutant WPRE |
| LV 13.7 | 277 | EF1α | wild-type |
| LV 13.8 | 277 | EF1α | no PRE |
| LV 13.9 | 277 | EF1α | mutant WPRE |
| LV19 | | MNDU3 | no PRE |

### Infectious Titer

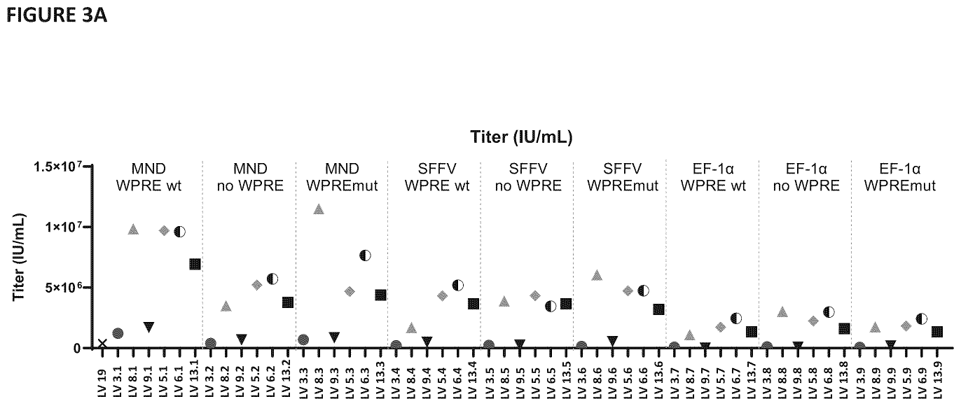

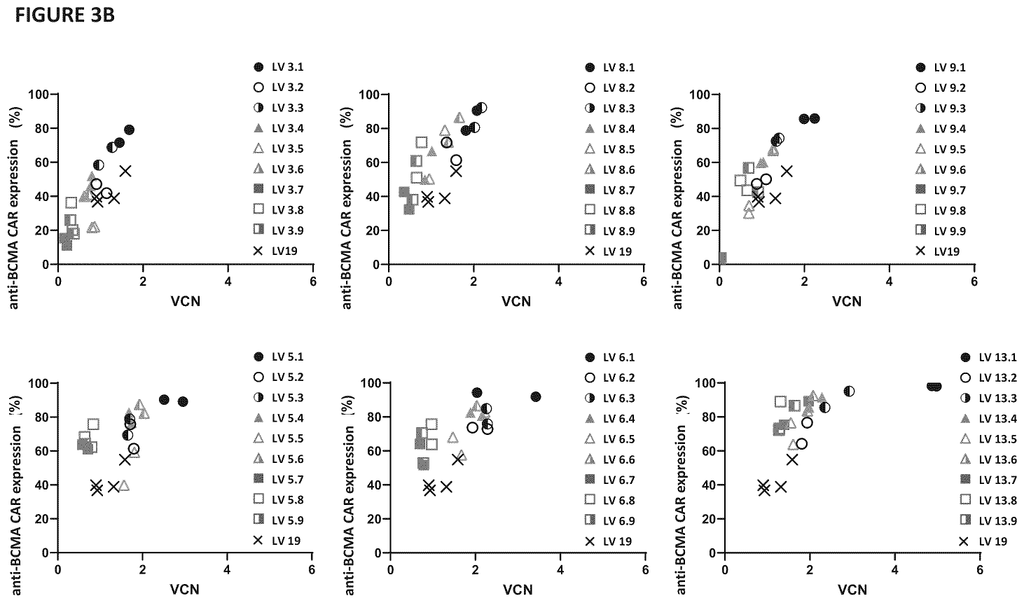

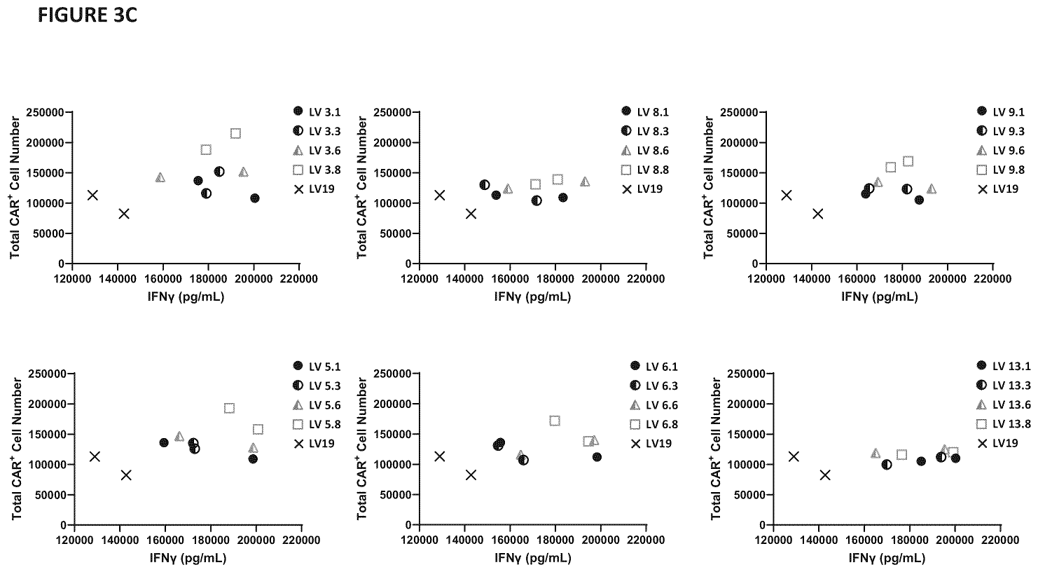

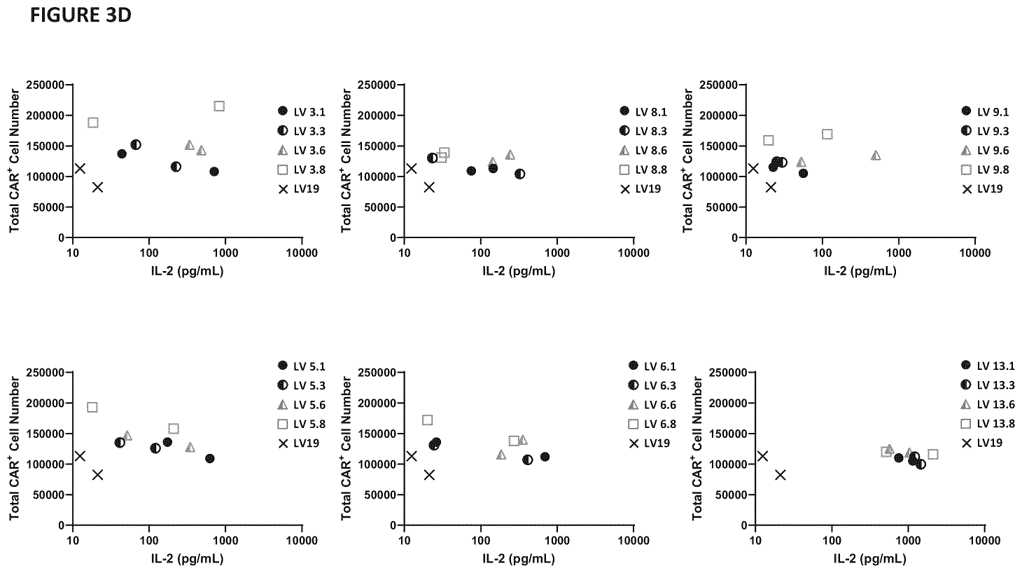

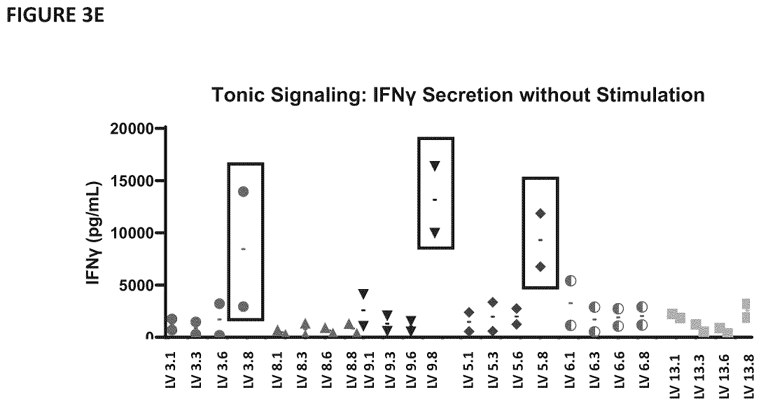

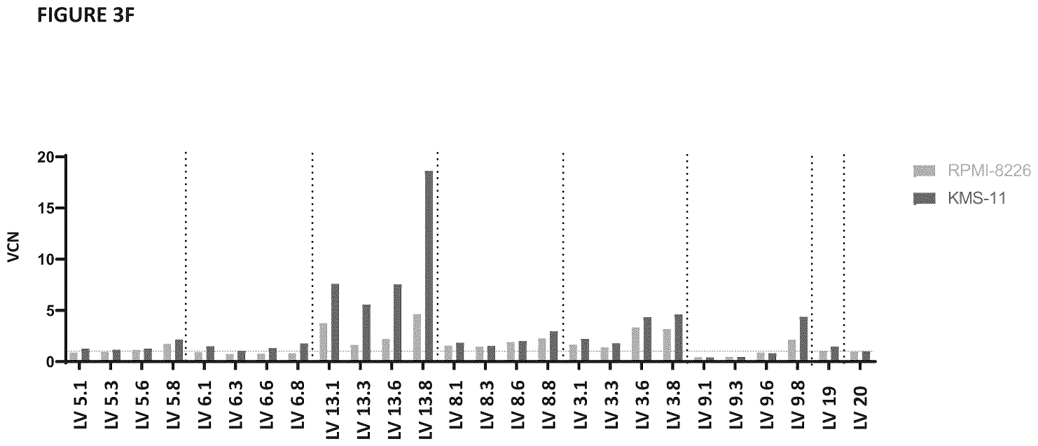

1×10⁵ Jurkat cells were plated in each well of a 96-well plate and transduced with the recombinant lentiviruses listed in Table 17 including LV 19, which harbors a lentiviral vector encoding a control anti-BCMA CAR obtained from the literature. Three days post-transduction, the cells were passaged. Seven days post-transduction the cells were harvested. Genomic DNA was isolated and purified from the harvested cells and used in a quantitative PCR (qPCR) assay to determine vector copy number (VCN) and subsequently, IU/mL. FIG. 3A.

All lentiviral vector architectures examined produced infectious titers and were subsequently used to transduce PBMCs.

### VCN and Anti-BCMA CAR Expression

5×10⁵ human PBMCs were plated in each well of a 24-well plate and transduced with volume matched recombinant lentiviruses listed in Table 17.

Four days post-transduction, PBMCs were passaged to a 24-well GREX plate. Seven days post-transduction, PBMCs were harvested, one aliquot of cells was stained with BCMA-PE and analyzed by flow cytometry to assess the percentage of anti-BCMA CAR expressing cells and another aliquot was used to isolate and purify genomic DNA for a quantitative PCR (qPCR) assay to determine vector copy number (VCN). FIG. 3B.

These data show that different lentiviral vector architectures tested in combination with different anti-BCMA CARs result in a spectrum of transduction and anti-BCMA CAR expression.

### Anti-BCMA CAR Activity

5×10⁵ human PBMCs were plated in each well of a 24-well plate and transduced with recombinant lentiviruses listed in Table 17 that have the following lentiviral vector architectures: MNDU3 promoter and wild-type WPRE, MNDU3 promoter and a mutated WPRE, SFFV promoter and a mutated WPRE, and EF1α promoter and no WPRE. PBMCs were transduced at an MOI of 1 (based on IU/mL determined in Jurkat cells), except for LV 3.6, LV 3.8, LV 9.8, and LV 13.8, in which volume matched lentivirus was used. Four days post-transduction, PBMCs were passaged to a 24-well GREX plate. Seven days post-transduction, PBMCs were harvested, one aliquot of cells was stained with BCMA-PE and analyzed by flow cytometry to assess the number of anti-BCMA CAR expressing cells and another aliquot was used in co-culture assays to assess anti-BCMA CAR function.

5×10⁴ transduced PBMCs were co-cultured with 5×10⁴ RPMI-8226 cells for 24 hours. Anti-BCMA CAR activity was assessed by harvesting PBMC/RPMI-8226 cell co-culture supernatants and measuring IFNγ and IL-2 levels using an MSD assay. IFNγ and IL-2 levels produced in co-culture were plotted against the percentage of anti-BCMA CAR positive cells. FIGS. 3C and 3D.

Antigen independent anti-BCMA CAR activity was assessed by culturing 5×10⁴ transduced PBMCs in the absence of target cells for 24 hours. After 24 hours, the supernatants were harvested and IFNγ levels measured using an MSD assay. IFNγ levels were plotted against lentiviral architectures used to express the anti-BCMA CARs. FIG. 3E.

These data indicate that combinations of different lentiviral architectures and anti-BCMA CARs can be selected to modulate anti-BCMA CAR expression and activity. Further, the data show that PBMCs expressing the anti-BCMA CARs set forth in SEQ ID NOs: 259, 263, 266, 270, 273, and 277 show comparable or increased cell expansion and comparable or increased activity compared to the control anti-BCMA CAR and that only three combinations showed high levels of antigen independent (tonic) signaling.

### Off-Target Transduction

Off-target transduction of multiple myeloma cells was evaluated in two BCMA-expressing multiple myeloma cell lines, RPMI-8226 cells and KMS-11 cells. 1×10⁵ RPMI-8226 or 1×10⁵ KMS-11 cells were plated in each well of a 96-well plate and treated at an MOI of 1 with recombinant lentiviruses listed in Table 17 that have the following lentiviral vector architectures, MNDU3 promoter and wild-type WPRE, MNDU3 promoter and a mutated WPRE, SFFV promoter and a mutated WPRE, and EF1α promoter and no WPRE; LV 19; and with LV 20. LV 20 is a recombinant lentiviral particle comprising a viral envelope that expresses a non-viral membrane bound tropism molecule comprising an anti-CD3 scFv fused to a CD8α hinge and transmembrane domain (e.g., SEQ ID NO: 324); a mutant VSIV-G fusogen (e.g., SEQ ID NOs: 335); and a lentiviral vector comprising an MNDU3 promoter (SEQ ID NO: 319), operably linked to a polynucleotide encoding a CD8α signal peptide and GFP and a wild-type WPRE (SEQ ID NO: 315) operably linked to the 3′ end of the polynucleotide encoding GFP.

Three days post-treatment, the cells were passaged. Seven days post-treatment, the cells were harvested and genomic DNA was isolated and purified for a qPCR assay to determine vector integration using VCN. VCN values for anti-BCMA CARs were normalized to VCN for LV 20, which expresses GFP rather than an anti-BCMA CAR.

The data show that differences in off-target multiple myeloma transduction were largely driven by the particular anti-BCMA CAR being expressed, rather than any particular lentiviral vector architecture. Several architectures used to express the anti-BCMA CARs in LV 3, LV 5, LV 6, LV 8, and LV 9 showed low levels of off-target transduction that were comparable to or less than LV 19, which expresses a control anti-BCMA CAR. In contrast, LV 13 exhibited the highest rates of off-target transduction compared to other LVs. FIG. 3F.

### Example 3

### In Vivo Administered Lentivirus Demonstrates Anti-Tumor Efficacy in a Multiple Myeloma Mouse Model

The anti-tumor efficacy of in vivo administered recombinant lentiviral particles comprising an envelope that expresses an anti-CD3-based tropism molecule and a mutant VSIV-G fusogen and a lentiviral vector encoding an anti-BCMA CAR was investigated in multiple myeloma mouse models.

Recombinant lentivirus for in vivo administration was produced by transient transfection of HEK293T cells with plasmids encoding an anti-CD3-based tropism molecule (SEQ ID NO: 324); a mutant VSIV-G fusogen (e.g., SEQ ID NO: 335); lentiviral GAG/POL; lentiviral REV; and a transfer plasmid encoding a lentiviral vector comprising: (i) an MNDU3 promoter (SEQ ID NO: 319) operably linked to a polynucleotide encoding a CD8α signal peptide and an anti-BCMA CAR, and a wild-type WPRE (SEQ ID NO: 315) operably linked to the 3′ end of the polynucleotide encoding the anti-BCMA CAR; (ii) an MNDU3 promoter (SEQ ID NO: 319) operably linked to a polynucleotide encoding a CD8α signal peptide and an anti-BCMA CAR, and a mutated WPRE (SEQ ID NO: 316) operably linked to the 3′ end of the polynucleotide encoding the anti-BCMA CAR; (iii) an SFFV promoter (SEQ ID NO: 322) operably linked to a polynucleotide encoding a CD8α signal peptide and an anti-BCMA CAR, and a mutated WPRE (SEQ ID NO: 316) operably linked to the 3′ end of the polynucleotide encoding the anti-BCMA CAR; or (iv) an EF1α promoter (SEQ ID NO: 320) operably linked to a polynucleotide encoding a CD8α signal peptide and an anti-BCMA CAR without a PRE.

The recombinant lentivirus reference number, the SEQ ID NO of the anti-BCMA CAR polypeptide and the corresponding lentiviral architectures shown in Table 18 were used in this Example.

TABLE 18

| Ref. | SEO ID NO. | Promoter | WPRE |
|---|---|---|---|
| LV 3.1 | 259 | MNDU3 | wild-type |
| LV 3.3 | 259 | MNDU3 | mutant WPRE |
| LV 3.6 | 259 | SFFV | mutant WPRE |
| LV 3.8 | 259 | EF1α | no PRE |
| LV 5.1 | 263 | MNDU3 | wild-type |
| LV 5.3 | 263 | MNDU3 | mutant WPRE |
| LV 5.6 | 263 | SFFV | mutant WPRE |
| LV 5.8 | 263 | EF1α | no PRE |
| LV 6.1 | 266 | MNDU3 | wild-type |
| LV 6.3 | 266 | MNDU3 | mutant WPRE |
| LV 6.6 | 266 | SFFV | mutant WPRE |
| LV 6.8 | 266 | EF1α | no PRE |
| LV 8.1 | 270 | MNDU3 | wild-type |
| LV 8.3 | 270 | MNDU3 | mutant WPRE |
| LV 8.6 | 270 | SFFV | mutant WPRE |
| LV 8.8 | 270 | EF1α | no PRE |
| LV 9.1 | 273 | MNDU3 | wild-type |
| LV 9.3 | 273 | MNDU3 | mutant WPRE |
| LV 9.6 | 273 | SFFV | mutant WPRE |
| LV 9.8 | 273 | EF1α | no PRE |
| LV 13.1 | 277 | MNDU3 | wild-type |
| LV 13.3 | 277 | MNDU3 | mutant WPRE |
| LV 13.6 | 277 | SFFV | mutant WPRE |
| LV 13.8 | 277 | EF1α | no PRE |

Ex vivo anti-BCMA CAR T cells were also prepared. Briefly, HEK293T cells were transiently transfected with plasmids encoding a wild-type VSIV-G fusogen; lentiviral GAG/POL; lentiviral REV; and a transfer plasmid encoding a lentiviral vector comprising an MNDU3 promoter operable linked to a CD8α signal peptide and a control anti-BCMA CAR obtained from the literature, and a wild-type WPRE (SEQ ID NO: 315) operably linked to the 3′ end of the polynucleotide encoding the anti-BCMA CAR. PBMCs were then transduced with the recombinant lentivirus and cultured for 7 days to generate anti-BCMA CAR T cells.

### First Daudi Model Study

NSG mice were intravenously injected with 2×10⁶ Daudi cells labeled with firefly luciferase. After four days, four out of five groups of mice were intravenously administered 1×10⁶ human PBMCs. The next day mice that received the 1×10⁶ human PBMCs were administered vehicle control (DMEM); or 2.2×10⁸ IU of LV 3.1, LV 6.1, LV 8.1, or LV 13.1. Mice that were not administered PBMCs were administered 5×10⁶ ex vivo anti-BCMA CAR T cells. All groups of mice then received three doses of 2×10⁵ IU recombinant human IL-2 at 6, 24, and 48 hours post LV administration. Tumor volume was measured by using a bioluminescence imaging system.

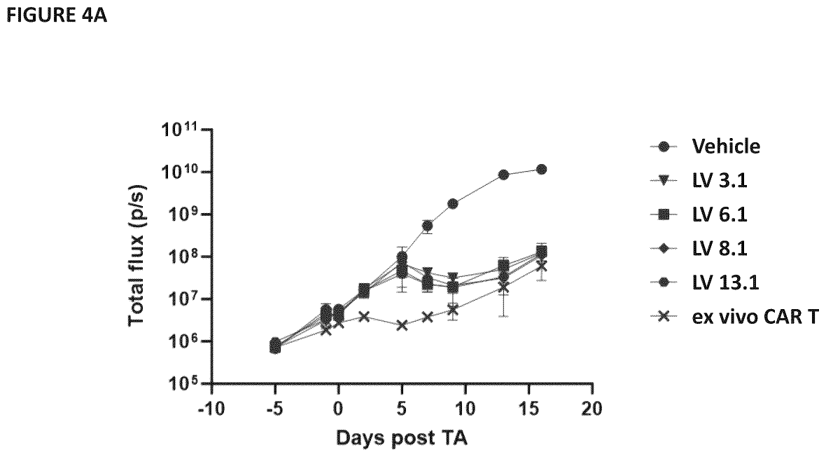

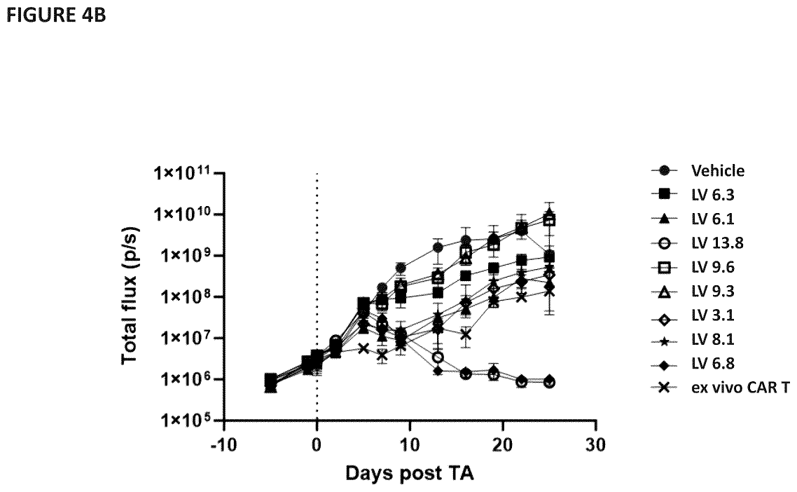

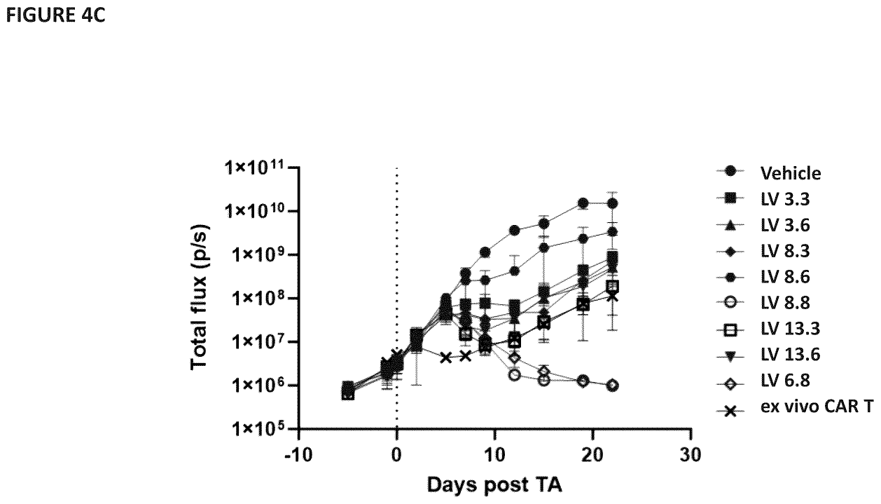

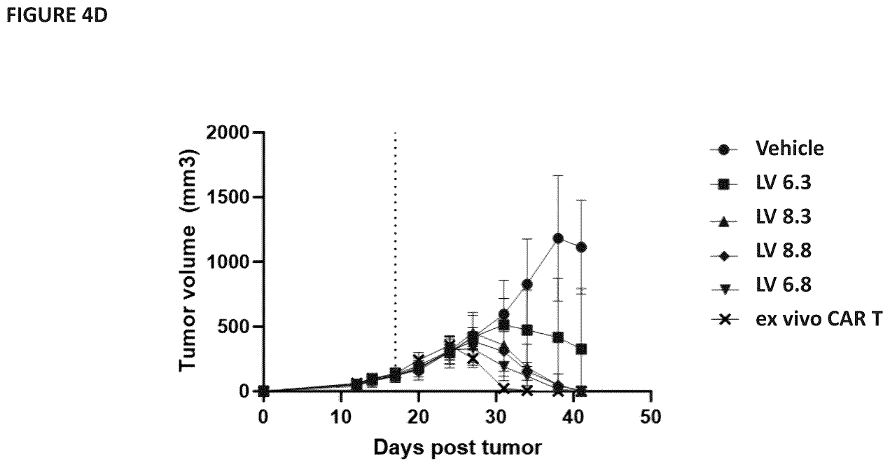

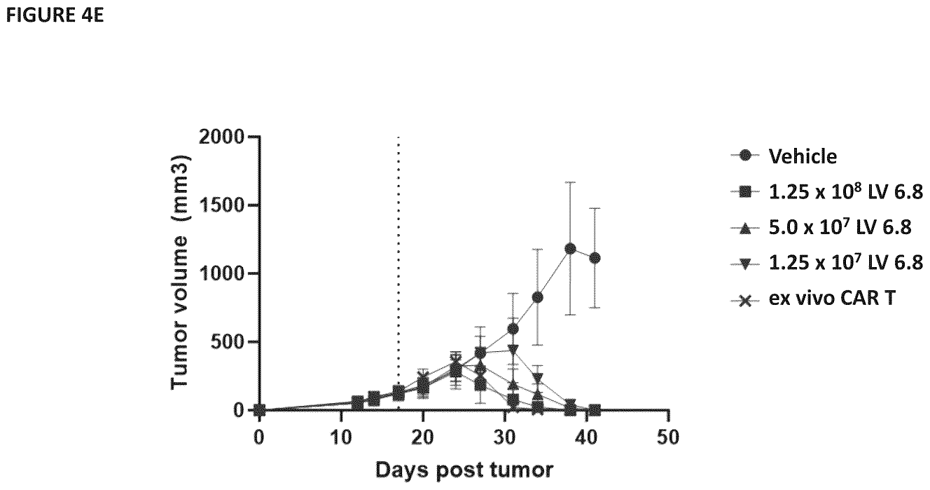

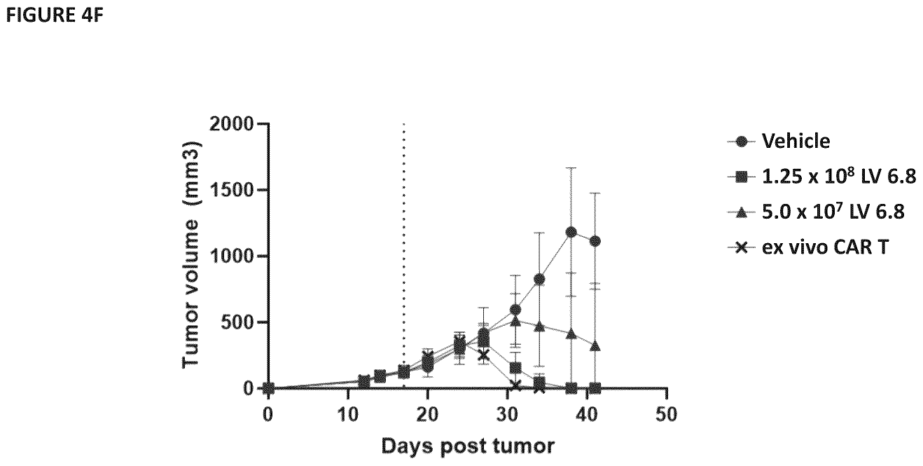

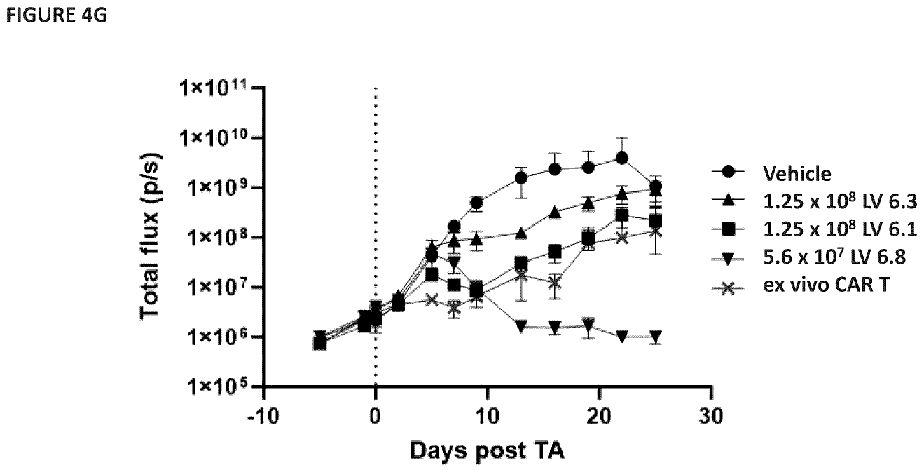

Tumor size increased in mice treated with vehicle. Mice treated with ex vivo anti-BCMA CAR T cells and in vivo with LV anti-BCMA CAR experienced tumor regression. FIG. 4A.

### Second Daudi Model Study

NSG mice were intravenously injected with 2×10⁶ Daudi cells labeled with firefly luciferase. After four days, eight out of nine groups of mice were intravenously administered 1×10⁶ human PBMCs. The next day mice that received the 1×10⁶ human PBMCs were administered vehicle control(DMEM); 1.25×10⁸ IU of LV 3.1, LV 6.1, LV 6.3, LV 8.1, LV 9.3, LV 9.6, or LV 13.8; or 5.6×10⁷ IU of LV 6.8. Mice that were not administered PBMCs were administered 5×10⁶ ex vivo anti-BCMA CAR T cells. All groups of mice then received three doses of 2×10⁵ IU recombinant human IL-2 at 6, 24, and 48 hours post LV administration. Tumor volume was measured by using a bioluminescence imaging system.

Tumor size increased in mice treated with vehicle. Mice treated with ex vivo anti-BCMA CAR T cells and in vivo with some LV anti-BCMA CARs experienced mild control of tumor growth, whereas LV 6.8 and LV 13.8 experienced durable tumor regression. FIG. 4B.

### Third Daudi Model Study

NSG mice were intravenously injected with 2×10⁶ Daudi cells labeled with firefly luciferase. After four days, eight out of nine groups of mice were intravenously administered 1×10⁶ human PBMCs. The next day mice that received the 1×10⁶ human PBMCs were administered vehicle control(DMEM); 1.25×10⁸ IU of LV 3.3, LV 3.6, LV 8.3, LV 8.6, LV 8.8, LV 13.3, or LV 13.6; or 5.6×10⁷ IU of LV 6.8. Mice that were not administered PBMCs were administered 5×10⁶ ex vivo anti-BCMA CAR T cells. All groups of mice then received three doses of 2×10⁵ IU recombinant human IL-2 at 6, 24, and 48 hours post LV administration. Tumor volume was measured by using a bioluminescence imaging system.

Tumor size increased in mice treated with vehicle. Mice treated with ex vivo anti-BCMA CAR T cells and in vivo with some LV anti-BCMA CARs experienced mild control of tumor growth, whereas LV 6.8 and LV 8.8 experienced durable tumor regression. FIG. 4C.

### First RPMI Model Study

NOD scid gamma (NSG) mice were subcutaneously injected with 1×10⁶ RPMI-8226 cells (a BCMA positive tumor cell line). Tumors were allowed to grow to a size of about 110 mm³ to 140 mm³ (about two and a half weeks).

Five out of six groups of mice were then intravenously administered 1×10⁶ human PBMCs. The next day, mice that received the 1×10⁶ human PBMCs were administered vehicle control (DMEM); 5.0×10⁷ IU of LV 6.3, LV 6.8, LV 8.3, or LV 8.8. The sixth group of mice was administered 2×10⁶ unmodified ex vivo anti-BCMA CAR T cells. All groups of mice then received three doses of 2×10⁵ IU recombinant human IL-2 at 6, 24, and 48 hours post LV administration. Tumor volume was measured externally using calipers and mice were euthanized at pre-determined humane endpoints based on tumor size and body condition.

Tumor size increased in mice treated with vehicle control. Mice treated with LV 6.3 experienced moderate tumor regression, whereas mice treated with ex vivo anti-BCMA CAR T cells or in vivo with LV 6.8, LV 8.3, or LV 8.8 experienced complete and durable tumor regression. FIG. 4D.

Mice that were not administered PBMCs were administered 5×10⁶ ex vivo anti-BCMA CAR T cells. All groups of mice then received three doses of 2×10⁵ IU recombinant human IL-2 at 6, 24, and 48 hours post LV administration.

### Second RPMI Model Study

NOD scid gamma (NSG) mice were subcutaneously injected with 1×10⁶ RPMI-8226 cells (a BCMA positive tumor cell line). Tumors were allowed to grow to a size of about 110 mm³ to 140 mm³ (about two and a half weeks).

Four out of five groups of mice were then intravenously administered 1×10⁶ human PBMCs. The next day, mice that received the 1×10⁶ human PBMCs were administered vehicle control (DMEM); 1.25×10⁷ IU of LV 6.8, 5.0×10⁷ IU of LV 6.8, or 1.25×10⁸ IU of LV 6.8. The fifth group of mice was administered 2×10⁶ unmodified ex vivo anti-BCMA CAR T cells. All groups of mice then received three doses of 2×10⁵ IU recombinant human IL-2 at 6, 24, and 48 hours post LV administration. Tumor volume was measured externally using calipers and mice were euthanized at pre-determined humane endpoints based on tumor size and body condition.

Tumor size increased in mice treated with vehicle control. Mice treated with all three doses of LV 6.8 experienced dose-dependent but complete and durable tumor regression. Mice treated with ex vivo anti-BCMA CAR T cells also experienced complete and durable tumor regression. FIG. 4E.

### Third RPMI Model Study

NOD scid gamma (NSG) mice were subcutaneously injected with 1×10⁶ RPMI-8226 cells (a BCMA positive tumor cell line). Tumors were allowed to grow to a size of about 110 mm³ to 140 mm³ (about two and a half weeks).

Three out of four groups of mice were then intravenously administered 1×10⁶ human PBMCs. The next day, mice that received the 1×10⁶ human PBMCs were administered vehicle control (DMEM); 5.0×10⁷ IU of LV 6.3 or 1.25×10⁸ IU of LV 6.3. The fourth group of mice was administered 2×10⁶ unmodified ex vivo anti-BCMA CAR T cells. All groups of mice then received three doses of 2×10⁵ IU recombinant human IL-2 at 6, 24, and 48 hours post LV administration. Tumor volume was measured externally using calipers and mice were euthanized at pre-determined humane endpoints based on tumor size and body condition.

Tumor size increased in mice treated with vehicle control. Mice treated with both doses of LV 6.3 experienced dose-dependent tumor regression. Mice treated with ex vivo anti-BCMA CAR T cells experienced complete and durable tumor regression. FIG. 4F.

### Fourth Daudi Model Study

NSG mice were intravenously injected with 2×10⁶ Daudi cells labeled with firefly luciferase. After four days, four out of five groups of mice were intravenously administered 1×10⁶ human PBMCs. The next day mice that received the 1×10⁶ human PBMCs were administered vehicle control (DMEM); 1.25×10⁸ IU of LV 6.1 or LV6.3; or 5.6×10⁷ IU of LV 6.8. Mice that were not administered PBMCs were administered 5×10⁶ ex vivo anti-BCMA CAR T cells. All groups of mice then received three doses of 2×10⁵ IU recombinant human IL-2 at 6, 24, and 48 hours post LV administration. Tumor volume was measured by using a bioluminescence imaging system.

Tumor size increased in mice treated with vehicle. Mice treated with ex vivo anti-BCMA CAR T cells and in vivo with LV 6.1 and LV 6.3 experienced mild control of tumor growth, whereas LV 6.8 experienced complete and durable tumor regression. FIG. 4G.

### Example 4

### Comparative Anti-Tumor Efficacy in a Multiple Myeloma Mouse Model in Both In Vivo and Ex Vivo Formats

The anti-tumor efficacy of recombinant lentiviral particles comprising an envelope that expresses an anti-CD3-based tropism molecule and a mutant VSIV-G fusogen and a lentiviral vector encoding various anti-BCMA CARs was investigated in multiple myeloma mouse models. The recombinant lentiviruses were formulated as in vivo administered lentiviral particles and were also used to manufacture ex vivo anti-BCMA CAR T cells.

Recombinant lentivirus was produced by transient transfection of HEK293T cells with plasmids encoding a non-viral membrane bound tropism molecule comprising an anti-CD3 scFv fused to a CD8α hinge and transmembrane domain; a mutant VSIV-G fusogen comprising K47Q and R354A amino acid substitutions; lentiviral GAG/POL; lentiviral REV; and a transfer plasmid encoding a lentiviral vector encoding an anti-BCMA CAR set forth in SEQ ID NO: 266, SEQ ID NO: 340, or SEQ ID NO: 341 or a GFP control.

TABLE 19

| SEQ | |
|---|---|
| ID | |
| NO: | NUCLEIC ACID SEQUENCE |
| 340 | DIVLTQSPPSLAMSLGKRATISCRASESVTILGSHLIHWYQQKPGQPPTLLIQLASNVQTG |
| | VPARFSGSGSRTDFTLTIDPVEEDDVAVYYCLQSRTIPRTFGGGTKLEIKGSTSGSGKPGS |
| | GEGSTKGQIQLVQSGPELKKPGETVKISCKASGYTFTDYSINWVKRAPGKGLKWMGWINTE |
| | TREPAYAYDERGRFAFSLETSASTAYLQINNLKYEDTATYFCALDYSYAMDYWGQGTSVTV |
| | SSAAATTTPAPRPPTPAPTIASQPLSLRPEACRPAAGGAVHTRGLDFACDIYIWAPLAGTC |
| | GVLLLSLVITLYCKRGRKKLLYIFKQPFMRPVQTTQEEDGCSCRFPEEEEGGCELRVKFSR |
| | SADAPAYQQGQNQLYNELNLGRREEYDVLDKRRGRDPEMGGKPRRKNPQEGLYNELQKDKM |
| | AEAYSEIGMKGERRRGKGHDGLYQGLSTATKDTYDALHMQALPPR |
| 341 | QVKLEESGGGLVQAGRSLRLSCAASEHTFSSHVMGWFRQAPGKERESVAVIGWRDISTSYA |
| | DSVKGRFTISRDNAKKTLYLQMNSLKPEDTAVYYCAARRIDAADFDSWGQGTQVTVSSGGG |
| | GSEVQLVESGGGLVQAGGSLRLSCAASGRTFTMGWFRQAPGKEREFVAAISLSPTLAYYAE |
| | SVKGRFTISRDNAKNTVVLQMNSLKPEDTALYYCAADRKSVMSIRPDYWGQGTQVTVSSTS |
| | TTTPAPRPPTPAPTIASQPLSLRPEACRPAAGGAVHTRGLDFACDIYIWAPLAGTCGVLLL |
| | SLVITLYCKRGRKKLLYIFKQPFMRPVQTTQEEDGCSCRFPEEEEGGCELRVKFSRSADAP |
| | AYQQGQNQLYNELNLGRREEYDVLDKRRGRDPEMGGKPRRKNPQEGLYNELQKDKMAEAYS |
| | EIGMKGERRRGKGHDGLYQGLSTATKDTYDALHMQALPPR |

The recombinant lentivirus reference number, the SEQ ID NO of the anti-BCMA CAR polypeptide and the corresponding lentiviral architectures shown in Table 12 were used in this Example.

TABLE 20

| Ref. | SEQ ID NO. | Promoter | WPRE |
|---|---|---|---|
| LV 6.8 | 266 | EF1α | none |
| LV A | 340 | MNDU3 | WT WPRE |
| LV B | 341 | EF1α | none |
| LV19 | GFP | MNDU3 | none |

Ex vivo anti-BCMA CAR T cells were also prepared by transducing PBMCs with the recombinant lentivirus and culturing the transduced cell for 7 days to generate anti-BCMA CAR T cells.

### In Vivo Daudi Model Study

NSG mice were intravenously injected with 2×10⁶ Daudi cells labeled with firefly luciferase. After four days, four out of five groups of mice were intravenously administered 1×10⁶ human PBMCs. The next day, mice that did not receive PBMCs were administered vehicle control (DMEM) and mice that received the PBMCs were administered 5.0×10⁷ IU of LV 6.8, LV A, LV B, or LV 19 (GFP control). Tumor volume was measured by using a bioluminescence imaging system.

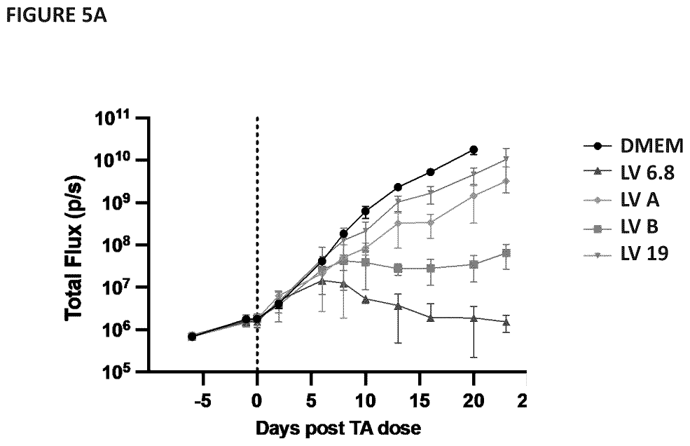

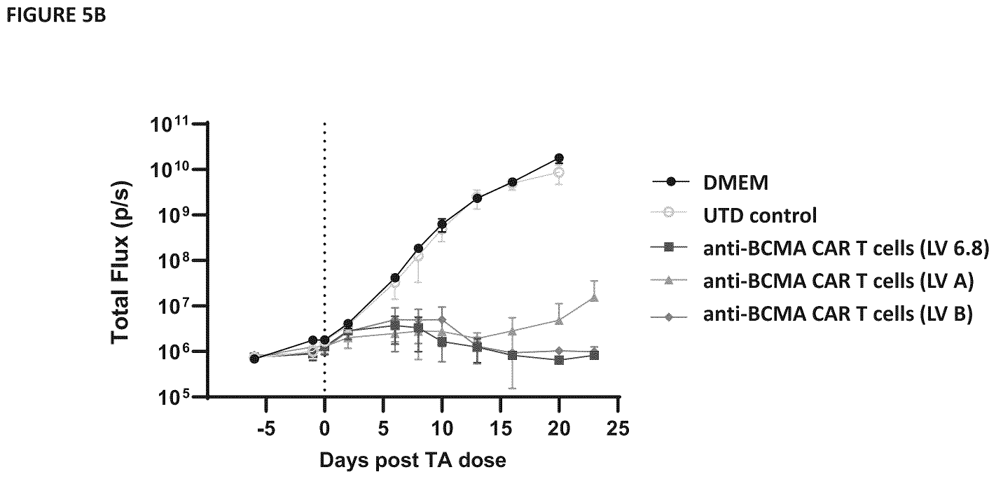

Tumor size increased in mice treated with vehicle, mice treated with the GFP control, and mice treated with a lentivirus expressing an anti-BCMA CAR comprising the binding domain used in idecabtagene vicleucel. Mice treated with a lentivirus expressing an anti-BCMA CAR comprising the binding domains like those used in ciltacabtagene autoleucel experienced suppression of tumor growth. Only mice treated with an anti-BCMA CAR comprising SEQ ID NO: 266 experienced tumor regression. FIG. 5A.

### Ex Vivo Daudi Model Study

NSG mice were intravenously injected with 2×10⁶ Daudi cells labeled with firefly luciferase. After five days, three out of five groups of mice were intravenously administered 2×10⁶ human anti-BCMA CAR T cells. Mice that did not receive anti-BCMA CAR T cells were administered vehicle control (DMEM) or 2×10⁶ untransduced control human T cells (UTD) and mice that received the anti-BCMA CAR T cells were administered 2×10⁶ anti-BCMA CAR T cells expressing the CAR encoded by SEQ ID NO: 266, SEQ ID NO: 340 or SEQ ID NO: 341. Tumor volume was measured by using a bioluminescence imaging system.

Tumor size increased in mice treated with vehicle and with untransduced control T cells. Mice treated with CAR T cells expressing an anti-BCMA CAR comprising the binding domain used in idecabtagene vicleucel showed a transient decrease in tumor burden whereas mice treated with CAR T cells expressing an anti-BCMA CAR comprising SEQ ID NO: 266 or an anti-BCMA CAR comprising the binding domains like those used in ciltacabtagene autoleucel experienced comparable and complete tumor regression. FIG. 5B.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A recombinant lentiviral particle comprising:
(a) a viral envelope comprising (i) a mutated cocal virus envelope glycoprotein (COCV-G) or a mutated vesicular stomatitis Indiana virus envelope glycoprotein (VSIV-G), wherein the mutated COCV-G or VSIV-G comprises amino acid substitutions selected from the group consisting of: K47A and R354A; K47A and R354Q; K47Q and R354A; and K47Q and R354Q; and (ii) a non-viral membrane-bound tropism polypeptide comprising an anti-CD3s scFv and a human CD8α hinge and transmembrane domain; and
(b) a recombinant lentiviral vector comprising a polynucleotide encoding a myeloproliferative sarcoma virus enhancer, negative control region deleted, dl587rev primer-binding site substituted (MND) U3 promoter or an EF1α promoter operably linked to a polynucleotide encoding a signal peptide and an anti-BCMA chimeric antigen receptor comprising an anti-BCMA scFv comprising a heavy chain variable region (VH) comprising a CDRH1, a CDRH2, and a CDRH3 comprising the amino acid sequences set forth in SEQ ID NOs: 82, 83, and 84, and a light chain variable region (VL) comprising a CDRL1, a CDRL2, and a CDRL3 comprising the amino acid sequences set forth in SEQ ID NOs: 86, 87, and 88; a CD8α hinge and transmembrane domain; a CD137 costimulatory domain; and a CD3ζ primary signaling domain.

2. The particle of claim 1, wherein the viral envelope comprises the mutated VSIV-G.

3. The particle of claim 1, wherein the anti-CD3s scFv is isolated from TR66 or variants thereof.

4. The particle of claim 1, wherein the anti-CD3s scFv comprises an amino acid sequence set forth in any one of SEQ ID NOs: 153, 154, 163, 164, 173, 174, 183, 184, 193, 194, 203, 204, 213, 214, 223, and 224.

5. An immune effector cell transduced with the particle of claim 1.

6. A composition comprising the particle of claim 1.

7. A composition comprising the immune effector cell of claim 5.

8. A pharmaceutical composition comprising a pharmaceutically acceptable carrier and the particle of claim 1.

9. A method of transducing an immune effector cell in vivo, comprising administering to a subject a pharmaceutical composition comprising a pharmaceutically acceptable carrier and an effective amount of the particle of claim 1.

10. A kit comprising the particle of claim 1, a pharmaceutically acceptable carrier, and instructions for use.

11. A recombinant lentiviral particle comprising:
(a) a viral envelope comprising (i) a mutated viral envelope glycoprotein comprising an amino acid sequence set forth in any one of SEQ ID NOs: 332, 333, 334, and 335 and (ii) a non-viral membrane-bound tropism polypeptide comprising an amino acid sequence set forth in any one of SEQ ID NOs: 324, 325, 326, 327, 328, 329, 330, and 331; and
(b) a recombinant lentiviral vector comprising a 5′ long terminal repeat (LTR) comprising R and U5 regions; a Psi (Ψ) packaging signal, a cPPT/FLAP, a rev response element (RRE); a polynucleotide encoding an MND promoter or an EF1α promoter operably linked to a polynucleotide encoding a signal peptide and an anti-BCMA chimeric antigen receptor (CAR) comprising an amino acid sequence set forth in SEQ ID NO: 270; optionally a WPRE; a 3′ LTR comprising U3 and R regions; a polyadenylation signal and a poly(A) tail.

12. The particle of claim 11, wherein the mutated viral envelope glycoprotein comprises an amino acid sequence set forth in SEQ ID NO: 335.

13. The particle of claim 11, wherein the non-viral membrane-bound tropism polypeptide comprises an amino acid sequence set forth in SEQ ID NO: 324.

14. An immune effector cell transduced with the particle of claim 11.

15. A composition comprising the particle of claim 11.

16. A pharmaceutical composition comprising a pharmaceutically acceptable carrier and the particle of claim 11.

17. A method of transducing an immune effector cell in vivo, comprising administering to a subject a pharmaceutical composition comprising a pharmaceutically acceptable carrier and an effective amount of the particle of claim 11.

18. A kit comprising the particle of claim 11, a pharmaceutically acceptable carrier, and instructions for use.

19. A recombinant lentiviral particle comprising:
(a) a viral envelope comprising (i) a mutated viral envelope glycoprotein comprising an amino acid sequence set forth in SEQ ID NO: 335 and (ii) a non-viral membrane-bound tropism polypeptide comprising an amino acid sequence set forth in SEQ ID NO: 324; and
(b) a recombinant lentiviral vector comprising a 5′ long terminal repeat (LTR) comprising R and U5 regions; a Psi (Ψ) packaging signal, a cPPT/FLAP, a rev response element (RRE); a polynucleotide encoding an EF1α promoter operably linked to a polynucleotide encoding an anti-BCMA chimeric antigen receptor (CAR) comprising an amino acid sequence set forth in SEQ ID NO: 270; optionally a WPRE; a 3′ LTR comprising U3 and R regions; a polyadenylation signal and a poly(A) tail.

20. (canceled)

21. The particle of claim 19, wherein the EF1α promoter comprises the polynucleotide sequence set forth in SEQ ID NO: 319.

22. The particle of claim 19, wherein the recombinant lentiviral vector is derived from HIV-1 or HIV-2.

23. The particle of claim 19, wherein the polynucleotide encoding the anti-BCMA CAR comprises the polynucleotide sequence set forth in SEQ ID NO: 302.

24. The particle of claim 19, wherein the signal peptide is isolated from a polypeptide selected from the group consisting of: CD8α, mIgGK, hIgGk, CD33, tPA, SEAP, hGM-CSF, CSF2R, and B2M.

25. The particle of claim 19, wherein the lentiviral vector further comprises a WPRE operably linked to the 3′ end of the polynucleotide encoding the anti-BCMA CAR.

26. An immune effector cell transduced with the particle of claim 19.

27. The immune effector cell of claim 26, wherein the immune effector cell is a T cell or natural killer (NK) cell.

28. A composition comprising the particle of claim 19.

29. A pharmaceutical composition comprising a pharmaceutically acceptable carrier and the particle of claim 19.

30. A method of transducing an immune effector cell in vivo, comprising administering to a subject a pharmaceutical composition comprising a pharmaceutically acceptable carrier and an effective amount of the particle of claim 19.
